SEQUENCE LISTING (164 sequences)

SEQ ID NO: 1 - protein, 262 aa, synthetic construct
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKAL

SEQ ID NO: 2 - DNA, 786 bp, synthetic construct
  source 1..786: mol_type = other DNA
atggactggatctggcgcatcctgtttctcgtgggagccgccacaggcgcccattctgaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcaaagctcta

SEQ ID NO: 3 - protein, 415 aa, synthetic construct
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - DNA, 1245 bp, synthetic construct
  source 1..1245: mol_type = other DNA
atggactggatctggcgcatcctgtttctcgtgggagccgccacaggcgcccattctgaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcaaagctctagatctcgagcccaaatcttgtgacaaaactcacacatgcccaccgtgcccggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 5 - protein, 462 aa, synthetic construct
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - DNA, 1386 bp, synthetic construct
  source 1..1386: mol_type = other DNA
atggactggatctggcgcatcctgtttctcgtgggagccgccacaggcgcccattctgaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcaaagctctagatctcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 7 - protein, 267 aa, synthetic construct
MDWIWRILFLVGAATGAHSQVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIK

SEQ ID NO: 8 - DNA, 801 bp, synthetic construct
  source 1..801: mol_type = other DNA
atggactggatctggcgcatcctgtttctcgtgggagccgccacaggcgcccattctcaggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaag

SEQ ID NO: 9 - protein, 467 aa, synthetic construct
MDWIWRILFLVGAATGAHSQVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIKDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 1401 bp, synthetic construct
  source 1..1401: mol_type = other DNA
atggactggatctggcgcatcctgtttctcgtgggagccgccacaggcgcccattctcaggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaaggatctcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 11 - protein, 323 aa, synthetic construct
MDWIWRILFLVGAATGAHSQVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIKDLEPKSCDKTHTCPPCPDPKFWVLVVVGGVLACYSLLVTVAFIIFWVRSKRSRLLH

SEQ ID NO: 12 - DNA, 969 bp, synthetic construct
  source 1..969: mol_type = other DNA
atggactggatctggcgcatcctgtttctcgtgggagccgccacaggcgcccattctcaggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaaggatctcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcac

SEQ ID NO: 13 - protein, 810 aa, synthetic construct
MDWIWRILFLVGAATGAHSQVQLLESGGGLVQPGGSLRLSCAASGFTFSSYTMSWVRQAPGQALEWMGTISSGGTYTYYPDSVKGRFTISRDNAKNSLYLQMNSLRAEDTAVYYCAREAIFTYWGRGTLVTSSGGGGSGGGGSGGGGSDIQLTQSPSSLSASVGDRVTITCKASQDINNYLSWYQQKPGQAPRLLIYRANRLVDGVPDRFSGSGYGTDFTLTINNIESEDAAYYFCLKYDVFPYTFGQGTKVEIKDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRAEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 14 - DNA, 2433 bp, synthetic construct
  source 1..2433: mol_type = other DNA
atggactggatctggcggattctgttcctcgtgggagccgccacaggcgctcactcacaggtgcagctgctggaatctggcggcggactggtgcagcctggcggcagcctgagactgagctgcgccgccagcggcttcaccttcagcagctacaccatgagctgggtccggcaggctcctggacaggccctggaatggatgggcaccatcagcagcggcggcacctacacctactaccccgacagcgtgaagggccggttcaccatcagccgggacaacgccaagaacagcctgtacctgcagatgaacagcctgagagccgaggacacagccgtgtactactgcgccagagaggccatcttcacctactggggcagaggcaccctggtcacaagcagcggaggcggaggaagtggagggggaggatcaggcggcggaggcagcgatatccagctgacccagagccctagcagcctgagcgccagcgtgggcgacagagtgaccatcacatgcaaggccagccaggacatcaacaactacctgagctggtatcagcagaagcccggccaggcccccagactgctgatctaccgggccaacagactggtggacggcgtgcccgatagattcagcggcagcggctacggcaccgacttcaccctgaccatcaacaacatcgagtccgaggacgccgcctactacttctgcctgaagtacgacgtgttcccctacaccttcggccagggcaccaaggtggagatcaaggatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagccgagggcagaggcagcctgctgacatgtggcgacgtggaagagaacccaggccccatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 15 - protein, 19 aa, synthetic construct
MDWIWRILFLVGAATGAHS

SEQ ID NO: 16 - DNA, 57 bp, synthetic construct
  source 1..57: mol_type = other DNA
atggactggatctggcgcatcctgtttctcgtgggagccgccacaggcgcccattct

SEQ ID NO: 17 - protein, 243 aa, synthetic construct
EVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKAL

SEQ ID NO: 18 - DNA, 729 bp, synthetic construct
  source 1..729: mol_type = other DNA
gaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcaaagctcta

SEQ ID NO: 19 - protein, 17 aa, synthetic construct
DLEPKSCDKTHTCPPCP

SEQ ID NO: 20 - DNA, 51 bp, synthetic construct
  source 1..51: mol_type = other DNA
gatctcgagcccaaatcttgtgacaaaactcacacatgcccaccgtgcccg

SEQ ID NO: 21 - protein, 20 aa, synthetic construct
DLEPKSCDKTHTCPPCPDPK

SEQ ID NO: 22 - DNA, 60 bp, synthetic construct
  source 1..60: mol_type = other DNA
gatctcgagcccaaatcttgtgacaaaactcacacatgcccaccgtgcccggatcccaaa

SEQ ID NO: 23 - protein, 21 aa, synthetic construct
LCYLLDGILFIYGVILTALFL

SEQ ID NO: 24 - DNA, 63 bp, synthetic construct
  source 1..63: mol_type = other DNA
ctctgctacctgctggatggaatcctcttcatctatggtgtcattctcactgccttgttcctg

SEQ ID NO: 25 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 27 - protein, 133 aa, synthetic construct
LCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - DNA, 399 bp, synthetic construct
  source 1..399: mol_type = other DNA
ctctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 29 - protein, 248 aa, synthetic construct
QVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIK

SEQ ID NO: 30 - DNA, 744 bp, synthetic construct
  source 1..744: mol_type = other DNA
caggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaag

SEQ ID NO: 31 - protein, 27 aa, synthetic construct
FWVLVVVGGVLACYSLLVTVAFIIFWV

SEQ ID NO: 32 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 33 - protein, 41 aa, synthetic construct
RSKRSRLLHSDYMNMTPRRPGPTRKHYQPYAPPRDFAAYRS

SEQ ID NO: 34 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 35 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 36 - DNA, 204 bp, synthetic construct
  source 1..204: mol_type = other DNA
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 37 - DNA, 57 bp, synthetic construct
  source 1..57: mol_type = other DNA
atggactggatctggcggattctgttcctcgtgggagccgccacaggcgctcactca

SEQ ID NO: 38 - protein, 236 aa, synthetic construct
QVQLLESGGGLVQPGGSLRLSCAASGFTFSSYTMSWVRQAPGQALEWMGTISSGGTYTYYPDSVKGRFTISRDNAKNSLYLQMNSLRAEDTAVYYCAREAIFTYWGRGTLVTSSGGGGSGGGGSGGGGSDIQLTQSPSSLSASVGDRVTITCKASQDINNYLSWYQQKPGQAPRLLIYRANRLVDGVPDRFSGSGYGTDFTLTINNIESEDAAYYFCLKYDVFPYTFGQGTKVEIK

SEQ ID NO: 39 - DNA, 708 bp, synthetic construct
  source 1..708: mol_type = other DNA
caggtgcagctgctggaatctggcggcggactggtgcagcctggcggcagcctgagactgagctgcgccgccagcggcttcaccttcagcagctacaccatgagctgggtccggcaggctcctggacaggccctggaatggatgggcaccatcagcagcggcggcacctacacctactaccccgacagcgtgaagggccggttcaccatcagccgggacaacgccaagaacagcctgtacctgcagatgaacagcctgagagccgaggacacagccgtgtactactgcgccagagaggccatcttcacctactggggcagaggcaccctggtcacaagcagcggaggcggaggaagtggagggggaggatcaggcggcggaggcagcgatatccagctgacccagagccctagcagcctgagcgccagcgtgggcgacagagtgaccatcacatgcaaggccagccaggacatcaacaactacctgagctggtatcagcagaagcccggccaggcccccagactgctgatctaccgggccaacagactggtggacggcgtgcccgatagattcagcggcagcggctacggcaccgacttcaccctgaccatcaacaacatcgagtccgaggacgccgcctactacttctgcctgaagtacgacgtgttcccctacaccttcggccagggcaccaaggtggagatcaag

SEQ ID NO: 40 - protein, 15 aa, synthetic construct
EPKSCDKTHTCPPCP

SEQ ID NO: 41 - DNA, 45 bp, synthetic construct
  source 1..45: mol_type = other DNA
gagcccaaatcttgtgacaaaactcacacatgcccaccgtgcccg

SEQ ID NO: 42 - DNA, 60 bp, synthetic construct
  source 1..60: mol_type = other DNA
gatctcgagcccaaatcttgtgacaaaactcacacatgcccaccgtgcccggatcccaag

SEQ ID NO: 43 - DNA, 204 bp, synthetic construct
  source 1..204: mol_type = other DNA
ttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtcc

SEQ ID NO: 44 - DNA, 336 bp, synthetic construct
  source 1..336: mol_type = other DNA
agagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 45 - protein, 18 aa, Thosea asigna virus
EGRGSLLTCGDVEENPGP

SEQ ID NO: 46 - DNA, 54 bp, Thosea asigna virus
  source 1..54: mol_type = genomic DNA
gagggcagaggcagcctgctgacatgtggcgacgtggaagagaacccaggcccc

SEQ ID NO: 47 - protein, 333 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 48 - DNA, 999 bp, synthetic construct
  source 1..999: mol_type = other DNA
atgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttc

SEQ ID NO: 49 - protein, 30 aa, synthetic construct
CDIYIWAPLAGTCGVLLLSLVITLYCNHRN

SEQ ID NO: 50 - DNA, 90 bp, synthetic construct
  source 1..90: mol_type = other DNA
tgcgacatctacatctgggcccctctggccggcacatgtggcgtgctgctgctgagcctcgtgatcaccctgtactgcaaccaccggaac

SEQ ID NO: 51 - protein, 31 aa, synthetic construct
QPMALIVLGGVAGLLLFIGLGIFFCVRCRHR

SEQ ID NO: 52 - DNA, 93 bp, synthetic construct
  source 1..93: mol_type = other DNA
cagccaatggccctgattgtgctggggggcgtcgccggcctcctgcttttcattgggctaggcatcttcttctgtgtcaggtgccggcaccga

SEQ ID NO: 53 - protein, 21 aa, Thosea asigna virus
GSGEGRGSLLTCGDVEENPGP

SEQ ID NO: 54 - protein, 63 aa, Foot-and-mouth disease virus
GSGSRVTELLYRMKRAETYCPRPLLAIHPTEARHKQKIVAPVKQLLNFDLLKLAGDVESNPGP

SEQ ID NO: 55 - protein, 19 aa, Amphimedon sp.
LLCFLLLLLSGDVELNPGP

SEQ ID NO: 56 - protein, 20 aa, Amphimedon sp.
HHFMFLLLLLAGDIELNPGP

SEQ ID NO: 57 - protein, 20 aa, Saccoglossus kowalevskii
WFLVLLSFILSGDIEVNPGP

SEQ ID NO: 58 - protein, 20 aa, Branchiostoma floridae
KNCAMYMLLLSGDVETNPGP

SEQ ID NO: 59 - protein, 20 aa, Branchiostoma floridae
MVISQLMLKLAGDVEENPGP

SEQ ID NO: 60 - protein, 22 aa, Teschovirus A
GSGATNFSLLKQAGDVEENPGP

SEQ ID NO: 61 - protein, 23 aa, Equine rhinitis A virus
GSGQCTNYALLKLAGDVESNPGP

SEQ ID NO: 62 - protein, 8 aa, synthetic construct
  VARIANT 2: note = Any amino acid
  VARIANT 4: note = Any amino acid
DXEXNPGP

SEQ ID NO: 63 - DNA, 23 bp, synthetic construct
  source 1..23: mol_type = other DNA
cctgcatgatgcgcggcccangg

SEQ ID NO: 64 - DNA, 23 bp, synthetic construct
  source 1..23: mol_type = other DNA
caagtagtcggggatgtcggngg

SEQ ID NO: 65 - DNA, 23 bp, synthetic construct
  source 1..23: mol_type = other DNA
agtagtcggggatgtcggcgngg

SEQ ID NO: 66 - protein, 9 aa, synthetic construct
RSKRSRLLH

SEQ ID NO: 67 - DNA, 27 bp, synthetic construct
  source 1..27: mol_type = other DNA
aggagtaagaggagcaggctcctgcac

SEQ ID NO: 68 - DNA, 23 bp, synthetic construct
  source 1..23: mol_type = other DNA
gcatgatgcgcggcccaaggngg

SEQ ID NO: 69 - DNA, 57 bp, synthetic construct
  source 1..57: mol_type = other DNA
cactctttccctacacgacgctcttccgatcttcccgatgaccctgtcttcccgtgc

SEQ ID NO: 70 - DNA, 59 bp, synthetic construct
  source 1..59: mol_type = other DNA
gtgactggagttcagacgtgtgctcttccgatctaggtagaagccattagtgagctggc

SEQ ID NO: 71 - DNA, 57 bp, synthetic construct
  source 1..57: mol_type = other DNA
atggactggatctggcggatcctgtttcttgtgggagccgccacaggcgcccattct

SEQ ID NO: 72 - protein, 121 aa, synthetic construct
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVS

SEQ ID NO: 73 - DNA, 363 bp, synthetic construct
  source 1..363: mol_type = other DNA
gaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctct

SEQ ID NO: 74 - protein, 16 aa, synthetic construct
SGGGGSGGGGSGGGGS

SEQ ID NO: 75 - DNA, 48 bp, synthetic construct
  source 1..48: mol_type = other DNA
tctggtggcggaggaagcggaggcggaggttcaggcggcggaggatct

SEQ ID NO: 76 - protein, 107 aa, synthetic construct
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 77 - DNA, 321 bp, synthetic construct
  source 1..321: mol_type = other DNA
gatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaag

SEQ ID NO: 78 - protein, 45 aa, synthetic construct
TTTPAPRPPTPAPTIASQPLSLRPEACRPAAGGAVHTRGLDFACD

SEQ ID NO: 79 - DNA, 135 bp, synthetic construct
  source 1..135: mol_type = other DNA
accaccacaccagctcctcggcctccaactcctgctcctacaattgccagccagcctctgtctctgaggcccgaagcttgtagacctgctgctggcggagccgtgcatacaagaggactggatttcgcctgcgac

SEQ ID NO: 80 - protein, 39 aa, synthetic construct
IEVMYPPPYLDNEKSNGTIIHVKGKHLCPSPLFPGPSKP

SEQ ID NO: 81 - DNA, 117 bp, synthetic construct
  source 1..117: mol_type = other DNA
atcgaagtgatgtacccgcctccttacctggacaacgagaagtccaacggcaccatcatccacgtgaagggaaagcacctgtgtccttctccactgttccccggacctagcaagcct

SEQ ID NO: 82 - protein, 24 aa, synthetic construct
IYIWAPLAGTCGVLLLSLVITLYC

SEQ ID NO: 83 - DNA, 72 bp, synthetic construct
  source 1..72: mol_type = other DNA
atctacatctgggcccctctggctggaacatgtggcgttctgctgctgagcctggtcatcaccctgtactgc

SEQ ID NO: 84 - DNA, 81 bp, synthetic construct
  source 1..81: mol_type = other DNA
ttctgggtgctcgttgttgttggcggcgtgctggcctgttacagcctgctggttaccgtggccttcatcatcttttgggtc

SEQ ID NO: 85 - DNA, 123 bp, synthetic construct
  source 1..123: mol_type = other DNA
cggtccaagagaagcagactgctgcacagcgactacatgaacatgacccctagacggcccggacctaccagaaagcactaccagccttacgctcctcctagagatttcgccgcctaccggtcc

SEQ ID NO: 86 - protein, 42 aa, synthetic construct
KRGRKKLLYIFKQPFMRPVQTTQEEDGCSCRFPEEEEGGCEL

SEQ ID NO: 87 - DNA, 126 bp, synthetic construct
  source 1..126: mol_type = other DNA
aagcggggcagaaagaagctgctgtacatcttcaagcagcccttcatgcggcccgtgcagaccacacaagaggaagatggctgctcctgcagattccccgaggaagaagaaggcggctgcgagctg

SEQ ID NO: 88 - DNA, 336 bp, synthetic construct
  source 1..336: mol_type = other DNA
agagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 89 - protein, 4 aa, synthetic construct
KRGR

SEQ ID NO: 90 - DNA, 12 bp, synthetic construct
  source 1..12: mol_type = other DNA
aagcggggcaga

SEQ ID NO: 91 - protein, 244 aa, synthetic construct
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 92 - DNA, 732 bp, synthetic construct
  source 1..732: mol_type = other DNA
gaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaag

SEQ ID NO: 93 - protein, 263 aa, synthetic construct
MDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 94 - DNA, 789 bp, synthetic construct
  source 1..789: mol_type = other DNA
atggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaag

SEQ ID NO: 95 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 96 - DNA, 207 bp, synthetic construct
  source 1..207: mol_type = other DNA
accaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggcccctctggctggaacatgtggcgttctgctgctgagcctggtcatcaccctgtactgc

SEQ ID NO: 97 - protein, 66 aa, synthetic construct
IEVMYPPPYLDNEKSNGTIIHVKGKHLCPSPLFPGPSKPFWVLVVVGGVLACYSLLVTVAFIIFWV

SEQ ID NO: 98 - DNA, 198 bp, synthetic construct
  source 1..198: mol_type = other DNA
atcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtc

SEQ ID NO: 99 - protein, 153 aa, synthetic construct
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - DNA, 459 bp, synthetic construct
  source 1..459: mol_type = other DNA
cggtccaagagaagcagactgctgcacagcgactacatgaacatgacccctagacggcccggacctaccagaaagcactaccagccttacgctcctcctagagatttcgccgcctaccggtccagagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 101 - protein, 154 aa, synthetic construct
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - DNA, 462 bp, synthetic construct
  source 1..462: mol_type = other DNA
aagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctccagatccgccgacgctcctgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 103 - protein, 195 aa, synthetic construct
RSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - DNA, 585 bp, synthetic construct
  source 1..585: mol_type = other DNA
cggtccaagagaagcagactgctgcacagcgactacatgaacatgacccctagacggcccggacctaccagaaagcactaccagccttacgctcctcctagagatttcgccgcctaccggtccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 105 - protein, 485 aa, synthetic construct
MDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - DNA, 1455 bp, synthetic construct
  source 1..1455: mol_type = other DNA
atggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagaccaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggcccctctggctggaacatgtggcgttctgctgctgagcctggtcatcaccctgtactgccggtccaagagaagcagactgctgcacagcgactacatgaacatgacccctagacggcccggacctaccagaaagcactaccagccttacgctcctcctagagatttcgccgcctaccggtccagagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 107 - protein, 486 aa, synthetic construct
MDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - DNA, 1458 bp, synthetic construct
  source 1..1458: mol_type = other DNA
atggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagaccaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggcccctctggctggaacatgtggcgttctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctccagatccgccgacgctcctgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 109 - protein, 482 aa, synthetic construct
MDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - DNA, 1446 bp, synthetic construct
  source 1..1446: mol_type = other DNA
atggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagatttcgccgcctaccggtccagagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaaga

SEQ ID NO: 111 - protein, 483 aa, synthetic construct
MDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - DNA, 1449 bp, synthetic construct
  source 1..1449: mol_type = other DNA
atggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctccagatccgccgacgctcctgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaaga

SEQ ID NO: 113 - protein, 336 aa, synthetic construct
MDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKTTTPAPRPPTPAPTIASQPLSLRPEACRPAAGGAVHTRGLDFACDIYIWAPLAGTCGVLLLSLVITLYCKRGR

SEQ ID NO: 114 - DNA, 1008 bp, synthetic construct
  source 1..1008: mol_type = other DNA
atggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagaccaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggcccctctggctggaacatgtggcgttctgctgctgagcctggtcatcaccctgtactgcaagcggggcaga

SEQ ID NO: 115 - protein, 527 aa, synthetic construct
MDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - DNA, 1581 bp, synthetic construct
  source 1..1581: mol_type = other DNA
atggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagaccaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggcccctctggctggaacatgtggcgttctgctgctgagcctggtcatcaccctgtactgccggtccaagagaagcagactgctgcacagcgactacatgaacatgacccctagacggcccggacctaccagaaagcactaccagccttacgctcctcctagagatttcgccgcctaccggtccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 117 - protein, 19 aa, synthetic construct
ATNFSLLKQAGDVEENPGP

SEQ ID NO: 118 - DNA, 57 bp, synthetic construct
  source 1..57: mol_type = other DNA
gccaccaatttcagcctgctgaaacaggccggcgacgtggaagagaatcctggacct

SEQ ID NO: 119-120 - skipped (2 entries)

SEQ ID NO: 121 - protein, 8 aa, synthetic construct
GGGSGGGS

SEQ ID NO: 122 - protein, 12 aa, synthetic construct
GGGSGGGSGGGS

SEQ ID NO: 123 - protein, 16 aa, synthetic construct
GGGSGGGSGGGSGGGS

SEQ ID NO: 124 - protein, 20 aa, synthetic construct
GGGSGGGSGGGSGGGSGGGS

SEQ ID NO: 125 - protein, 10 aa, synthetic construct
GGGGSGGGGS

SEQ ID NO: 126 - protein, 20 aa, synthetic construct
GGGGSGGGGSGGGGSGGGGS

SEQ ID NO: 127 - protein, 25 aa, synthetic construct
GGGGSGGGGSGGGGSGGGGSGGGGS

SEQ ID NO: 128 - protein, 119 aa, synthetic construct
QVKLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSSD

SEQ ID NO: 129 - DNA, 357 bp, synthetic construct
  source 1..357: mol_type = other DNA
caagtgaaactgcagcagagcggagccgaactcgtgaagccaggcgccagcgtgaagctgtcttgcaaggcctccggctatacctttaccaactacgacatcaactgggtgcgccagaggcccgagcagggactggaatggattggatggatcttccccggcgacggcagcacccagtacaatgagaagtttaaggggaaggctacactgacaaccgataccagcagctccacagcttatatgcagctgtcccggctgacctccgaggactccgctgtgtacttctgtgccagacagaccaccgccacttggtttgcctattggggacagggaaccactgtgaccgtgtcctctgat

SEQ ID NO: 130 - protein, 15 aa, synthetic construct
GGGGSGGGGSGGGGS

SEQ ID NO: 131 - DNA, 42 bp, synthetic construct
  source 1..42: mol_type = other DNA
ggcggaggcagcgggggagggggctcagggggcggaggctct

SEQ ID NO: 132 - protein, 110 aa, synthetic construct
DIELTQSPTTLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKQAA

SEQ ID NO: 133 - DNA, 330 bp, synthetic construct
  source 1..330: mol_type = other DNA
gatattgagctgacacagtcccccaccaccctgagcgtgacccctggcgatagagtgtccctgagctgtagggccagccagagcatcagcgactacctgcattggtatcagcagaaaagccacgagagccctcggctgctgatcaaatacgccagccagtccatctccggcatccccagcagattcagcggctctggaagcggcagcgacttcaccctgtccatcaacagcgtggaacctgaggatgtgggcgtgtactattgccagaacggccacagcttcccactgaccttcggcgctggaacaaaactggaactgaaacaggccgcc

SEQ ID NO: 134 - protein, 244 aa, synthetic construct
QVKLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSSDGGGGSGGGGSGGGGSDIELTQSPTTLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKQAA

SEQ ID NO: 135 - DNA, 729 bp, synthetic construct
  source 1..729: mol_type = other DNA
caagtgaaactgcagcagagcggagccgaactcgtgaagccaggcgccagcgtgaagctgtcttgcaaggcctccggctatacctttaccaactacgacatcaactgggtgcgccagaggcccgagcagggactggaatggattggatggatcttccccggcgacggcagcacccagtacaatgagaagtttaaggggaaggctacactgacaaccgataccagcagctccacagcttatatgcagctgtcccggctgacctccgaggactccgctgtgtacttctgtgccagacagaccaccgccacttggtttgcctattggggacagggaaccactgtgaccgtgtcctctgatggcggaggcagcgggggagggggctcagggggcggaggctctgatattgagctgacacagtcccccaccaccctgagcgtgacccctggcgatagagtgtccctgagctgtagggccagccagagcatcagcgactacctgcattggtatcagcagaaaagccacgagagccctcggctgctgatcaaatacgccagccagtccatctccggcatccccagcagattcagcggctctggaagcggcagcgacttcaccctgtccatcaacagcgtggaacctgaggatgtgggcgtgtactattgccagaacggccacagcttcccactgaccttcggcgctggaacaaaactggaactgaaacaggccgcc

SEQ ID NO: 136 - protein, 117 aa, synthetic construct
EVQLVESGGGLVKPGGSLKLSCEASRFTFSSYAMSWVRQTPEKRLEWVAAISGGGRYTYYPDSMKGRFTISRDNAKNFLYLQMSSLRSEDTAMYYCARHYDGYLDYWGQGTTLTVSS

SEQ ID NO: 137 - DNA, 351 bp, synthetic construct
  source 1..351: mol_type = other DNA
gaagtgcagctggtggagtctgggggaggcttagtgaagcctggagggtccctgaaactctcctgtgaagcctctagattcactttcagtagctatgccatgtcttgggttcgccagactccggagaagaggctggagtgggtcgcagccattagtggaggtggtaggtacacctactatccagacagtatgaagggtcgattcaccatctccagagacaatgccaagaatttcctgtacctgcaaatgagcagtctgaggtctgaggacacggccatgtattactgtgcaagacactatgatggttatcttgactactggggccaaggcaccactctcacagtctcctca

SEQ ID NO: 138 - protein, 108 aa, synthetic construct
DIVMTQSHKFMSTSIGARVSITCKASQDVRTAVAWYQQKPGQSPKLLIYSASYRYTGVPDRFTGSGSGTDFTFTISSVQAEDLAVYYCQQHYGTPPWTFGGGTKLEIK

SEQ ID NO: 139 - DNA, 324 bp, synthetic construct
  source 1..324: mol_type = other DNA
gacattgtgatgacccagtctcacaaattcatgtccacatcaattggagccagggtcagcatcacctgcaaggccagtcaggatgtgagaactgctgtagcctggtatcaacagaaaccaggccagtctcctaaactactaatttactcggcatcctaccggtacactggagtccctgatcgcttcactggcagtggatctgggacggatttcactttcaccatcagcagtgtgcaggctgaagacctggcagtttattactgtcagcaacattatggtactcctccgtggacgttcggtggaggcaccaagctggaaatcaaa

SEQ ID NO: 140 - protein, 240 aa, synthetic construct
DIVMTQSHKFMSTSIGARVSITCKASQDVRTAVAWYQQKPGQSPKLLIYSASYRYTGVPDRFTGSGSGTDFTFTISSVQAEDLAVYYCQQHYGTPPWTFGGGTKLEIKGGGGSGGGGSGGGGSEVQLVESGGGLVKPGGSLKLSCEASRFTFSSYAMSWVRQTPEKRLEWVAAISGGGRYTYYPDSMKGRFTISRDNAKNFLYLQMSSLRSEDTAMYYCARHYDGYLDYWGQGTTLTVSS

SEQ ID NO: 141 - DNA, 720 bp, synthetic construct
  source 1..720: mol_type = other DNA
gacattgtgatgacccagtctcacaaattcatgtccacatcaattggagccagggtcagcatcacctgcaaggccagtcaggatgtgagaactgctgtagcctggtatcaacagaaaccaggccagtctcctaaactactaatttactcggcatcctaccggtacactggagtccctgatcgcttcactggcagtggatctgggacggatttcactttcaccatcagcagtgtgcaggctgaagacctggcagtttattactgtcagcaacattatggtactcctccgtggacgttcggtggaggcaccaagctggaaatcaaaggcggcggaggatctggcggaggcggaagtggcggagggggctctgaagtgcagctggtggagtctgggggaggcttagtgaagcctggagggtccctgaaactctcctgtgaagcctctagattcactttcagtagctatgccatgtcttgggttcgccagactccggagaagaggctggagtgggtcgcagccattagtggaggtggtaggtacacctactatccagacagtatgaagggtcgattcaccatctccagagacaatgccaagaatttcctgtacctgcaaatgagcagtctgaggtctgaggacacggccatgtattactgtgcaagacactatgatggttatcttgactactggggccaaggcaccactctcacagtctcctca

SEQ ID NO: 142 - DNA, 20 bp, synthetic construct
  source 1..20: mol_type = other DNA
ccacttaccagatatgcccc

SEQ ID NO: 143 - RNA, 20 nt, synthetic construct
  source 1..20: mol_type = other RNA
ccacttaccagatatgcccc

SEQ ID NO: 144 - protein, 12 aa, synthetic construct
ERKCCVECPPCP

SEQ ID NO: 145 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 146 - protein, 12 aa, synthetic construct
ESKYGPPCPSCP

SEQ ID NO: 147 - protein, 238 aa, synthetic construct
QVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKS

SEQ ID NO: 148 - DNA, 714 bp, synthetic construct
  source 1..714: mol_type = other DNA
caggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagc

SEQ ID NO: 149 - protein, 244 aa, synthetic construct
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELK

SEQ ID NO: 150 - DNA, 732 bp, synthetic construct
  source 1..732: mol_type = other DNA
cagattcagctggtgcagtctggccccgagctgaagaaacccggcgagacagtgaagatcagctgcaaggccagcggctacatcttcaccaactacggcatgaactgggtcaagcaggcccctggcaagagcttcaagtggatgggctggatcaacacctacaccggcgagagcacctacagcgccgacttcaagggcagattcgccttcagcctggaaaccagcgccagcaccgcctacctgcacatcaacgacctgaagaacgaggacaccgccacctacttttgcgccagaagcggcggctacgaccctatggattattggggccagggcaccagcgtgaccgtgtctagcggaggcggaggaagtggcggcggaggatctgggggaggcggatctgatatcgtgctgacccagagccctgccagcctggctgtgtctctgggacagagagccaccatcagctgtcgggccagcgagagcgtggacaattacggcaacaccttcatgcactggtatcagcagaagcccggccagccccccaagctgctgatctacagagccagcaacctggaaagcggcatccccgccagattttccggcagcggcagcagaaccgacttcaccctgaccatcaaccccgtggaagccgacgacgtggccacctattactgccagcagagcaacgaggacccccctacctttggagccggcaccaagctggaactgaag

SEQ ID NO: 151 - protein, 482 aa, synthetic construct
MALPVTALLLPLALLLHAARPQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - DNA, 1449 bp, synthetic construct
  source 1..1449: mol_type = other DNA
atggccttaccagtgaccgccttgctcctgccgctggccttgctgctccacgccgccaggccgcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcaccacgacgccagcgccgcgaccaccaacgccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 153 - protein, 481 aa, synthetic construct
MALPVTALLLPLALLLHAARPQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - DNA, 1446 bp, synthetic construct
  source 1..1446: mol_type = other DNA
atggccttaccagtgaccgccttgctcctgccgctggccttgctgctccacgccgccaggccgcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcaccacgacgccagcgccgcgaccaccaacgccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccgaagcaagcggagccggctgctgcacagcgactacatgaacatgacccctagacggcccggaccaaccagaaagcactaccagccttacgctcctcctagagatttcgccgcctaccggtccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 155 - protein, 520 aa, synthetic construct
MALPVTALLLPLALLLHAARPQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 156 - DNA, 1563 bp, synthetic construct
  source 1..1563: mol_type = other DNA
atggccttaccagtgaccgccttgctcctgccgctggccttgctgctccacgccgccaggccgcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagatttcgccgcctaccggtccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 157 - protein, 478 aa, synthetic construct
MALPVTALLLPLALLLHAARPQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - DNA, 1437 bp, synthetic construct
  source 1..1437: mol_type = other DNA
atggccttaccagtgaccgccttgctcctgccgctggccttgctgctccacgccgccaggccgcaggtgcagctgcagcagcctggcgctgaactcgtgcggccaggcgcttctgtgaagctgagctgtaaagccagcggctacaccttcaccagctactggatgaactgggtcaagcagcggcccgaccagggcctggaatggatcggaagaatcgacccctacgacagcgagacacactacaaccagaagttcaaggacaaggccatcctgaccgtggacaagagcagcagcaccgcctacatgcagctgtccagcctgaccagcgaggacagcgccgtgtactactgcgccagaggcaactgggacgactactggggccagggcacaaccctgacagtgtctagcggaggcggaggatcaggcggcggaggaagtgggggaggcggatctgatgtgcagattacccagtcccccagctacctggccgcctctcctggcgagacaatcaccatcaactgccgggccagcaagagcatctccaaggacctggcctggtatcaggaaaagcccggcaagaccaacaagctgctgatctacagcggctccaccctgcagtccggcatccccagcagattttccggcagcggctctggcaccgacttcaccctgaccatcagctccctggaacccgaggactttgccatgtactattgccagcagcacaacaagtacccttacaccttcggcggaggcaccaagctggaaatcaagagcatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagatttcgccgcctaccggtccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 159 - protein, 487 aa, synthetic construct
MALPVTALLLPLALLLHAARPQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - DNA, 1464 bp, synthetic construct
  source 1..1464: mol_type = other DNA
atggccttaccagtgaccgccttgctcctgccgctggccttgctgctccacgccgccaggccgcagattcagctggtgcagtctggccccgagctgaagaaacccggcgagacagtgaagatcagctgcaaggccagcggctacatcttcaccaactacggcatgaactgggtcaagcaggcccctggcaagagcttcaagtggatgggctggatcaacacctacaccggcgagagcacctacagcgccgacttcaagggcagattcgccttcagcctggaaaccagcgccagcaccgcctacctgcacatcaacgacctgaagaacgaggacaccgccacctacttttgcgccagaagcggcggctacgaccctatggattattggggccagggcaccagcgtgaccgtgtctagcggaggcggaggaagtggcggcggaggatctgggggaggcggatctgatatcgtgctgacccagagccctgccagcctggctgtgtctctgggacagagagccaccatcagctgtcgggccagcgagagcgtggacaattacggcaacaccttcatgcactggtatcagcagaagcccggccagccccccaagctgctgatctacagagccagcaacctggaaagcggcatccccgccagattttccggcagcggcagcagaaccgacttcaccctgaccatcaaccccgtggaagccgacgacgtggccacctattactgccagcagagcaacgaggacccccctacctttggagccggcaccaagctggaactgaagaccacgacgccagcgccgcgaccaccaacgccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggtccaagagaagcagactgctgcacagcgactacatgaacatgacccctagacggcccggacctaccagaaagcactaccagccttacgctcctcctagagatttcgccgcctaccggtccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 161 - DNA, 20 bp, synthetic construct
  source 1..20: mol_type = other DNA
ccattgggagatctattagg

SEQ ID NO: 162 - RNA, 20 nt, synthetic construct
  source 1..20: mol_type = other RNA
ccattgggagatctattagg

SEQ ID NO: 163 - DNA, 20 bp, synthetic construct
  source 1..20: mol_type = other DNA
gatgcaagtcaggctaagac

SEQ ID NO: 164 - RNA, 20 nt, synthetic construct
  source 1..20: mol_type = other RNA
gatgcaagtcaggctaagac

(19) United States
(12) Patent Application Publication
(10) Pub. No.: US 2026/0091115 A1
(43) Pub. Date: Apr. 2, 2026

(54) DISRUPTION OF ASXL1 IN T CELLS TO ENHANCE IMMUNOTHERAPY

(71) Applicants: St. Jude Children's Research Hospital, Inc., Memphis, TN (US); Rigshospitalet, Copenhagen (DK)

(72) Inventors: Benjamin YOUNGBLOOD, Memphis, TN (US); Caitlin ZEBLEY, Memphis, TN (US); Tae Gun KANG, Memphis, TN (US); Kirsten Grønbæk, Frederiksberg (DK)

(73) Assignees: St. Jude Children's Research Hospital, Inc., Memphis, TN (US); Rigshospitalet, Copenhagen (DK)

(21) Appl. No.: 19/112,045

(22) PCT Filed: Sep. 15, 2023
(86) PCT No.: PCT/US2023/074291

§ 371 Date: Mar. 14, 2025

Related U.S. Application Data
(60) Provisional application No. 63/407,322, filed on Sep. 16, 2022.

(51) Int. Cl. A61K 40/42; A61K 40/11; A61K 40/31; C07K 14/725; C07K 16/28; C07K 16/32; C12N 9/22; C12N 15/11; C12N 15/90

(52) U.S. Cl. CPC A61K 40/4217; A61K 40/11; A61K 40/31; A61K 40/4205; C07K 14/7051; C07K 16/2866; C07K 16/32; C12N 9/226; C12N 15/11; C12N 15/907; C07K 2317/622; C12N 2310/20

(57) ABSTRACT

The application relates to modified immune effector cells with enhanced immune cell function, as well as related pharmaceutical compositions. The application further relates to methods for generating the modified immune effector cell and methods for using the modified immune effector cell for treatment of diseases (e.g., adoptive cell therapy).

### CROSS-REFERENCE TO RELATED APPLICATIONS

This patent application claims priority to U.S. Provisional Application No. 63/407,322, filed Sep. 16, 2022, the disclosure of which is herein incorporated by reference in its entirety.

### SEQUENCE LISTING

The instant application contains a Sequence Listing which has been submitted electronically in XML file format and is hereby incorporated by reference in its entirety. Said XML copy, created on Jul. 31, 2023, is named 243734_000190_SL.xml and is 194,765 bytes in size.

### FIELD

The application relates to modified immune effector cells with enhanced immune cell function, as well as related pharmaceutical compositions. The application further relates to methods for generating the modified immune effector cell and methods for using the modified immune effector cell for treatment of diseases (e.g., adoptive cell therapy).

### BACKGROUND

The success of T cell-based immunotherapies relies on the ability of T cells to undergo robust proliferation and sustain effector function in the presence of tumor antigen. Similarly with immune checkpoint blockade (ICB), the endogenous population of T cells must retain a capacity to proliferate in response to the blocking antibody. Based on these commonalities, a recent consensus among the field has centered on less differentiated CD8 T cells driving therapeutic responses for both ICB and CAR T cell approaches (3, 8-13). The robust T cell expansion in both therapeutic settings has been associated with the effector population arising from a pool of stem-like CD8 T cells that have not yet undergone terminal differentiation or exhaustion. CD8 CD19-CAR T cells have been shown to undergo exhaustion in patients (14). Therefore, successful application of cellular therapy requires an understanding of cell-intrinsic mechanisms that restrict the developmental potential of CD8 T cells during their activation and sustained stimulation. Generally, there exists a need in the art for developing improved antigen-specific T cell therapy. This need can be met with a modified immune effector cell with enhanced effector cell function as disclosed herein.

### SUMMARY

As specified in the Background section above, there is a great need in the art for modified immune effector cells with enhanced immune cell function (e.g., preserved developmental potential) for use in cell therapy for cancer and other disease (e.g., infectious or autoimmune diseases). The present application addresses these and other needs.

In one aspect, provided herein is a modified immune effector cell, wherein an Additional Sex Combs Like Transcriptional Regulator 1 (ASXL1) gene or gene product is modified in the cell so that the expression and/or function of ASXL1 in the cell is reduced or eliminated.

In some embodiments, the level of functional ASXL1 protein in the cell is reduced by 50% or more.

In some embodiments, the ASXL1 gene is deleted so that no detectable functional ASXL1 protein is produced.

In some embodiments, the immune effector cell is a T cell.

In some embodiments, the T cell is selected from a CD8+ T cell, a CD4+ T cell, a cytotoxic T cell, an αβ T cell, a natural killer T (NKT) cell, a γδ T cell, a memory T cell, a T-helper cell, and a regulatory T cell (Treg).

In some embodiments, the immune effector cell is a stem cell that is capable of differentiating into an immune cell.

In some embodiments, the stem cell is an induced pluripotent stem cell (iPSC).

In some embodiments, the immune effector cell is a natural killer (NK) cell.

In some embodiments, the cell further comprises at least one surface molecule capable of binding specifically to an antigen.

In some embodiments, the antigen is selected from a tumor antigen, a viral antigen, a bacterial antigen, a fungal antigen, a parasite antigen, a prion antigen, and an antigen associated with an inflammation or an autoimmune disease.

In some embodiments, the tumor antigen is human epidermal growth factor receptor 2 (HER2), IL 13Rα2, erythropoietin-producing human hepatocellular receptor A2 (EphA2), B7 homolog 3 protein (B7-H3), Cluster of Differentiation (CD) 19 (CD19), CD22, or CD123.

In some embodiments, the cell further comprises a chimeric antigen receptor (CAR), an antigen specific T-cell receptor, or a bispecific antibody.

In some embodiments, the cell further comprises a CAR.

In some embodiments, the CAR comprises (i) an extracellular antigen-binding domain, (ii) a transmembrane domain, and (iii) a cytoplasmic domain.

In some embodiments, the extracellular antigen-binding domain comprises an antibody or an antibody fragment.

In some embodiments, the extracellular antigen binding domain comprises an scFv capable of binding to HER2, IL13Rα2, EphA2, B7-H3, CD19, CD22, or CD123.

In some embodiments, the extracellular antigen-binding domain further comprises a leader sequence.

In some embodiments, the transmembrane domain is derived from CD3ζ, CD28, CD4, or CD8α.

In some embodiments, the CAR further comprises a linker domain between the extracellular antigen-binding domain and the transmembrane domain.

In some embodiments, the linker domain comprises a hinge region.

In some embodiments, the CAR cytoplasmic domain comprises one or more lymphocyte activation domains.

In some embodiments, the lymphocyte activation domain is derived from DAP10, DAP12, Fc epsilon receptor I γ chain (FCER1G), CD3δ, CD3ε, CD3γ, CD3ζ, CD27, CD28, CD40, CD134, CD137, CD226, CD79A, ICOS, or MyD88.

In some embodiments, the CAR cytoplasmic domain comprises one or more co-stimulatory domains.

In some embodiments, a DNA (cytosine-5)-methyltransferase 3A (DNMT3A) gene or gene product is modified in the cell so that the expression and/or function of DNMT3A in the cell is reduced or eliminated.

In some embodiments, a Tet Methylcytosine Dioxygenase 2 (TET2) gene or gene product is modified in the cell so that the expression and/or function of TET2 in the cell is reduced or eliminated.

In some embodiments, the immune effector cell has been activated and/or expanded ex vivo.

In some embodiments, the immune effector cell is an allogeneic cell.

In some embodiments, the immune effector cell is an autologous cell.

In some embodiments, the immune effector cell is isolated from a subject having a disease.

In some embodiments, the disease is a cancer, an infectious disease, an inflammatory disorder, or an autoimmune disease.

In some embodiments, the cancer is a cancer expressing is a cancer expressing HER2, IL 13Rα2, EphA2, B7-H3, CD19, CD22, or CD123.

In some embodiments, the cancer is a HER2-positive breast cancer.

In some embodiments, the cancer is an IL13Rα2-positive glioblastoma.

In some embodiments, the immune effector cell is derived from a blood, marrow, tissue, or a tumor sample.

In another aspect, provided herein is a pharmaceutical composition comprising a modified immune effector cell described herein and a pharmaceutically acceptable carrier and/or excipient.

In another aspect, provided herein is a method for generating a modified immune effector cell described herein, the method comprising modifying an ASXL1 gene or gene product in the cell so that the expression and/or function of ASXL1 in the cell is reduced or eliminated.

In another aspect, provided herein is a method of preserving developmental potential of an immune effector cell, the method comprising modifying an ASXL1 gene or gene product in the cell so that the expression and/or function of ASXL1 in the cell is reduced or eliminated.

In some embodiments, the immune effector cell is a T cell.

In some embodiments, the T cell is selected from a CD8+ T cell, a CD4+ T cell, a cytotoxic T cell, an αβ T cell, a natural killer T (NKT) cell, a γδ T cell, a memory T cell, a T-helper cell, and a regulatory T cell (Treg).

In some embodiments, the method further comprises modifying the immune effector cell to express a chimeric antigen receptor (CAR) that is capable of binding to an antigen.

In some embodiments, the ASXL1 gene in the immune effector cell is modified as a result of an activity of a site-specific nuclease.

In some embodiments, the site-specific nuclease is an RNA-guided endonuclease.

In some embodiments, the RNA-guided endonuclease is a Cas9 protein, Cpf1 (Cas12a) protein, C2c1 protein, C2c3 protein, or C2c2 protein.

In some embodiments, the RNA-guided endonuclease is a Cas9 protein.

In some embodiments, the Cas9 protein is programmed with a guide RNA (gRNA) that comprises a nucleotide sequence of CCACUUACCAGAUAUGCCCC (SEQ ID NO: 143), CCAUUGGGAGAUCUAUUAGG (SEQ ID NO: 162), or GAUGCAAGUCAGGCUAAGAC (SEQ ID NO: 164).

In some embodiments, the site-specific nuclease is a zinc finger nuclease, a TALEN nuclease, or mega-TALEN nuclease.

In some embodiments, the ASXL1 gene product in the immune effector cell is modified as a result of an activity of an RNA interference (RNAi) molecule or an antisense oligonucleotide.

In some embodiments, the RNAi molecule is a small interfering RNA (siRNA) or a small hairpin RNA (shRNA).

In some embodiments, the site-specific nuclease or the RNAi molecule or the antisense oligonucleotide is introduced into the immune effector cell via a viral vector, a non-viral vector or a physical means.

In some embodiments, the CAR is expressed from a transgene introduced into the immune effector cell.

In some embodiments, the CAR-expressing transgene is introduced into the immune effector cell using a viral vector, a non-viral vector or a physical means.

In some embodiments, the viral vector is a retroviral vector, an adenoviral vector, an adeno-associated viral (AAV) vector, a herpes viral vector, or a baculoviral vector.

In some embodiments, the retroviral vector is a lentiviral vector.

In some embodiments, the non-viral vector is a transposon.

In some embodiments, the transposon is a sleeping beauty transposon or PiggyBac transposon.

In some embodiments, the physical means is electroporation, microinjection, magnetofection, ultrasound, a ballistic or hydrodynamic method, or a combination thereof.

In some embodiments, the modified immune effector cell is activated and/or expanded ex vivo.

In another aspect, provided herein is a method of treating a disease in a subject in need thereof comprising administering to the subject an effective amount of a modified immune effector cell described herein or a pharmaceutical composition described herein.

In some embodiments, the modified immune effector cell is an autologous cell.

In some embodiments, the modified immune effector cell is an allogeneic cell.

In some embodiments, the disease is a cancer, an infectious disease, an inflammatory disorder, or an autoimmune disease.

In some embodiments, the cancer is a solid tumor.

In some embodiments, the cancer is a hematologic cancer.

In some embodiments, the cancer is a cancer expressing HER2, IL13Rα2, EphA2, B7-H3, CD19, CD22, or CD123.

In some embodiments, the method described herein comprises:
i. isolating an immune effector cell from the subject or a donor;
ii. modifying an ASXL1 gene or gene product in the immune effector cell so that the expression and/or function of ASXL1 in the cell is reduced or eliminated; and
iii. introducing the modified immune effector cell into the subject.

In some embodiments, the method described herein further comprises modifying the immune effector cell to express a chimeric antigen receptor (CAR) that is capable of binding specifically to an antigen.

In some embodiments, the subject is a human or a mouse.

In another aspect, provided herein is a guide RNA (gRNA) targeting ASXL1 comprising a nucleotide sequence of CCACUUACCAGAUAUGCCCC (SEQ ID NO: 143), CCAUUGGGAGAUCUAUUAGG (SEQ ID NO: 162), or GAUGCAAGUCAGGCUAAGAC (SEQ ID NO: 164).

In another aspect, provided herein is a ribonucleoprotein complex comprising a gRNA of disclosed herein and a Cas9 protein.

### BRIEF DESCRIPTION OF THE DRAWINGS

FIGS. 1A-1B demonstrate Additional Sex Combs Like Transcriptional Regulator 1 (ASXL1) undergoes demethylation during both murine and human CD8 T cell differentiation. Whole genome bisulfite sequencing nucleotide-resolution methylation profiling of murine Axl1 (FIG. 1A) and human ASXL1 (FIG. 1B). Individual CpG sites are represented by vertical lines with gray indicating methylation and black indicating lack of methylation. Differentially methylated regions (DMRs) are represented by a dashed box. Stem cell memory (Tscm), Central memory (Tcm), Effector memory (Tem).

FIGS. 2A-2E show Asxl1 disruption preserves a stem-like phenotype during chronic T cell stimulation. Experimental schema for Clustered Regularly Interspaced Short Palindromic Repeats (CRISPR) disruption of the Asxl1 gene in lymphocytic choriomeningitis virus (LCMV)-specific P14 T cells that differentiate in response to chronic LCMV infection and Programmed death-ligand 1 (PD-L1) blockade therapy (FIG. 2A). Longitudinal analysis of Asxl1 KO P14 cell frequency during chronic LCMV infection and after PD-L1 blockade (FIG. 2B). Representative Fluorescence-activated Cell Sorting (FACS) plots and summary graphs showing enrichment of a stem-like T cell phenotype (T-cell immunoglobulin mucin-3 [Tim-3]-Cluster of Differentiation 101 [CD101]- and C-X3-C motif chemokine receptor 1 [Cx3cr1]-) among Asxl1 knockout (KO) P14 cells after chronic LCMV stimulation and expansion after PD-L1 blockade (FIG. 2C and FIG. 2D). Representative FACS plots showing cytokine production of wild-type (WT) and Asxl1 KO P14 cells after 5 hours of ex vivo peptide stimulation (FIG. 2E).

FIG. 3 is a schematic depiction of how the DNA methylation-based T cell multipotency index (MPI) can inform on clinical response to T cell-based immunotherapies. Determining the differentiation status of CD8 T cells by using the multipotency index can identify a ‘therapeutic window’ of CD8 T cell differentiation that is most likely to induce a clinical response in both immune checkpoint blockade (ICB) and chimeric antigen receptor (CAR) T cell therapy (2). FM is functional memory, TE is terminal effector, Exh. is exhausted.

FIGS. 4A-4B illustrate Asxl1 KO CD8 T cells have a heightened capacity to proliferate. Experimental schema showing T cell receptor (TCR) transgenic P14 cells edited for disruption of Asxl1 (FIG. 4A). Edited P14 cells were adoptively transferred into mice and the mice were then chronically infected with LCMV. P14 frequency was tracked longitudinally in the blood of the infected mice (FIG. 4B).

FIGS. 5A-5F demonstrate ASXL1 KO effector P14 cells preserve a stem-like phenotype (Day 7). Representative FACS analysis of P14 cells showing stem-like phenotype (Tim3-CD101-) (FIG. 5A). Summary graph of Tim3 expression among P14s for cells among all mice in the blood (n=10) and spleen (n=6) (FIG. 5B). Representative FACS analysis of P14 Granzyme B expression (FIG. 5C). Summary graph of Granzyme B expression for all mice (n=6) (FIG. 5D). Representative histogram analysis of KI67 (proliferation marker) and Tox (FIG. 5E). Summary graph of KI67 and Tox expression for P14 cells among all mice (n=6) (FIG. 5F).

FIGS. 6A-6C show ASXL1 KO P14 cells have a heightened capacity to respond to programmed cell death protein 1 (PD-1) blockade. Experimental schema showing TCR transgenic P14 cells edited for disruption of Asxl1. Edited P14 cells were adoptively transferred into mice and the mice were then chronically infected with LCMV (FIG. 6A). P14 frequency was tracked longitudinally in the blood of the infected mice and compared in mice treated with and without PD-L1 (FIG. 6B). Summary graph quantifying the percent and absolute number of WT and Asxl1 KO P14s isolated from mice treated with and without PD-L1 (FIG. 6C).

FIGS. 7A-7D show ablation of ASXL1 preserves a stem-like phenotype during chronic T cell stimulation (Day 42). Representative FACS analysis of P14 cells showing stem-like phenotype (Tim3-CD101-) (FIG. 7A). Summary graph of Tim3 expression among P14s for cells among all mice (n=10) (FIG. 7B). Summary graph of Tim3 and CD101 expression among P14s for cells among all mice (n=10) (FIG. 7C). Summary graph of Tim3 and CD101 expression for all mice (n=10) (FIG. 7D).

FIGS. 8A-8D illustrate ablation of ASXL1 preserves a stem-like phenotype during chronic T cell stimulation. Representative FACS analysis of P14 cells showing a stem-like phenotype (Tim3-CD101-) (FIG. 8A). Summary graph of IFNg expression among P14s for cells among all mice (FIG. 8B). Representative histogram analysis of Tox and GzmB (FIG. 8C). Summary graph of Tox and GzmB expression for P14 cells among all mice (FIG. 8D).

### DETAILED DESCRIPTION

Recent efforts to better understand the poised nature of endogenous and adoptively transferred tumor specific CD8 T cells has revealed that the stem-like nature of the cells is coupled to epigenetic programs that preserve T cell memory potential (2, 15, 16). The present disclosure, among other things, describes ASXL1 as a prevalent driver of CHIP and supports a critical role for ASXL1 in regulating CD8 T cell stemness and homeostasis. Data described herein, in particular, show that ASXL1 KO T cells retain a stem-like state of T cell differentiation. Exploration of the subclonal biology is required to identify the contribution of CHIP-associated molecules, e.g., ASXL1, to the epigenetic programs regulating the checkpoint between homeostasis of functional memory T cells and uncontrolled cellular outgrowth.

### Definitions

The term “immune effector cell” as used herein refers to a cell that is involved in an immune response, e.g., in the promotion of an immune effector response. Non-limiting examples of immune effector cells include T cells (e.g., αβ T cells and γδ T cells), B cells, natural killer (NK) cells, natural killer T (NKT) cells, mast cells, and myeloid-derived phagocytes. Immune effector cells include stem cells, such as induced pluripotent stem cells (iPSCs), that are capable of differentiating into immune cells.

The terms “T cell” and “T lymphocyte” are interchangeable and used synonymously herein. As used herein, T cell includes thymocytes, naïve T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a CD8+ T cell, a CD4+ T cell, a helper T cell or T-helper cell (HTL; CD4+ T cell), a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naïve T cells and memory T cells. Also included are “αβ T cell receptor (TCR) T cells”, which refer to a population of T cells that possess a TCR composed of α- and β-TCR chains. Also included are “NKT cells”, which refer to a specialized population of T cells that express a semi-invariant αβ T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8+ cytotoxic T cell response. Also included are “regulatory T cells” or “Tregs”, which refer to T cells that suppress an abnormal or excessive immune response and play a role in immune tolerance. Tregs cells are typically transcription factor Foxp3-positive CD4+ T cells and can also include transcription factor Foxp3-negative regulatory T cells that are IL-10-producing CD4+ T cells.

The terms “natural killer cell” and “NK cell” are used interchangeably and synonymously herein. As used herein, NK cell refers to a differentiated lymphocyte with a CD 16+CD56+ and/or CD57+ TCR-phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

The term “chimeric antigen receptor” or “CAR” as used herein is defined as a cell-surface receptor comprising an extracellular antigen-binding domain, a transmembrane domain and a cytoplasmic domain comprising a lymphocyte activation domain and, optionally, at least one co-stimulatory signaling domain, all in a combination that is not naturally found together on a single protein. This particularly includes receptors wherein the extracellular domain and the cytoplasmic domain are not naturally found together on a single receptor protein. The chimeric antigen receptors of the present invention are intended primarily for use with lymphocyte such as T cells and natural killer (NK) cells.

As used herein, the term “antigen” refers to any agent (e.g., protein, peptide, polysaccharide, glycoprotein, glycolipid, nucleic acid, portions thereof, or combinations thereof) molecule capable of being bound by a T-cell receptor. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The term “antigen-binding moiety” refers to a target-specific binding element that may be any ligand that binds to the antigen of interest or a polypeptide or fragment thereof, wherein the ligand is either naturally derived or synthetic. Examples of antigen-binding moieties include, but are not limited to, antibodies; polypeptides derived from antibodies, such as, for example, single chain variable fragments (scFv), Fab, Fab′, F(ab′)2, and Fv fragments; polypeptides derived from T cell receptors, such as, for example, TCR variable domains; secreted factors (e.g., cytokines, growth factors) that can be artificially fused to signaling domains (e.g., “zytokines”); and any ligand or receptor fragment (e.g., CD27, NKG2D) that binds to the antigen of interest. Combinatorial libraries could also be used to identify peptides binding with high affinity to the therapeutic target.

The terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910, each of which are incorporated by reference in their entirety for all purposes. Antibodies useful as a TCR-binding molecule include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgM1, IgM2, IgAQ1 and IgA2) or subclass. Also included are “bispecific antibodies”, which refer to antibodies that are capable of binding to two different antigens or different epitopes of the same antigen.

The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12.

Host cells of the present invention include T cells and natural killer cells that contain the DNA or RNA sequences encoding the CAR and express the CAR on the cell surface. Host cells may be used for enhancing T cell activity, natural killer cell activity, treatment of cancer, and treatment of autoimmune disease.

The terms “activation” or “stimulation” means to induce a change in their biologic state by which the cells (e.g., T cells and NK cells) express activation markers, produce cytokines, proliferate and/or become cytotoxic to target cells. All these changes can be produced by primary stimulatory signals. Co-stimulatory signals can amplify the magnitude of the primary signals and suppress cell death following initial stimulation resulting in a more durable activation state and thus a higher cytotoxic capacity. A “co-stimulatory signal” refers to a signal, which in combination with a primary signal, such as TCR/CD3 ligation, leads to T cell and/or NK cell proliferation and/or upregulation or downregulation of key molecules.

The term “proliferation” refers to an increase in cell division, either symmetric or asymmetric division of cells. The term “expansion” refers to the outcome of cell division and cell death.

The term “differentiation” refers to a method of decreasing the potency or proliferation of a cell or moving the cell to a more developmentally restricted state.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The term “transfection” means the introduction of a “foreign” (i.e., extrinsic or extracellular) nucleic acid into a cell using recombinant DNA technology. The term “genetic modification” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

The term “transduction” means the introduction of a foreign nucleic acid into a cell using a viral vector.

The terms “genetically modified” or “genetically engineered” refers to the addition of extra genetic material in the form of DNA or RNA into a cell.

As used herein, the term “derivative” in the context of proteins or polypeptides (e.g., CAR constructs or domains thereof) refer to: (a) a polypeptide that has at least 40%, 45%, 50%, 55%, 60%, 65%, 70%, 75%, 80%, 85%, 90%, 95%, 98% or 99% sequence identity to the polypeptide it is a derivative of; (b) a polypeptide encoded by a nucleotide sequence that has at least 40%, 45%, 50%, 55%, 60%, 65%, 70%, 75%, 80%, 85%, 90%, 95%, 98% or 99% sequence identity to a nucleotide sequence encoding the polypeptide it is a derivative of; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a derivative of; (d) a polypeptide encoded by nucleic acids can hybridize under high, moderate or typical stringency hybridization conditions to nucleic acids encoding the polypeptide it is a derivative of; (e) a polypeptide encoded by a nucleotide sequence that can hybridize under high, moderate or typical stringency hybridization conditions to a nucleotide sequence encoding a fragment of the polypeptide, it is a derivative of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a derivative of.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Patent Application Publication No. US 2005/0048549 (e.g., paragraphs 72-73).

Percent sequence identity can be determined using a global alignment between two sequences. As used herein, the term “global alignment” refers to an alignment of residues between two amino acid or nucleic acid sequences along their entire length, introducing gaps as necessary if the two sequences do not have the same length, to achieve a maximum percent identity. A global alignment can be created using the global alignment tool “Needle” from the online European Molecular Biology Open Software Suite (EMBOSS) (see www.ebi.ac.uk/Tools/psa/emboss_needle/) or the global alignment tool “BLAST®»Global Alignment” from the National Center for Biotechnology Information (NCBI) (see blast.ncbi.nlm.nih.gov/Blast.cgi?PROGRAM=blastn&PAGE_TYPE=BlastSearch&PROG_DEFAULTS=on&BLAST_INIT-GlobalAln&BLAST_SPEC-GlobalAln&BLAST_PROGRAMS=blastn). Both of these global alignment tools incorporate the Needleman-Wunsch algorithm (Needleman, S. B. & Wunsch, C. D. (1970) “A general method applicable to the search for similarities in the amino acid sequences of two proteins.” J. Mol. Biol. 48:443-453). In a preferred embodiment, a global alignment of nucleotide sequences using BLAST Global Alignment uses the following default parameters: match score=2; mismatch score=−3; Gap Cost Existence score=5; Gap Cost Extension Score=2. In a preferred embodiment, a global alignment of protein sequences using BLAST Global Alignment uses the following default parameters: Gap Cost Existence=11; Gap Cost Extension=1.

The term “variant” as used herein refers to a modified polypeptide, protein, or polynucleotide that has substantial or significant sequence identity or similarity to a wild-type polypeptide, protein, or polynucleotide. The variant may retain the same, or have altered (e.g., improved, reduced or abolished) biological activity relative to the wild-type polypeptide, protein, or polynucleotide of which it is a variant. The variant may contain an insertion, a deletion, a substitution of at least one amino acid residue or nucleotide.

The terms “vector”, “cloning vector” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In some embodiments, the vector is a viral vector such as, but not limited to, viral vector is an adenoviral, adeno-associated, alphaviral, herpes, lentiviral, retroviral, baculoviral, or vaccinia vector.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “site-specific nuclease” as used herein refers to a nuclease capable of specifically recognizing and cleaving a nucleic acid (DNA or RNA) sequence. Suitable site-specific nucleases for use in the present invention include, but are not limited to, RNA-guided endonuclease (e.g., CRISPR-associated (Cas) proteins), zinc finger nuclease, a TALEN nuclease, or mega-TALEN nuclease.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, effector function, persistence, and/or an increase in antitumor activity (e.g., cancer cell death or cancer cell killing ability), among others apparent from the understanding in the art and the description herein. In some embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In some embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The terms “inhibit” or “inhibition” as used herein refer to reducing a function or activity to an extent sufficient to achieve a desired biological or physiological effect. Inhibition may be complete or partial.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

Singular forms “a”, “an”, and “the” include plural references unless the context clearly dictates otherwise. Thus, for example, a reference to “a method” includes one or more methods, and/or steps of the type described herein, which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

If aspects of the disclosure are described as “comprising” a feature, or versions there of (e.g., comprise), embodiments also are contemplated “consisting of” or “consisting essentially of” the feature.

The practice of the present invention employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

### Modified Immune Effector Cells

In one aspect, the invention provides a modified immune effector cell with enhanced immune cell function, e.g., preserved developmental potential (i.e., preserved stem-like state of differentiation). In particular, the immune effector cell is modified such that the expression and/or function of Additional Sex Combs Like Transcriptional Regulator 1 (ASXL1) in the cell is reduced or eliminated. In some embodiments, an ASXL1 gene or gene product is modified in the cell so that the expression and/or function of ASXL1 in the cell is reduced or eliminated.

In some embodiments, the immune effector cell is a T cell. T cells may include, but are not limited to, thymocytes, naïve T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naïve T cells, memory T cells, and NKT cells.

In some embodiments, the T cell may be a CD8+ T cell, a CD4+ T cell, a cytotoxic T cell, an αβ T cell receptor (TCR) T cell, a natural killer T (NKT) cell, a γδ T cell, a memory T cell, a T-helper cell, or a regulatory T cell (Treg).

The modification may be applied to all forms of T cell therapies, which include but are not limited to, therapies with: i) T cells that express a chimeric antigen receptor (CAR); ii) T cells that express an endogenous αβ TCR or an endogenous γδ TCR, which may be specific for, e.g., a peptide derived from viral or tumor-associated antigens (including neoantigens); iii) T cells that transgenically express an αβ TCR or a γδ TCR, which may be specific for, e.g., a peptide derived from viral or tumor-associated antigens (including neoantigens); iv) T cells that transgenically express bispecific antibodies, which recognize viral or tumor-associated antigens (including neoantigens)/or a peptide derived from them and an activating molecule expressed on T cells such as CD3; and/or v) T cells that are generated via stimulation with for examples but not limited to peptides, antigen presenting and/or artificial antigen presenting cells (in vitro sensitized [IVS] T cell therapy). Lastly, T cell therapies in which the therapeutic genes are delivered in vivo are also included (in vivo T cell therapy).

In some embodiments, the immune effector cell is a T cell. Non-limiting examples of a T cell include a CD8+ T cell, a CD4+ T cell, a cytotoxic T cell, an αβ T cell receptor (TCR) T cell, a natural killer T (NKT) cell, a γδ T cell, a memory T cell, a T-helper cell, and a regulatory T cell (Treg).

In some embodiments, the immune effector cell is a natural killer (NK) cell. NK cell refers to a differentiated lymphocyte with a CD3−CD16+, CD3−CD56+, CD16+CD56+ and/or CD57+ TCR− phenotype.

In some embodiments, the immune effector cell is a stem cell that is capable of differentiating into an immune cell. The stem cell may be an induced pluripotent stem cell (iPSC).

Without wishing to be bound by theory, Additional sex combs-like (ASXL)1, ASXL2 and ASXL3, the human homologues of the Drosophila Asx gene, are involved in the regulation or recruitment of the Polycomb-group repressor complex (PRC) and trithorax-group (trxG) activator complex. ASXL1 interacts with KDM1A (LSD1), BAP1, NCOA1 and nuclear hormone receptors (NHRs). The members of the ASXL family assemble epigenetic regulators and transcription factors to specific genomic loci with histone modifications. ASXL1 is involved in transcriptional repression through an interaction with PRC2 and to transcriptional regulation through interactions with BAP1ASXL1 is overexpressed in cervical cancer and mutations of ASXL1 are detected in prostate and breast cancers, colorectal cancers with microsatellite instability (MSI), head and neck squamous cell carcinoma, malignant myeloid diseases, chronic lymphocytic leukemia, and liver cancer.

In some embodiments, an ASXL1 gene or gene product is modified in a cell disclosed herein so that the expression and/or function of ASXL1 in the cell is reduced or eliminated. In some embodiments the level of functional ASXL1 protein in the cell is reduced by about 50% or more. The level of functional ASXL1 protein in the cell may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level of functional ASXL1 protein in the cell may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the ASXL1 gene is deleted or defective so that no detectable wild-type ASXL1 protein is produced. The ASXL1 gene may be deleted or become defective using the methods described herein.

In some embodiments, an ASXL1 gene or gene product and a DNMT3A gene or gene product are both modified in the same cell so that the expression and/or function of ASXL1 and DNMT3A in the cell is reduced or eliminated. In some embodiments, the ASXL1 and the DNMT3A gene are deleted and/or defective in the cell so that no detectable wild-type ASXL1 and DNMT3A protein are produced.

In some embodiments, an ASXL1 gene or gene product and a Tet Methylcytosine Dioxygenase 2 (TET2) gene or gene product are both modified in the same cell so that the expression and/or function of ASXL1 and TET2 in the cell is reduced or eliminated. In some embodiments, the ASXL1 and the TET2 gene are deleted and/or defective in the cell so that no detectable wild-type ASXL1 and TET2 protein are produced.

In some embodiments, an ASXL1 gene or gene product, a DNMT3A gene or gene product, and a TET2 gene or gene product are modified in the same cell so that the expression and/or function of ASXL1, DNMT3A, and TET2 in the cell is reduced or eliminated. In some embodiments, the ASXL1, DNMT3A, and/TET2 gene are deleted and/or defective in the cell so that no detectable wild-type ASXL1, DNMT3A, and TET2 protein are produced.

DNA (cytosine-5)-methyltransferase 3A (DNMT3A) is an enzyme that catalyzes the addition of methyl groups to cytosine residues of CpG structures in DNA. The enzyme is encoded in humans by the DNMT3A gene. This enzyme is responsible for de novo DNA methylation. Such function may be different from maintenance DNA methylation which ensures the fidelity of replication of inherited epigenetic patterns. The DNMT3A-mediated de novo DNA methylation is critical in DNA imprinting and modulation of gene expression. Compositions and methods for modulating DNMT3A gene or gene products is described in PCT publication WO 2020/222987, which is incorporated by reference in its entirety for all purposes.

In some embodiments, the enzymatic activity of the DNMT3A protein is inhibited in the cell. The enzymatic activity of the DNMT3A protein may be inhibited by exposing the cell to a DNMT3A active site inhibitor. Although not wishing to be bound by theory, the methyl-transfer reaction carried out by a DNA methyltransferase is typically initiated by nucleophilic attack from a catalytic cysteine in the active site. The catalytic cysteine is highly conserved among cytosine methyltransferases. When the catalytic cysteine is mutated or blocked the enzymatic activity of the DNMT3A protein can be inhibited, although binding may still occur. The catalytic cysteine of human DNMT3A has been identified to be C710 (Zhang, Z. M. et al., Nature. 2018; 554 (7692): 387-391, which is incorporated herein by reference in its entirety for all purposes). Examples of DNMT3A active site inhibitors that may be used in the present invention include 5-azacytidine, Decitabine, Zebularine, 5-fluoro-2′-deoxycytidine, as well as other cytidine analogs known in the art. A further example of a DNMT3A active site inhibitor includes RG108.

In some embodiments, the DNMT3A gene is mutated in the DNMT3A catalytic domain so that the enzymatic activity of the DNMT3A protein is inhibited. As a non-limiting example, a catalytic cysteine in the catalytic domain may be mutated in a way that the enzymatic reaction can no longer occur.

In some embodiments, the level of functional DNMT3A protein in the cell is decreased by about 50% or more. The level of functional DNMT3A protein in the cell may be decreased by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level of functional DNMT3A protein in the cell may be decreased by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the DNMT3A gene is deleted or defective so that no detectable wild-type DNMT3A protein is produced. The DNMT3A gene may be deleted or become defective using the methods described herein.

In some embodiments, the ASXL1 gene and the DNMT3A gene may be deleted (e.g., knocked out) in the same cell using the methods described herein.

TET2 (Tet Methylcytosine Dioxygenase 2) is a protein coding gene. Although not wishing to be bound by theory, TET proteins, e.g., TET2, play major roles in the regulation of DNA-methylation status, e.g., by oxidizing 5-methylcytosine (5mC) to generate 5-hydroxymethylcytosine (5hmC) which can both act as a stable epigenetic mark and participate in active demethylation. TET2 has been classified as a tumor suppressor. As such, in cancer, loss of TET2 function, e.g., via TET2 deletion, TET2 mutation, and/or Isocitrate dehydrogenase 1 (IDH1) and/or IDH2 mutation, have been associated with myeloid and lymphoid transformations. By way of a non-limiting example, mutations in TET2 have been identified in acute myeloid leukemia (AML), myelodysplastic syndrome (MDS), acute lymphocytic leukemia (ALL), and other various hematologic malignancies.

In some embodiments, the level of functional TET2 protein in the cell is decreased by about 50% or more. The level of functional TET2 protein in the cell may be decreased by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level of functional TET2 protein in the cell may be decreased by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the TET2 gene is deleted or defective so that no detectable wild-type TET2 protein is produced. The TET2 gene may be deleted or become defective using the methods described herein.

In some embodiments, the ASXL1 gene and the TET2 gene may be deleted (e.g., knocked out) in the same cell using the methods described herein.

In some embodiments, the ASXL1 gene, the DNMT3A gene, and/or the TET2 gene may be deleted (e.g., knocked out) in the same cell using the methods described herein.

In some embodiments, in addition to modifying an ASXL1 gene or gene product and/or a DNMT3A gene or gene product and/or a TET2 gene or gene product, a STAT5 signaling pathway is activated in the immune effector cell. In some embodiments, the STAT5 signaling pathway is activated by a signaling molecule. The signaling molecule may be a common gamma chain cytokine. Non-limiting examples of cytokines that may be used in the methods described herein include IL-15, IL-7, IL-2, IL-4, IL-9, and IL-21. The cytokine may be a native or modified cytokine. In some embodiments, the signaling molecule is IL-15. In some embodiments, the signaling molecule is IL-7.

In some embodiments, the STAT5 signaling pathway is activated by modifying the immune effector cell to express a constitutively active cytokine receptor or a switch receptor. Constitutively active cytokine receptors may trigger the activation of a cytokine signaling cascade even in the absence of extracellular cytokine. This may circumvent the need for providing extracellular cytokines to the immune effector cell. A non-limiting example of a constitutively active cytokine receptor is a constitutively active IL7 receptor (C7R). Such constitutively active cytokine receptor may be generated using methods described in Shum T et al. Cancer Discov. 2017; 7(11):1238-1247, which is incorporated herein in its entirety for all purposes.

A switch receptor (also known as inverted cytokine receptor), which is capable of converting a potentially inhibitory signal into a positive signal, is also contemplated by the present invention. Non-limiting examples of switch receptors that may also be used in the methods described herein include an IL4/IL7 receptor and an IL4/IL2 receptor. Such receptors may be generated as described in Bajgain, P. et al., J Immunother Cancer. 2018; 6(1):34 and Wilkie, S. et al., J Biol Chem. 2010; 285(33):25538-44, both of which are incorporated herein by reference in their entirety for all purposes. In some embodiments, the modified immune effector cell further comprises at least one surface molecule capable of binding specifically to an antigen. The antigen may be a tumor antigen, a viral antigen, a bacterial antigen, a fungal antigen, a parasite antigen, a prion antigen, or an antigen associated with an inflammation or an autoimmune disease.

In some embodiments, the antigen is a tumor antigen. Non-limiting examples of tumor antigens that may be targeted by the modified immune effector cell described herein include human epidermal growth factor receptor 2 (HER2), interleukin-13 receptor subunit alpha-2 (IL-13Ra2), ephrin type-A receptor 2 (EphA2), A kinase anchor protein 4 (AKAP-4), adrenoceptor beta 3 (ADRB3), anaplastic lymphoma kinase (ALK), immunoglobulin lambda-like polypeptide 1 (IGLL1), androgen receptor, angiopoietin-binding cell surface receptor 2 (Tie 2), B7-H3 (CD276), bone marrow stromal cell antigen 2 (BST2), carbonic anhydrase IX (CAIX), CCCTC-binding factor (Zinc Finger Protein)-like (BORIS), CD171, CD179a, CD24, CD300 molecule-like family member f (CD300LF), CD3, CD44v6, CD72, CD79a, CD79b, CD97, chromosome X open reading frame 61 (CXORF61), claudin 6 (CLDN6), CS-1 (CD2 subset 1, CRACC, SLAMF7, CD319, or 19A24), C-type lectin domain family 12 member A (CLEC12A), C-type lectin-like molecule-1 (CLL-1), Cyclin B 1, Cytochrome P450 1B 1 (CYP1B 1), EGF-like module-containing mucin-like hormone receptor-like 2 (EMR2), epidermal growth factor receptor (EGFR), ERG (transmembrane protease, serine 2 (TMPRSS2) ETS fusion gene), ETS translocation-variant gene 6, located on chromosome 12p (ETV6-AML), Fc fragment of IgA receptor (FCAR), Fc receptor-like 5 (FCRL5), Fms-like tyrosine kinase 3 (FLT3), Folate receptor beta, Fos-related antigen 1, Fucosyl GM1, G protein-coupled receptor 20 (GPR20), G protein-coupled receptor class C group 5, member D (GPRC5D), ganglioside GD3, ganglioside GM3, glycoceramide (GloboH), Glypican-3 (GPC3), Hepatitis A virus cellular receptor 1 (HAVCR), hexasaccharide portion of globoH, high molecular weight-melanoma-associated antigen (HMWMAA), human Telomerase reverse transcriptase (hTERT), interleukin 11 receptor alpha (IL-11Ra), KIT (CD117), leukocyte-associated immunoglobulin-like receptor 1 (LAIR1), leukocyte immunoglobulin-like receptor subfamily A member 2 (LILRA2), Lewis (Y) antigen, lymphocyte antigen 6 complex, locus K 9 (LY6K), lymphocyte antigen 75 (LY75), lymphocyte-specific protein tyrosine kinase (LCK), mammary gland differentiation antigen (NY-BR-1), melanoma cancer testis antigen-1 (MAD-CT-1), melanoma cancer testis antigen-2 (MAD-CT-2), melanoma inhibitor of apoptosis (ML-IAP), mucin 1, cell surface associated (MUC1), N-acetyl glucosaminyl-transferase V (NA17), neural cell adhesion molecule (NCAM), o-acetyl-GD2 ganglioside (OAcGD2), olfactory receptor 51E2 (OR51E2), p53 mutant, paired box protein Pax-3 (PAX3), paired box protein Pax-5 (PAX5), pannexin 3 (PANX3), placenta-specific 1 (PLAC1), platelet-derived growth factor receptor beta (PDGFR-beta), Polysialic acid, proacrosin binding protein sp32 (OY-TES 1), prostate stem cell antigen (PSCA), Protease Serine 21 (PRSS21), Proteasome (Prosome, Macropain) Subunit, Beta Type, 9 (LMP2), Ras Homolog Family Member C (RhoC), sarcoma translocation breakpoints, sialyl Lewis adhesion molecule (sLe), sperm protein 17 (SPA17), squamous cell carcinoma antigen recognized by T cells 3 (SART3), stage-specific embryonic antigen-4 (SSEA-4), synovial sarcoma, X breakpoint 2 (SSX2), TCR gamma alternate reading frame protein (TARP), TGS5, thyroid stimulating hormone receptor (TSHR), Tn antigen (Tn Ag), tumor endothelial marker 1 (TEMI/CD248), tumor endothelial marker 7-related (TEM7R), uroplakin 2 (UPK2), vascular endothelial growth factor receptor 2 (VEGFR2), v-myc avian myelocytomatosis viral oncogene neuroblastoma derived homolog (MYCN), Wilms tumor protein (WT1), and X Antigen Family, Member 1A (XAGE1), or a fragment or variant thereof.

Additional antigens that may be targeted by the extracellular target-binding domain include, but are not limited to, carbonic anhydrase EX, alpha-fetoprotein, A3, antigen specific for A33 antibody, Ba 733, BrE3-antigen, CA125, CD1, CD1a, CD3, CD5, CD15, CD16, CD19, CD20, CD21, CD22, CD23, CD25, CD30, CD33, CD3δ, CD45, CD74, CD79a, CD80, CD123, CD138, colon-specific antigen-p (CSAp), CEA (CEACAM5), CEACAM6, CSAP, EGFR, EGP-I, EGP-2, Ep-CAM, EphA1, EphA3, EphA4, EphA5, EphA6, EphA7, EphA8, EphA10, EphB1, EphB2, EphB3, EphB4, EphB6, Flt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC1, MUC2, MUC3, MUC4, NCA66, NCA95, NCA90, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker or an oncogene product.

In some embodiments, the tumor antigen targeted by the modified immune effector cell is human epidermal growth factor receptor 2 (HER2), IL13Rα2, erythropoietin-producing human hepatocellular receptor A2 (EphA2), B7 homolog 3 protein (B7-H3), Cluster of Differentiation (CD) 19 (CD19), CD22, or CD123.

In some embodiments, the tumor antigen targeted by the modified immune effector cell is CD19, CD22, CD123, CD33, or a fragment or variant thereof.

In some embodiments, the tumor antigen targeted by the modified immune effector cell is HER2, IL13Rα2, or EphA2, or a fragment or variant thereof.

In some embodiments, the tumor antigen targeted by the modified immune effector cell is HER2. Human epidermal growth factor receptor 2 (HER2), also referred to as HER2/neu, receptor tyrosine-protein kinase erbB-2, CD340 (cluster of differentiation 340), proto-oncogene Neu, or ERBB2, a membrane tyrosine kinase and oncogene that is overexpressed in some types of cancer.

In some embodiments, the tumor antigen targeted by the modified immune effector cell is IL13Rα2. Interleukin-13 receptor subunit alpha-2 (IL13Rα2), also referred to as CD213A2 (cluster of differentiation 213A2), is a membrane bound protein that, in humans, is encoded by the IL 13RA2 gene.

In some embodiments, the tumor antigen targeted by the modified immune effector cell is EphA2. Ephrin type-A receptor 2 (EphA2), also referred to as Eck (epithelial cell kinase), Myk2, or Sek2, a member of the Eph receptor tyrosine kinase family which binds Ephrins A1, 2, 3, 4, and 5.

In some embodiments, the tumor antigen targeted by the modified immune effector cell is B7-H3 (CD276), or a fragment or variant thereof. B7 Homolog 3 (B7-H3) or CD276 (cluster of differentiation 276) is a type I transmembrane protein that is an immune checkpoint molecule and a costimulatory/coinhibitory immunoregulatory protein. Without wishing to be bound by theory, B7-H3 is highly expressed in tumor tissues (e.g., breast cancer, lung cancer, ovarian cancer, brain tumor, gastric cancer, and squamous cell carcinoma) where it participates in shaping and development of the tumor microenvironment, while showing limited expression in normal tissues. B7-H3 may also support pro-tumorigenic functions, e.g., enhanced invasive and migratory properties, and has been correlated with worsened prognosis, poor survival, and recurrence rate. In some embodiments, the modified immune effector cell further comprises a chimeric antigen receptor (CAR), an antigen specific T-cell receptor, or a bispecific antibody.

In some embodiments, the modified immune effector cell further comprises an antigen specific T-cell receptor. Antigen specific T-cell receptors are T-cell receptors (TCRs) that are specific for recognizing a particular antigen. In some embodiments, the modified immune effector cell comprises a T cell receptor (TCR), or a functional fragment thereof. By way of a non-limiting example, a functional fragment of a TCR may immunospecifically bind to a particular antigen (or epitope) while retaining the capability to immunospecifically bind to the antigen (or epitope). In various embodiments, a functional fragment of a TCR may comprise at least one complementary determining region (CDR) of the alpha chain and/or beta chain of the TCR. In various embodiments, a functional fragment of a TCR may comprise two or more complementary determining regions (CDRs) of the alpha chain and/or beta chain of the TCR. In various embodiments, a functional fragment of a TCR may comprise at least one complementary determining region (CDR) of the gamma chain and/or delta chain of the TCR. In various embodiments, a functional fragment of a TCR may comprise two or more complementary determining regions (CDRs) of the gamma chain and/or delta chain of the TCR.

In some embodiments, the TCR disclosed herein may comprise, for example, one or more of an alpha (α) chain of a TCR, a beta (β) chain of a TCR, a delta (δ) chain of a TCR, a gamma (γ) chain of a TCR, or a combination thereof. In some embodiments, the TCR may further comprise a constant region. The constant region may be derived from any suitable species such as, e.g., human or mouse.

In some embodiments, the TCR may comprise an alpha chain and/or a beta chain of the TCR. In some embodiments, the TCR may comprise, e.g., constant regions of alpha and/or beta chains of the TCR.

In some embodiments, the antigen specific TCR may recognize, without limitation, any of the antigens (e.g., an antigen(s) on a cancer cell) disclosed herein. In various embodiments, the TCR of the disclosure may specifically bind to an antigen selected from, for example, CD7, CD74, CDS, CEA, EGP-2, EGP-40, EpCAM, erb-B2,3,4, FBP, fetal acetylcholine receptor, folate receptor-α, GD2, GD3, HER2, hTERT, IL-13R-α2, KDR, K-light chain, LeY, L1 cell, MAGE-A1, Mesothelin, MUC1, MUC16, NKG2D ligands, NY-ESO-1, oncofetal antigen (h5T4), PSCA, PSMA, ROR1, TAG-72, VEGF-R2, and WT-1.

In some embodiments, the modified immune effector cell further comprises a bispecific antibody. Bispecific antibodies are antibodies that are capable of binding to two different antigens or two different epitopes of the same antigen. For example, the modified immune effector cell may comprise a bispecific antibody that is capable of binding to a molecule on the immune effector cell and is also capable of binding to an antigen on a target cell.

### Chimeric Antigen Receptor (CAR)

In some embodiments, the modified immune effector cell further comprises a chimeric antigen receptor (CAR).

CARs are typically comprised primarily of 1) an extracellular antigen-binding domain comprising an antigen-binding moiety, such as a single-chain variable fragment (scFv) derived from an antigen-specific monoclonal antibody, and 2) a cytoplasmic domain comprising a lymphocyte activation domain, such as the ζ-chain from the T cell receptor CD3. These two regions are fused together via a transmembrane domain. Upon transduction, the lymphocyte expresses the CAR on its surface, and upon contact and ligation with the target antigen, it signals through the lymphocyte activation domain (e.g., CD3ζ chain) inducing cytotoxicity and cellular activation.

In some embodiments, the modified immune effector cell disclosed herein may comprise a CAR comprising, for example, (i) an extracellular antigen-binding domain, (ii) a transmembrane domain, and (iii) a cytoplasmic domain.

Constructs with only the antigen-specific binding region together with the lymphocyte activation domain are termed first-generation CARs. While activation of lymphocytes through a lymphocyte activation domain such as CD3ζ is sufficient to induce tumor-specific killing, such CARs fail to optimally induce T cell proliferation and survival in vivo. The second-generation CARs added co-stimulatory polypeptides to boost the CAR-induced immune response. For example, the co-stimulating polypeptide CD28 signaling domain was added to the CAR construct. This region generally contains the transmembrane region of the co-stimulatory peptide (in place of the CD3ζ transmembrane domain) with motifs for binding other molecules such as PI3K and Lck. T cells expressing CARs with only CD3ζ vs CARs with both CD3ζ and a co-stimulatory domain (e.g., CD28) demonstrated the CARs expressing both domains achieve greater activity. The most commonly used co-stimulating molecules include CD28 and 4-1BB, which promote both T cell proliferation and cell survival. The third-generation CAR includes three signaling domains (e.g., CD3ζ, CD28, and 4-1BB), which further improves lymphocyte cell survival and efficacy. Examples of third-generation CARs include CD19 CARs, most notably for the treatment of chronic lymphocytic leukemia (Milone, M. C., et al., (2009) Mol. Ther. 17:1453-1464; Kalos, M., et al., Sci. Transl. Med. (2011) 3:95ra73; Porter, D., et al., (2011) N. Engl. J. Med. 365:725-533, each of which is herein incorporated by reference in their entirety for all purposes). Studies in three patients showed impressive function, expanding more than a 1000-fold in vivo, and resulted in sustained remission in all three patients.

In some embodiments, the CAR expressed by a modified immune effector cell described herein comprises an extracellular antigen-binding domain and a transmembrane domain. In some embodiments, the CAR further comprises a cytoplasmic domain. Each domain is fused in frame.

In some embodiments, the CAR expressed by a modified immune effector cell described herein is a first-generation CAR. In some embodiments, the CAR expressed by a modified immune effector cell described herein is a second-generation CAR.

### Extracellular Antigen-Binding Domain of the CAR

The choice of extracellular antigen-binding domain depends upon the type and number of antigens that define the surface of a target cell. For example, the extracellular antigen-binding domain may be chosen to recognize an antigen that acts as a cell surface marker on target cells associated with a particular disease state. In some embodiments, the CARs can be genetically modified to target a tumor antigen of interest by way of engineering a desired extracellular antigen-binding domain that specifically binds to an antigen (e.g., on a cancer cell). Non-limiting examples of cell surface markers that may act as targets for the extracellular antigen-binding domain of the CAR include those associated with viral, bacterial and parasitic infections, autoimmune disease, and cancer cells.

In some embodiments, the extracellular antigen-binding domain comprises an antigen-binding polypeptide or functional variant thereof that binds to an antigen. In some embodiments, the antigen-binding polypeptide is an antibody or an antibody fragment that binds to an antigen.

In some embodiments, the antigen-binding polypeptide can be monomeric or multimeric (e.g., homodimeric or heterodimeric), or associated with multiple proteins in a non-covalent complex. In some embodiments, the extracellular antigen-binding domain may consist of an Ig heavy chain. In some embodiments, the Ig heavy chain can be covalently associated with Ig light chain (e.g., via the hinge and optionally the CH1 region). In some embodiments, the Ig heavy chain may become covalently associated with other Ig heavy/light chain complexes (e.g., by the presence of hinge, CH2, and/or CH3 domains). In the latter case, the heavy/light chain complex that becomes joined to the chimeric construct may constitute an antibody with a specificity distinct from the antibody specificity of the chimeric construct. In some embodiments, the entire chain may be used. In some embodiments, a truncated chain may be used, where all or a part of the CH1, CH2, or CH3 domains may be removed, or all or part of the hinge region may be removed. Non-limiting examples of antigen-binding polypeptides include antibodies and antibody fragments such as, e.g., murine antibodies, rabbit antibodies, human antibodies, fully humanized antibodies, single chain variable fragments (scFv), single chain antibodies, Fab fragments, F(ab′) fragments, disulfide-linked Fvs (sdFv), intrabodies, minibodies, or diabodies, camelid antibody variable domains and humanized versions, shark antibody variable domains and humanized versions, single domain antibody variable domains, nanobodies (VHHs), and camelized antibody variable domains. In some embodiments, the antigen-binding polypeptide includes an scFv.

An extracellular antigen-binding domain of the present disclosure comprises an extracellular antigen-binding moiety. In some embodiments, the extracellular antigen-binding moiety comprises an antibody or an antibody fragment that binds to an antigen. Antigen-binding moieties may comprise antibodies and/or antibody fragments such as monoclonal antibodies, multispecific antibodies, chimeric antibodies, single-chain Fvs (scFv), single chain antibodies, Fab fragments, F(ab′) fragments, disulfide-linked Fvs (sdFv), intrabodies, minibodies, single domain antibody variable domains, nanobodies (VHHs), diabodies and anti-idiotypic (anti-Id) antibodies (including, e.g., anti-Id antibodies to antigen specific TCR), and epitope-binding fragments of any of the above. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments the extracellular antigen-binding moiety comprises an scFv capable of binding to, e.g., CD19, CD22, CD123, CD33, B7-H3 (CD276), HER2, IL13Rα2, and/or EphA2.

In some embodiments, the antigen-binding moiety comprises a ligand. Non-limiting examples of CARs comprising an antigen-binding moiety comprising a ligand include IL-13 mutein-CARs or CD27-CARs. In some embodiments, the antigen-binding moiety may comprise a peptide sequence. Non-limiting examples of CARs comprising an antigen-binding moiety comprising a peptide sequence include chlorotoxin and GRP78-CARs. See, for example, PCT Patent Application WO/2021/216994, which is herein incorporated by reference in its entirety.

In some embodiments, the antigen-binding moiety binds to at least one tumor antigen. In some embodiments, the antigen-binding moiety binds to two or more tumor antigens. In some embodiments, the two or more tumor antigens are associated with the same tumor. In some embodiments, the two or more tumor antigens are associated with different tumors.

In some embodiments, the antigen-binding moiety binds to at least one antigen of an extracellular matrix. In some embodiments, the antigen-binding moiety binds to two or more antigens of the extracellular matrix. In some embodiments, the two or more tumor antigens are associated with the same extracellular matrix. In some embodiments, the two or more tumor antigens are associated with different extracellular matrices.

In some embodiments, the antigen-binding moiety binds to at least one antigen present on cells within the tumor microenvironment. In some embodiments, the antigen-binding moiety binds to two or more antigens present on cells within the tumor microenvironment. In some embodiments, the two or more antigens are associated with the same cell. In some embodiments, the two or more tumor antigens are associated with different cells.

In some embodiments, the antigen-binding moiety binds to at least one autoimmune antigen. In some embodiments, the antigen-moiety domain binds to two or more autoimmune antigens. In some embodiments, the two or more autoimmune antigens are associated with the same autoimmune disease. In some embodiments, the two or more autoimmune antigens are associated with different autoimmune diseases.

In some embodiments, the antigen-binding moiety binds to at least one infectious antigen. In some embodiments, the antigen-binding moiety binds to two or more infectious antigens. In some embodiments, the two or more infectious antigens are associated with the same infectious disease. In some embodiments, the two or more infectious antigens are associated with different infectious diseases.

In some embodiments, the tumor antigen is associated with glioblastoma, ovarian cancer, cervical cancer, head and neck cancer, liver cancer, prostate cancer, pancreatic cancer, renal cell carcinoma, bladder cancer, or hematologic malignancy. Non-limiting examples of the tumor antigens associated with cervical cancer or head and neck cancer include MUC1, Mesothelin, HER2, GD2, and EGFR. Non-limiting examples of tumor antigens associated with ovarian cancer include FOLR1, FSHR, MUC16, MUC1, Mesothelin, CA125, EpCAM, EGFR, PDGFRα, Nectin-4, B7-H3 and B7-H4. Non-limiting examples of tumor antigens associated with hematological malignancies include BCMA, GPRC5D, SLAM F7, CD33, CD19, CD22, CD79, CLL1, CD123, and CD70. Non-limiting examples of tumor antigens associated with bladder cancer include Nectin-4 and SLITRK6. Non-limiting examples of tumor antigens associated with renal cancer include CD70 and FOLR1. Non-limiting examples of tumor antigen associated with glioblastoma include FGFR1, FGFR3, MET, CD70, ROBO1, IL 13Rα2, HER2, EGFRVIII, EGFR, CD133, and PDGFRA. Non-limiting examples of tumor antigen associated with liver cancer include, EpCAM, cMET, AFP, Claudin 18.2, and GPC-3.

Additional examples of antigens that may be targeted by the antigen-binding moiety include, but are not limited to, alpha-fetoprotein, A3, antigen specific for A33 antibody, Ba 733, BrE3-antigen, carbonic anhydrase Ep-CAM, EphA1, EphA2, EphA3, EphA4, EphA5, EphA6, EphA7, EphA8, EphA10, EphB1, EphB2, EphB3, EphB4, EphB6, Flt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC2, MUC3, MUC4, NCA66, NCA95, NCA90, EX, EGFR, EGP-I, EGP-2, antigen specific for PAM-4 antibody, placental growth factor, p53, prostatic acid phosphatase, PSA, PSMA, RS5, S100, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAM5), CEACAM6, CSAp, TAC, TAG-72, tenascin, VEGF, ED-B fibronectin, COL11A1, 17-1A-antigen, TRAIL receptors, Tn antigen, Thomson-Friedenreich antigens, tumor necrosis antigens, an oncogene marker, an oncogene product, or an angiogenesis marker.

In some embodiments, the antigen is associated with an autoimmune disease or disorder. Such antigens may be derived from cell receptors and cells which produce “self”-directed antibodies. In some embodiments, the antigen is associated with an autoimmune disease or disorder such as, psoriasis, vasculitis, Wegener's granulomatosis, Hashimoto's thyroiditis, Graves' disease, chronic inflammatory demyelinating polyneuropathy, Guillain-Barre syndrome, Crohn's disease, ulcerative colitis, Rheumatoid arthritis (RA), multiple sclerosis (MS), Sjögren's syndrome, Systemic lupus erythematosus, sarcoidosis, Type 1 diabetes mellitus, insulin dependent diabetes mellitus (IDDM), autoimmune thyroiditis, reactive arthritis, ankylosing spondylitis, scleroderma, polymyositis, dermatomyositis, or Myasthenia gravis.

In some embodiments, autoimmune antigens that may be targeted by the CAR disclosed herein include, but are not limited, to islet cell antigen, platelet antigens, Sm antigens in snRNPs, myelin protein antigen, Rheumatoid factor, and anticitrullinated protein, glucose-6-phosphate isomerase, receptors such as lipocortin 1, neutrophil nuclear proteins such as lactoferrin and 25-35 kD nuclear protein, granular proteins such as bactericidal permeability increasing protein (BPI), elastase fibrinogen, fibrin, vimentin, filaggrin, collagen I and II peptides, alpha-enolase, citrullinated proteins and peptides such as CCP-1, CCP-2 (cyclical citrullinated peptides), translation initiation factor 4G1, perinuclear factor, keratin, Sa (cytoskeletal protein vimentin), circulating serum proteins such as RFs (IgG, IgM), fibrinogen, plasminogen, components of articular cartilage such as collagen II, IX, and XI, ferritin, nuclear components such as RA33/hnRNP A2, Sm, stress proteins such as HSP-65, -70, -90, BiP, inflammatory/immune factors such as B7-H1, IL-1 alpha, and IL-8, enzymes such as calpastatin, alpha-enolase, eukaryotic translation elongation factor 1 alpha 1aldolase-A, dipeptidyl peptidase, osteopontin, cathepsin G, myeloperoxidase, proteinase 3, antigen, islet cell antigen, rheumatoid factor, histones, ribosomal P proteins platelet antigens, myelin protein, cardiolipin, vimentin, nucleic acids such as, and RNA, ribonuclear particles and proteins such as Sm antigens (including but not limited to SmD's and SmB′/B), U1RNP, A2/B1 hnRNP, Ro (SSA), and La (SSB) antigens, dsDNA, and ssDNA.

In some embodiments, the antigen targeted by CARs of the present disclosure is an antigen expressed in the tumor stroma. Exemplary antigens expressed in the tumor stroma that may be targeted by CARs of the present disclosure include, but are not limited to, oncofetal splice variants of fibronectin and tenascin C, tumor-specific splice variants of collagen, and fibroblast activating protein (FAP).

In some embodiments, the antigen targeted by CARs of the present disclosure is an antigens expressed on endothelial cell. Exemplary antigens expressed on endothelial cells that may be targeted by CARs of the present disclosure include, but are not limited to, VEGF receptors, and tumor endothelial markers (TEMs).

Exemplary infectious associated antigens that may be targeted by the modified immune effector cells of the present disclosure include those derived from Adenoviridae (most adenoviruses); Arena viridae (hemorrhagic fever viruses); Birnaviridae; Bungaviridae (e.g., Hantaan viruses, bunga viruses, phleboviruses and Nairo viruses); Calciviridae (e.g., strains that cause gastroenteritis); Coronoviridae (e.g., coronaviruses); Filoviridae (e.g., ebola viruses); Flaviridae (e.g., dengue viruses, encephalitis viruses, yellow fever viruses); Hepadnaviridae (Hepatitis B virus; HBsAg); Herpesviridae (herpes simplex virus (HSV) 1 and 2, varicella zoster virus, cytomegalovirus (CMV), herpes virus); Iridoviridae (e.g., African swine fever virus); Norwalk and related viruses, and astroviruses; Orthomyxoviridae (e.g., influenza viruses); Papovaviridae (papilloma viruses, polyoma viruses); Paramyxoviridae (e.g., parainfluenza viruses, mumps virus, measles virus, respiratory syncytial virus); Parvoviridae (parvoviruses); Picornaviridae (e.g., polio viruses, hepatitis A virus; enteroviruses, human Coxsackie viruses, rhinoviruses, echoviruses); Poxviridae (variola viruses, vaccinia viruses, pox viruses); Reoviridae (e.g., reoviruses, orbiviurses and rotaviruses); Retroviridae (e.g., human immunodeficiency viruses, such as HIV-1 (also referred to as HTLV-III, LAV or HTLV-III/LAV, or HIV-III); and other isolates, such as HIV-LP); Rhabdoviradae (e.g., vesicular stomatitis viruses, rabies viruses); Togaviridae (e.g., equine encephalitis viruses, rubella viruses); and unclassified viruses (e.g., the etiological agents of Spongiform encephalopathies, the agent of delta hepatitis, the agents of non-A, non-B hepatitis (i.e., Hepatitis C)).

Additional infectious antigens that may be targeted by the modified immune effector cells of the present disclosure include bacterial antigens, fungal antigens, parasite antigens, or prion antigens, or the like. Non-limiting examples of infectious bacteria include but are not limited to: Actinomyces israelli, Bacillus antracis, Bacteroides sp., Borelia burgdorferi, Chlamydia., Clostridium perfringens, Clostridium tetani, Corynebacterium diphtheriae, Corynebacterium sp., Enterobacter aerogenes, Enterococcus sp., Erysipelothrix rhusiopathiae, Fusobacterium nucleatum, Haemophilus influenzae, Helicobacter pyloris, Klebsiella pneumoniae, Legionella pneumophilia, Leptospira, Listeria monocytogenes, Mycobacteria sps. (e.g., M tuberculosis, M avium, M gordonae, M intracellulare, M kansaii), Neisseria gonorrhoeae, Neisseria meningitidis, Pasteurella multocida, pathogenic Campylobacter sp., Rickettsia, Staphylococcus aureus, Streptobacillus monihformis, Streptococcus (anaerobic sps.), Streptococcus (viridans group), Streptococcus agalactiae (Group B Streptococcus), Streptococcus bovis, Streptococcus faecalis, Streptococcus pneumoniae, Streptococcus pyogenes (Group A Streptococcus), Treponema pallidium, and Treponema pertenue. Non-limiting examples of infectious fungi include: Cryptococcus neoformans, Histoplasma capsulatuin, Coccidioides immitis, Blastomyces dernatitidis, Chlamydia trachomatis and Candida albicans. Other infectious organisms (i.e., protists) include: Plasmodium such as Plasmodium falciparum, Plasmodium malariae, Plasmodium ovale, Plasmodium vivax, Toxoplasma gondii and Shistosoma. Other medically relevant microorganisms have been descried extensively in the literature, e.g., see C. G. A. Thomas, “Medical Microbiology”, Bailliere Tindall, Great Britain 1983, which is hereby incorporated by reference in its entirety.

Other examples of antigens that may be targeted by the modified immune cells of the present disclosure include antigens expressed on immune and/or stem cells to deplete these cells such as CD45RA and c-kit.

In some embodiments, the extracellular antigen-binding domain is specific for B7-H3, or a fragment or variant thereof. In some embodiments, the scFv capable of binding to B7-H3 may be derived from, for example, without limitation, antibodies MGA271, 376.96, 8H9, or humanized 8H9.

In a specific embodiment, the extracellular antigen binding domain comprises an scFv capable of binding to B7-H3 which may be derived from antibody MGA271. The scFv capable of binding to B7-H3 derived from antibody MGA271 may comprise the amino acid sequence of SEQ ID NO: 91, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 91. In some embodiments, the nucleotide sequence encoding the anti-B7-H3 scFV derived from antibody MGA271 comprises the nucleotide sequence that encodes the amino acid sequence of SEQ ID NO: 91, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 91. In some embodiments, the nucleotide sequence encoding the anti-B7-H3 scFV derived from antibody MGA271 comprises the sequence set forth in SEQ ID NO: 92, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 92. In some embodiments, the anti-B7-H3 scFV derived from antibody MGA271 comprises the amino acid sequence of SEQ ID NO: 91. In some embodiments, the nucleotide sequence encoding the anti-B7-H3 scFV derived from antibody MGA271 comprises the nucleotide sequence set forth in SEQ ID NO: 92.

In a specific embodiment, the extracellular antigen binding domain comprises an scFv capable of binding to B7-H3 which may be derived from antibody 8H9. The scFv capable of binding to B7-H3 derived from antibody 8H9 may comprise the amino acid sequence of SEQ ID NO: 134, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 134. In some embodiments, the nucleotide sequence encoding the anti-B7-H3 scFV derived from antibody 8H9 comprises the nucleotide sequence that encodes the amino acid sequence of SEQ ID NO: 134, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 134. In some embodiments, the nucleotide sequence encoding the anti-B7-H3 scFV derived from antibody 8H9 comprises the sequence set forth in SEQ ID NO: 135, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 135. In some embodiments, the anti-B7-H3 scFV derived from antibody 8H9 comprises the amino acid sequence of SEQ ID NO: 134. In some embodiments, the nucleotide sequence encoding the anti-B7-H3 scFV derived from antibody 8H9 comprises the nucleotide sequence set forth in SEQ ID NO: 135.

In a specific embodiment, the extracellular antigen binding domain comprises an scFv capable of binding to B7-H3 which may be derived from antibody 376.96. The scFv capable of binding to B7-H3 derived from antibody 376.96 may comprise the amino acid sequence of SEQ ID NO: 140, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 140. In some embodiments, the nucleotide sequence encoding the anti-B7-H3 scFV derived from antibody 376.96 comprises the nucleotide sequence that encodes the amino acid sequence of SEQ ID NO: 140, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 140. In some embodiments, the nucleotide sequence encoding the anti-B7-H3 scFV derived from antibody 376.96 comprises the sequence set forth in SEQ ID NO: 141, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 141. In some embodiments, the anti-B7-H3 scFV derived from 376.96 comprises the amino acid sequence of SEQ ID NO: 140. In some embodiments, the nucleotide sequence encoding the anti-B7-H3 scFV derived from antibody 376.96 comprises the nucleotide sequence set forth in SEQ ID NO: 141.

In some embodiments, the extracellular antigen-binding domain is specific for HER2, or a fragment or variant thereof. In some embodiments, the extracellular antigen-binding domain is specific for IL13Rα2, or a fragment or variant thereof. In some embodiments, the extracellular antigen-binding domain is specific for EphA2, or a fragment or variant thereof. In some embodiments, the extracellular antigen-binding domain is specific for CD123.

In a specific embodiment, the extracellular antigen-binding domain comprises an scFv capable of binding to HER2. The scFv capable of binding to HER2 may comprise the amino acid sequence of SEQ ID NO: 17, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 17. In some embodiments, the nucleotide sequence encoding the anti-HER2 scFV comprises the nucleotide sequence that encodes the amino acid sequence of SEQ ID NO: 17, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 17. In some embodiments, the nucleotide sequence encoding the anti-HER2 scFV comprises the sequence set forth in SEQ ID NO: 18, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 18. In some embodiments, the anti-HER2 scFV comprises the amino acid sequence of SEQ ID NO: 17. In some embodiments, the nucleotide sequence encoding the anti-HER2 scFV comprises the nucleotide sequence set forth in SEQ ID NO: 18.

In a specific embodiment, the extracellular antigen binding domain comprises an scFv capable of binding to IL13Rα2. The scFv capable of binding to IL 13Rα2 may comprise the amino acid sequence of SEQ ID NO: 29, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 29. In some embodiments, the nucleotide sequence encoding the anti-IL13Rα2 scFV comprises the nucleotide sequence that encodes the amino acid sequence of SEQ ID NO: 29, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 29. In some embodiments, the nucleotide sequence encoding the anti-IL13Rα2 scFV comprises the sequence set forth in SEQ ID NO: 30, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 30. In some embodiments, the anti-IL 13Rα2 scFV comprises the amino acid sequence of SEQ ID NO: 29. In some embodiments, the nucleotide sequence encoding the anti-IL 13Rα2 scFV comprises the nucleotide sequence set forth in SEQ ID NO: 30.

In a specific embodiment, the extracellular antigen binding domain comprises an scFv capable of binding to EphA2. The scFv capable of binding to EphA2 may comprise the amino acid sequence of SEQ ID NO: 38, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 38. In some embodiments, the nucleotide sequence encoding the anti-EphA2 scFV comprises the nucleotide sequence that encodes the amino acid sequence of SEQ ID NO: 38, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 38. In some embodiments, the nucleotide sequence encoding the anti-EphA2 scFV comprises the sequence set forth in SEQ ID NO: 39, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 39. In some embodiments, the anti-EphA2 scFV comprises the amino acid sequence of SEQ ID NO: 38. In some embodiments, the nucleotide sequence encoding the anti-EphA2 scFV comprises the nucleotide sequence set forth in SEQ ID NO: 39.

In a specific embodiment, the extracellular antigen-binding domain comprises an scFv capable of binding to CD123. In some embodiments, the anti-CD123 scFv is derived from antibody 26292 (scFV (292)). In some embodiments, the anti-CD123 scFv is derived from antibody 26716 (scFV (716)).

In a specific embodiment, the extracellular antigen binding domain comprises an scFv capable of binding to CD123 which may be derived from antibody 26292. The scFv capable of binding to CD123 derived from antibody 26292 may comprise the amino acid sequence of SEQ ID NO: 147, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 147. In some embodiments, the nucleotide sequence encoding the anti-CD123 scFV derived from antibody 26292 comprises the nucleotide sequence that encodes the amino acid sequence of SEQ ID NO: 147, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 147. In some embodiments, the nucleotide sequence encoding the anti-CD123 scFV derived from antibody 26292 comprises the sequence set forth in SEQ ID NO: 148, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 148. In some embodiments, the anti-CD123 scFV derived from antibody 26292 comprises the amino acid sequence of SEQ ID NO: 147. In some embodiments, the nucleotide sequence encoding the anti-CD123 scFV derived from antibody 26292 comprises the nucleotide sequence set forth in SEQ ID NO: 148.

In a specific embodiment, the extracellular antigen binding domain comprises an scFv capable of binding to CD123 which may be derived from antibody 26716. The scFv capable of binding to CD123 derived from antibody 26716 may comprise the amino acid sequence of SEQ ID NO: 149, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 149. In some embodiments, the nucleotide sequence encoding the anti-CD123 scFV derived from antibody 26716 comprises the nucleotide sequence that encodes the amino acid sequence of SEQ ID NO: 149, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 149. In some embodiments, the nucleotide sequence encoding the anti-CD123 scFV derived from antibody 26716 comprises the sequence set forth in SEQ ID NO: 150, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 150. In some embodiments, the anti-CD123 scFV derived from antibody 26716 comprises the amino acid sequence of SEQ ID NO: 149. In some embodiments, the nucleotide sequence encoding the anti-CD123 scFV derived from antibody 26716 comprises the nucleotide sequence set forth in SEQ ID NO: 150.

Various non-limiting exemplary antigen targets are also displayed in Tables 1-3.

In some embodiments, the antigen-binding moiety may comprise a VH sequence, a VL sequence, and/or CDRs thereof, such as those described in the cited publications, the contents of each publication are incorporated herein by reference in their entirety for all purposes (Table 1).

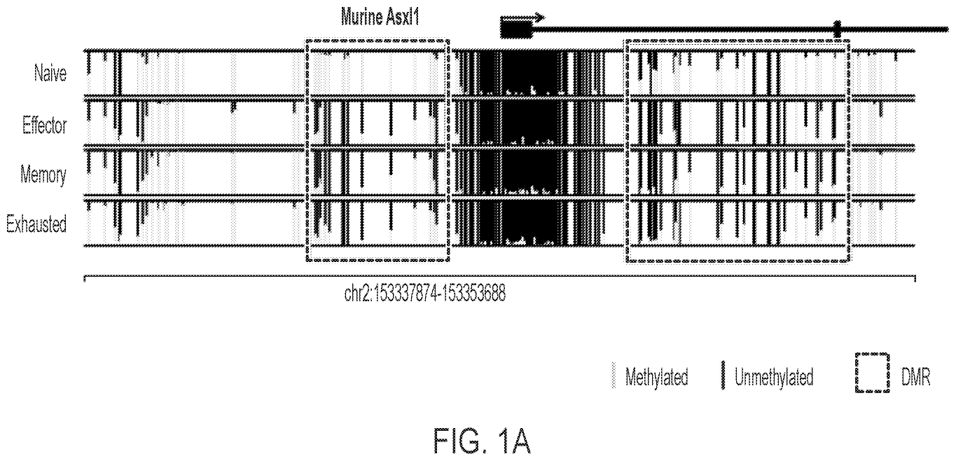

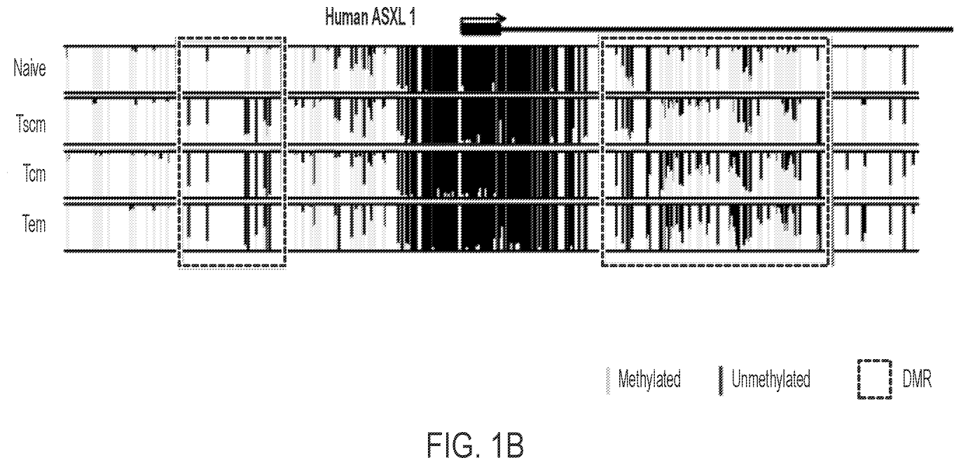

TABLE 1
Exemplary antigen-binding moieties comprising a VH sequence, a VL sequence, and/or CDRs thereof

| Antigen target | Type | Examples of Source | Type | Examples of Source |
|---|---|---|---|---|
| Oncofetal | — | — | VL | Identifier 1, 2, 7 in |
| fibronectin | | | | US20070202103A1 |
| VMS2 | VH | FIG. 1 in WO2000058363 | — | — |
| GCC1 | VH | Identifier 1 in | VL | Identifier 4, 2 in |
| | | US20160030595A1 | | US20160030595A1 |
| MVR | VH | Identifier 1 in | VL | Identifier 5 in |
| | | US20160257762A1 | | US20160257762A1 |
| OlfmB | VH | Identifier 1 in | VL | Identifier 2 in |
| | | WO2015054441A1 | | WO2015054441A1 |
| TRBC1 | VH | Identifier 1 in | VL | Identifier 2 in |
| | | WO2015132598 | | WO2015132598 |
| Malignant | VH | Identifier 1 in | VL | Identifier 5 in |
| Variable | | WO2015133817A1 | | WO2015133817A1 |
| Receptor | | | | |
| CD40 | VH | Identifier 1 in | VL | Identifier 2 in |
| | | WO2016069919; Identifier | | WO2016069919; Identifier |
| | | 5, 7, 8 in WO2015091655 | | 6 in WO2015091655 |
| CDIM | VH | Identifier 1, 10, 11, 12, 13, | VL | Identifier 28, 29, 30, 31, |
| | | 14, 15, 16, 17, 18, 19, 2, 20, | | 32, 33, 34, 35, 36, 37, 38, |
| | | 21, 22, 3, 4, 5, 6, 7, 8, 9 in | | 39, 40, 41, 42, 43, 44, 45, |
| | | WO2013120012 | | 46, 47, 48, 49 in |
| | | | | WO2013120012 |
| ILIRAP | VH | Identifier 1, 10, 19, 8, 9 in | VL | Identifier 14, 15, 17, 18, 2, |
| | | WO2016020502; Identifier | | 20 in WO2016020502; |
| | | 120, 122, 124 in | | Identifier 121, 123, 125 in |
| | | WO2016179319A1 | | WO2016179319A1 |
| ALK | VH | Identifier 1, 11, 13, 15, 3, 5, | VL | Identifier 10, 12, 14, 16, 2, |
| | | 7, 9 in US20160280798A1; | | 4, 6, 8 in |
| | | Identifier 9, 1, 3, 5, 11, 13, | | US20160280798A1; |
| | | 15, 7, 9 in WO2015069922 | | Identifier 10, 12, 14, 16, 8 |
| | | | | in WO2015069922; |
| | | | | Identifier 2, 4, 6 in |
| | | | | WO2015069922 |
| LHR | VH | Identifier 1, 2, 3, 4, 5, 6, 7, 8 | — | — |
| | | in WO2016160618A3 | | |
| MUC16 | VH | Identifier 1, 21, 41, 81, 61 in | VL | Identifier 2, 22, 42, 62, 82 |
| | | WO2016149368; Identifier | | in WO2016149368 |
| | | 11, 4, 6, 8 in | | |
| | | US20130171152 | | |
| PTK7 | VH | Identifier 1, 25, 49 in | VL | Identifier 20, 22, 24, 26, |
| | | US20150315293; Identifier | | 28, 30, 32, 34, 36, 38, 40, |
| | | 21, 23, 25, 27, 29, 31, 33, | | 42, 44, 46, 48, 50, 52, 54, |
| | | 35, 37, 39, 41, 43, 45, 47, | | 56, 58, 60, 62, 64, 66, 68 in |
| | | 49, 51, 53, 55, 57, 59, 61, | | WO2012112943A1; |
| | | 63, 65, 67, 69 in | | Identifier 15, 39, 63 in |
| | | WO2012112943A1 | | US20150315293 |
| ANG2 | VH | Identifier 1, 3 in | VL | Identifier 2, 4 in |
| | | WO2015091655 | | WO2015091655 |
| CD71 | VH | Identifier 1, 3, 325, 4, 5, 699 | VL | Identifier 2,327, 329, 331, |
| | | in US20160355599 | | 333, 335, 337, 6, 650, 652, |
| | | | | 654, 656, 658, 660, 670, |
| | | | | 671, 672, 673, 7, 701, 702, |
| | | | | 703, 704, 705, 706, 707, |
| | | | | 708, 709, 710, 711, 712, |
| | | | | 721, 722, 723, 724, 725, |
| | | | | 726, 727, 728, 729, 730, |
| | | | | 731, 732, 733, 734, 735, |
| | | | | 736, 737, 738, 739, 740, |
| | | | | 741, 742, 743, 744, 745, |
| | | | | 746, 747, 748, 749, 750, |
| | | | | 751, 752, 753, 754, 755, |
| | | | | 756, 757, 758, 759, 760, |
| | | | | 761, 762, 763, 764, 765, |
| | | | | 766, 767, 768, 769, 770, |
| | | | | 771, 772, 773, 774, 775, |
| | | | | 776, 777, 778, 779, 780, |
| | | | | 781, 782, 783, 784 785, |
| | | | | 786, 787, 788, 8, 810, 811, |
| | | | | 812, 813, 814, 815, 816, |
| | | | | 817, 818, 819, 820, 821, |
| | | | | 822, 823, 824, 825, 826, |
| | | | | 827, 828, 829, 830, 831, |
| | | | | 832, 833, 834, 835, 836, |
| | | | | 810, 842, 843, 844, 845, |
| | | | | 846, 847, 848, 849, 850, |
| | | | | 851, 852, 853, 854, 855, |
| | | | | 856, 857, 858, 859, 860, |
| | | | | 861, 862, 863, 864, 865, |
| | | | | 866, 867, 868, 869, 870, |
| | | | | 871, 872, 873, 874, 875, |
| | | | | 876, 877, 879, 879, 880, |
| | | | | 881, 882, 883, 884, 885, |
| | | | | 886, 887, 888, 889, 890, |
| | | | | 891, 892, 893, 894, 895, |
| | | | | 896, 897, 898, 899, 900, |
| | | | | 901, 902, 903, 904, 905, |
| | | | | 906, 907, 908 in |
| | | | | US20160355599 |
| TEM8 | VH | Identifier 1, 3, 5, 7 in | VL | Identifier 4, 6, 8 in |
| | | US20160264662A1 | | US20160264662A1 |
| IGFI | VH | Identifier 1, 3, 7 in | VL | Identifier 2, 4, 6, 8 in |
| | | WO2007118214; Identifier | | WO2007118214 |
| | | 7 in WO2015073575 | | |
| FAP | VH | Identifier 1, 5 in | VL | Identifier 2, 6 in |
| | | WO2015118030; Identifier | | WO2015118030; Identifier |
| | | 170, 172 in | | 171, 173 in |
| | | WO2016120216; Identifier | | WO2016120216; Identifier |
| | | 8 in US20160326265 A1 | | 9 in US20160326265A1 |
| Mesothelin | VH | Identifier 1, 6 in | VL | Identifier 3, 5 |
| | | WO2015188141; Identifier | | WO2015188141; Identifier |
| | | 119, 50 in | | 1, 2, 3 in WO2013142034; |
| | | US20160333114A1; | | Identifier 11, 15, 19, 23, 27 |
| | | Identifier 5, 6 in | | in US20160229919A1; |
| | | WO2013142034; Identifier | | Identifier 120, 47, 49 in |
| | | 15, 2 in U.S. Pat. No. 9,416,190B2; | | US20160333114A1 |
| | | Identifier 13, 17, 21, 25, 29, | | |
| | | 9 in US20160229919A1 | | |
| Frizzled | VH | Identifier 10 in | VL | Identifier 12, 14 in |
| Receptor | | WO2010037041 | | WO2010037041 |
| APCDD1 | VH | Identifier 10, 102, 106, 110, | VL | Identifier 136, 100, 104, |
| | | 114, 118, 122, 126, 130, | | 108, 112, 116, 12, 120, |
| | | 134, 14, 6, 98 in | | 124, 128, 132, 16, 8 in |
| | | WO2012019061 | | WO2012019061 |
| CSF | VH | Identifier 10, 102, 14, 18, 2, | VL | Identifier 12, 32, 44, 48, 60 |
| | | 22, 26, 30, 34, 38, 46, 50, | | in US20050059113A1 |
| | | 54, 58, 6, 62, 66, 70, 74, 78, | | |
| | | 82, 86, 90, 94, 98 in | | |
| | | US20050059113A1 | | |
| TIGIT | VH | Identifier 10, 11, 12, 124, | VL | Identifier 130, 131, 132, |
| | | 125, 126, 127, 128, 129, 13, | | 133, 137, 139, 145, 146, |
| | | 136, 138, 14, 143, 144, 149, | | 151, 152, 25, 26, 27, 28, |
| | | 15, 150, 16, 17, 18, 19, 20, | | 29, 30, 50, 51, 52, 64, 95, 8 |
| | | 21, 22, 23, 24, 37, 38, 39, | | in US20160355589 |
| | | 40, 41, 42, 43, 44, 45, 46, | | |
| | | 47, 63, 94, 7, 9 in | | |
| | | US20160355589 | | |
| B7H3 | VH | Identifier 10, 11, 12, 13, 14, | VL | Identifier 1, 2, 3, 4, 5, 6, 7, |
| | | 15, 16, 9 in WO2016033225 | | 8 in WO2016033225 |
| IL4R | VH | Identifier 10, 11, 14, 15, 9 in | VL | Identifier 13, 7, 8 in |
| | | WO2009121847 | | WO2009121847 |
| Lymphotoxin | VH | Identifier 10, 12, 14, 16, 2 in | VL | Identifier 1, 15, 4, 6, 8 in |
| beta receptor | | WO2004002431 | | WO2004002431 |
| LGR5 | VH | Identifier 10, 12, 16, 18, 20, | VL | Identifier 15, 19, 21, 23, |
| | | 22, 24, 26, 4 in | | 25, 3 in US20160102146 |
| | | US20160102146 | | |
| CD148 | VH | Identifier 10, 14, 18, 2, 22, | VL | Identifier 12, 16, 20, 24, |
| | | 26, 30, 6 in WO2005118643 | | 28, 32, 4, 8 in |
| | | | | WO2005118643 |
| GPC3 | VH | Identifier 10, 14, 2, 3, 4, 5, | VL | Identifier 10, 14, 18, 22, |
| | | 6, 7, 8, 9 in | | 24, 26 in U.S. Pat. No. 9409994B2; |
| | | US20160208015 A1; | | Identifier 16, 31 in |
| | | Identifier 22 in | | US20160208015A1; |
| | | WO2016049459; Identifier | | Identifier 23 in |
| | | 12, 16, 20, 37, 8 in | | WO2016049459 |
| | | U.S. Pat. No. 9,409,994B2 | | |
| CSPG4 | VH | Identifier 10, 16, 18, 4, 6, 8 | VL | Identifier 7 in |
| | | in WO2016077638; | | WO2016164429; Identifier |
| | | Identifier 8 in | | 12, 14 in WO2016077638 |
| | | WO2016164429 | | |
| AGR2 | VH | Identifier 10, 18 in | VL | Identifier 11, 19 in |
| | | WO2016040321 | | WO2016040321 |
| Tissue factor | VH | Identifier 10, 19, 23, 27, 29, | VL | Identifier 25. 31 in |
| | | 6 in WO2004094475; | | US20040229301A1; |
| | | Identifier 38 in | | Identifier 12, 21, 25, 31, 8 |
| | | US20160333114A1 | | in WO2004094475; |
| | | | | Identifier 35, 37 in |
| | | | | US20160333114A1 |
| SEMAPHORIN | VH | Identifier 10, 25, 9 in | VL | Identifier 17, 18, 29 in |
| 4D | | US20160115240A1 | | US20160115240A1 |
| PDL1 | VH | Identifier 10, 32, 8 in | VL | Identifier 22, 26, 34, 42, |
| | | US20160319022; Identifier | | 58, 66, 74, 82, 86 in |
| | | 18, 30, 38, 46, 50, 54, 62, | | WO2016061142; Identifier |
| | | 70, 78 in WO2016061142; | | 30, 8, 9 in |
| | | Identifier 29, 7 in | | US20150190506; Identifier |
| | | US20150190506; Identifier | | 7, 9 in US20160319022; |
| | | 16, 18, 197, 247, 248, 250, | | Identifier 17, 22, 24, 249, |
| | | 251, 252, 253, 254, 255, | | 26, 28, 309, 311, 313, 320, |
| | | 256, 257, 258, 259, 260, 30, | | 325, 34, 340, 357, 359, 36, |
| | | 308, 310, 312, 319, 32, 324, | | 42, 44, 58, 60, 66, 68, 74, |
| | | 339, 356, 38, 40, 46, 48, 50, | | 76, 8, 82, 84, 86, 88 in |
| | | 52, 54, 6, 62, 70, 72, 78, 80, | | US20160108123 |
| | | 91, 96 in US20160108123; | | |
| | | Identifier 358, 56, 64 in | | |
| | | US20160108123 | | |
| HLAG | VH | Identifier 10, 8 in | VL | Identifier 18, 20 in |
| | | WO2016160622A2 | | WO2016160622A2 |
| B7H4 | VH | Identifier 100, 101, 102, | VL | Identifier 104, 11, 126, |
| | | 103, 107, 108, 109, 110, | | 134, 138, 19, 27, 3, 35, 55, |
| | | 11l, 112, 113, 114, 12, 127, | | 93, 95, 97, 98, 145, 146, |
| | | 130, 131, 132, 133, 137, 2, | | 147, 148 in |
| | | 20, 28, 36, 37, 38, 4, 56, 99, | | US20160159910; Identifier |
| | | 144 in US20160159910; | | 29, 31, 33 in |
| | | Identifier 13, 15, 17 in | | WO2016160620 |
| | | WO2016160620 | | |
| VEGFR2 | VH | Identifier 100, 101, 102, | VL | Identifier 107, 108, 109, |
| | | 103, 114, 115, 116, 117, | | 110, 111, 112, 113, 86, 87, |
| | | 118, 119, 120, 121, 122, | | 88, 89, 90, 91, 92, 93, 94 in |
| | | 123, 124, 95, 96, 97, 98, 99 | | WO2017004254 |
| | | in WO2017004254 | | |
| CD73 | VH | Identifier 100, 103, 107, | VL | Identifier 12. 20, 44, 72, |
| | | 109, 112, 114, 116, 119, | | 76, 8, 84, 92 in |
| | | 121, 16, 32, 4, 52, 60, 68, | | US20160145350; Identifier |
| | | 80, 88 in US20160145350; | | 22, 29, 37, 4 in |
| | | Identifier 135, 40, 21, 3, 28, | | WO2016055609A1; |
| | | 36 in WO2016055609A1 | | Identifier 101, 102, 104, |
| | | | | 106, 110, 117, 118, 120, |
| | | | | 122 in US20160145350 |
| Claudin | VH | Identifier 101, 103, 105, | VL | Identifier 114, 116, 118, |
| | | 107, 109, 111, 113, 115, | | 120, 22, 25, 29, 33, 37, 41, |
| | | 117, 119, 121, 122, 123, | | 45, 49, 53, 57, 61, 65, 69, |
| | | 124, 125, 126, 127, 128, | | 73, 77 in |
| | | 129, 130, 131, 132, 133, | | WO2016073649A1 |
| | | 134, 135, 136, 137, 138, | | |
| | | 139, 140, 141, 142, 143, | | |
| | | 144, 145, 146, 147, 148, | | |
| | | 149, 150, 23, 27, 31, 35, 39, | | |
| | | 43, 47, 51, 55, 59, 63, 67, | | |
| | | 71, 75, 79, 81, 83, 85, 87, | | |
| | | 89, 91, 93, 95, 97, 99 in | | |
| | | WO2016073649A1 | | |
| CLL3 | VH | Identifier 101, 103, 105, | VL | Identifier 100, 102, 104, |
| | | 107, 109, 111, 113, 115, | | 106, 108, 110, 112, 114, |
| | | 117, 119, 121, 123, 125, | | 116, 118, 120, 122, 124, |
| | | 127, 129, 131, 133, 135, | | 126, 128, 130, 132, 134, |
| | | 137, 139, 141, 145, 147, | | 136, 138, 140, 144, 146, |
| | | 149, 151, 153, 155, 157, | | 148, 150, 152, 154, 156, |
| | | 159, 161, 163, 165, 167, | | 158, 160, 162, 164, 166, |
| | | 169, 171, 173, 175, 177, | | 170, 172, 174, 176, 178, |
| | | 179, 181, 183, 185, 187, | | 180, 182, 184, 186, 190, |
| | | 191, 193, 195, 197, 199, | | 192, 194, 196, 198, 20, |
| | | 201, 203, 205, 207, 209, 21, | | 200, 202, 204, 206, 208, |
| | | 211, 213, 23, 25, 27, 29, 31, | | 210, 212, 22, 24, 26, 28, |
| | | 33, 35, 37, 39, 41, 43, 45, | | 30, 32, 34, 36, 38, 40, 42, |
| | | 47, 49, 51, 53, 55, 57, 59, | | 44, 46, 48, 50, 54, 56, 58, |
| | | 61, 63, 65, 67, 69, 71, 73, | | 60, 62, 64, 66, 68, 70, 72, |
| | | 75, 77, 79, 81, 83, 85, 87, | | 74, 76, 78, 80, 82, 84, 86, |
| | | 89, 91, 93, 95, 97, 99 in | | 88, 90, 92, 94, 96, 98 in |
| | | US20170000901 | | US20170000901 |
| OX40 | VH | Identifier 101, 103, 105, | VL | Identifier 10, 45, 47, 49, 8 |
| | | 107, 109, 111, 113, 115, | | in U.S. Pat. No. 8,283,450; Identifier |
| | | 117, 119, 121, 123, 124, | | 11, 7 in U.S. Pat. No. 9,428,570; |
| | | 125, 17, 28, 318, 37, 48, 50, | | Identifier 116, 120, 122, |
| | | 58, 66,, 74, 85, 93, 95, 97, | | 30, 38, 49, 57, 65, 73, 84, |
| | | 99 in WO2016196228; | | 86, 94, 98 in |
| | | Identifier 31, 34, 36, 38, 40, | | WO2016196228; Identifier |
| | | 42, 44, 46, 48, 50, 53, 54, | | 24, 26, 27, 28, 30, 60, 8, |
| | | 55, 58, 59, 61 in | | 81, 82, 83, 84, 85, 86, 87, |
| | | US20150190506; Identifier | | 88, 89 in U.S. Pat. No. 8,748,585; |
| | | 33, 35, 37, 39, 41, 43, 45, | | Identifier 30, 32 in |
| | | 47, 49, 51, 53, 55, 57, 59, | | US20160137740; Identifier |
| | | 61, 63, 65, 67, 71 in | | 32, 35, 39, 41, 43, 45, 47, |
| | | US20160137740; Identifier | | 49, 51, 52, 56, 57, 62 in |
| | | 44, 46, 48, 7, 9 in | | US20150190506; Identifier |
| | | U.S. Pat. No. 8,283,450; Identifier 9, 15 | | 29, 37 in US20160137740 |
| | | in U.S. Pat. No. 9,428,570; Identifier | | |
| | | 19, 21, 22, 23, 29, 58, 59, 7, | | |
| | | 77, 78, 79, 80 in | | |
| | | U.S. Pat. No. 8,748,585 | | |
| MUCIN1 | VH | Identifier 101, 106, 109, | VL | Identifier 148, 158, 162, |
| | | 115, 119, 123, 127, 141, 15, | | 167, 170, 174, 184, 190, |
| | | 23, 28, 33, 39, 42, 47, 5, 57, | | 193, 203, 208, 211, 220, |
| | | 66, 70, 75, 80, 83, 87, 92 in | | 225, 229, 234, 242, 246, |
| | | EP3049812A2 | | 250, 255, 261, 270, 275, |
| | | | | 279, 283, 291, 297, 303, |
| | | | | 308, 315, 319, 323, 333, |
| | | | | 340 in EP3049812A2 |
| MCSF | VH | Identifier 102, 10, 14, 18, 2, | VL | Identifier 8, 32, 52, 60, 28, |
| | | 22, 26, 30, 34, 38, 46, 50, | | 36, 4, 44, 48, 56, 62, 12, |
| | | 54, 58, 6, 66, 70, 74, 78, 82, | | 16, 20, 24 in |
| | | 86, 90, 94, 98 in | | WO2005030124 |
| | | WO2005030124 | | |
| LAG3 | VH | Identifier 102, 106, 110, | VL | Identifier 32, 36, 40, 44, |
| | | 113, 122, 18, 30, 66, 70, 74, | | 48, 52, 56, 60, 84, 88, 92, |
| | | 78 in US20150259420; | | 96, 134, 34, 38, 42, 46, 50, |
| | | Identifier 100, 104, 108, 28, | | 54, 58, 60, 86, 90, 94, 98 in |
| | | 64, 68, 72, 76, 8, 80 in | | US20150259420; Identifier |
| | | US20150259420; Identifier | | 2 in WO2015042246 |
| | | 1 in WO2015042246 | | |
| TIM3 | VH | Identifier 102, 112, 12, 2, | — | — |
| | | 22, 32, 42, 52, 62, 72, 82, 91 | | |
| | | in US20150086574; | | |
| | | Identifier 82 in | | |
| | | WO2013006490; Identifier | | |
| | | 13, 21, 29, 37, 45, 5, 53, 61, | | |
| | | 69, 77, 85, 93 in | | |
| | | WO2016179319A1; | | |
| | | Identifier 7 in | | |
| | | WO2013006490; Identifier | | |
| | | 107, 117, 17, 27, 37, 47, 57, | | |
| | | 67, 7, 77, 87, 97 in | | |
| | | US20150086574; Identifier | | |
| | | 17, 25, 33, 41, 49, 57, 65, | | |
| | | 73, 81, 89, 9, 97 in | | |
| | | WO2016179319A1 | | |
| CD2 | VH | Identifier 103, 117, 119 in | VL | Identifier 102, 116 in |
| | | WO2016122701 | | WO2016122701 |
| CD52 | VH | Identifier 103, 136, 137 in | VL | Identifier 102, 138 in |
| | | WO2010132659 | | WO2010132659 |
| WT1/HLA | VH | Identifier 104, 111, 128, 14, | VL | Identifier 106, 112, 130, |
| Bispecific | | 32, 50, 68, 86 in | | 34, 52, 70, 88 in |
| | | WO2015070061 | | WO2015070061 |
| CD3 | VH | Identifier 108, 112, 115 in | VL | Identifier 104 in |
| | | WO2016122701; Identifier | | WO2016122701; Identifier |
| | | 29 in WO2014144722 A2; | | 13 in WO2016126213A1 |
| | | Identifier 12 in | | |
| | | WO2016126213A1 | | |
| PG9 | VH | Identifier 11 in | VL | Identifier 10 in |
| | | EP3074419A2 | | EP3074419A2 |
| V2 | VH | Identifier 11 in | — | — |
| | | US20160194375A1 | | |
| HSP70 | VH | Identifier 11, 12 in | VL | Identifier 16, 17 in |
| | | WO2016120217 | | WO2016120217 |
| CD37 | VH | Identifier 11, 12, 18 in | VL | Identifier 14, 15 in |
| | | US20170000900 | | US20170000900 |
| CD123 | VH | Identifier 11, 13, 14, 21 in | VL | Identifier 9, 11, 18, 19, 20, |
| | | WO2015140268A1; | | 21, 22, 23 in |
| | | Identifier 113, 115, 57, 59, | | WO2016120220; Identifier |
| | | 63 in WO2016120216; | | 12, 16, 18, 19, 22 in |
| | | Identifier 12, 123, 24, 25, | | WO2015140268A1; |
| | | 26, 27, 28, 29, 30, 9 in | | Identifier 275, 276, 277, |
| | | WO2016120220; Identifier | | 278, 307, 308, 309, 310 in |
| | | 216, 217, 218, 219, 274 in | | WO2016028896; Identifier |
| | | WO2016028896 | | 5 in US20160333108A1; |
| | | | | Identifier 114, 116, 58, 60, |
| | | | | 64 in WO2016120216 |
| GD3 | VH | Identifier 11, 13, 15, 17 in | VL | Identifier 12, 14, 16, 18 in |
| | | WO2016185035A1 | | WO2016185035A1 |
| TAG72 | VH | Identifier 115 in | VL | Identifier 116 in |
| | | US20160333114A1 | | US20160333114A1 |
| MCAM | VH | Identifier 115, 116, 117, | VL | Identifier 109, 110, 111, |
| | | 118, 119, 157, 158, 159, | | 112, 121, 122, 123 in |
| | | 160, 161, 178, 179 in | | US20150259419; Identifier |
| | | US20150259419; Identifier | | 30, 40, 50, 60, 70, 71, 72 in |
| | | 35, 45, 55, 65, 77, 89 in | | US20150239980 |
| | | US20150239980; Identifier | | |
| | | 101, 102, 103, 104, 105, | | |
| | | 106, 107 in | | |
| | | US20150259419 | | |
| CA19.9 | VH | Identifier 117 in | VL | Identifier 118 in |
| | | US20160333114A1 | | US20160333114A1 |
| BMPR1A | VH | Identifier 12 in | — | — |
| | | WO2011116212 | | |
| LGR4 | VH | Identifier 12, 13, 5, 9 in | VL | Identifier 10, 11, 6 in |
| | | US20160046723 | | US20160046723 |
| APRIL | VH | Identifier 12, 14, 16. 18. 3. | VL | Identifier 20, 22, 24, 26, |
| | | 32, 34, 36, 38, 40, 42, 44, | | 28, 30, 4, 50 in |
| | | 46, 48, 52 in | | US2016026467 |
| | | US20160264674 | | |
| FcRL5 | VH | Identifier 12, 16, 20, 24, 28, | VL | Identifier 11, 15, 19, 23, |
| (FcReceptorLike | | 32, 36, 4, 40, 44, 48, 8, 915, | | 27, 3, 31, 35, 39, 43, 47, 7, |
| 5) | | 919 in WO2016090337 | | 917, 921 WO2016090337 |
| CLDN18.2 | VH | Identifier 12, 2 in | VL | Identifier 13, 3 in |
| | | US20160347815A1 | | US20160347815A1 |
| ROR1 | VH | Identifier 12, 20, 28, 36, 44, | VL | Identifier 16, 24, 32, 40, |
| | | 60, 68 WO2016016343A1; | | 56, 64, 72, 36, 62, 23, 49, |
| | | Identifier 57, 19, 31, 45, 53, | | 58 WO2016016343A1; |
| | | 71 in WO2016016344A1; | | Identifier 86, 88, 90 in |
| | | Identifier 85, 87, 89 in | | WO2016120216; Identifier |
| | | WO2016120216; Identifier | | 126, 127, 234, 235, 236, |
| | | 122, 125, 175, 176, 179, | | 237, 238, 240, 241, 242, |
| | | 180, 181, 182, 183, 184, | | 243, 244, 245, 246, 247, |
| | | 185, 186, 187, 188, 189, | | 248 in US20160208018A1; |
| | | 190, 191, 192, 193, 194, | | Identifier 56 in |
| | | 195, 196, 197, 197, 199, | | EP3083671A1; Identifier |
| | | 200, 201, 202, 203, 204, | | 103, 111, 127, 135, 143, |
| | | 205, 206, 207, 208, 209 in | | 15, 151, 159, 167, 175, |
| | | US20160208018A1; | | 183, 191, 199, 207, 215, |
| | | Identifier 55 in | | 223, 23, 231, 239, 247, |
| | | EP3083671Al; Identifier | | 255, 263, 271, 279, 287, |
| | | 104, 112, 120, 128, 152, 16, | | 295, 303, 31, 311, 319, |
| | | 160, 168, 176, 184, 192, | | 327, 335, 343, 351, 359, |
| | | 200, 208, 216, 224, 232, 24, | | 39, 47, 55, 63, 7, 71, 79, |
| | | 240, 248, 256, 264, 272, | | 87, 95 in |
| | | 280, 288, 296, 304, 312, 32, | | WO2016187216A1 |
| | | 320, 336, 344, 352, 360, 40, | | |
| | | 48, 56, 64, 72, 8, 80, 88 in | | |
| | | WO2016187216A1 | | |
| B7H1 | VH | Identifier 12, 32, 42, 52, 72, | VL | Identifier 17, 37, 47, 57, 7, |
| | | 2, 62 in US20130034559 | | 77, 27, 67 in |
| | | | | US20130034559 |
| CD32B | VH | Identifier 127 in | VL | Identifier 126 in |
| | | WO2016122701 | | WO2016122701 |
| CD64 | VH | Identifier 129 in | VL | Identifier 128 in |
| | | WO2016122701 | | WO2016122701 |
| PG16 | VH | Identifier 13 in | VL | Identifier 12 in |
| | | EP3074419A2 | | EP3074419A2 |
| MPER | VH | Identifier 13 in | VL | Identifier 12 in |
| | | US20160194375A1 | | US20160194375A1 |
| CD105 | VH | Identifier 13, 14, 16 in | VL | Identifier 1, 17, 20, 22, 23 |
| | | WO2014039682 | | in WO2014039682 |
| CLL1 | VH | Identifier 13, 14, 15, 17, 19, | VL | Identifier 16, 18, 20, 22, |
| | | 21, 23, 25, 27, 29, 31, 33, 35 | | 24, 26, 28, 30, 32, 24, 36 in |
| | | in WO2016120219; | | WO2016120219; Identifier |
| | | Identifier 195, 65, 66, 67, | | 196, 78, 79, 80, 81, 82, 83, |
| | | 68, 69, 70, 71, 72, 73, 74, | | 84, 85, 86, 87, 88, 89, 90 in |
| | | 75, 76, 77 in | | WO2016014535; Identifier |
| | | WO2016014535; Identifier | | 30, 32, 35, 37, 39, 41 in |
| | | 31, 33, 34, 36, 38, 40, 42, 46 | | US20160075787; Identifier |
| | | in US20160075787; | | 152, 104, 106, 108, 110, |
| | | Identifier 150, 103, 105, | | 112, 114, 116, 118 in |
| | | 107, 109, 111, 113, 115, 117 | | WO2016179319A1 |
| | | in WO2016179319A1 | | |
| GPRC5D | VH | Identifier 13, 17, 21, 25, 29, | VL | Identifier 10, 14, 18, 2, 22, |
| | | 314, 326, 33, 338, 350, 362, | | 26, 30, 303, 315, 327, 339, |
| | | 37, 374, 386, 41, 45, 49, 5, | | 34, 351, 363, 375, 38, 387, |
| | | 53, 57, 61, 65, 69, 73, 77, | | 42, 46, 50, 54, 58, 6, 62, |
| | | 81, 85, 89, 93, 1, 9 in | | 66, 70, 74, 78, 82, 86, 94 in |
| | | WO2016090312 | | WO2016090312 |
| EFNA4 | VH | Identifier 13, 39 in | — | — |
| | | US20150125472 | | |
| CD79 | VH | Identifier 131 in | VL | Identifier 130 in |
| | | WO2016122701 | | WO2016122701 |
| FGFR3 | VH | Identifier 132, 134, 136 in | VL | Identifier 133, 135, 137, |
| | | U.S. Pat. No. 9,499,623 | | 139 in U.S. Pat. No. 9,499,623 |
| TCR | VH | Identifier 133 in | VL | Identifier 132 in |
| | | WO2016122701 | | WO2016122701 |
| MN | VH | Identifier 133, 135, 137, | VL | Identifier 134, 136, 138, |
| | | 139, 141, 143, 145, 147, | | 140, 142, 144, 146, 148, |
| | | 149, 151 in WO2007070538 | | 150, 152 in |
| | | | | WO2007070538 |
| IL33 | VH | Identifier 134, 136, 138, | VL | Identifier 135, 137, 139, |
| | | 185, 187, 189, 216, 218, | | 184, 188, 217, 219, 237, |
| | | 220, 221,236, 246, 282, 284, | | 247, 283, 285, 287, 37, 39, |
| | | 286, 36, 38, 40, 84, 86, 88 | | 41, 87 in US20160168242 |
| | | in US20160168242 | | |
| NKG2D | VH | Identifier 135, 137 in | VL | Identifier 134, 136 in |
| | | WO2016122701 | | WO2016122701 |
| CD30 | VH | Identifier 14, 16 in | VL | Identifier 13, 15 in |
| | | WO2016134284 | | WO2016134284 |
| EGFR | VH | Identifier 14, 50, 9 in | VL | Identifier 15 in |
| | | WO2015143382; Identifier | | WO2015143382; Identifier |
| | | 12, 14, 15, 21 in | | 14 in WO2014143765; |
| | | US20100008978A1; | | Identifier 4051, 4052, |
| | | Identifier 2123 in | | 4053, 4054, 4055, 4056, |
| | | WO2018231759 | | 4057, 4058, 4059, 4060, |
| | | | | 4061, 4062, 4063, 4064, |
| | | | | 4065, 4066, 4067, 4068, |
| | | | | 4069, 4070, 4071, 4072, |
| | | | | 4073, 4074, 4075, 4076, |
| | | | | 4077, 4078, 4079, 4080, |
| | | | | 4081, 4082, 4083, 4084, |
| | | | | 4085, 4086, 4087, 4088, |
| | | | | 4089. 4090, 4091, 4092, |
| | | | | 4093, 4094, 4095, 4096, |
| | | | | 4097, 4098, 4099, 4100, |
| | | | | 4101, 4102, 4103, 4104, |
| | | | | 4105, 4106, 4107, 4108, |
| | | | | 4109, 4110, 4111, 4112, |
| | | | | 4113, 4114, 4115, 4116, |
| | | | | 4117, 4118, 4119, 4120, |
| | | | | 4121, 4122, 4123, 4124, |
| | | | | 4125, 4126, 4127, 4128, |
| | | | | 4129, 4130, 4131, 4132, |
| | | | | 4133, 4134, 4135, 4136, |
| | | | | 4137, 4138, 4139, 4140, |
| | | | | 4141, 4142, 4143, 4144, |
| | | | | 4145, 4146, 4147, 4148, |
| | | | | 4149, 4150, 4151, 4152, |
| | | | | 4153, 4154, 4155, 4156, |
| | | | | 4157, 4158, 4159, 4160, |
| | | | | 4161, 4162, 4163, 4164, |
| | | | | 4165, 4166, 4167, 4168, |
| | | | | 4169, 4170, 4171, 4172, |
| | | | | 4173, 4174, 4175, 4176, |
| | | | | 4177, 4178, 4179, 4180, |
| | | | | 4181, 4182, 4183, 4184, |
| | | | | 4185, 4186, 4187, 4188, |
| | | | | 4189, 4190, 4191, 4192, |
| | | | | 4193, 4194, 4195, 4196, |
| | | | | 4197, 4198, 4199, 4200, |
| | | | | 4201, 4202, 4203, 4204, |
| | | | | 4205, , 4206, 4207, 4208, |
| | | | | 4209, 4210, 4211, 4212, |
| | | | | 4213, 4214, 4215, 4216, |
| | | | | 4217, 4218, 4219, 4220, |
| | | | | 4221, 4222, 4223, 4224, |
| | | | | 4225, 4226, 4227, 4228, |
| | | | | 4229, 4230, 4231, 4232, |
| | | | | 4233, 4234, 4235, 4236, |
| | | | | 4237, 4238, 4239, 4240, |
| | | | | 4241, 4242, 4243, 4244, |
| | | | | 4245, 4246, 4247, 4248, |
| | | | | 4249, 4250, 4251, 4252, |
| | | | | 4253, 4254, 4255, 4256, |
| | | | | 4257, 4258, 4259, 4260, |
| | | | | 4261, 4262, 4263, 4264, |
| | | | | 4265, 4266, 4267, 4268, |
| | | | | 4269, 4270, 4271, 4272, |
| | | | | 4273, 4274, 4275, 4276, |
| | | | | 4277, 4278, 4279, 42870, |
| | | | | 4281, 4282, 42834284, |
| | | | | 4285, 4286, 4287, 4288, |
| | | | | 4289, 4290, 4291, 4292, |
| | | | | 4293, 4294, 4295, 4296, |
| | | | | 4297, 4298, 4299, 4300, |
| | | | | 4301, 4302, 4303, 4304, |
| | | | | 4305, 4306, 4307, 4308, |
| | | | | 4309, 4310, 4311, 4312, |
| | | | | 4313, 4314, 4315, 4316, |
| | | | | 4317, 4318, 4319, 4320, |
| | | | | 4321, 4322, 4323, 4324, |
| | | | | 4325, 4326, 4327, 4328, |
| | | | | 4329, 4330, 4331, 4332, |
| | | | | 4333, 4334, 4335, 4336, |
| | | | | 4337, 4338, 4339, 4340, |
| | | | | 4341, 4342, 4343, 4344, |
| | | | | 4345, 4346, 4347, 4348, |
| | | | | 4349, 4350, 4351, 4352, |
| | | | | 4353, 4354, 4355, 4356, |
| | | | | 4357, 4358, 4359, 4360, |
| | | | | 4361, 4362, 4363, 4364, |
| | | | | 4365, 4366, 4367, 4368, |
| | | | | 4369, 4370, 4371, 4372, |
| | | | | 4373, 4374, 4375, 4376, |
| | | | | 4377, 4378, 4379, 4380, |
| | | | | 4381, 4382, 4383, 4384, |
| | | | | 4385, 4386, 4387, 4388, |
| | | | | 4389, 4390, 4391, 4392, |
| | | | | 4393, 4394, 4395, 4396, |
| | | | | 4397, 4398, 4399, 4400, |
| | | | | 4401, 4402, 4403, 4404, |
| | | | | 4405, 4406, 4407, 4408, |
| | | | | 4409, 4410, 4411, 4412, |
| | | | | 4413, 4414, 4415, 4416, |
| | | | | 4417, 4418, 4419, 4420, |
| | | | | 4421, 4422, 4423, 4424, |
| | | | | 4425, 4426, 4427, 4428, |
| | | | | 4429, 4430, 4431, 4432, |
| | | | | 4433, 4434, 4435, 4436, |
| | | | | 4437, 4438, 4439, 4440, |
| | | | | 4441, 4442, 4443, 4444, |
| | | | | 4445, 4446, 4447, 4448, |
| | | | | 4449, 4450, 4451, 4452, |
| | | | | 4453, 4454, 4455, 4456, |
| | | | | 4457, 4458, 4459, 4460, |
| | | | | 4461, 4462, 4463, 4464, |
| | | | | 4465, 4466, 4467, 4468, |
| | | | | 4469, 4470, 4471, 4472, |
| | | | | 4473, 4474, 4475, 4476, |
| | | | | 4477, 4478, 4479, 4480, |
| | | | | 4481, 4482, 4483, 4484, |
| | | | | 4485, 4486, 4487, 4488, |
| | | | | 4489, 4490, 4491, 4492, |
| | | | | 4493, 4494, 4495, 4496, |
| | | | | 4497, 4498, 4499, 4500, |
| | | | | 4501, 4502, 4503, 4504, |
| | | | | 4505, 4506, 4507, 4508, |
| | | | | 4509, 4510, 4511, 4512, |
| | | | | 4513, 4514, 4515, 4516, |
| | | | | 4517, 4518, 4519, 4520, |
| | | | | 4521, 4522, 4523, 4524, |
| | | | | 4525, 4526, 4527, 4528, |
| | | | | 4529, 4530, 4531, 4532, |
| | | | | 4533, 4534, 4535, 4536, |
| | | | | 4537, 4538, 4539, 4540, |
| | | | | 4541, 4542, 4543, 4544, |
| | | | | 4545, 4546, 4547, 4548, |
| | | | | 4549, 4550, 4551, 4552, |
| | | | | 4553, 4554, 4555, 4556, |
| | | | | 4557, 4558, 4559, 4560, |
| | | | | 4561, 4562, 4563, 4564, |
| | | | | 4565, 4566, 4567, 4568, |
| | | | | 4569, 4570, 4571, 4572, |
| | | | | 4573, 4574, 4575, 4576, |
| | | | | 4577, 4578, 4579, 4580, |
| | | | | 4581, 4582, 4583, 4584, |
| | | | | 4585, 4586, 4587, 4588, |
| | | | | 4589, 4590, 4591, 4592, |
| | | | | 4593, 4594, 4595, 4596, |
| | | | | 4597, 4598, 4599, 4600, |
| | | | | 4601, 4602, 4603, 4604, |
| | | | | 4605, 4606, 4607, 4608, |
| | | | | 4609, 4610, 4611, 4612, |
| | | | | 4613, 4614, 4615, 4616, |
| | | | | 4617, 4618, 4619, 4620, |
| | | | | 4621, 4622, 4623, 4624, |
| | | | | 4625, 4626, 4627, 4629, |
| | | | | 4629, 4630, 4631, 4632, |
| | | | | 4633, 4634, 4635, 4636, |
| | | | | 4637, 4638, 4639 in |
| | | | | WO2018231759 |
| Her2 | VH | Identifier 141, 262, 264, | VL | Identifier 140 in |
| | | 266, 268, 270 in | | WO2016054555A2; |
| | | WO2016168773A3; | | Identifier 261, 263, 265, |
| | | Identifier 11 in U.S. Pat. No. 9,518,118; | | 267, 269 in |
| | | Identifier 62 in | | WO2016168773A3; |
| | | US20160333114Al; | | Identifier 10, 18, 23 in |
| | | Identifier 19, 24 in | | U.S. Pat. No. 9,518,118; Identifier 3 in |
| | | U.S. Pat. No. 9,518,118 | | WO2016168769A1; |
| | | | | Identifier 59, 61 in |
| | | | | US20160333114A1 |
| EFNA | VH | Identifier 149, 153, 157, 161 | VL | Identifier 151, 155, 159, |
| | | in US20150125472 | | 163 in WO2012118547; |
| | | | | Identifier 27, 53 in |
| | | | | US20150125472 |
| PGT1 | VH | Identifier 15 in EP307441 | — | — |
| Factor XII | VH | Identifier 15 in | VL | Identifier 17 in |
| | | WO2014089493 | | WO2014089493 |
| E7MC | VH | Identifier 15, 16, 17, 18, 19, | VL | Identifier 238, 239, 240, |
| | | 20, 21, 22, 23, 233, 234, | | 241, 242, 243, 36, 37, 38, |
| | | 235, 236, 237, 24, 25, 26, | | 39, 41, 42, 43, 44, 45, 46, |
| | | 27, 28, 29, 30, 31, 32, 33, | | 47, 48, 49, 50, 51, 52, 53, |
| | | 34, 35 in | | 54, 55, 56 in |
| | | WO2016182957A1 | | WO2016182957A1 |
| ICOS | VH | Identifier 15, 16, 19, 23, 7 in | VL | Identifier 17, 18, 20, 24, 8 |
| | | US20160215059 | | in US20160215059 |
| CD76b | VH | Identifier 15, 17, 19, 23, 27, | VL | Identifier 16, 18, 22, 38, |
| | | 29, 37, 57, 59, 61 in | | 58, 60, 62 in |
| | | US20160159906 | | US20160159906 |
| MUC1C ECD | VH | Identifier 15, 19, 23, 60, 64, | VL | Identifier 17, 21, 25, 62, |
| | | 68, 72 in | | 66, 70, 75 in |
| | | US20160340442A1 | | US20160340442Al |
| CD4BS | VH | Identifier 15, 3 in | VL | Identifier 14, 2 in |
| | | US20160194375A1 | | US20160194375A1 |
| CD7 | VH | Identifier 16, 20 in | VL | Identifier 17, 21 in |
| | | WO2016126213A1 | | WO2016126213A1 |
| PGT2 | VH | Identifier 17 in | VL | Identifier 16 in |
| | | EP3074419A2 | | EP3074419A2 |
| OTK3 | VH | Identifier 17 in | VL | Identifier 18 in |
| | | WO2015158868 | | WO2015158868 |
| GD2 | VH | Identifier 17 in | VL | Identifier 10, 2, 5, 7, 9 in |
| | | WO2016134284; Identifier | | US20130216528; Identifier |
| | | 1 in US20130216528; | | 11, 12 in WO2015132604; |
| | | Identifier 10, 9 in | | Identifier 18 in |
| | | WO2015132604; Identifier | | WO2016134284 |
| | | 3, 4, 6, 8 in | | |
| | | US20130216528 | | |
| Trophoblast | VH | Identifier 17, 13, 15, 11 in | VL | Identifier 18, 12, 14, 16 in |
| Glycoprotein | | WO2016034666A1 | | WO2016034666A1 |
| 5T4 | | | | |
| AMC | VH | Identifier 17, 18, 19, 20, 21, | VL | Identifier 27, 28, 29, 31, |
| | | 22, 23, 24, 25, 26 in | | 32, 33, 34, 35, 36 in |
| | | WO2016161390 | | WO2016161390 |
| Factor D | VH | Identifier 17, 20, 27, 29, 30, | VL | Identifier 16, 18, 19, 26, 3 |
| | | 31, 32, 33, 4 in | | in US20160017052 |
| | | US20160017052 | | |
| CD276 | VH | Identifier 17, 26, 7 in | VL | Identifier 18, 27 in |
| | | US20160053017 | | US20160053017 |
| B7H3 (CD276) | VH | Identifier 17, 26, 7 in | VL | Identifier 18, 27, 8 in |
| | | WO2016044383 | | WO2016044383 |
| RAS | VH | Identifier 17, 47, 57, 67, 7, | VL | Identifier 19, 49, 59, 69, |
| | | 77 in WO2016154047 | | 79, 9 in WO2016154047 |
| DR5 | VH | Identifier 18, 82, 90, 98, 8 in | VL | Identifier 13, 23, 25, 27, 3, |
| | | WO2016122701 | | 78, 86, 94, 29 in |
| | | | | WO2016122701; Identifier |
| | | | | 62 in WO2016122701; |
| | | | | Identifier 54 in |
| | | | | WO2016122701; Identifier |
| | | | | 70 in WO2016122701 |
| PGT3 | VH | Identifier 19 in | VL | Identifier 18 in |
| | | EP3074419A2 | | EP3074419A2 |
| PD1 | VH | Identifier 19 in | VL | Identifier 2, 39, 7, 8, 9 in |
| | | US20150290316; Identifier | | US 20160159905; |
| | | 25, 26, 27, 28, 29 in | | Identifier 21 in |
| | | US20130291136; Identifier | | US20150290316; Identifier |
| | | 29, 3, 38 in US | | 30, 31, 32, 33 in |
| | | 20160159905; Identifier 38, | | US20130291136; Identifier |
| | | 50 in WO2015112900; | | 42, 46, 54 in |
| | | Identifier 4, 4, 6 in US | | WO2015112900; Identifier |
| | | 20160159905; Identifier 82, | | 58, 62, 66, 70, 74, 78 in |
| | | 86 in WO2015112900; | | WO2015112900; Identifier |
| | | Identifier 17 in | | 18 in WO2014055648; |
| | | WO2014055648; Identifier | | Identifier 5 in |
| | | 4 in WO2016040892; | | WO2016040892; Identifier |
| | | Identifier 12 in | | 13 in US20150190506 |
| | | US20150190506 | | |
| NYBR1 | VH | Identifier 19 in | VL | Identifier 18 in |
| | | US20160333422A1 | | US20160333422A1 |
| CD28 | VH | Identifier 19 in | VL | Identifier 20 in |
| | | WO2015158868 | | WO2015158868 |
| Tn Glycopeptide | VH | Identifier 19, 20 in | — | — |
| | | WO2015120180 | | |
| humanERBB3 | VH | Identifier 19, 29, 38, 45, 55, | VL | Identifier 10, 20, 30, 39, |
| | | 61, 9 in WO2013052745 | | 46, 56, 62 in |
| | | | | WO2013052745 |
| Olfml3 | VH | Identifier 19, 3 in | VL | Identifier 20, 4 in |
| | | WO2015054441A1 | | WO2015054441A1 |
| RHAMM | VH | Identifier 2 in | VL | Identifier 4 in |
| antagonist body | | WO2000029447 | | WO2000029447 |
| CD38 | VH | Identifier 2 in | VL | Identifier 1, 11 in |
| | | WO2009080830; Identifier | | WO2009080830 |
| | | 10 in WO2015121454 | | |
| PDl (Nivolumab) | VH | Identifier 2 in | VL | Identifier 11 in |
| | | WO2016040892; Identifier | | US20150190506 |
| | | 10 in US20150190506 | | |
| PDK1 | VH | Identifier 2 in | VL | Identifier 9 in |
| | | WO2016090365 | | WO2016090365 |
| IL21 | VH | Identifier 2, 3 in | — | — |
| | | US20160145332 | | |
| ERBB2 | VH | Identifier 2, 4 in | VL | Identifier 1 in |
| | | US20110129464; Identifier | | US20110129464; Identifier |
| | | 10, 2, 26, 30, 38, 4, 40, 42, | | 12, 16, 20, 24, 32, 36, 44, |
| | | 52, 54, 56, 57, 58, 6 in | | 50, 51, 53, 8 in |
| | | US20130089544; Identifier | | US20130089544; Identifier |
| | | 8 in US20130266564; | | 7 in US20130266564; |
| | | Identifier 1 in | | Identifier 3 in |
| | | US20150104443 | | US20110129464 |
| 5T4 | VH | Identifier 2, 4 in | VL | Identifier 1, 3 in |
| | | WO2016022939 | | WO2016022939 |
| GPDL1 | VH | Identifier 20 in | — | — |
| | | US20160108123 | | |
| GM2 | VH | Identifier 20, 22, 23, 26, 27, | VL | Identifier 21, 24, 25, 31, |
| | | 28, 29, 30 in | | 32, 33, 34, 35 in |
| | | US20090028877 | | US20090028877 |
| EphA2 receptor | VH | Identifier 20, 22, 24, 32, 34, | VL | Identifier 26, 28, 30, 47, |
| | | 36, 37, 38, 40, 42, 43, 45, | | 48, 49, 50, 52, 78, 80 in |
| | | 74, 76 in US20150274824 | | US20150274824 |
| KDR | VH | Identifier 20, 24, 26, 29, 31, | VL | Identifier 22 in |
| | | 33 in WO2003075840 | | WO2003075840 |
| PGT4 | VH | Identifier 21 in | VL | Identifier 20 in |
| | | EP3074419A2 | | EP3074419A2 |
| CD324 | VH | Identifier 21, 23, 25, 27, 29, | VL | Identifier 20, 22, 24, 26, |
| | | 31, 33, 35, 37, 39, 41, 43, | | 28, 30, 32, 34, 36, 38, 40, |
| | | 45, 47, 49, 51, 53, 55, 57, | | 42, 44, 46, 48, 50, 52, 54, |
| | | 59, 61, 63, 65, 67, 69, 71 in | | 56, 58, 60, 62, 64, 66, 68, |
| | | U.S. Pat. No. 9,534,058 | | 70 in U.S. Pat. No. 9,534,058 |
| AXL | VH | Identifier 21, 3, 45 in | VL | Identifier 22, 4 in |
| | | WO2016097370 | | WO2016097370 |
| collagen | VH | Identifier 21, 4, 15, 17, 18, | VL | Identifier 11, 12, 14, 23, |
| | | 19, 20, 5, 6, 7, 1, 2, 3 in | | 25, 26, 27, 8, 9 in |
| | | WO2007024921 | | WO2007024921 |
| EGFR | VH | Identifier 2124 in | VL | 4640, 4641, 4642, 4643, |
| (EGFRvIII) | | WO2018231759; Identifier | | 4644, 4645, 4646, 4647, |
| | | 13 in WO2016016341; | | 4648, 4649, 4650, 4651, |
| | | Identifier 24 in | | 4652, 4653, 4654, 4655, |
| | | WO2016168773 A3; | | 4656, 4657, 4658 in |
| | | Identifier 34 in | | WO2018231759; Identifier |
| | | US20160304615; Identifier | | 14 in WO2016016341; |
| | | 2 in US20160200819A1; | | Identifier 23 in |
| | | Identifier 91, 93 in | | WO2016168773 A3; |
| | | WO2016120216 | | Identifier 42 in |
| | | | | US20160304615; Identifier |
| | | | | 1 in US20160200819A1; |
| | | | | Identifier 92, 94 in |
| | | | | WO2016120216 |
| IL3alpha | VH | Identifier 22 in | VL | Identifier 27, 37 in |
| | | WO2008127735 | | WO2008127735 |
| CS1 | VH | Identifier 22 in | VL | Identifier 104, 106, 108 in |
| | | WO2016168773 A3; 103, | | WO2016120216; Identifier |
| | | 105, 107, 109 in | | 14, 16, 18, 20, 22 in |
| | | WO2016120216; Identifier | | WO2015166056A1; |
| | | 13, 15, 17, 19 in | | Identifier 39, 41, 43, 45, 47 |
| | | WO2015166056A1; | | in WO2015121454; |
| | | Identifier 38, 40, 42, 44, 46 | | Identifier 110, 112 in |
| | | in WO2015121454; | | WO2016120216 |
| | | Identifier 26 in | | |
| | | US20160075784A1 | | |
| KMA | VH | Identifier 22 in | VL | Identifier 2, 21 in |
| | | WO2016172703 A2 | | WO2016172703A2 |
| PGT5 | VH | Identifier 23 in | VL | Identifier 22 in |
| | | EP3074419A2 | | EP3074419A2 |
| CD45 | VH | Identifier 24 in | VL | Identifier 25 in |
| | | WO2016126213A1 | | WO2016126213A1 |
| leukocytegenA2 | VH | Identifier 25 in | — | — |
| | | WO2010065962 A2 | | |
| CD16 | VH | Identifier 25 in | VL | Identifier 26 in |
| | | WO2015158868 | | WO2015158868 |
| BCMA | VH | Identifier 26 in | VL | Identifier 25 in |
| | | WO2016168773 A3; | | WO2016168773 A3; |
| | | Identifier 142, 148, 154, | | Identifier 42 in |
| | | 160, 166, 172, 178, 184, | | WO2016097231; Identifier |
| | | 190, 196, 202, 208, 214, | | 143, 149, 155, 161, 167, |
| | | 220, 226, 232, 238, 244, | | 173, 179, 185, 191, 197, |
| | | 250, 256, 262, 268, 274, | | 203, 209, 215, 221, 227, |
| | | 280, 286, 292, 298, 304, | | 233, 239, 245, 251, 257, |
| | | 310, 316, 322, 328, 334, | | 263, 269, 275, 281, 287, |
| | | 340, 346, 352 in | | 293, 299, 305, 311, 317, |
| | | WO2016168595A1; | | 323, 329, 335, 341, 347, |
| | | Identifier 8 in | | 353 in WO2016168595 Al; |
| | | WO2016094304A3; | | Identifier 192, 193, 194, |
| | | Identifier 171, 172, 173, | | 195, 196, 197, 198, 199, |
| | | 174, 175, 176, 177, 178, | | 200, 201, 204, 205, 207, |
| | | 179, 180, 181, 182, 183, | | 208, 211, 259, 260, 84, 85, |
| | | 184, 185, 186, 187, 190, | | 86, 87, 88, 89, 90, 91, 92, |
| | | 255, 257, 258, 69, 70, 71, | | 93, 94, 95, 96, 97, 98 in |
| | | 72, 73, 74, 75, 76, 77, 78, | | WO2016014565; Identifier |
| | | 79, 80, 81 WO2016014565; | | 53 in WO2016187349A1; |
| | | Identifier 38 in | | Identifier 7 in |
| | | EP3057994A1; Identifier 55 | | WO2016094304 A3; |
| | | in WO2016187349A1; | | Identifier 10, 14, 18, 2, 22, |
| | | Identifier 1, 13, 17, 21, 25, | | 26, 30, 34, 38, 42, 46, 50, |
| | | 29, 33, 37, 41, 45, 49, 5, 53, | | 54, 58, 6, 62, 66 in |
| | | 57, 61, 65, 9 in | | WO2016090320; Identifier |
| | | WO2016090320; Identifier | | 100, 102, 175, 96, 98 in |
| | | 101, 743, 174, 758, 95, 759, | | WO2016120216; Identifier |
| | | 97, 760, 99 in | | 12, 14, 16, 18 in |
| | | WO2016120216; Identifier | | WO2015158671A1; |
| | | 11, 741, 17 in | | Identifier 7, 8, 9 in |
| | | WO2015158671Al; | | WO2016014789; Identifier |
| | | Identifier 10, 11, 12, 13, 14 | | 14 in WO2016168766A1 |
| | | in WO2016014789; | | |
| | | Identifier 15 in | | |
| | | WO2016168766A1 | | |
| humanCD79b | VH | Identifier 27 in | VL | Identifier 28, 30 in |
| | | WO2016112870 | | WO2016112870 |
| | | humanCD79b VH 2304 | | |
| | | Identifier 29 in | | |
| | | WO2016112870 | | |
| B2MG | VH | Identifier 28 in | VL | Identifier 29 in |
| | | WO2016126213A1 | | WO2016126213A1 |
| CD19H 803 | VH | Identifier 28, 29, 32, 33, 34, | — | — |
| | | 35 in WO2016168773 A3; | | |
| | | Identifier 51 in | | |
| | | WO2016187349A1; | | |
| | | Identifier 20 in | | |
| | | US20160039942; Identifier | | |
| | | 1 in WO2014184143; | | |
| | | Identifier 5 in | | |
| | | US20160145337Al; | | |
| | | Identifier 166, 167, 168, | | |
| | | 172, 176, 177, 181, 183, | | |
| | | 184, 185, 62 in | | |
| | | US20160152723; Identifier | | |
| | | 15 US20160319020; | | |
| | | Identifier 17, 33, 34, 35 in | | |
| | | EP3057994Al; Identifier 62 | | |
| | | in WO2016097231; | | |
| | | Identifier 12 in | | |
| | | WO2016134284; Identifier | | |
| | | 111, 113 in | | |
| | | US20160333114A1 | | |
| NOTCH 2/3 | VH | Identifier 29 in | VL | Identifier 31 in |
| | | WO2013074596 | | WO2013074596 |
| KIR (Lirilumab) | VH | Identifier 3 in | VL | Identifier 5 in |
| | | US20150290316; 2371 | | US20150290316; Identifier |
| | | Identifier 1 in | | 2 in WO2014055648 |
| | | WO2014055648 | | |
| CD22 | VH | Identifier 3 in | VL | Identifier 17, 8, 14, 15 in |
| | | WO2013059593; Identifier | | US20150239974; Identifier |
| | | 10, 11, 12, 7, 9, 8 in | | 7 in US20150299317; |
| | | US20150299317; Identifier | | Identifier 681 in |
| | | 201 in WO2016164731; | | WO2016164731; Identifier |
| | | CD22 VH 869 Identifier | | 682 in WO2016164731; |
| | | 671 in WO2016164731; | | Identifier 683, 2020 in |
| | | Identifier 672 in | | WO2016164731; Identifier |
| | | WO2016164731; Identifier | | 684 in WO2016164731; |
| | | 673 in WO2016164731; | | Identifier 685 in |
| | | Identifier 676 in | | WO2016164731; Identifier |
| | | WO2016164731; Identifier | | 686 in WO2016164731; |
| | | 678 in WO2016164731; | | Identifier 687 in |
| | | Identifier 679 in | | WO2016164731; Identifier |
| | | WO2016164731; Identifier | | 688 in WO2016164731; |
| | | 680 in WO2016164731; | | Identifier 690 in |
| | | Identifier 700 in | | WO2016164731; Identifier |
| | | WO2016164731; Identifier | | 740 in WO2016164731; |
| | | 701 in WO2016164731; | | Identifier 741 in |
| | | Identifier 702 in | | WO2016164731; Identifier |
| | | WO2016164731; Identifier | | 742 in WO2016164731; |
| | | 703 in WO2016164731; | | Identifier 743 in |
| | | Identifier 704 in | | WO2016164731; Identifier |
| | | WO2016164731; Identifier | | 744 in WO2016164731; |
| | | 705 in WO2016164731; | | Identifier 745 in |
| | | Identifier 706 in | | WO2016164731; Identifier |
| | | WO2016164731; Identifier | | 746 in WO2016164731; |
| | | 707 in WO2016164731; | | Identifier 747 in |
| | | Identifier 708 in | | WO2016164731, Identifier |
| | | WO2016164731; Identifier | | 748 in WO2016164731; |
| | | 709 in WO2016164731; | | Identifier 749 in |
| | | Identifier 711 in | | WO2016164731; Identifier |
| | | WO2016164731; Identifier | | 750 in WO2016164731; |
| | | 712 in WO2016164731; | | Identifier 752 in |
| | | Identifier 713 in | | WO2016164731; Identifier |
| | | WO2016164731; Identifier | | 753 in WO2016164731; |
| | | 714 in WO2016164731; | | Identifier 754 in |
| | | Identifier 715 in | | WO2016164731; Identifier |
| | | WO2016164731; Identifier | | 755 in WO2016164731; |
| | | 716 in WO2016164731; | | Identifier 756 in |
| | | Identifier 717 in | | WO2016164731; Identifier |
| | | WO2016164731; Identifier | | 757 in WO2016164731; |
| | | 718 in WO2016164731; | | Identifier 758 in |
| | | Identifier 719 in | | WO2016164731; Identifier |
| | | WO2016164731; Identifier | | 759 in WO2016164731; |
| | | 720 in WO2016164731; | | Identifier 760 in |
| | | Identifier 721 in | | WO2016164731; Identifier |
| | | WO2016164731; Identifier | | 761 in WO2016164731; |
| | | 722 in WO2016164731; | | Identifier 762 in |
| | | Identifier 723 in | | WO2016164731; Identifier |
| | | WO2016164731; Identifier | | 763 in WO2016164731; |
| | | 724 in WO2016164731; | | Identifier 764 in |
| | | Identifier 725 in | | WO2016164731; Identifier |
| | | WO2016164731; Identifier | | 765 in WO2016164731; |
| | | 726 in WO2016164731; | | Identifier 766 in |
| | | Identifier 727 in | | WO2016164731; Identifier |
| | | WO2016164731; Identifier | | 767 in WO2016164731; |
| | | 728 in WO2016164731; | | Identifier 768 in |
| | | Identifier 729 in | | WO2016164731; Identifier |
| | | WO2016164731; Identifier | | 769 in WO2016164731; |
| | | 730 in WO2016164731; | | Identifier 770 in |
| | | Identifier 731 in | | WO2016164731; Identifier |
| | | WO2016164731; Identifier | | 771 in WO2016164731; |
| | | 732 in WO2016164731; | | Identifier 772 in |
| | | Identifier 733 in | | WO2016164731; Identifier |
| | | WO2016164731; Identifier | | 773 in WO2016164731; |
| | | 734 in WO2016164731; | | Identifier 774 in |
| | | Identifier 735 in | | WO2016164731; Identifier |
| | | WO2016164731; Identifier | | 775 in WO2016164731; |
| | | 736 in WO2016164731; | | Identifier 776 in |
| | | Identifier 737 in | | WO2016164731; Identifier |
| | | WO2016164731; Identifier | | 777 in WO2016164731; |
| | | 738 in WO2016164731 | | Identifier 124 in |
| | | | | WO2016122701 |
| CTLA4 | VH | Identifier 3, 31, 32, 33, 34, | VL | Identifier 36, 37, 38, 39, |
| | | 35, 41, 42, 43, 44, 45, 7 in | | 40, 46, 47, 48, 49, 50, 8, 4 |
| | | US20140105914; Identifier | | in US20140105914; |
| | | 4 in U.S. Pat. No. 8,697,845; Identifier | | Identifier 2 in U.S. Pat. No. 8,697,845; |
| | | 19 in US20150283234; | | Identifier 20 in |
| | | Identifier 17 in | | US20150283234; Identifier |
| | | WO2014066532 | | 18 in WO2014066532 |
| Integrin | VH | Identifier 3, 4, 5 in US | VL | Identifier 10, 11, 8, 9 in US |
| | | 20140161794 | | 20140161794 |
| IL13 | VH | Identifier 302 in | VL | Identifier 303 in |
| | | US20160168242 | | US20160168242 |
| Human collagen | VH | Identifier 31 in | VL | Identifier 32 in |
| VII | | WO2016112870 | | WO2016112870 |
| NKG2A | VH | Identifier 32 in | VL | Identifier 33 in |
| | | WO2016126213A1; | | WO2016126213A1; |
| | | Identifier 2, 3, 4, 5, 6 in | | Identifier 7 in |
| | | WO2016041947 | | WO2016041947 |
| Campath 1 | VH | Identifier 34 in | VL | Identifier 31, 33 in |
| | | US20160333114Al | | US20160333114A1 |
| KIR2DL 1 | VH | Identifier 36 in | VL | Identifier 37 in |
| | | WO2016126213A1 | | WO2016126213A1 |
| KIR2DL 2/3 | VH | Identifier 36 in | VL | Identifier 37 in |
| | | WO2016126213A1 | | WO2016126213A1 |
| VISTA | VH | Identifier 37, 38, 39, 40 in | VL | Identifier 41, 42, 43, 44, 45 |
| | | WO2015097536 | | in WO2015097536 |
| CD46 | VH | Identifier 39, 47, 59, 15, 19, | VL | Identifier 41, 61, 21, 25, |
| | | 23, 27, 31, 35, 43, 51, 55, | | 29, 33, 37, 45, 49, 53, 57, |
| | | 63, 67, 71, 75, 79, 83, 69, | | 65, 69, 73, 77, 81, 85, 17, |
| | | 71, 83 in WO2012031273; | | 73, 77 in WO2012031273; |
| | | Identifier 1, 10, 11, 12, 13, | | Identifier 23, 24, 25, 26, |
| | | 14, 15, 16, 17, 3, 5, 6, 7, 9, | | 27, 28, 29, 30, 31, 32, 33, |
| | | 18, 19, 20, 21 in | | 34, 35, 36, 37, 38, 39, 40, |
| | | WO2016040683 | | 41, 42 in WO2016040683 |
| RHAMM | VH | Identifier 4 in | — | — |
| | | US20020127227A1 | | |
| VEGF | VH | Identifier 4, 8, in | VL | Identifier 2, 6 in |
| | | WO2000034337; Identifier | | WO2000034337; Identifier |
| | | 12, 20, 4, 44 in | | 9 in US20030175276Al; |
| | | WO2006012688A1; | | Identifier 11, 19, 27, 28, 3, |
| | | Identifier 7 in | | 43 in WO2006012688A1; |
| | | US20030175276Al; | | Identifier 160, 161, 162, |
| | | Identifier 152, 153, 154, | | 163, 164, 165, 166, 167 in |
| | | 155, 156, 157, 158, 159 in | | US20160090427 |
| | | US20160090427 | | |
| Endoglin | VH | Identifier 41, 42, 43, 71, 73, | VL | Identifier 103, 88, 89, 90, |
| | | 75, 88, 89, 90, 91, 92 in | | 91, 92, 93, 94, 95, 96, 97, |
| | | US20160009811 | | 102, 100 in |
| | | | | WO2011041441; Identifier |
| | | | | 100, 102, 103, 3, 4, 5, 70, |
| | | | | 72, 74, 93, 94, 95, 96, 97 in |
| | | | | US20160009811 |
| PRP | VH | Identifier 42 in | VL | Identifier 39, 41 in |
| | | US20160333114A1 | | US20160333114Al |
| Lysyloxidase- | VH | Identifier 42, 44 in | VL | Identifier 43, 45 in |
| like 2 | | WO2011097513 | | WO2011097513 |
| PSMA | VH | Identifier 43 in | VL | Identifier 44 in |
| | | WO2016097231 | | WO2016097231; Identifier |
| | | | | 44 in WO2016097231 |
| PDL2 | VH | Identifier 43, 44, 56, 46 in | VL | Identifier 47, 48, 49, 50, 51 |
| | | US20130291136 | | in US20130291136 |
| Daclizumab | VH | Identifier 44, 46 in | VL | Identifier 43, 45 in |
| | | US20160333114A1 | | US20160333114Al |
| CD20 | VH | Identifier 45 in | VL | Identifier 46 in |
| | | WO2016097231; Identifier | | WO2016097231; Identifier |
| | | 11, 13, 14, 15, 16, 17, 18, | | 10, 12, 8 in |
| | | 19, 20, 21, 22, 23, 24, 25, | | WO2017004091; Identifier |
| | | 26, 27, 28, 29, 30, 31, 32, | | 51 in US20160333114Al |
| | | 33, 7, 9 in WO2017004091; | | |
| | | Identifier 26 in | | |
| | | US20170000900; Identifier | | |
| | | 54 in US20160333114Al; | | |
| | | Identifier 25 in | | |
| | | US20170000900; Identifier | | |
| | | 24 in US20170000900; | | |
| | | Identifier 23 in | | |
| | | US20170000900 | | |
| Klon43 | VH | Identifier 47 in | VL | Identifier 48 in |
| | | WO2016097231 | | WO2016097231 |
| MUC1 | VH | Identifier 5 in | VL | Identifier 7 in |
| | | US20160130357; Identifier | | US20160130357; Identifier |
| | | 2, 14 in WO2013023162; | | 16, 7 in WO2013023162; |
| | | Identifier 15, 19, 23, 60, 64, | | Identifier 17, 21, 25, 62, |
| | | 68 in WO2015116753 | | 66, 70 in WO2015116753 |
| CD4i | VH | Identifier 5 in | VL | Identifier 4 in |
| | | US20160194375Al | | US20160194375A1 |
| BAT1 | VH | Identifier 5, 6, 7, 8, 9 in | VL | Identifier 1, 2, 3, 4 in |
| | | WO2013014668 | | WO2013014668 |
| PRAME | VH | Identifier 50, 52, 54, 56, 58, | VL | Identifier 49, 51, 53, 55, |
| | | 60, 62 in | | 57, 59, 61 in |
| | | WO2016191246A2 | | WO2016191246A2 |
| CD19 | VH | Identifier 53, 55 in | VL | Identifier 27, 31 in |
| | | WO2016120216 | | WO2016168773 A3; |
| | | | | Identifier 49 in |
| | | | | WO2016187349A1; |
| | | | | Identifier 11 in |
| | | | | WO2016134284; Identifier |
| | | | | 194 in US20140134142Al; |
| | | | | Identifier 54, 56 in |
| | | | | WO2016120216; Identifier |
| | | | | 13, 14, 15, 16, 17, 186, |
| | | | | 187, 188, 189, 192, 196, |
| | | | | 197, 198, 199, 200, 201, |
| | | | | 202, 203, 204, 205, 64, 66, |
| | | | | 67, 68, 69, 70, 71, 91 |
| | | | | US20160152723; Identifier |
| | | | | 22 in US20160039942; |
| | | | | 3361 Identifier 63 in |
| | | | | WO2016097231; Identifier |
| | | | | 3 in US20160145337Al; |
| | | | | Identifier 112 in |
| | | | | US20160333114Al; |
| | | | | Identifier 114 in |
| | | | | US20160333114A1; |
| | | | | Identifier 13, 6 |
| | | | | US20160319020 |
| Notum | VH | Identifier 56, 331 in | VL | Identifier 332, 58 in |
| | | WO2012027723 | | WO2012027723 |
| NOTCH 1 | VH | Identifier 58 in | VL | Identifier 16, 20 in |
| | | US20160333114A1; | | WO2013074596; Identifier |
| | | Identifier 12 in | | 55, 57 in |
| | | WO2013074596 | | US20160333114Al; |
| Osteonectin | VH | Identifier 58 in | VL | Identifier 59 in |
| | | WO2016112870 | | WO2016112870 |
| CD74 | VH | Identifier 6 in | VL | Identifier 25, 29, 31, 35 in |
| | | US20100284906Al; | | US20130171064; Identifier |
| | | Identifier 10, 11, 9 in | | 12, 13, 14, 11, 4 in |
| | | US20040115193Al; | | US20040115193Al |
| | | Identifier 23, 27, 30, 33 in | | |
| | | US20130171064 | | |
| CD33 | VH | Identifier 65, 67, 69, 71, 77, | VL | Identifier 12, 14, 16, 18 in |
| | | 79, 81, 83, 84 in | | WO2015150526A2; |
| | | WO2016120216; Identifier | | Identifier 66, 68, 70, 72, |
| | | 11, 13, 15; 17 in | | 78, 80, 82 in |
| | | WO2015150526A2; | | WO2016120216; Identifier |
| | | Identifier 57, 58, 59, 60, 61, | | |
| | | 62, 63, 64, 65 in | | 66, 67, 68, 69, 70, 71, 72, |
| | | WO2016014576 | | 73, 74 in WO2016014576 |
| Rituximab | VH | Identifier 66 in | VL | Identifier 63, 65 in |
| | | US20160333114Al | | US20160333114Al; |
| GAH | VH | Identifier 7 in | VL | Identifier 8 in |
| | | US20060057147A1 | | US20060057147A1 |
| Glyco epitope | VH | Identifier 7 in | VL | Identifier 10 in |
| and ErbBB I | | WO2012007167A1 | | WO2012007167A1 |
| Specific | | | | |
| CD3s | VH | Identifier 7 in | VL | Identifier 8 in |
| | | WO2014144722A2 | | WO2014144722A2 |
| IGFR1 | VH | Identifier 7 in | VL | Identifier 8 in |
| | | WO2015073575 A2 | | WO2015073575 A2 |
| IL13Ra2 | VH | Identifier 7, 8 in | — | — |
| | | WO2016123143 | | |
| N Glycan | VH | Identifier 7, 9 in | VL | Identifier 6, 8 in |
| | | US20160194375A1 | | US20160194375A1 N |
| Upar | VH | Identifier 72 in | VL | Identifier 71, 73 in |
| | | US20160333114Al; | | US20160333114Al; |
| CXCR4 | VH | Identifier 72, 73, 74, 75, 84 | VL | Identifier 76, 77, 78, 79, |
| | | in US20110020218 | | 80, 81, 82, 87, 88, 90, 91, |
| | | | | 92, 93 in US20110020218 |
| Tie | VH | Identifier 723 in | VL | Identifier 724 in |
| | | US20060057138Al | | US20060057138Al |
| CEA | VH | Identifier 8 in | VL | Identifier 10, 38, 39, 7, 9 in |
| | | U.S. Pat. No. 8,287,865 | | U.S. Pat. No. 8,287,865 |
| Herl/her3 | VH | Identifier 8 of | VL | Identifier 4 of |
| | | WO2016073629 | | WO2016073629 |
| CD70 | VH | Identifier 81, 85, 89 in | VL | Identifier 83, 87, 91 in |
| | | WO2015121454 | | WO2015121454; Identifier |
| | | | | 83 in WO2015121454; |
| | | | | Identifier 87 in |
| | | | | WO2015121454; Identifier |
| | | | | 91 in WO2015121454 |
| leukocytegenA | VH | Identifier 9 in | VL | Identifier 24 in |
| | | WO2010065962 A2 | | WO2010065962 A2 |

In some embodiments, the antigen-binding moiety may comprise an scFv derived from an antibody or antibody fragment that binds to an antigen target such as those described in the cited publications, the contents of each publication are incorporated herein by reference in their entirety for all purposes (Table 2).

TABLE 2
Exemplary antigen-binding moieties comprising an scFv derived from
an antibody or antibody fragment that binds to an antigen target

| Antigen Target | Examples of Source |
|---|---|
| CEA | Identifier 1 in US20160303166A1; Identifier 22 in US20140242701A1 |
| GPC3 | Identifier 1 in WO2016049459; Identifier 12 in US20160208015A1 |
| CS1 | Identifier 1 of WO2016090369; Identifier 17 in WO2014179759A1 |
| VEGFR2 | Identifier 1, 2 in US20120213783 |
| TSLPR | Identifier 1, 2 in US20160311910A1; Identifier 1, 2 in WO2015084513 |
| B7H4 | Identifier 1, 2, 3, 4 in WO2013067492; Identifier 1 in U.S. Pat. No. 9,422,351B2 |
| CD276 | Identifier 10, 19, 28 in US20160053017 |
| GPRC5D | Identifier 100, 101, 102, 103, 104, 105, 106, 107, 108, 109, 110, 111, 112, 113, 114, |
| | 115, 116, 117, 118, 119, 120, 121, 122, 123,301, 313, 325, 337, 349, 361, 373, 385 in |
| | WO2016090312 |
| WT1/HLA | Identifier 108, 113, 18, 36, 54, 72, 90 in WO2015070061 |
| bispecific | |
| CMet | Identifier 11, 12, 13, 14, 15, 16, 17, 18, 19, 2, 21, 22, 23, 25, 26, 27, 28, 3, 30, 31, 33, |
| | 34, 35, 36, 37, 38, 39, 4; 40, 41, 42, 43, 44, 48, 49, 5, 50, 51, 52, 53, 54, 55, 56, 57, |
| | 58, 6, 60, 7, 9, 29 in US20040166544; Identifier 26, 27, 28, 29, 30, 32 in |
| | US20150299326; Identifier 32 in US20130034559 |
| FcRL | Identifier 11, 15, 19, 23, 27, 31, 35, 39, 3, 43, 7, 594, 596, 598, 600, 602, 604, 606, |
| | 608, 610, 612, 614, 616, 618, 620, 622, 624, 626, 628, 630, 632, 634, 636, 638, 640, |
| | 642, 644, 646, 648, 652, 654, 656; 658, 660, 662, 664, 666, 668, 670, 672, 674, 676, |
| | 680, 682, 684, 686, 688, 690, 692, 694, 696, 700, 702, 704, 706, 708, 710, 712, 714, |
| | 716, 718, 720, 722, 724, 726, 728, 730, 732, 734, 736, 738, 740, 742, 744, 746, 748, |
| | 750, 752, 754, 756, 758, 760, 762, 764, 766, 768, 770, 772, 774, 776, 778, 780, 782, |
| | 784, 786, 788, 790, 792, 794, 796, 798, 800, 802, 804, 806, 808, 810, 812, 814, 816, |
| | 818, 820, 822, 824, 826, 828, 830, 832, 834, 836, 838, 840, 842, 844; 846, 848, 850, |
| | 852, 854, 856, 858, 860, 862, 864, 866, 868, 870, 874, 876, 878, 880, 882, 884, 886, |
| | 888, 890, 892, 894, 896, 650, 678 in WO2016090337 |
| EGFR | Identifier 11, 38, 41, 44, 47, 50, 53, 56, 59, 62, 65, 68, 71, 74, 77, 80, 83, 88, 91, 94 |
| | in WO2014130657 |
| Claudin | Identifier 11, 5, 7, 9 in WO2016073649A1; Identifier 17 in WO2014179759A1 |
| PSMA diabody | Identifier 12, 13, 14, 15 in WO2011069019 |
| TRBC1 | Identifier 13, 14, 15, 16, 17, 18, 19, 20, 21, 22, 3 in WO2015132598 |
| CD19/CD22 | Identifier 1303, 1307 in WO2016164731A2 |
| Bispecific | |
| CD46 | Identifier 1-42 in WO2016040683 |
| MUC1 | Identifier 15 in US20160130357 |
| MUC3 | Identifier 15 in US20160130357 |
| Folate receptor | Identifier 15 in US20170002072A1 |
| PDK1 | Identifier 15 in WO2016090365 |
| Folate receptor | Identifier 15, 23 in WO2012099973 |
| alpha | |
| BCMA | Identifier 152, 158, 176, 185, 188, 200, 212, 218, 224, 284, 290, 296, 302, 314, 326, |
| | 344, 129, 130, 131, 132, 133, 134, 135, 136, 138, 139, 140, 141, 142, 143, 144, 145, |
| | 146, 147, 148, 149, 263, 264, 265, 266, 271, 273, 39, 40, 41, 42, 43, 44, 45, 46, 47, |
| | 48, 49, 50, 51, 52, 53, 64, 129, 130, 131, 132, 133, 134, 135, 136, 137, 138, 139, 140, |
| | 141, 142, 143, 144, 145, 146, 147, 148, 149, 263, 264, 265, 266, 39, 40, 41, 42, 43, |
| | 44, 45, 46, 47, 48, 49, 50, 51, 52, 53 in WO2016014565; Identifier 214, 215, 216, |
| | 217, 218, 219, 220, 221, 222, 223, 224, 225, 226, 227, 228, 229, 230, 231, 232, 233, |
| | 234, 235, 236, 237, 238, 239, 240, 241, 242, 243, 244, 245, 246, 247, 248, 249, 251 |
| | in US20160311907A1 |
| CD123 | Identifier 157, 158, 159, 160, 184, 185, 186, 187, 188, 189, 190, 191, 192, 193, 194, |
| | 195, 196, 197, 198, 199, 200, 201, 202, 203, 204, 205, 206, 207, 208, 209, 210, 211, |
| | 212, 213, 214, 215, 478, 480, 483, 485 in WO2016028896; Identifier 36 in |
| | WO2015092024A2; Identifier 57 in WO2016115482A1; Identifier 36 in |
| | EP3083691A2; Identifier 157 in US20160311907A1 |
| CD124 | Identifier 158 in US20160311907A1 |
| CD125 | Identifier 159 in US20160311907A1 |
| CD5 | Identifier 16 in WO2016138491 |
| CD126 | Identifier 160 in US20160311907A1 |
| CD127 | Identifier 161 in US20160311907A1 |
| CD128 | Identifier 162 in US20160311907A1 |
| CD129 | Identifier 163 in US20160311907A1 |
| CD130 | Identifier 164 in US20160311907A1 |
| Claudin6 | Identifier 164 in WO2016115482A1 |
| CD131 | Identifier 165 in US20160311907A1 |
| Claudin7 | Identifier 165 in WO2016115482A1 |
| GCN4 | Identifier 165, 166, 167, 168, 169, 170 in WO2016168773 A3 |
| Claudin8 | Identifier 166 in WO2016115482A1 |
| VEGF | Identifier 168, 169, 170, 171, 172, 173, 174, 175 in US20160090427; Identifier 498, |
| | 500, 502, 504, 506, 508 in US20110177074A1 |
| MUC2 | Identifier 17 in US20160130357 |
| MUC4 | Identifier 17 in US20160130357 |
| CD44 | Identifier 17 in WO2016042461A1 |
| IL4 | Identifier 17, 16 in WO2009121847 |
| ALK | Identifier 17, 18, 19, 20, 21, 22, 23 in WO2015069922; Identifier 17, 18, 19, 20, 21, |
| | 22, 23, 24 in US20160280798A1; Identifier 24 in WO23015069922 |
| ESKAVT | Identifier 173 in WO2016115482A1 |
| GD2 | Identifier 19, 20, 21, in WO2016134284; Identifier 8 in WO2015132604 |
| PSMA | Identifier 19, 21, 30, 31, 34, 35 in WO2012145714 |
| TOSO | Identifier 2 in EP3098237A1 |
| CSPG4 | Identifier 2 in WO2015080981; Identifier 2 in EP3074025A1 |
| CLDN6 | Identifier 2 in WO2016150400 |
| Integrin bivalent | Identifier 2, 1 in WO2009070753 |
| CD30 | Identifier 20 in WO2016116035A1; Identifier 2 in US20160200824A1 |
| NYBR1 | Identifier 21 in US20160333422A1; Identifier 21, 18, 19 in WO2015112830 |
| CD37 | Identifier 21, 22 in US20170000900 |
| PDL1 nanobody | Identifier 22, 23, 24, 25, 26, 27 in US20110129458 |
| Radiation inducible | Identifier 22, 24 in WO2005042780A1 |
| neoantigen | |
| E7MC | Identifier 223, 224, 225, 226, 227, 228, 229, 230, 231, 232 in WO2016182957A1 |
| TRBC2 | Identifier 23, 24, 25, 26, 27, 28, 29, 30, 31, 32 in WO2015132598 |
| GPC4 | Identifier 24 in WO2016049459 |
| ERBB2 | Identifier 26, 27 in US20110059076A1; Identifier 1, 2 in U.S. Pat. No. 7,244,826 |
| CD33 | Identifier 262, 263, 264, 265, 266, 267, 268, 39, 40, 41, 42, 43, 44, 45, 46, 47 in |
| | WO2016014576; Identifier 37 in WO2015092024 A2; Identifier 37 in |
| | EP3083691A2; Identifier 153, 154, 155, 156, 157, 158, 159, 160, 161, 162, 163 in |
| | WO2016115482A1 |
| PDL2 nanobody | Identifier 28, 29, 30, 31, 32, 33 in US20110129458 |
| 6462 | |
| O-acetylated GD2 | Identifier 29, 31 in US20150140023 |
| ganglioside | |
| OX40 | Identifier 33 in US20150190506 |
| CD79b | Identifier 33 in US20160208021 |
| Human CD79b | Identifier 33 in WO2016112870 |
| CD33/CD3s | Identifier 33, 34, 84 in WO2014144722A2 |
| bispecifc | |
| RORI | Identifier 34 in EP3083691A2; Identifier 249, 250 251, 252, 253, 254, 255, 256, 257, |
| | 258, 259, 260, 261, 262, 263, 264, 265, 266, 267, 268 in US20160208018A1; |
| | Identifier 57 in EP3083671A1; Identifier 1, 2 in US20160304619A1; Identifier 34 in |
| | WO2015092024A2 |
| Human collagen VII | Identifier 34 in WO2016112870 |
| Trastuzumab | Identifier 35 in US20160208021; Identifier 35 in WO2016112870 |
| Rituximab | Identifier 36 in US20160208021; Identifier 36 in WO2016112870 |
| CD138 | Identifier 36 in WO2016130598A1 |
| Cetuximab | Identifier 37 in WO2016112870; Identifier 37 in US20160208021 |
| Nivolumab | Identifier 38 in US20160208021; Identifier 38 in WO2016112870 |
| Ipilimumab | Identifier 39 in US20160208021; Identifier 39 in WO2016112870 |
| PD1 | Identifier 39, 40, 41, 42, 43, 44, 45, 46, 47, 48, 49, 50, 51, 52, 53, 54, 55, 56, 57, 58, |
| | 59, 60, 61 in US20160311917A1 |
| CLL1 | Identifier 39, 40, 41, 42, 43, 44, 45, 46, 48, 49, 50, 51 in WO2016014535; Identifier |
| | 200, 201, 202, 203, 204, 205, 206, 207, 208, 209, 210, 211, 212, 213 in |
| | US20160311907A1 |
| CLDN7 | Identifier 4 in WO2016150400 |
| Ranibizumab | Identifier 40 in US20160208021; Identifier 40 in WO2016112870 |
| Adalimumab | Identifier 41 in US20160208021; Identifier 41 in WO2016112870 |
| Teplizumab | Identifier 42 in US20160208021 |
| (mutated) | |
| Teplizumab | Identifier 42 in WO2016112870 |
| CD3 | Identifier 46, 47 in WO2015153912A1 |
| EGFR VIII | Identifier 5 in US20140037628; Identifier 174 in US20160311907A1; Identifier 38 in |
| | U.S. Pat. No. 9,394,368B2; Identifier 5 in US20160200819A1 |
| CD22 | Identifier 5, 6 in WO2013059593; Identifier 9 in US20150299317; Identifier 131, |
| | 132, 133, 134, 135, 136, 137, 138, 139, 140, 203, 209, 215, 221, 227, 232, 238, 244, |
| | 250, 256, 262, 268, 274, 280, 286, 292, 298, 304, 310, 316, 322, 328, 334, 340, 346, |
| | 353, 358, 364, 370, 376, 383, 388, 394, 400, 406, 412, 418, 423 in |
| | WO2016164731A2 |
| CD19 | Identifier 53, 54, 37 in EP3083671A1; Identifier 1, 10, 11, 12, 2 in WO2015157252; |
| | Identifier 10, 2, 206, 207, 208, 209, 210, 211, 213, 214, 215, 216, 217, 218, 219, 220, |
| | 221, 222, 223, 224, 225, 4, 45, 47, 49, 51, 53, 55, 57, 51, 53, 55, 57, 59, 6, 8, 87 in |
| | WO2016033570; Identifier 3, 4, 5, 59, 6, 7, 8, 9 in WO2015157252; 5754 Identifier 5 |
| | in WO2015155341A1; Identifier 7 in WO2014184143; Identifier 9 in |
| | WO2016139487; Identifier 10, 2, 206, 207, 208, 209, 210, 211, 212, 213, 214, 215, |
| | 216, 217, 218, 219, 220, 221, 222, 223, 224, 225, 4, 45, 47, 49, 51, 53, 55, 57, 59, 6, |
| | 8, 87, 89 in US20160152723; Identifier 32, 35, 38 in EP3083691A2; Identifier 174 in |
| | WO2016115482A1; Identifier 20 in WO2012079000; Identifier 32, 33, 35, 38 in |
| | WO2015092024A2; Identifier 39, 40, 41, 42, 43, 44, 45, 46, 47, 48, 49, 50, 51 in |
| | WO2016109410; Identifier 5, 6 in WO2015155341A1; Identifier 7, 9 in |
| | US20160145337A1; Identifier 20 in U.S. Pat. No. 9,499,629B2; Identifier 73 in WO2016164580; |
| | Identifier 10, 2, 206, 207, 209, 210, 212, 216, 218, 219, 220, 221, 222, 223, 224, 225, |
| | 4, 45, 47, 49, 51, 53, 55, 57, 59, 6, 8, 87, 89 in US20160152723; Identifier 5 in |
| | WO2016055551 |
| END 0180 | Identifier 6 in WO2013098813 |
| CLDN8 | Identifier 6 in WO2016150400 |
| PRAME | Identifier 63, 64, 65, 66, 67, 68, 69 in WO2016191246A2 |
| CD20 | Identifier 691 in WO2016164731A100; Identifier 692 in WO2016164731A101; |
| | Identifier 693 in WO2016164731A102; Identifier 694 in WO2016164731A103; |
| | Identifier 695 in WO2016164731A104; Identifier 696 in WO2016164731A105; |
| | Identifier 175 in WO2016115482A1 |
| Mesothelin | Identifier 7 WO2015188141; Identifier NO 47, 46, 57, 48, 49, 50, 51, 53, 54, 55, 56, |
| | 58, 59, 62, 64, 65, 66, 67, 68, 69, 70, 52, 60, 61, 63 in WO2016090034; Identifier 10, |
| | 11, 12 in WO2013142034; Identifier 11 in WO2013063419 |
| CCR4 | Identifier 7, 9 in WO2015191997 |
| Mec/CD3s | Identifier 78 in WO2014144722A2 |
| bispecific | |
| Activated alpha v | Identifier 8, 2, 4 in US20090117096A1 |
| beta 3 | |
| RAS | Identifier 81 in WO2016154047 |
| CXCR4 | Identifier 83, 85, 86, 89 in US20110020218 |
| HER2/CD3 | Identifier 9 in WO2014144722 A2 |
| FOLR1/CD3s | Identifier 90 in WO2014144722A2 |
| bispecific | |
| B7H3 | Identifier 99, 100, 101, 102, 103, 104, 102, 17, 18, 19, 20, 21, 22, 23, 24, 25, 26, 27, |
| | 87, 88, 89, 90, 91, 92, 94, 95, 96, 97, 98 in WO2016033225 |

In some embodiments, the antigen-binding moiety may comprise an antigen-binding moiety derived from a CAR that binds to an antigen target, such as those described in the cited publications, the contents of each publication are incorporated herein by reference in their entirety for all purposes (Table 3).

TABLE 3
Exemplary antigen-binding moieties comprising an antigen-binding
moiety derived from a CAR that binds to an antigen target

| Antigen Target | Examples of Source |
|---|---|
| CAR or gate (DC19 or | Identifier 1 in US20160296562 |
| DC33) | |
| CD19 or CD33 | Identifier 1 in WO2015075468 which recognizes CD19 OR CD33 |
| CD19/IL13 bispecific | Identifier 10 in US20160340649A1 |
| CAT19, campana | Identifier 10 in WO2016139487 |
| architecture | |
| CD2 | Identifier 10, 11 in WO2016138491 |
| CD70 | Identifier 100, 93, 94, 96, 101, 95, 97, 98 in WO2015121454 |
| VNAR | Identifier 105, 106, 107, 108, 109, 110 in US20160333094A1 |
| FcRL5 | Identifier 11 in US20170008963A1 |
| CAT19 CAR with OX40 | Identifier 11 in WO2016139487 |
| zeta endodomain | |
| Folate receptor | Identifier 12 in US20170008963A1 |
| GD2 | Identifier 12 in U.S. Pat. No. 9,446,105B2; Identifier 273, 274 in WO2016168773A3; |
| | Identifier 26, 27, 28, 29, 30, 31, 32, 33, 34, 35, 36, 37 in WO2015132604; |
| | WO2016134284 (no Identifier) |
| CD19 | Identifier 12 in U.S. Pat. No. 9,499,629B2; Identifier 24 in US20160333108A1; Identifier |
| | 25, 29 in US20160333108A1; Identifier 27 in US20160333108A1; Identifier 1 |
| | in EP2997134A4; Identifier 19, 20 in EP3071687A1; Identifier 181 in |
| | WO2016168773A3; Identifier 2 in WO2015157399A9; Identifier 56, 62 in |
| | WO2016174409A1; Identifier 145, 293, 294, 295, 296, 297, 298 in |
| | WO2016179319A1; Identifier 73 in WO2013176915A1; Identifier 73 in |
| | WO2013176916A1; Identifier 73 in US20130315884A1; Identifier 73 in |
| | US20140134142A1; Identifier 73 in US20150017136A1; Identifier 73 in |
| | US20150203817A1; Identifier 73 in US20160120905A1; Identifier 73 in |
| | US20160120906A1; Identifier 8, 5 in WO2015124715; Identifier 73 in |
| | WO2014184744; Identifier 73 in WO2014184741; Identifier 14, 15 in |
| | US20160145337A1; Identifier 14, 15 in WO2014184143; Identifier 15, 16 in |
| | WO2015075175; Identifier 16 in US20160145337A1; Identifier 16 in |
| | WO2014184143; Identifier 12 in WO2012079000; Identifier 31, 32, 33, 34, 35, |
| | 36, 37, 38, 39, 40, 41, 42, 58 in WO2016164580; Identifier 14, 15 in |
| | US20160296563A1; Identifier 31, 32, 33, 34, 35, 36, 37, 38, 39, 40, 41, 42 in |
| | WO2015157252; Identifier 14, 15 in WO2016139487; Identifier 53, 54, 55, 56, |
| | 57, 58 in US20160250258A1; Identifier 1, 2, 3, 4, 5, 6, 7, 8, 9, 10, 11, 12, 13 in |
| | WO2015187528; Identifier 31, 32, 33, 34, 35, 36, 37, 38, 39, 40, 41, 42, 58 in |
| | WO2015157252; Identifier 31, 32, 33, 34, 35, 36, 37, 38, 39, 40, 41, 42 in |
| | WO2014153270; WO2016134284 (no Identifier); Identifier 13 in |
| | WO2016139487 |
| CD3 | Identifier 12 in WO2016138491 |
| CAT19 CAR with CD28 | Identifier 12 in WO2016139487 |
| zeta endodomain | |
| FRa | Identifier 13, 14 in US20120213783 |
| CD4 | Identifier 13, 14 in WO2016138491 |
| FR beta | Identifier 13, 22 in U.S. Pat. No. 9,402,865B2; Identifier 2, 4, 6 in U.S. Pat. No. 9,446,105B2 |
| CD19/CD20 bispecific | Identifier 1308 in WO2016164731A2; Identifier 2, 8, 11 in U.S. Pat. No. 9,447,194B2 |
| CLL1 | Identifier 148 in WO2016179319A1; Identifier 35, 36, 37, 38, 39, 40, 41, 42, |
| | 43, 44, 45, 46, 47, 48, 49, 50, 51, 52, 53, 54, 55, 56, 57, 58, 59, 60, 61, 62, 63, |
| | 64, 65, 66, 67, 68, 69, 70, 71, 72, 73, 74, 75, 76, 77, 78, 79, 80, 81, 82, 83, 84, |
| | 85, 86, 87, 88, 89, 90, 91, 92, 93, 94, 95, 96, 97, 98, 99, 100, 101, 102, 103, 104, |
| | 105, 106, 107, 108, 109, 110, 111, 112 in WO2016120218; Identifier 91, 92, 93, |
| | 94, 95, 96, 97, 98, 99, 100, 101, 102, 103, 197 in WO2016014535 |
| CD5 | Identifier 15, 13 in WO2016138491 |
| EGFR vIII | Identifier 15, 16, 17, 18, 24, 25, 26, 27 in WO2016016341; Identifier 5, 10, 12, |
| | 8, 31, 30, 3 in US20160311907A1; Identifier 10 in US20160200819A1; |
| | Identifier 43, 49, 55, 61, 67, 73, 79, 85, 90, 96 in U.S. Pat. No. 9,394,368B2; Identifier 49, |
| | 55, 61, 67, 73, 79, 85, 90, 2, 1 in US20170008963A1; Identifier 10, 11 in |
| | US20140037628 |
| CD7 | Identifier 17 in WO2016138491 |
| CD52 | Identifier 18 in WO2016138491 |
| BCMA | Identifier 180, 162, 168, 174, 144, 150, 186, 192, 198, 204, 210, 156, 216, 222, |
| | 228, 234, 240, 246, 252, 258, 264, 270, 276 330, 282, 300, 306, 336, 354, 288, |
| | 312, 294, 342, 324, 318, 348 in WO2016168595A1; Identifier 48, 49, 50, 51, |
| | 52, 53, 54, 55, 56, 57, 58, 59, 19, 20, 21, 22, 23, 24, 25, 26, 27, 28, 29, 30, 31, |
| | 32, 33, 34, 35, 36, 37, 38, 39, 40, 41, 42 in WO2015158671A1; Identifier 124, |
| | 114, 115, 116, 117, 118, 119, 120, 121, 122, 123, 125, 126, 127, 128, 234, 235, |
| | 236, 237, 238, 239, 240, 241, 242, 243, 244, 245, 246, 247, 248, 249, 250, 251, |
| | 252, 253, 254, 267, 268, 269, 270 in WO2016014565; Identifier 1, 2, 3, 4, 5, 20 |
| | in WO2015052538; Identifier 1, 2, 3, 4, 5, 6 in US20160237139A1; Identifier 9 |
| | in WO2016094304 A3; Identifier 4, 5, 6, 8, 9, 10, 11, 12 in WO2013154760; |
| | Identifier 15, 16, 17, 18, 19, 20, 21, 22, 23, 24, 25, 26, 27, 28, 29, 71, 73 in |
| | WO2016014789; Identifier 125, 126, 127, 128, 129, 130, 131, 132, 133, 134, |
| | 135, 136, 137, 138, 139, 140, 141, 145, 146, 147, 148, 149, 150 in |
| | WO2016120216; Identifier 102, 106, 107, 108, 109, 110, 111, 112, 129, 130, |
| | 131, 132, 133, 134, 135, 136, 113, 114, 115, 116, 117, 118, 101, 100, 137, 119, |
| | 120, 121, 122, 123, 124, 125, 126, 127, 128, 103, 104, 105, 213 in |
| | WO2016097231 |
| Tan | Identifier 19 in US20160311907A1 recognizes CD19 AND CD33 using a CD45 |
| | phosphatase; Identifier 5 in WO2016026742A1 recognizes CD19 AND CD33 |
| | using a CD148 phosphatase; Identifier 6 in WO2016026742A1 which |
| | recognizes CD19 AND NOT CD33 and is based on an ITIM containing |
| | endodomain from LAIR1; Identifier 3 in WO2015075468 which recognizes |
| | CD19 AND NOT CD33 based on PTPN6 phosphatase; Identifier 2 in |
| | WO2015075468 which recognizes CD19 AND NOT CD33 and recruits a |
| | PTPN6/CD148 fusion protein to an ITIM containing endodomain |
| GD3 | Identifier 19 in WO2016185035A1; Identifier 20, 21, 22, 23, 24, 25, 26 in |
| | WO2016185035A1; WO2016134284 (no Identifier) |
| CAR and gate (CD19 and | Identifier 2 in US20160296562 |
| CD33) CD148 | |
| phosphatase | |
| CD30 | Identifier 20 in WO2016008973A1; Identifier 1 in WO2016116035A1; |
| | WO2016134284 (no Identifier); Identifier 2 in WO2016008973 |
| CD44 | Identifier 21, 22, 23, 24, 25, 26, 27, 28, 31, 32, 33, 34, 35 in WO2016042461A1 |
| ROR1 | Identifier 216, 217, 215 in WO2016097231; Identifier 79, 80, 81, 82, 83, 84, 85, |
| | 86, 87, 88, 89, 90, 91, 92, 93, 94, 95, 96, 103, 104, 105, 106, 107, 108, 109, 110, |
| | 111, 112, 113, 114, 115, 116, 117, 118, 119, 120, 127, 128, 129, 130, 131,132, |
| | 133,134, 1335, 136, 137, 138, 97, 98, 99, 100, 101, 102, 121, 122, 123, 124, |
| | 125, 126 in WO2016016344A1; Identifier 386, 387, 388, 389, 390, 391, 392, |
| | 393, 394 in WO2016187216A1 |
| FR | Identifier 22 in US20170002072A1 |
| CLDN6 | Identifier 22, 23, 24 in WO2016150400 |
| PD1 | Identifier 23 in WO2016014565; Identifier 26 in WO2015142675 |
| Her1/Her3 bispecific | Identifier 23, 24 in US20160215261A1 |
| HER2 | Identifier 25 in US20160215261A1; Identifier 9, 10 of WO2016073629; |
| | Identifier 17, 28, 98, 110 in US20160333114A1; Identifier 271, 272 in |
| | WO2016168773A3; Identifier 5 in WO2016168769A1 |
| CD20 | Identifier 25 in WO2015157399A9; Identifier 177, 181, 182, 183, 184, 185, |
| | 186, 187, 205, 206, 207, 208, 209, 210, 211, 188, 189, 190, 191, 192, 193, 176, |
| | 212, 194, 195, 196, 197, 198, 199, 200, 201, 202, 203, 178, 179, 180 in |
| | WO2016097231 |
| P5A | Identifier 26, 29, 60 in US20160333422A1 |
| GFR alpha | Identifier 27 in WO2016185035A1 |
| GPC3 | Identifier 28, 29 in WO2016185035A1; Identifier 3, 27, 10, 29, 14, 30, 31, 18, |
| | 33 in WO2016049459; Identifier 22 in US20160215261A1 |
| CD22/CD19 bispecific | Identifier 29, 30 in WO2016149578; Identifier 1304 in WO2016164731A2 |
| CAR and gate (CD19 or | Identifier 3 in US20160296562 |
| CD33) CD45 phosphatase | |
| EGFR | Identifier 3, 2in WO2014130657; Identifier 36, 37, 38, 39, 35 in |
| | US20140242701 A; Identifier 43, 96, 49, 55, 61, 67, 73, 79, 85, 90, 1 in |
| | WO2014130657 |
| IL13 | Identifier 30, 31, 32 in WO2016120217 |
| Acid/base leucine zipper | Identifier 34, 35 in WO2016124930 |
| P5AC1 | Identifier 343, 344, 345, 346 in US20160297884A1 |
| P5AC16 | Identifier 347, 396, 348 in US20160297884A1 |
| P6AP | Identifier 349, 350, 351 in US20160297884A1 |
| PC1C12 | Identifier 352, 353, 354 in US20160297884A1 |
| PD1 | Identifier 355, 356, 357 in US20160297884A1; Identifier 119 in |
| | WO2014153270; Identifier 121 in WO2014153270; Identifier 22, 24, 63, 64, |
| | 65, 66, 67, 68, 69, 70, 71, 72, 73, 74, 75, 76, 77, 78, 79, 80, 81, 82, 83, 84, 85, |
| | 86 in US20160311917A1; Identifier 26, 39 in WO2016172537A1; Identifier 40 |
| | in US20160311907A1; Identifier 121, 119 in WO2015157252; Identifier 24 in |
| | WO2016014565; Identifier 22 in WO2016014565 |
| COM22 | Identifier 358, 359, 360 in US20160297884A1 |
| DDD1/AD1-based zip | Identifier 36 in WO2016124930 |
| PCI | Identifier 361, 362, 363 in US20160297884A1 |
| P6DY | Identifier 364, 365, 366 in US20160297884A1 |
| DDD1/AD1 zip | Identifier 37 in WO2016124930 |
| CD22 | Identifier 380, 204, 260, 266, 272, 278, 284, 290, 296, 302, 308, 341, 213, 320, |
| | 326, 332, 338, 347, 350, 356, 362, 368, 374, 219, 386, 392, 398, 404, 410, 416, |
| | 421, 427, 225, 230, 1109, 236, 242, 248, 254 in WO2016164731A2; Identifier |
| | 15, 16, 17, 18, 19, 20, 32 in WO2013059593; Identifier 22, 23, 24 in |
| | US20150299317 |
| PSMA | Identifier 39 in WO2015142675; Identifier 28, 29 in US20160311907A1; |
| | Identifier 140, 144, 145, 146, 147, 148, 149, 150, 167, 168, 169, 170, 171, 172, |
| | 173, 174, 151, 152, 153, 154, 155, 156, 139, 138, 175, 157, 158, 159, 160, 161, |
| | 162, 163, 164, 165, 166, 141, 142, 143, 214 in WO2016097231 |
| CD276 | Identifier 39, 40, 41, 42, 43, 44, 45, 46, 47, 122, 123, 124, 125, 126, 127, 128, |
| | 129, 130 in US20160053017 |
| SNAP | Identifier 395 in WO2016187216A1 |
| SSEA4 | Identifier 396, 397 in WO2016187216A1 |
| CEA | Identifier 4 in WO2016008973A1; Identifier 29, 30 in US20140242701A |
| CAR and not gate (CD19 | Identifier 4, 5 in US20160296562 |
| and not CD33) | |
| IL13Ra2specific | Identifier 4, 5, 6 in WO2016089916A1; Identifier 47, 49 in WO2016123143; |
| | Identifier 51, 53, 55 in WO2016123143; Identifier 1, 31, 32, 33, 34, 35, 36, 37, |
| | 38, 39, 40, 41, 42, 43, 44, 45 in US20160340649A1 |
| TSLPR | Identifier 40, 41, 42 in WO2016034666A1; Identifier 39, 40, 41, 42, 43, 44, 45, |
| | 46 in WO2015084513; Identifier 39, 40, 41, 42, 43 in US20160311910A1 |
| CAR and gate (CD19 and | Identifier 41 in US20160296562 |
| GD2) | |
| CAR and gate (CD19 and | Identifier 43 in US20160296562 |
| CD5) | |
| ALK | Identifier 43, 44, 45, 46, 47, 48, 49, 50, 51, 52, 53, 54, 55, 56, 57, 58, 59, 60, 61, |
| | 62, 63, 64, 65, 66, 67, 68, 69, 70, 71, 72, 73, 74, 75, 76, 77, 78, 79, 80, 81, 82, |
| | 84, 85, 86, 87, 88, 89, 90 in WO2015069922 |
| VEGFR2 | Identifier 44, 45, 46 in US20160311910A1; Identifier 10, 11, 12 in |
| | US20120213783 |
| CAR and gate (CD19 and | Identifier 45 in US20160296562 |
| EGFR VIII) | |
| KMA | Identifier 46 in US20160340649A1 |
| Mesothelin | Identifier 47, 48 in US20160340649A1; Identifier 48 in US20160340649A1; |
| | Identifier 27 in WO2016172703A2; Identifier 18, 19, 20, 21, 22, 23 in |
| | WO2013142034; Identifier 3 in WO2013067492 |
| CAR and not gate 1 | Identifier 48 in US20160296562 |
| HIV Env | Identifier 48, 49 in WO2016168766A1; Identifier 4 in EP2997134A4; Identifier |
| | 7, 9, 47, 49 in WO2015077789 |
| CD33 | Identifier 48, 49, 50, 51, 52, 53, 54, 55, 83 in WO2016014576; Identifier 19, 20, |
| | 21, 22, 23, 24, 25, 26, 27, 28, 29, 30, 31, 32, 33, 34, 35, 36, 37, 38, 39, 40, 41, |
| | 42, 48, 49, 50, 51, 52, 53, 54, 55, 56, 57, 58, 59, 60, 61, 62, 63, 64, 65, 66, 67, |
| | 68, 69, 70, 71 in WO2015150526A2 |
| CAR and not gate 2 | Identifier 49 in US20160296562 |
| CD435 | Identifier 5 in EP3074419A2 |
| TOSO | Identifier 5, 4 in WO2015075468 |
| MUC1 | Identifier 5, 7 in WO2013063419; Identifier 51 in US20160340406A1; |
| | Identifier 30, 32, 34 in US20160130357; Identifier 295, 298, 301, 304, 307, 607, |
| | 609, 611, 613 in WO2016130726 |
| CAR and not gate 3 | Identifier 50 in US20160296562 |
| CD8 stalk APRIL | Identifier 51 in US20160296562A1 |
| HSP70 | Identifier 51, 53, 5 in WO2015077789; Identifier 21, 22, 23, 24, 25, 26, 27, 28, |
| | 29 in WO2016120217 |
| APRIL-based CAR | Identifier 53 in US20160296562A1; Identifier 52 in US20160296562A1 |
| CS1 | Identifier 55, 57, 60, 54, 56, 48, 49, 50, 51, 52, 53, 58, 59, 61, 62 in |
| | WO2015121454; Identifier 28 in WO2014179759A1 |
| CAR and not gate (CD19 | Identifier 6 in US20160296562A1 |
| and not CD33) | |
| Trophoblast Glycoprotein | Identifier 6 in WO2015075468; Identifier 4 in US20160347854A1; Identifier 4 |
| 5T4 | in EP3098237A1; Identifier 19, 20, 21, 22, 23, 24, 25, 26, 27, 28, 29, 30, 31, 32, |
| | 33, 34, 35, 36, 37, 38, 39 in WO2016034666A1 |
| HERVK | Identifier 6 in WO2016168769A1 |
| NCAR with RQR82 ACD | Identifier 615 in WO2016130726 |
| 19 | |
| NYBR1 | Identifier 617, 619 in WO2016130726; Identifier 218 in WO2016097231; |
| | Identifier 26, 29, 60 in WO2015112830; Identifier 1 in US20160333422A1 |
| CD123 | Identifier 69 in WO2016142532; Identifier 23, 24, 25, 26, 27, 28, 29, 30, 31, 32, |
| | 33, 34, 35, 36, 37, 38, 39, 40, 41, 42, 44, 45, 46, 47, 48 in WO2015140268A1; |
| | Identifier 9, 10, 11, 12 in US20140271582; Identifier 56, 57, 58, 59, 60, 61 in |
| | WO2016097231; Identifier 98, 99, 100, 101, 125, 126, 127, 128, 129, 130, 131, |
| | 132, 133, 134, 135, 136, 137, 138, 139, 140, 141, 142, 143, 144, 145, 146, 147, |
| | 148, 149, 150, 151, 152, 153, 154, 155, 156 in WO2016028896; Identifier 31, |
| | 32, 33, 34, 35, 36, 37, 38, 39, 40, 41, 42, 43, 44, 45, 46, 47, 48, 49, 50, 51, 52, |
| | 53, 54, 55, 56, 57, 58, 59, 60, 61, 62, 63, 64, 65, 66, 67, 68, 69, 70, 71, 72, 73, |
| | 74, 75, 76, 77, 78, 79, 80, 81, 82, 83, 84, 85, 86, 87, 88, 89, 90, 91, 92, 93, 94, |
| | 95, 96, 97, 98, 99, 100, 101, 102, 103, 104, 105, 106, 107, 108, 109, 110, 111, |
| | 112, 113, 114, 115, 116, 117, 118, 119, 120, 121, 122, 123, 124, 125, 126, 127, |
| | 128, 129, 130, 131, 132, 133, 134, 135, 136, 137, 138, 139, 140, 141, 142, 143, |
| | 144, 145, 146, 147, 148, 149, 150, 151, 152, 153, 154, 155, 156, 157, 158, 159, |
| | 160, 172, 173, 174, 175, 176, 177, 178, 179, 180, 181, 182, 183, 184, 185, 186, |
| | 187, 188, 189, 190, 191, 193, 194, 195, 196, 197 in WO2016120220; Identifier |
| | 1, 2, 3, 4, 5, 6, 7, 8, 9, 10, 142 in WO2016120216 |
| CD410 | Identifier 7 in EP3074419A2 |
| CD38 | Identifier 70, 71, 72, 64, 65, 66, 67, 68, 69 in WO2016097231; Identifier 35, 36, |
| | 37 in WO2015121454 |
| GCN4 | Identifier 8, 10 in U.S. Pat. No. 9,446,105B2 |
| CD4-DDY3 | Identifier 9 in EP3074419A2 |
| Fra | Identifier 959 in WO2016090337; Identifier 13 in US20170002072A1 |
| CD70 | Identifier 99 in WO2015121454 |

### Leader Sequence of the CAR

In some embodiments, the extracellular antigen-binding domain further comprises a leader sequence. The leader sequence may be located at the amino-terminus of the extracellular antigen-binding domain. The leader sequence may be optionally cleaved from the antigen-binding moiety during cellular processing and localization of the CAR to the cellular membrane.

In some embodiments, the leader sequence comprises the amino acid sequence of SEQ ID NO: 15, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 15. In some embodiments, the nucleotide sequence encoding the leader comprises the nucleotide sequence that encodes the amino acid sequence of SEQ ID NO: 15, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 15. In some embodiments, the nucleotide sequence encoding the leader sequence comprises the sequence set forth in SEQ ID NO: 16, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 16. In some embodiments, the leader sequence comprises the amino acid sequence of SEQ ID NO: 15. In some embodiments, the nucleotide sequence encoding the leader sequence comprises the nucleotide sequence set forth in SEQ ID NO: 16. In some embodiments, the nucleotide sequence encoding the leader sequence comprises the sequence set forth in SEQ ID NO: 37, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 37. In some embodiments, the nucleotide sequence encoding the leader sequence comprises the nucleotide sequence set forth in SEQ ID NO: 37.

### Transmembrane Domain of the CAR

In some embodiments, the CARs expressed by the modified immune effector cell comprise a transmembrane domain. The transmembrane domain may be fused in frame between the extracellular target-binding domain and the cytoplasmic domain.

The transmembrane domain may be derived from the protein contributing to the extracellular target-binding domain, the protein contributing the signaling or co-signaling domain, or by a totally different protein. In some instances, the transmembrane domain can be selected or modified by amino acid substitution, deletions, or insertions to minimize interactions with other members of the CAR complex. In some instances, the transmembrane domain can be selected or modified by amino acid substitution, deletions, or insertions to avoid-binding of proteins naturally associated with the transmembrane domain. In some embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains of particular use in this invention may be derived from (i.e. comprise at least the transmembrane region(s) of) the α, β or ζ chain of the T-cell receptor, CD28, CD3 epsilon, CD45, CD4, CD5, CD8, CD9, CD16, CD22, CD33, CD37, CD40, CD64, CD80, CD86, CD134, CD137, CD154. Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In some embodiments, the transmembrane domain may be derived from CD8α, CD28, CD8, CD4, CD3ζ, CD40, CD134 (OX-40), NKG2A/C/D/E, or CD7. In some embodiments, the transmembrane domain may be derived from CD28.

In some embodiments, it will be desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid-binding of such domains to the transmembrane domains of the same or different surface membrane proteins to minimize interactions with other members of the receptor complex. In other cases, it will be desirable to employ the transmembrane domain of ζ, η or FcεR1γ and -β, MB1 (Igα.), B29 or CD3-γ, ζ, or η, in order to retain physical association with other members of the receptor complex.

In some embodiments, the transmembrane domain is derived from CD3ζ, CD28, CD4, or CD8α.

In a specific embodiment, the transmembrane domain is derived from the CD3 transmembrane domain. In some embodiments, the CD3ζ transmembrane domain comprises the amino acid sequence set forth in SEQ ID NO: 23, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 23. In some embodiments, the nucleotide sequence that encodes the CD3ζ transmembrane domain comprises the nucleotide sequence that encodes the amino acid sequence of SEQ ID NO: 23, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 23. In some embodiments, the nucleotide sequence that encodes the CD3ζ transmembrane domain comprises the nucleotide sequence set forth in SEQ ID NO: 24, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 24. In some embodiments, the CD3ζ transmembrane domain comprises the amino acid sequence set forth in SEQ ID NO: 23. In some embodiments, the nucleotide sequence that encodes the CD3ζ transmembrane domain comprises the nucleotide sequence set forth in SEQ ID NO: 24.

In a specific embodiment, the transmembrane domain is derived from the CD28 transmembrane domain. In some embodiments, the CD28 transmembrane domain comprises the amino acid sequence set forth in SEQ ID NO: 31, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 31. In some embodiments, the nucleotide sequence that encodes the CD28 transmembrane domain comprises the nucleotide sequence that encodes the amino acid sequence of SEQ ID NO: 31, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 31. In some embodiments, the nucleotide sequence that encodes the CD28 transmembrane domain comprises the nucleotide sequence set forth in SEQ ID: 32, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 32. In some embodiments, the CD28 transmembrane domain comprises the amino acid sequence set forth in SEQ ID NO: 31. In some embodiments, the nucleotide sequence that encodes the CD28 transmembrane domain comprises the nucleotide sequence set forth in SEQ ID NO: 32.

In a specific embodiment, the transmembrane domain is derived from the CD8a transmembrane domain. In some embodiments, the CD8a transmembrane domain comprises the amino acid sequence set forth in SEQ ID NO: 49, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 49. In some embodiments, the nucleotide sequence that encodes the CD8a transmembrane domain comprises the nucleotide sequence that encodes the amino acid sequence of SEQ ID NO: 49, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 49. In some embodiments, the nucleotide sequence that encodes the CD8a transmembrane domain comprises the nucleotide sequence set forth in SEQ ID NO: 50, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 50. In some embodiments, the CD8a transmembrane domain comprises the amino acid sequence set forth in SEQ ID NO: 49. In some embodiments, the nucleotide sequence that encodes the CD8a transmembrane domain comprises the nucleotide sequence set forth in SEQ ID NO: 50.

In a specific embodiment, the transmembrane domain is derived from the CD8a transmembrane domain. In some embodiments, the CD8a transmembrane domain comprises the amino acid sequence set forth in SEQ ID NO: 82, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 82. In some embodiments, the nucleotide sequence that encodes the CD8a transmembrane domain comprises the nucleotide sequence that encodes the amino acid sequence of SEQ ID NO: 82, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 82. In some embodiments, the nucleotide sequence that encodes the CD8α transmembrane domain comprises the nucleotide sequence set forth in SEQ ID NO: 83, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 83. In some embodiments, the nucleotide sequence that encodes the CD8α transmembrane domain comprises the nucleotide sequence set forth in SEQ ID NO: 84, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 84. In some embodiments, the CD8α transmembrane domain comprises the amino acid sequence set forth in SEQ ID NO: 82. In some embodiments, the nucleotide sequence that encodes the CD8α transmembrane domain comprises the nucleotide sequence set forth in SEQ ID NO: 83. In some embodiments, the nucleotide sequence that encodes the CD8α transmembrane domain comprises the nucleotide sequence set forth in SEQ ID NO: 84

In a specific embodiment, the transmembrane domain is derived from the CD4 transmembrane domain. In some embodiments, the CD4 transmembrane domain comprises the amino acid sequence set forth in SEQ ID NO: 51, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 51. In some embodiments, the nucleotide sequence that encodes the CD4 transmembrane domain comprises the nucleotide sequence that encodes the amino acid sequence of SEQ ID NO: 51, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 51. In some embodiments, the nucleotide sequence that encodes the CD4 transmembrane domain comprises the nucleotide sequence set forth in SEQ ID NO: 52, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 52. In some embodiments, the CD4 transmembrane domain comprises the amino acid sequence set forth in SEQ ID NO: 51. In some embodiments, the nucleotide sequence that encodes the CD4 transmembrane domain comprises the nucleotide sequence set forth in SEQ ID NO: 52.

### Linker Domain of the CAR

In some embodiments, the CAR further comprises a linker domain between the extracellular antigen-binding domain and the transmembrane domain, wherein the antigen-binding domain, linker, and the transmembrane domain are in frame with each other.

The term “linker domain” as used herein generally means any oligo- or polypeptide that functions to link the antigen-binding moiety to the transmembrane domain. A linker domain can be used to provide more flexibility and accessibility for the antigen-binding moiety. A linker domain may comprise up to 300 amino acids, preferably 10 to 100 amino acids and most preferably 25 to 50 amino acids. A linker domain may be derived from all or part of naturally occurring molecules, such as from all or part of the extracellular region of CD8, CD4 or CD28, or from all or part of an antibody constant region. Alternatively, the linker domain may be a synthetic sequence that corresponds to a naturally occurring linker domain sequence, or may be an entirely synthetic linker domain sequence. Non-limiting examples of linker domains which may be used in accordance with the invention include a part of human CD8α chain, partial extracellular domain of CD28, FcγRllla receptor, IgG, IgM, IgA, IgD, IgE, an Ig hinge, or functional fragment thereof. In some embodiments, additional linking amino acids are added to the linker domain to ensure that the antigen-binding moiety is an optimal distance from the transmembrane domain. In some embodiments, when the linker is derived from an Ig, the linker may be mutated to prevent Fc receptor binding.

In some embodiments, the linker domain comprises a hinge region. In some embodiments, the hinge region comprises the amino acid sequence SEQ ID NO: 19, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 19. In some embodiments, the nucleotide sequence encoding the hinge region comprises the nucleotide sequence that encodes the amino acid sequence of SEQ ID NO: 19, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 19. In some embodiments, the nucleotide sequence encoding the hinge region comprises the sequence set forth in SEQ ID NO: 20, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 20. In some embodiments, the hinge region comprises the amino acid sequence of SEQ ID NO: 19. In some embodiments, the nucleotide sequence encoding the hinge region comprises the nucleotide sequence set forth in SEQ ID NO: 20.

In some embodiments, the hinge region comprises the amino acid sequence SEQ ID NO: 78, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 78. In some embodiments, the nucleotide sequence encoding the hinge region comprises the nucleotide sequence that encodes the amino acid sequence of SEQ ID NO: 78, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 78. In some embodiments, the nucleotide sequence encoding the hinge region comprises the sequence set forth in SEQ ID NO: 79, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 79. In some embodiments, the hinge region comprises the amino acid sequence of SEQ ID NO: 78. In some embodiments, the nucleotide sequence encoding the hinge region comprises the nucleotide sequence set forth in SEQ ID NO: 79.

In some embodiments, the hinge region comprises the amino acid sequence SEQ ID NO: 80, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 80. In some embodiments, the nucleotide sequence encoding the hinge region comprises the nucleotide sequence that encodes the amino acid sequence of SEQ ID NO: 80, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 80. In some embodiments, the nucleotide sequence encoding the hinge region comprises the sequence set forth in SEQ ID NO: 81, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 81. In some embodiments, the hinge region comprises the amino acid sequence of SEQ ID NO: 80. In some embodiments, the nucleotide sequence encoding the hinge region comprises the nucleotide sequence set forth in SEQ ID NO: 81.

Other hinge regions suitable for use in the present invention may be derived from an immunoglobulin IgG hinge or functional fragment, including IgG1, IgG2, IgG3, IgG4, IgM1, IgM2, IgA1, IgA2, IgD, IgE, or a chimera or variant thereof.

In some embodiments, the linker domain comprises a hinge region which is an IgG1 hinge. In some embodiments, the IgG1 hinge comprises the amino acid sequence SEQ ID NO: 40, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 40. In some embodiments, the nucleotide sequence encoding the IgG1 hinge comprises the nucleotide sequence that encodes the amino acid sequence of SEQ ID NO: 40, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 40. In some embodiments, the nucleotide sequence encoding the IgG1 hinge comprises the sequence set forth in SEQ ID NO: 41, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 41. In some embodiments, the hinge region comprises the amino acid sequence of SEQ ID NO: 40. In some embodiments, the nucleotide sequence encoding the IgG1 hinge comprises the nucleotide sequence set forth in SEQ ID NO: 41.

In some embodiments, the linker domain comprises the amino acid sequence SEQ ID NO: 21. or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 21. In some embodiments, the nucleotide sequence encoding the linker domain comprises the nucleotide sequence that encodes the amino acid sequence of SEQ ID NO: 21, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 21. In some embodiments, the nucleotide sequence encoding the linker domain comprises the sequence set forth in SEQ ID NO: 22, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 22. In some embodiments, the linker domain comprises the amino acid sequence of SEQ ID NO: 21. In some embodiments, the nucleotide sequence encoding the linker domain comprises the nucleotide sequence set forth in SEQ ID NO: 22. In some embodiments, the nucleotide sequence encoding the linker domain comprises the sequence set forth in SEQ ID NO: 42, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 42. In some embodiments, the nucleotide sequence encoding the linker domain comprises the nucleotide sequence set forth in SEQ ID NO: 42.

In some embodiments, the linker domain comprises the amino acid sequence SEQ ID NO: 119. or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 119. In some embodiments, the nucleotide sequence encoding the linker domain comprises the nucleotide sequence that encodes the amino acid sequence of SEQ ID NO: 119, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 119. In some embodiments, the nucleotide sequence encoding the linker domain comprises the sequence set forth in SEQ ID NO: 120, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 120. In some embodiments, the linker domain comprises the amino acid sequence of SEQ ID NO: 119. In some embodiments, the nucleotide sequence encoding the linker domain comprises the nucleotide sequence set forth in SEQ ID NO: 120.

### Cytoplasmic Domain of the CAR

In some embodiments, the CAR expressed by the immune effector cell described herein further comprises a cytoplasmic domain. In some embodiments, the cytoplasmic domain of the CAR comprises one or more lymphocyte activation domains.

The cytoplasmic domain, which comprises the lymphocyte activation domain of the CAR, is responsible for activation of at least one of the normal effector functions of the lymphocyte in which the CAR has been placed in. The term “effector function” refers to a specialized function of a cell. Effector function of a T cell, for example, may be cytolytic activity or helper activity including the secretion of cytokines. Thus, the term “lymphocyte activation domain” refers to the portion of a protein which transduces the effector function signal and directs the cell to perform a specialized function. While usually the entire lymphocyte activation domain is present, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the lymphocyte activation domain sufficient to transduce the effector function signal.

Non-limiting examples of lymphocyte activation domains which can be used in the CARs described herein include those derived from DAP10, DAP12, Fc epsilon receptor I γ chain (FCER1G), CD3δ, CD3ε, CD3γ, CD3ζ, CD27, CD28, CD40, CD134, CD137, CD226, CD79A, ICOS, and MyD88.

In some embodiments, the lymphocyte activation domain is derived from CD3ζ and comprises the amino acid sequence SEQ ID NO: 25. In some embodiments, the CD3ζ signaling domain comprises the amino acid sequence set forth in SEQ ID NO: 25 or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 25. In some embodiments, the nucleotide sequence that encodes the CD3 signaling domain comprises the nucleotide sequence that encodes the amino acid sequence of SEQ ID NO: 25, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 25. In some embodiments, the nucleotide sequence that encodes the CD3ζ signaling domain comprises the nucleotide sequence set forth in SEQ ID NO: 26, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 26. In some embodiments, the CD3ζ signaling domain comprises the amino acid sequence set forth in SEQ ID NO: 25. In some embodiments, the nucleotide sequence that encodes the CD3ζ signaling domain comprises the nucleotide sequence set forth in SEQ ID NO: 26. In some embodiments, the nucleotide sequence that encodes the CD3ζ signaling domain comprises the nucleotide sequence set forth in SEQ ID NO: 44, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 44. In some embodiments, the nucleotide sequence that encodes the CD3ζ signaling domain comprises the nucleotide sequence set forth in SEQ ID NO: 88. In some embodiments, the nucleotide sequence that encodes the CD3ζ signaling domain comprises the nucleotide sequence set forth in SEQ ID NO: 88, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 88. In some embodiments, the nucleotide sequence that encodes the CD3ζ signaling domain comprises the nucleotide sequence set forth in SEQ ID NO: 88.

Non-limiting examples of co-stimulatory domains which can be used in the CARs of the present disclosure include, those derived from 4-1BB (CD137), CD28, CD40, ICOS, CD134 (OX-40), BTLA, CD27, CD30, GITR, CD226, CD79A, HVEM, MyD88, IL-2Rβ, or the STAT3-binding YXXQ. In some embodiments, the CAR of the present disclosure comprises one co-stimulatory domain. In some embodiments, the CAR of the present disclosure comprises a co-stimulatory domain derived from CD28.

In some embodiments, the co-stimulatory domains which can be used in the CARs of the present disclosure may be derived from CD28, 4-1BB, CD27, CD40, CD134, CD226, CD79A, ICOS, or MyD88, or any combination thereof.

In some embodiments, the CAR of the present disclosure comprises one or more co-stimulatory domains. In some embodiments, the CAR of the present disclosure comprises two or more co-stimulatory domains. In certain embodiments, the CAR of the present disclosure comprises two, three, four, five, six or more co-stimulatory domains. For example, the CAR of the present disclosure may comprise a co-stimulatory domain derived from 4-1BB and a co-stimulatory domain derived from CD28.

In certain embodiments, the CARs of the present disclosure comprise a cytoplasmic domain, which comprises a signaling domain, a MyD88 polypeptide or functional fragment thereof, and a CD40 cytoplasmic polypeptide region or a functional fragment thereof. In certain embodiments, the CAR lacks the CD40 transmembrane and/or CD40 extracellular domains. In certain embodiments, the CAR includes the CD40 transmembrane domain. In certain embodiments, the CAR includes the CD40 transmembrane domain and a portion of the CD40 extracellular domain, wherein the CD40 extracellular domain does not interact with natural or synthetic ligands of CD40.

In certain embodiments, the signaling domain is separated from the MyD88 polypeptide or functional fragment thereof and/or the CD40 cytoplasmic polypeptide region or a functional fragment thereof. In certain embodiments, the lymphocyte activation domain is separated from the MyD88 polypeptide or functional fragment thereof and/or the CD40 cytoplasmic polypeptide region or a functional fragment thereof by at least 1, 2, 3, 4, 5, 6, 7, 8, 9, 10, 11, 12, 13, 14, or 15 amino acids.

In some embodiments, the signaling domain(s) and co-stimulatory domain(s) can be in any order. In some embodiments, the signaling domain is upstream of the co-stimulatory domains. In some embodiments, the signaling domain is downstream from the co-stimulatory domains. In the cases where two or more co-stimulatory domains are included, the order of the co-stimulatory domains could be switched.

In some embodiments, the co-stimulatory domain is derived from CD28 and comprises the amino acid sequence SEQ ID NO: 33. In some embodiments, the CD28 co-stimulatory domain comprises the amino acid sequence set forth in SEQ ID NO: 33 or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 33. In some embodiments, the nucleotide sequence that encodes the CD28 co-stimulatory domain comprises the nucleotide sequence that encodes the amino acid sequence of SEQ ID NO: 33, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 33. In some embodiments, the nucleotide sequence that encodes the CD28 co-stimulatory domain comprises the nucleotide sequence set forth in SEQ ID NO: 34, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 34. In some embodiments, the nucleotide sequence that encodes the CD28 co-stimulatory domain comprises the nucleotide sequence set forth in SEQ ID NO: 85, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 85. In some embodiments, the CD28 co-stimulatory domain comprises the amino acid sequence set forth in SEQ ID NO: 33. In some embodiments, the nucleotide sequence that encodes the CD28 co-stimulatory domain comprises the nucleotide sequence set forth in SEQ ID NO: 34. In some embodiments, the nucleotide sequence that encodes the CD28 co-stimulatory domain comprises the nucleotide sequence set forth in SEQ ID NO: 85.

In some embodiments, the co-stimulatory domain is derived from 4-1BB (CD137) and comprises the amino acid sequence SEQ ID NO: 86. In some embodiments, the 4-1BB (CD137) co-stimulatory domain comprises the amino acid sequence set forth in SEQ ID NO: 86 or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 86. In some embodiments, the nucleotide sequence that encodes the 4-1BB (CD137) co-stimulatory domain comprises the nucleotide sequence that encodes the amino acid sequence of SEQ ID NO: 86, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 86. In some embodiments, the nucleotide sequence that encodes the 4-1BB (CD137) co-stimulatory domain comprises the nucleotide sequence set forth in SEQ ID NO: 87, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 87. In some embodiments, the 4-1BB (CD137) co-stimulatory domain comprises the amino acid sequence set forth in SEQ ID NO: 86. In some embodiments, the nucleotide sequence that encodes the 4-1BB (CD137) co-stimulatory domain comprises the nucleotide sequence set forth in SEQ ID NO: 87.

In some embodiments, the cytoplasmic domain comprises both the CD3ζ lymphocyte activation domain and the CD28 co-stimulatory domain, which are fused in frame. The CD3ζ lymphocyte activation domain and the CD28 co-stimulatory domain can be in any order. In some embodiments, the CD3ζ lymphocyte activation domain is downstream of the CD28 co-stimulatory domain.

### Accessory Genes of the CAR

In addition to the CAR construct, the CAR may further comprise at least one additional gene that encodes an additional peptide. Examples of additional genes can include a transduced host cell selection marker, an in vivo tracking marker, cellular marker, epitope tag, a cytokine, a suicide gene, safety switch, or some other functional gene. In certain embodiments, the functional additional gene can induce the expression of another molecule. In certain embodiments, the functional additional gene can increase the safety of the CAR. For example, the CAR construct may comprise an additional gene which is truncated CD19 (tCD19). The tCD19 can be used as a tag. Expression of tCD19 may also help determine transduction efficiency.

Other examples of additional genes include genes that encode polypeptides with a biological function; examples include, but are not limited to, cytokines, chimeric cytokine receptors, dominant negative receptors, safety switches (CD20, truncated EGFR or HER2, inducible caspase 9 molecules). As another example, the CAR construct may comprise an additional gene which is a synNotch receptor. Once activated, the synNotch receptor can induce the expression of a target gene (e.g., a second CAR and/or bispecific molecule).

In some embodiments, the CAR may comprise one or more additional nucleotide sequences encoding one or more additional polypeptide sequences. As a non-limiting example, the one or more additional polypeptide sequences may be selected from one or more cellular markers, epitope tags, cytokines, safety switches, dimerization moieties, or degradation moieties.

In certain embodiments, the CAR comprises at least one additional gene (i.e., a second gene). In certain embodiments, the CAR comprises one second gene. In other embodiments, the CAR comprises two additional genes (i.e., a third gene). In yet another embodiment, the CAR comprises three additional genes (i.e., a fourth gene). In certain embodiments, the additional genes are separated from each other and the CAR construct. For example, they may be separated by 2A sequences and/or an internal ribosomal entry sites (IRES). In certain examples, the CAR can be at any position of the polynucleotide chain (for example construct A: CAR, second gene, third gene, fourth gene; construct B: second gene, CAR, third gene, fourth gene; etc.).

Non-limiting examples of classes of accessory genes that can be used to increase the effector function of CAR containing immune effector cells, include i) secretable cytokines (e.g., but not limited to, IL-7, IL-12, IL-15, IL-18), ii) membrane bound cytokines (e.g., but not limited to, IL-15), iii) chimeric cytokine receptors (e.g., but not limited to, IL-2/IL-7, IL-4/IL-7), iv) constitutive active cytokine receptors (e.g., but not limited to, C7R), v) dominant negative receptors (DNR; e.g., but not limited to TGFRII DNR), vi) ligands of co-stimulatory molecules (e.g., but not limited to, CD80, 4-1BBL), vii) antibodies, including fragments thereof and bispecific antibodies (e.g., but not limited to, bispecific T-cell engagers (BiTEs)), or vii) a second CAR.

In some embodiments, the accessory gene included herein is a truncated CD19 molecule (tCD19). In some embodiments, the tCD19 molecule comprises the amino acid sequence set forth in SEQ ID NO: 49 or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 49. In some embodiments, the nucleotide sequence that encodes the tCD19 molecule comprises the nucleotide sequence that encodes the amino acid sequence of SEQ ID NO: 49, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 49. In some embodiments, the nucleotide sequence that encodes the tCD19 molecule comprises the nucleotide sequence set forth in SEQ ID NO: 50, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 50. In some embodiments, the tCD19 molecule comprises the amino acid sequence set forth in SEQ ID NO: 49. In some embodiments, the nucleotide sequence that encodes the tCD19 molecule comprises the nucleotide sequence set forth in SEQ ID NO: 50.

tCD19 may be separated from the CAR-encoding sequence by a separation sequence (e.g., a 2A sequence). tCD19 could also be replaced with two accessory genes separated by a separation sequence (e.g., a 2A sequence) using a combination of the classes of molecules listed above (e.g., CAR-2A-CD20-2A-IL15). In addition, the use of two separation sequences (e.g., 2A sequences) would allow the expression of TCR (e.g., CAR-2A-TCRα-2A-TCRβ). In the constructs with a CAR and two or three accessory genes, the order of the CAR and the 2nd or 3rd transgene could be switched.

In certain embodiments, the additional gene may be regulated by an NFAT dependent-promoter. Activation of the T-cell or other lymphocyte leads to activation of the transcription factor NFAT resulting in the induction of the expression of the protein encoded by the gene linked with the NFAT dependent promoter. One or more members of the NFAT family (i.e., NFATc1, NFATc2, NFATc3, NFATc4, and NFAT5) is expressed in most cells of the immune system. NFAT-dependent promoters and enhancers tend to have three to five NFAT binding sites.

In certain embodiments, the functional additional gene can be a suicide gene. A suicide gene is a recombinant gene that will cause the host cell that the gene is expressed in to undergo programmed cell death or antibody mediated clearance at a desired time. Suicide genes can function to increase the safety of the CAR. In another embodiment, the additional gene is an inducible suicide gene. Non-limiting examples of suicide genes include i) molecules that are expressed on the cell surface and can be targeted with a clinical grade monoclonal antibody including CD20, EGFR or a fragment thereof, HER2 or a fragment thereof, and ii) inducible suicide genes (e.g., but not limited to inducible caspase 9 (see Straathof et al. (2005) Blood. 105(11): 4247-4254; US Publ. No. 2011/0286980, each of which are incorporated herein by reference in their entirety for all purposes)).

In certain aspects, CARs of the present disclosure may be regulated by a safety switch. As used herein, the term “safety switch” refers to any mechanism that is capable of removing or inhibiting the effect of a CAR from a system (e.g., a culture or a subject). Safety switches can function to increase the safety of the CAR.

The function of the safety switch may be inducible. Non-limiting examples of safety switches include (a) molecules that are expressed on the cell surface and can be targeted with a clinical grade monoclonal antibody including CD20, EGFR or a fragment thereof, HER2 or a fragment thereof, and (b) inducible suicide genes (e.g., but not limited to herpes simplex virus thymidine kinase (HSV-TK) and inducible caspase 9 (see Straathof et al. (2005) Blood. 105(11): 4247-4254; US Publ. No. 2011/0286980, each of which are incorporated herein by reference in their entirety for all purposes).

In some embodiments, the safety switch is a CD20 polypeptide. Expression of human CD20 on the cell surface presents an attractive strategy for a safety switch. The inventors and others have shown that cells that express CD20 can be rapidly eliminated with the FDA approved monoclonal antibody rituximab through complement-mediated cytotoxicity and antibody-dependent cell-mediated cytotoxicity (see e.g., Griffioen, M., et al. Haematologica 94, 1316-1320 (2009), which is incorporated herein by reference in its entirety for all purposes). Rituximab is an anti-CD20 monoclonal antibody that has been FDA approved for Chronic Lymphocytic Leukemia (CLL) and Non-Hodgkin's Lymphoma (NHL), among others (Storz, U. MAbs 6, 820-837 (2014), which is incorporated herein by reference in its entirety for all purposes). The CD20 safety switch is non-immunogenic and can function as a reporter/selection marker in addition to a safety switch (Bonifant, C. L., et al. Mol Ther 24, 1615-1626 (2016); van Loenen, M. M., et al. Gene Ther 20, 861-867 (2013); each of which is incorporated herein by reference in its entirety for all purposes).

In some embodiments, the polynucleotide sequence(s) encoding the CARs of the present disclosure may be expressed in an inducible fashion, for example, as may be achieved with an inducible promoter, an inducible expression system, an artificial signaling circuits, and/or drug-induced splicing.

In some embodiments, the polynucleotide sequence(s) encoding the CARs of the present disclosure may be expressed in an inducible fashion, such as that which may be achieved with i) an inducible promoter, for example, but not limited to promotors that may be activated by T cell activation (e.g. NFAT, Nur66, IFNg) or hypoxia; ii) an inducible expression system, for example, but not limited to doxycycline- or tamoxifen-inducible expression system; iii) artificial signaling circuits including, but not limited to, SynNotch, and/or iv) drug-induced splicing.

In some embodiments, the polynucleotide sequence(s) encoding the CARs disclosed herein may be expressed as a ‘split molecule’ in which for example, transmembrane and intracellular signaling regions, or any other domains or regions of the CAR, may be assembled only in the presence of a heterodimerizing small molecule (e.g., small organic molecule, nucleic acid, polypeptide, or a fragment, isoform, variant, analog, or derivative thereof).

In some embodiments, the polynucleotide sequence(s) encoding the CARs herein may further encode a moiety so that the stability of CAR may be regulated with a small molecule, including but not limited to, the “SWIFF” technology or an immunomodulatory drug (IMiD)-inducible degron.

A “separation sequence” refers to a peptide sequence that causes a ribosome to release the growing polypeptide chain that it is being synthesizes without dissociation from the mRNA. In this respect, the ribosome continues translating and therefore produces a second polypeptide. Non-limiting examples of separation sequences includes T2A (EGRGSLLTCGDVEENPGP (SEQ ID NO: 45) or GSGEGRGSLLTCGDVEENPGP (SEQ ID NO: 53)); the foot and mouth disease virus (FMDV) 2A sequence (GSGSRVTELLYRMKRAETYCPRPLLAIHPTEARHKQKIVAPVKQLLNFDLLKLAGD VESNPGP (SEQ ID NO: 54)); Sponge (Amphimedon queenslandica) 2A sequence (LLCFLLLLLSGDVELNPGP (SEQ ID NO: 55); or HHFMFLLLLLAGDIELNPGP (SEQ ID NO: 56)); acorn worm (Saccoglossus kowalevskii) 2A sequence (WFLVLLSFILSGDIEVNPGP (SEQ ID NO: 57)); amphioxus (Branchiostoma floridae) 2A sequence (KNCAMYMLLLSGDVETNPGP (SEQ ID NO: 58); or MVISQLMLKLAGDVEENPGP (SEQ ID NO: 59)); porcine teschovirus-1 2A sequence (GSGATNFSLLKQAGDVEENPGP (SEQ ID NO: 60)); and equine rhinitis A virus 2A sequence (GSGQCTNYALLKLAGDVESNPGP (SEQ ID NO: 61)). In some embodiments, the separation sequence is a naturally occurring or synthetic sequence. In some embodiments, the separation sequence includes the 2A consensus sequence D-X-E-X-NPGP (SEQ ID NO: 62), in which X is any amino acid residue.

In some embodiments, the separation sequence comprises a Peptide 2A (P2A) sequences disclosed herein. In some embodiments, the P2A separation sequence domain comprises the amino acid sequence SEQ ID NO: 117. or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 117. In some embodiments, the nucleotide sequence encoding the P2A separation sequence domain comprises the nucleotide sequence that encodes the amino acid sequence of SEQ ID NO: 117, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 117. In some embodiments, the nucleotide sequence encoding the P2A separation sequence domain comprises the sequence set forth in SEQ ID NO: 118, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 118. In some embodiments, the P2A separation sequence domain comprises the amino acid sequence of SEQ ID NO: 117. In some embodiments, the nucleotide sequence encoding the P2A separation sequence domain comprises the nucleotide sequence set forth in SEQ ID NO: 118.

Alternatively, an Internal Ribosome Entry Site (IRES) may be used to link the CAR and the additional gene. IRES is an RNA element that allows for translation initiation in a cap-independent manner. IRES can link two coding sequences in one bicistronic vector and allow the translation of both proteins in cells.

In certain embodiments, the immune effector cells can be genetically modified to express not only CARs as disclosed herein but to also express fusion protein with signaling activity (e.g., costimulation, T-cell activation). These fusion proteins can improve host cell activation and/or responsiveness. In certain embodiments, the fusion protein can enhance the host cell's response to the target antigen. In certain embodiments, the fusion protein can impart resistance to suppression signals.

In certain embodiments, fusion proteins can comprise portions of CD4, CD8α, CD28, portions of a T-cell receptor, or an antigen-binding moiety (e.g., scFv) linked to a MyD88, CD40, and/or other signaling molecules.

In certain embodiments, the fusion protein comprises an extracellular target-binding domain (as disclosed above), a transmembrane domain (as described above) and a cytoplasmic domain, wherein the cytoplasmic domain comprises at least one co-stimulatory protein (as described above). In certain embodiments, the co-stimulatory fusion protein does not comprise a lymphocyte activation domain (e.g., CD3ζ). In certain embodiments, the at least one co-stimulatory protein can be a MyD88 polypeptide or functional fragment thereof, and/or a CD40 cytoplasmic polypeptide region or a functional fragment thereof.

In certain embodiments, the fusion protein comprises an extracellular domain (such as, but not limited to CD19, CD34), a transmembrane domain (as described above) and a cytoplasmic domain, wherein the cytoplasmic domain comprises at least one co-stimulatory protein (as described above). In certain embodiments, the fusion protein does not comprise a lymphocyte activation domain (e.g., CD3ζ). In certain embodiments, the at least one portion of the fusion protein can be a MyD88 polypeptide or functional fragment thereof, and/or a CD40 cytoplasmic polypeptide region or a functional fragment thereof.

Non-limiting examples of fusion proteins include, but are not limited to, the constructs in the publication of WO2019222579 and WO2016073875, which are incorporated herein by reference in their entirety for all purposes.

In certain embodiments, the fusion proteins are introduced into the immune effector cells on a separate vector from the CAR. In certain embodiments, the fusion proteins are introduced into the immune effector cells on the same vector as the CAR. In certain embodiments, the fusion proteins are introduced into the immune effector cells on the same vector as the CAR but separated by a separation sequence such as 2A.

### Non-Limiting Examples of CARs

In some embodiments, the CAR can be encoded by one polynucleotide chain.

In some embodiments, the CAR of the invention is encoded by a nucleotide sequence comprising the nucleotide sequence of SEQ ID NO: 106, 108, 110, 112, or 116, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% sequence identity with SEQ ID NO: 106, 108, 110, 112, or 116. In some embodiments, the CAR comprises the amino acid sequence of SEQ ID NO: 105, 107, 109, 111, or 115, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% sequence identity with SEQ ID NO: 105, 107, 109, 111, or 115.

In some embodiments, the CAR of the invention is encoded by a nucleotide sequence comprising the nucleotides sequence of SEQ ID NO: 4, 6, 10, 12, or 14, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% sequence identity with SEQ ID NO: 4, 6, 10, 12, or 14. In some embodiments, the CAR comprises the amino acid sequence of SEQ ID NO: 3, 5, 9, 11, or 13, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% sequence identity with SEQ ID NO: 3, 5, 9, 11, or 13.

In some embodiments, the CAR of the invention is encoded by a nucleotide sequence comprising the nucleotides sequence of SEQ ID NO: 152, 154, 156, 158, or 160, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% sequence identity with SEQ ID NO: 152, 154, 156, 158, or 160. In some embodiments, the CAR comprises the amino acid sequence of SEQ ID NO: 151, 153, 155, 157, or 159, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% sequence identity with SEQ ID NO: 151, 153, 155, 157, or 159.

### Methods for Generating Modified Immune Effector Cells

In one aspect, the present invention provides a method for generating a modified immune effector cell described herein. In a related aspect, the present invention provides a method of preserving developmental potential of an immune effector cell. Such methods may comprise modifying an ASXL1 gene or gene product in the cell so that the expression and/or function of ASXL1 in the cell is reduced or eliminated. As a non-limiting example, the immune effector cell may be any of the various T cells disclosed herein. In particular, the T cell may be selected from, e.g., T cell a CD8+ T cell, a CD4+ T cell, a cytotoxic T cell, an αβ T cell receptor (TCR) T cell, a natural killer T (NKT) cell, a γδ T cell, a memory T cell, a T-helper cell, and a regulatory T cell (Treg). In some embodiments, the immune effector cell may be an NK cell. In some embodiments, the above-described methods may comprise modifying the immune effector cell to express a CAR disclosed herein that is capable of binding to an antigen, e.g., an antigen specific to tumor disclosed herein.

The methods may further comprise modifying a DNMT3A gene or gene product in the cell so that the expression and/or function of DNMT3A in the cell is reduced or eliminated. The methods may further comprise modifying a TET2 gene or gene product in the cell so that the expression and/or function of TET2 in the cell is reduced or eliminated. In some embodiments, the ASXL1 gene, the DNMT3A gene, and/or the TET2 gene may be deleted. In certain embodiments, when the DNMT3A gene is deleted or modified, for example, DNMT3A-mediated de novo DNA methylation of the cell genome is inhibited.

In some embodiments, the ASXL1, DNMT3A, and/or TET2 gene or gene product in the immune effector cell may be modified in the presence of one or more inhibitory signals or agents (e.g., compound, small molecule, e.g., small organic molecule, nucleic acid, polypeptide, or a fragment, isoform, variant, analog, or derivative thereof).

As used herein, “small molecule inhibitors” include, but are not limited to, small peptides or peptide-like molecules, soluble peptides, and synthetic non-peptidyl organic or inorganic compounds. A small molecule inhibitor or antagonist can have a molecular weight of any of about 100 to about 20,000 Daltons (Da), about 500 to about 15,000 Da, about 1000 to about 10,000 Da. In some embodiments, the small molecule may be, for example, a peptide and/or a peptidomimetic. A peptidomimetic may include, e.g., chemically modified peptides and peptide-like molecules that contain non-naturally occurring amino acids, peptoids, and the like. Methods for identifying a peptidomimetics are well known in the art and may comprise the screening of databases that contain libraries of possible peptidomimetics.

In some embodiments, the ASXL1, DNMT3A, and/or TET2 gene or gene product may be targeted using any number of various agents (e.g., a small molecule inhibitor). In some embodiments, the agent may be used to reduce the expression and/or activity of ASXL1, DNMT3A, and/or TET2 in a modified immune effector cell disclosed herein.

In some embodiments, the ASXL1 gene, DNMT3A gene, and/or TET2 gene in the immune effector cell may be deleted or modified as a result of an activity of a site-specific nuclease.

Site-specific nucleases may create double-strand breaks or single-strand breaks (i.e., nicks) in a genomic DNA of a cell. Although not wishing to be bound by theory, these breaks are typically repaired by the cell using one of two mechanisms: non-homologous end joining (NHEJ) and homology-directed repair (HDR). In NHEJ, the double-strand breaks are repaired by direct ligation of the break ends to one another. As a result, no new nucleic acid material is inserted into the site, although a few bases may be lost or added, resulting in a small insertions and deletion (indel). In HDR, a donor polynucleotide with homology to the cleaved target DNA sequence is used as a template to repair the cleaved target DNA sequence, resulting in the transfer of genetic information from the donor polynucleotide to the target DNA. As such, new nucleic acid material may be inserted or copied into the cleavage site. In some cases, an exogenous donor polynucleotide can be provided to the cell. The modifications of the target DNA due to NHEJ and/or HDR may lead to, for example, gene correction, gene replacement, gene tagging, transgene insertion, nucleotide deletion, gene disruption, gene mutation, sequence replacement, etc. Accordingly, cleavage of DNA by a site-directed nuclease may be used to delete nucleic acid material from a target DNA sequence by cleaving the target DNA sequence and allowing the cell to repair the sequence in the absence of an exogenously provided donor polynucleotide. Thus, the methods can be used to knock out a gene (resulting in complete lack of transcription or altered transcription) or to knock in genetic material (e.g., a transgene) into a locus of choice in the target DNA.

In some embodiments, the site-specific nuclease is an RNA-guided endonuclease. In particular, a group of RNA-guided endonucleases known as CRISPR-associated (Cas) proteins may be employed to genetically modify the immune effector cell. A Cas protein may form an RNA-protein complex (referred to as RNP) with a guide RNA (gRNA) and is capable of cleaving a target site bearing sequence complementarity to a short sequence (typically about 20-40nt) in the gRNA. In some embodiments, the RNA-guided endonuclease is a Cas9 protein, Cpf1 (Cas12a) protein, C2c1 protein, C2c3 protein, or C2c2 protein.

In a specific embodiment, the RNA-guided endonuclease is a Cas9 protein. The Cas9 protein may be from S. pyogenes, Streptococcus thermophilus, Neisseria meningitidis, F. novicida, S. mutans or Treponema denticola. The Cas9 may be a native or modified Cas9 protein.

In some embodiments, the Cas9 protein may be programmed with a gRNA that targets a locus within or near the ASXL1 gene. In some embodiments, the gRNA targets a nucleotide sequence comprising SEQ ID NO: 142. In some embodiments, the Cas9 protein is programmed with a gRNA that comprises a nucleotide sequence of SEQ ID NO: 143. In some aspects, the present invention provides a guide RNA (gRNA) targeting ASXL1 comprising a nucleotide sequence of SEQ ID NO: 143.

In some embodiments, the Cas9 protein may be programmed with a gRNA that targets a locus within or near the ASXL1 gene. In some embodiments, the gRNA targets a nucleotide sequence comprising SEQ ID NO: 161. In some embodiments, the Cas9 protein is programmed with a gRNA that comprises a nucleotide sequence of SEQ ID NO: 162. In some aspects, the present invention provides a guide RNA (gRNA) targeting ASXL1 comprising a nucleotide sequence of SEQ ID NO: 162.

In some embodiments, the Cas9 protein may be programmed with a gRNA that targets a locus within or near the ASXL1 gene. In some embodiments, the gRNA targets a nucleotide sequence comprising SEQ ID NO: 163. In some embodiments, the Cas9 protein is programmed with a gRNA that comprises a nucleotide sequence of SEQ ID NO: 164. In some aspects, the present invention provides a guide RNA (gRNA) targeting ASXL1 comprising a nucleotide sequence of SEQ ID NO: 164.

The Cas9 protein may be programmed with a gRNA that targets a locus within or near the DNMT3A gene. In some embodiments, the gRNA comprises a nucleotide sequence encoded by SEQ ID NO: 63 or SEQ ID NO: 68.

The Cas9 protein may be programmed with a gRNA that targets a locus within or near the TET2 gene.

In various embodiments, the ASXL1 gene, the DNMT3A gene, and/or the TET2 gene is derived from a mammal. In some embodiments, the ASXL1 gene, the DNMT3A gene, and/or the TET2 gene is derived from a mouse. In some embodiments, the ASXL1 gene, the DNMT3A gene, and/or the TET2 gene is derived from a human.

In certain aspects, the present invention provides a ribonucleoprotein complex comprising a gRNA disclosed herein and a Cas9 protein.

In alternative embodiments, the site-specific nuclease used in the methods described herein is a zinc finger nuclease, a TALEN nuclease, or a mega-TALEN nuclease.

In some embodiments, the ASXL1, DNMT3A, and/or TET2 gene product in the immune effector cell is deleted or modified as a result of an activity of an RNA interference (RNAi) molecule or an antisense oligonucleotide. RNA interference (RNAi) refers to the process of sequence-specific post-transcriptional gene silencing in animals mediated by small interfering RNAs (siRNAs) (Fire et al., 1998, Nature, 391, 806; Hamilton et al., 1999, Science, 286, 950-951). Any small nucleic acid molecules capable of mediating RNAi, such as a short interfering nucleic acid (siNA), a small interfering RNA (siRNA), a double-stranded RNA (dsRNA), a micro-RNA (miRNA), and a short hairpin RNA (shRNA), may be to inhibit the expression of the ASXL1, DNMT3A, and/or TET2 gene. An antisense oligonucleotide (ASO) is a short nucleotide sequence that can hybridize or bind (e.g., by Watson-Crick base pairing) in a complementary fashion to its target sequence.

In some embodiments, the RNAi molecule is a small interfering RNA (siRNA) or a small hairpin RNA (shRNA). siRNAs, also known as short interfering RNA or silencing RNA, are a class of double-stranded RNA molecules, 20-25 base pairs in length, and operating within the RNA interference (RNAi) pathway. shRNAs or short hairpin RNAs are a group of artificial RNA molecules with a tight hairpin turn that can be used to silence target gene expression via RNA interference (RNAi).

In various embodiments, the site-specific nuclease, the RNAi molecule or the antisense oligonucleotide as described above is introduced into the immune effector cell via a viral vector, a non-viral vector or a physical means.

The methods for generating a modified immune effector cell described herein may further include activating the STAT5 signaling pathway in the immune effector cell by a signaling molecule. In some embodiments, the signaling molecule is a common gamma chain cytokine. Non-limiting examples of cytokines that may be used in the methods described herein include IL-15, IL-7, IL-2, IL-4, IL-9, and IL-21.

In some embodiments, the STAT5 signaling pathway is activated by modifying the immune effector cell to express a constitutively active cytokine receptor or a switch receptor. Such constitutively active cytokine receptor may be a constitutively active IL 7 receptor (C7R). Such switch receptor may be an IL-4/IL-7 receptor or an IL-4/IL-2 receptor.

In some embodiments, the immune effector cell is contacted with an effective amount of the signaling molecule or a carrier containing the signaling molecule. Suitable carriers include, but are not limited to, polymers, micelles, reverse micelles, liposomes, emulsions, hydrogels, microparticles, nanoparticles, and microspheres. In some embodiments, the carrier is a nanoparticle.

In some embodiments, the immune effector cell is contacted with the signaling molecule more than once. The immune effector cell may be contacted with the signaling molecule 2 times, 3 times, 4 times, 5 times, 6 times, 7 times, 8 times, or more than 8 times. The immune effector cell may be contacted with the signaling molecule at a frequency of every 8 hours, every 12 hours, every 16 hours, every 24 hours, every 2 days, every 3 days, every 4 days, every 5 days, every 6 days, every 7 days, every 8 days, every 8 days, every 10 days, once a week, twice a week, biweekly, once a month, twice a month, 3 times a month, 4 times a month, or 5 times a month.

In some embodiments, the signaling molecule is expressed in the immune effector cell. The signaling molecule may be expressed from a transgene introduced into the immune effector cell. The signaling molecule-expressing transgene may be introduced into the immune effector cell using a viral vector, a non-viral vector, or a physical means. In some embodiments, the modified immune effector cell is further engineered to express a chimeric antigen receptor (CAR) as described herein. The CAR may comprise an extracellular antigen-binding domain, a transmembrane domain, and/or a cytoplasmic domain as described above. The CAR may be expressed from a transgene introduced into the immune effector cell. The CAR-expressing transgene may be introduced into the immune effector cell using a viral vector, a non-viral vector or a physical means. Non-limiting examples viral vectors include a retroviral vector, an adenoviral vector, an adeno-associated viral (AAV) vector, a herpes viral vector, or a baculoviral vector. In some embodiments, the retroviral vector is a lentiviral vector. In some embodiments, the non-viral vector is a transposon. In some embodiments, the transposon is a sleeping beauty transposon or PiggyBac transposon.

Physical means by which the CAR-expressing transgene may be introduced into the immune effector cells include, but are not limited to, electroporation, microinjection, magnetofection, ultrasound, a ballistic or hydrodynamic method, or a combination thereof.

In some embodiments, the immune effector cells are T cells.

In some embodiments, the immune effector cells are NK cells.

In some embodiments, the immune effector cells are stem cells that are capable of differentiating into immune cells, including induced pluripotent stem cells (iPSCs).

Modified immune effector cells can be activated and/or expanded ex vivo for use in adoptive cellular immunotherapy in which infusions of such cells have been shown to have anti-disease reactivity in a disease-bearing subject. The compositions and methods of this invention can be used to generate a population of immune effector cells (e.g., T lymphocytes or natural killer cells) with enhanced immune cell function for use in immunotherapy in the treatment of the disease.

### Isolation/Enrichment

The immune effector cells may be autologous/autogeneic (“self”) or non-autologous (“non-self,” e.g., allogeneic, syngeneic or xenogeneic). In some embodiments, the immune effector cells are obtained from a mammalian subject. In other embodiments, the immune effector cells are obtained from a primate subject. In some embodiments, the immune effector cells are obtained from a human subject.

Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In some embodiments, lymphocytes can be obtained from blood collected from a subject using techniques generally known to the skilled person, such as sedimentation, e.g., FICOLL™ separation.

In some embodiments, cells from the circulating blood of a subject are obtained by apheresis. An apheresis device typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In some embodiments, immune effector cells can be isolated from a subject (e.g., a donor). In some embodiments, the immune effector cell may be isolated from a subject having a disease. The disease may be, for example, a cancer, an infectious disease, an inflammatory disorder, or an autoimmune disease. As a non-limiting example, the cancer may be a cancer expressing B7-H3. In certain embodiments, the cancer may be a cancer expressing, e.g., HER2, IL 13Rα2, and/or EphA2. In some embodiments, the cancer may be a cancer expressing, e.g., CD19, CD22, CD123, and/or CD33.

In some embodiments, immune effector cells disclosed herein may be derived from a blood, marrow, tissue, or a tumor sample.

In some embodiments, immune effector cells can be isolated from peripheral blood mononuclear cells (PBMCs) by lysing the red blood cells and depleting the monocytes. As an example, the cells can be sorted by centrifugation through a PERCOLL™ gradient. In some embodiments, after isolation of PBMCs, both cytotoxic and helper T lymphocytes can be sorted into naïve, memory, and effector T cell subpopulations either before or after activation, expansion, and/or genetic modification.

In some embodiments, T lymphocytes can be enriched. For example, a specific subpopulation of T lymphocytes expressing one or more markers such as, but not limited to, CD3, CD4, CD8, CD14, CD15, CD16, CD19, CD27, CD28, CD34, CD36, CD45RA, CD45RO, CD56, CD62, CD62L, CD122, CD123, CD127, CD235a, CCR7, HLA-DR, or a combination thereof, can be enriched using either positive or negative selection techniques. In some embodiments, the T lymphocytes for use in the compositions of the invention do not express or do not substantially express one or more of the following markers: CD57, CD244, CD160, PD-1, CTLA4, TIM3, and LAG3.

In some embodiments, NK cells can be enriched. For example, a specific subpopulation of T lymphocytes expressing one or more markers such as, but not limited to, CD2, CD16, CD56, CD57, CD94, CD122, or a combination thereof, can be enriched using either positive or negative selection techniques.

### Stimulation/Activation

In order to reach sufficient therapeutic doses of immune effector cell compositions, immune effector cells are often subjected to one or more rounds of stimulation/activation. In some embodiments, a method of producing immune effector cells for administration to a subject comprises stimulating the immune effector cells to become activated in the presence of one or more stimulatory signals or agents (e.g., compound, small molecule, e.g., small organic molecule, nucleic acid, polypeptide, or a fragment, isoform, variant, analog, or derivative thereof). In some embodiments, a method of producing immune effector cells for administration to a subject comprises stimulating the immune effector cells to become activated and to proliferate in the presence of one or more stimulatory signals or agents.

Immune effector cells (e.g., T lymphocytes and NK cells) can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In some embodiments, the T cell based immune effector cells can be activated by binding to an agent that activates CD3ζ.

In other embodiments, a CD2-binding agent may be used to provide a primary stimulation signal to the T cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100, which is incorporated herein by reference in its entirety). Other antibodies which bind to the same epitopes as any of the above-described antibodies can also be used.

In some embodiments, the immune effector cells are activated by administering phorbol myristate acetate (PMA) and ionomycine. In some embodiments, the immune effector cells are activated by administering an appropriate antigen that induces activation and then expansion. In some embodiments, PMA, ionomycin, and/or appropriate antigen are administered with CD3 induce activation and/or expansion.

In general, the activating agents used in the present invention include, but are not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab) 2′-fragment, diabodies, triabodies (Iliades, P., et al., FEBS Lett (1997) 409, 437-441, which is incorporated herein by reference in its entirety), decabodies (Stone, E., et al., Journal of Immunological Methods (2007) 318, 88-94, which is incorporated herein by reference in its entirety) and other domain antibodies (Holt, L. J., et al., Trends Biotechnol. (2003), 21, 11, 484-490, which is incorporated herein by reference in its entirety). The divalent antibody fragment may be an (Fab) 2′-fragment, or a divalent single-chain Fv fragment while the monovalent antibody fragment may be selected from a Fab fragment, a Fv fragment, and a single-chain Fv fragment (scFv).

In some embodiments, one or more binding sites of the CD30 agents may be a bivalent proteinaceous artificial binding molecule such as a dimeric lipocalin mutein (i.e., duocalin). In some embodiments the receptor binding reagent may have a single second binding site (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to, a Fab fragment, a Fv fragment, and a single-chain Fv fragment (scFv), including a divalent single-chain Fv fragment.

The agent that specifically binds CD3 includes, but is not limited to, an anti-CD3-antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In some embodiments, the activating agent (e.g., CD3-binding agent) can be present in a concentration of about 0.1 to about 10 μg/ml. In some embodiments, the activating agent (e.g., CD3-binding agent) can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In some embodiments, the activating agent (e.g., CD3-binding agent) is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In some embodiments, the activating agent (e.g., CD3-binding agent) can be present in a concentration of 1 μg/ml.

NK cells can be activated generally using methods as described, for example, in U.S. Pat. Nos. 7,803,376, 6,949,520, 6,693,086, 8,834,900, 9,404,083, 9,464,274, 7,435,596, 8,026,097, and 8,877,182; U.S. Patent Applications US2004/0058445, US2007/0160578, US2013/0011376, US2015/0118207, and US2015/0037887; and PCT Patent Application WO2016/122147, each of which is incorporated herein by reference in its entirety.

In some embodiments, the NK based immune effector cells can be activated by, for example and not limitation, inhibition of inhibitory receptors on NK cells (e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LILRB5 receptor).

In some embodiments, the NK based immune effector cells can be activated by, for example and not limitation, feeder cells (e.g., native K562 cells or K562 cells that are genetically modified to express 4-1BBL and cytokines such as IL15 or IL21).

In other embodiments, interferons or macrophage-derived cytokines can be used to activate NK cells. For example and not limitation, such interferons include, but are not limited to, interferon alpha and interferon gamma, and such cytokines include but are not limited to IL-15, IL-2, IL-21.

In some embodiments, the NK activating agent can be present in a concentration of about 0.1 to about 10 g/ml. In some embodiments, the NK activating agent can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In some embodiments, the NK activating agent is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In some embodiments, the NK activating agent can be present in a concentration of 1 μg/ml.

In some embodiments, the activating agent is attached to a solid support such as, but not limited to, a bead, an absorbent polymer present in culture plate or well or other matrices such as, but not limited to, Sepharose or glass; or may be expressed (such as in native or recombinant forms) on cell surface of natural or recombinant cell line by means known to those skilled in the art.

Polynucleotide and/or Polypeptide Transfer

In some embodiments, the immune effector cells are genetically modified by introducing polynucleotides and/or polypeptide (e.g., a CAR, a signaling molecule, a site-specific nuclease, an RNAi molecule or an antisense oligonucleotide, or polynucleotides encoding the same). The immune effector cells can be genetically modified after stimulation/activation. In some embodiments, the immune effector cells are modified within 12 hours, 16 hours, 24 hours, 36 hours, or 48 hours of stimulation/activation. In some embodiments, the cells are modified within 16 to 24 hours after stimulation/activation. In some embodiments, the immune effector cells are modified within 24 hours.

In order to genetically modify the immune effector cell, the polynucleotides and/or polypeptide (e.g., a CAR, a signaling molecule, a site-specific nuclease, an RNAi molecule or an antisense oligonucleotide, or polynucleotides encoding the same) must be transferred into the cell. Polynucleotide and/or polypeptide transfer may be via viral, non-viral gene delivery methods, or a physical method. Suitable methods for polynucleotide and/or polypeptide delivery for use with the current methods include any method known by those of skill in the art by which a polynucleotide and/or polypeptide can be introduced into an organelle, cell, tissue or organism.

In various embodiments, polypeptides or polynucleotides (e.g., a CAR, a signaling molecule, a site-specific nuclease, an RNAi molecule or an antisense oligonucleotide, or polynucleotides encoding the same) described in the present invention are introduced to the immune effector cell via a recombinant vector.

In some embodiments, the recombinant vector encoding a CAR described above comprises the nucleotide sequence of SEQ ID NO: 4, 6, 10, 12, or 14, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% sequence identity with SEQ ID NO: 4, 6, 10, 12, or 14. In some embodiments, the recombinant vector comprises the nucleotide sequence that encodes the amino acid sequence of SEQ ID NO: 3, 5, 9, 11, or 13, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% sequence identity with SEQ ID NO: 3, 5, 9, 11, or 13.

In some embodiments, the vector is a viral vector. Suitable viral vectors that can be used in the present invention include, but are not limited to, a retroviral vector, an adenoviral vector, an adeno-associated viral (AAV) vector, a herpes viral vector, or a baculoviral vector. In one specific embodiment, the viral vector is a lentiviral vector.

In some embodiments, the immune effector cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993), each of which is incorporated herein by reference in its entirety.

One method of genetic modification includes ex vivo modification. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244 (4910): 1342-1344, 1989, both of which are incorporated herein by reference in their entity. In some embodiments, the immune effector cells may be removed from the subject and transfected ex vivo using the polynucleotides (e.g., expression vectors) of the invention. In some embodiments, the immune effector cells obtained from the subject can be transfected or transduced with the polynucleotides (e.g., expression vectors) of the invention and then administered back to the subject.

In some embodiments, polynucleotides and/or polypeptides are transferred to the cell in a non-viral vector. In some embodiments, the non-viral vector is a transposon. Exemplary transposons hat can be used in the present invention include, but are not limited to, a sleeping beauty transposon and a PiggyBac transposon.

Nucleic acid vaccines may also be used to transfer polynucleotides into the immune effector cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In some embodiments, the polynucleotide(s) is operatively linked to at least one regulatory element for expression of the gene product (e.g., a CAR, a signaling molecule, a site-specific nuclease, an RNAi molecule). The regulatory element can be capable of mediating expression of the gene product in the host cell (e.g., modified immune effector cell). Regulatory elements include, but are not limited to, promoters, enhancers, initiation sites, polyadenylation (polyA) tails, IRES elements, response elements, and termination signals. In some embodiments, the regulatory element regulates expression of the gene product. In some embodiments, the regulatory element increases the expression of the gene product. In some embodiments, the regulatory element increases the expression of the gene product once the host cell (e.g., modified immune effector cell) is activated. In some embodiments, the regulatory element decreases expression of the gene product. In some embodiments, the regulatory element decreases expression of the gene product once the host cell (e.g., modified immune effector cell) is activated.

In various embodiment, polypeptides or polynucleotides (e.g., a CAR, a signaling molecule, a site-specific nuclease, an RNAi molecule or an antisense oligonucleotide, or polynucleotides encoding the same) are introduced into the modified immune effector cell using a physical means. Suitable physical means include, but are not limited to, electroporation, microinjection, magnetofection, ultrasound, a ballistic or hydrodynamic method, or a combination thereof.

Electroporation is a method for polynucleotide and/or polypeptide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In some embodiments, cell wall-degrading enzymes, such as pectin-degrading enzymes, can be employed to render the immune effector cells more susceptible to genetic modification by electroporation than untreated cells. See e.g., U.S. Pat. No. 5,384,253, incorporated herein by reference in its entirety for all purposes.

In vivo electroporation involves a basic injection technique in which a vector is injected intradermally in a subject. Electrodes then apply electrical pulses to the intradermal site causing the cells localized there (e.g., resident dermal dendritic cells) to take up the vector. These tumor antigen-expressing dendritic cells activated by local inflammation can then migrate to lymph nodes.

Methods of electroporation for use with this invention include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

Another method for polynucleotide and/or polypeptide transfer includes injection. In some embodiments, a polypeptide, a polynucleotide or a viral vector may be delivered to a cell, tissue, or organism via one or more injections (e.g., a needle injection). Non-limiting methods of injection include injection of a composition (e.g., a saline-based composition). Polynucleotides and/or polynucleotides can also be introduced by direct microinjection. Non-limiting sites of injection include, subcutaneous, intradermal, intramuscular, intranodal (allows for direct delivery of antigen to lymphoid tissues), intravenous, intraprostatic, intratumor, intralymphatic (allows direct administration of DCs) and intraperitoneal. It is understood that proper site of injection preparation is necessary (e.g., shaving of the site of injection to observe proper needle placement).

Additional methods of polynucleotide and/or polypeptide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide and/or polypeptide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5(5):1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and/or polypeptide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

In further embodiments, host cells (e.g., modified immune effector cells) are genetically modified using gene editing with homology-directed repair (HDR). Homology-directed repair (HDR) is a mechanism used by cells to repair double strand DNA breaks. In HDR, a donor polynucleotide with homology to the site of the double strand DNA break is used as a template to repair the cleaved DNA sequence, resulting in the transfer of genetic information from the donor polynucleotide to the DNA. As such, new nucleic acid material may be inserted or copied into a target DNA cleavage site. Double strand DNA breaks in host cells may be induced by a site-specific nuclease. Suitable site-specific nucleases for use in the present disclosure include, but are not limited to, RNA-guided endonuclease (e.g., CRISPR-associated (Cas) proteins), zinc finger nuclease, a TALEN nuclease, or mega-TALEN nuclease. For example, a site-specific nuclease (e.g., a Cas9+guide RNA) capable of inducing a double strand break in a target DNA sequence is introduced to a host cell, along with a donor polynucleotide encoding a CAR of the present disclosure and optionally an additional protein (e.g., tCD19).

### Expansion/Proliferation

After the immune effector cells are activated and transduced, the cells are cultured to proliferate. T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of T cells can include interleukins, such as IL-2, IL-7, IL-15, or IL-21 (see for example Cornish et al. 2006, Blood. 108(2):600-8, Bazdar and Sieg, 2007, Journal of Virology, 2007, 81(22):12670-12674, Battalia et al, 2013, Immunology, 139 (1): 109-120, each of which is incorporated by reference in their entirety for all purposes). Other illustrative examples for agents that may be used for the expansion of T cells are agents that bind to CD8, CD45 or CD90, such as αCD8, αCD45 or αCD90 antibodies. Illustrative examples of T cell populations include antigen-specific T cells, T helper cells, cytotoxic T cells, memory T cell (an illustrative example of memory T cells are CD62L+CD8+ specific central memory T cells) or regulatory T cells (an illustrative example of Treg are CD4+CD25+CD45RA+ Treg cells).

Additional agents that can be used to expand T lymphocytes includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In some embodiments, the agent(s) used for expansion (e.g., IL-7, IL-15) are administered at about 20 units/ml to about 200 units/ml. In some embodiments, the agent(s) used for expansion (e.g., IL-7, IL-15) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In some embodiments, the agent(s) used for expansion (e.g., IL-7, IL-15) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In some embodiments, the agent(s) used for expansion (e.g., IL-7, IL-15) are administered at about 5 mg/ml to about 10 ng/ml. In some embodiments, the agent(s) used for expansion (e.g., IL-7, IL-15) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In some embodiments, the agent(s) used for expansion (e.g., IL-7, IL-15) are administered at about 5 ng/ml, 6 ng/ml, 7 ng/ml, 8 ng/ml, 9, ng/ml, or 10 ng/ml.

After the immune effector cells are activated and transduced, the cells are cultured to proliferate. NK cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of natural killer cells can include agents that bind to CD16 or CD56, such as, for example, αCD16 or αCD56 antibodies. In some embodiments, the binding agent includes antibodies (see for example Hoshino et al, Blood. 1991 Dec. 15; 78(12):3232-40.). Other agents that may be used for expansion of NK cells may be IL-15 (see for example Vitale et al. 2002. The Anatomical Record. 266:87-92, which is incorporated by reference in their entirety for all purposes).

Conditions appropriate for T cell culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion).

Examples of other additives for immune effector cell expansion include, but are not limited to, surfactant, piasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In certain embodiments, host cells of the present disclosure may be modified such that the expression of an endogenous TCR, MHC molecule, or other immunogenic molecule is decreased or eliminated. When allogeneic cells are used, rejection of the therapeutic cells may be a concern as it may cause serious complications such as the graft-versus-host disease (GvHD). Although not wishing to be bound by theory, immunogenic molecules (e.g., endogenous TCRs and/or MHC molecules) are typically expressed on the cell surface and are involved in self vs non-self-discrimination. Decreasing or eliminating the expression of such molecules may reduce or eliminate the ability of the therapeutic cells to cause GvHD.

In certain embodiments, expression of an endogenous TCR in the host cells is decreased or eliminated. In a particular embodiment, expression of an endogenous TCR (e.g., αβ TCR) in the host cells is decreased or eliminated. Expression of the endogenous TCR may be decreased or eliminated by disrupting the TRAC locus, TCR beta constant locus, and/or CD3 locus. In certain embodiments, expression of an endogenous TCR may be decreased or eliminated by disrupting one or more of the TRAC, TRBC1, TRBC2, CD3E, CD3G, and/or CD3D locus.

In certain embodiments, expression of one or more endogenous MHC molecules in the host cells is decreased or eliminated. Modified MHC molecules may be an MHC class I or class II molecule. In certain embodiments, expression of an endogenous MHC molecule may be decreased or eliminated by disrupting one or more of the MHC, β2M, TAP1, TAP2, CIITA, RFX5, RFXAP and/or RFXANK locus.

Expression of the endogenous TCR, an MHC molecule, and/or any other immunogenic molecule in the host cell can be disrupted using genome editing techniques such as Clustered regularly interspaced short palindromic repeats (CRISPR)/Cas, zinc finger nucleases (ZFNs), transcription activator-like effector nucleases (TALENs), and Meganucleases. These genome editing methods may disrupt a target gene by entirely knocking out all of its output or partially knocking down its expression. In a particular embodiment, expression of the endogenous TCR, an MHC molecule and/or any other immunogenic molecule in the host cell is disrupted using the CRISPR/Cas technique.

### Methods of Enhancing Immune Cell Function

In one aspect, the present invention provides a method of enhancing immune cell function (e.g. preserved developmental potential (i.e., preserved stem-like state of differentiation)) of an immune effector cell. In a specific aspect, the present invention provides a method of maintaining cytolytic potential of an immune effector cell. Such methods may comprise modifying an ASXL1 gene or gene product in the cell so that the expression and/or function of ASXL1 in the cell is reduced or eliminated. As a non-limiting example, the immune effector cell may be any of the various T cells disclosed herein. In particular, the T cell may be selected from, e.g., a CD8+ T cell, a CD4+ T cell, a cytotoxic T cell, an αβ T cell receptor (TCR) T cell, a natural killer T (NKT) cell, a γδ T cell, a memory T cell, a T-helper cell, and a regulatory T cell (Treg). In some embodiments, the immune effector cell may be an NK cell. In some embodiments, the above-described methods may comprise modifying the immune effector cell to express a CAR disclosed herein that is capable of binding to an antigen specific to tumor disclosed herein.

The methods may further comprise modifying a DNMT3A gene or gene product in the cell so that the expression and/or function of DNMT3A in the cell is reduced or eliminated. The methods may further comprise modifying a TET2 gene or gene product in the cell so that the expression and/or function of TET2 in the cell is reduced or eliminated. In some embodiments, the ASXL1 gene, the DNMT3A gene, and/or the TET2 gene may be deleted. In certain embodiments, when the DNMT3A gene is deleted or modified, for example, DNMT3A-mediated de novo DNA methylation of the cell genome is inhibited.

In some embodiments, the ASXL1, DNMT3A, and/or TET2 gene or gene product in the immune effector cell may be modified in the presence of one or more inhibitory signals or agents (e.g., compound, small molecule, e.g., small organic molecule, nucleic acid, polypeptide, or a fragment, isoform, variant, analog, or derivative thereof). In some embodiments, the ASXL1, DNMT3A, and/or TET2 gene or gene product may be targeted using any number of various agents (e.g., a small molecule inhibitor). In certain embodiments, the agent may be used to reduce the expression and/or activity of ASXL1, DNMT3A, and/or TET2 in a modified immune effector cell disclosed herein.

In some embodiments, the small molecule may be, for example, a peptide and/or a peptidomimetic. A peptidomimetic may include, e.g., chemically modified peptides and peptide-like molecules that contain non-naturally occurring amino acids, peptoids, and the like. Methods for identifying a peptidomimetics are well known in the art and may comprise the screening of databases that contain libraries of possible peptidomimetics.

In some embodiments, the ASXL1 gene in the immune effector cell is deleted or modified as a result of an activity of a site-specific nuclease. In some embodiments, the site-specific nuclease is an RNA-guided endonuclease. In some embodiments, the RNA-guided endonuclease is a Cas9 protein. In some embodiments, the Cas9 protein may be programmed with a gRNA that targets a locus within or near the ASXL1 gene. In some embodiments, the gRNA targets a nucleotide sequence comprising SEQ ID NO: 142. In some embodiments, the Cas9 protein is programmed with a gRNA that comprises a nucleotide sequence of SEQ ID NO: 143. In some aspects, the present invention provides a guide RNA (gRNA) targeting ASXL1 comprising a nucleotide sequence of SEQ ID NO: 143. In some embodiments, the gRNA targets a nucleotide sequence comprising SEQ ID NO: 161. In some embodiments, the Cas9 protein is programmed with a gRNA that comprises a nucleotide sequence of SEQ ID NO: 162. In some aspects, the present invention provides a guide RNA (gRNA) targeting ASXL1 comprising a nucleotide sequence of SEQ ID NO: 162. In some embodiments, the gRNA targets a nucleotide sequence comprising SEQ ID NO: 163. In some embodiments, the Cas9 protein is programmed with a gRNA that comprises a nucleotide sequence of SEQ ID NO: 164. In some aspects, the present invention provides a guide RNA (gRNA) targeting ASXL1 comprising a nucleotide sequence of SEQ ID NO: 164.

In some embodiments, the DNMT3A gene in the immune effector cell is deleted or modified as a result of an activity of a site-specific nuclease. In some embodiments, the site-specific nuclease is an RNA-guided endonuclease. In some embodiments, the RNA-guided endonuclease is a Cas9 protein. In some embodiments, the Cas9 protein may be programmed with a gRNA that targets a locus within or near the DNMT3A gene. In some embodiments, the gRNA comprises a nucleotide sequence encoded by SEQ ID NO: 63 or SEQ ID NO: 68.

In some embodiments, the TET2 gene in the immune effector cell is deleted or modified as a result of an activity of a site-specific nuclease. In some embodiments, the site-specific nuclease is an RNA-guided endonuclease. In some embodiments, the RNA-guided endonuclease is a Cas9 protein. In some embodiments, the Cas9 protein may be programmed with a gRNA that targets a locus within or near the TET2 gene.

In alternative embodiments, the site-specific nuclease used in the methods described herein is a zinc finger nuclease, a TALEN nuclease, or a mega-TALEN nuclease.

In some embodiments, the ASXL1, DNMT3A, and/or TET2 gene product in the immune effector cell is deleted or modified as a result of an activity of an RNA interference (RNAi) molecule or an antisense oligonucleotide. In some embodiments, the RNAi molecule is a small interfering RNA (siRNA) or a small hairpin RNA (shRNA).

In various embodiments, the site-specific nuclease, the RNAi molecule or the antisense oligonucleotide as described above is introduced into the immune effector cell via a viral vector, a non-viral vector, or a physical means described herein.

In some embodiments, the method further includes activation and/or expansion of the immune effector cell ex vivo.

### Pharmaceutical Compositions

In some embodiments, the compositions comprise one or more polypeptides, polynucleotides, vectors comprising same, and cell compositions, as disclosed herein. Compositions include, but are not limited to pharmaceutical compositions. In some embodiments, the compositions of the present invention comprise an amount of modified immune effector cells manufactured by the methods disclosed herein.

In one aspect, the present invention provides a pharmaceutical composition comprising a modified immune effector cell described herein and a pharmaceutically acceptable carrier and/or excipient. Examples of pharmaceutical carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising modified immune effector cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising modified immune effector cells disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated for parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile. In some embodiments, the composition is reconstituted from a lyophilized preparation prior to administration.

In some embodiments, the modified immune effector cells may be mixed with substances that adhere or penetrate then prior to their administration, e.g., but not limited to, nanoparticles.

### Therapeutic Methods

In one aspect, the present invention provides a method of treating a disease or disorder in a subject in need thereof, including administering to the subject an effective amount of the modified immune effector cells or the pharmaceutical composition described herein. In some embodiments, the modified immune effector cells are prepared by the methods as disclosed above.

In some embodiments, the modified immune effector cell is an autologous cell. In some embodiments, the modified immune effector cell is an allogeneic cell.

In some embodiments, the disease being treated by the therapeutic methods described herein is a cancer, an infectious disease, an inflammatory disorder, or an autoimmune disease.

The terms “cancer” and “cancerous” refer to or describe the physiological condition in mammals that is typically characterized by unregulated cell growth. The term “cancer” includes, for example, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of cancer can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In some embodiments, the cancer is a solid tumor. Non-limiting examples of solid tumors include osteosarcoma, medulloblastoma, glioblastoma ependymoma and high-grade gliomas. In some embodiments, the cancer is a breast, prostate, urinary bladder, skin, lung, ovary, sarcoma, or brain cancer.

In some embodiments, the cancer is a liquid tumor such as, but not limited to leukemia, including chronic leukemia, e.g., chronic myelocytic (granulocytic) leukemia and chronic lymphocytic leukemia, acute leukemia, e.g., acute lymphocytic leukemia, acute myelocytic leukemia, and myeloblastic, promyelocytic, myelomonocytic, monocytic and erythroleukemia, lymphoma, Waldenstrom's macroglobulinemia, Hodgkin's disease, non-Hodgkin's lymphoma, polycythemia vera, multiple myeloma, and heavy chain disease. In some embodiments, the liquid tumor is B-cell acute lymphoblastic leukemia.

In various embodiments, the liquid tumor may comprise a hematologic cancer, i.e., a blood cancer which may originate from or occur within blood-forming tissue, e.g., blood and/or bone marrow. In some embodiments a hematologic cancer may originate from or occur within lymph nodes.

The therapeutic methods described herein may be used to treat a cancer expressing, e.g., CD19, CD22, CD123, CD33, B7-H3, HER2, IL 13Rα2, or EphA2.

Cancers expressing B7-H3 may include, but are not limited to, osteosarcoma, rhabdomyosarcoma, Ewing's sarcoma and other Ewing's sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma. In some embodiments, the cancer expressing B7-H3 may include, without limitation, osteosarcoma, and glioblastoma. In some embodiments, the cancer expressing B7-H3 may be a brain tumor. Non-limiting examples of brain tumors include high-grade gliomas, medulloblastoma, ependymoma, and atypical teratoid rhabdoid tumors. The cancer expressing B7-H3 may include, without limitation, high-grade gliomas, medulloblastoma, ependymoma, and atypical teratoid rhabdoid tumors.

Cancers expressing HER2 may include, but are not limited to, sarcomas such as angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, or synovial sarcoma; brain cancers such as glioblastoma; breast, prostate, lung, and colon cancers or epithelial cancers/carcinomas such as breast cancer, colon cancer, prostate cancer, head and neck cancer, skin cancer; cancers of the genitourinary tract such as ovarian cancer, endometrial cancer, cervical cancer and kidney cancer; lung cancer, gastric cancer, cancer of the small intestine, liver cancer, pancreatic cancer, gall bladder cancer, cancers of the bile duct, esophagus cancer, cancer of the salivary glands and cancer of the thyroid gland. In some embodiments, the cancer is a HER2-positive breast cancer.

Cancers expressing IL 13Rα2 may include, but are not limited to, brain cancers such as glioblastoma, colon cancer, renal cell carcinoma, pancreatic cancer, melanoma, head and neck cancer, mesothelioma, and ovarian cancer. In some embodiments, the cancer is an IL 13Rα2-positive glioblastoma.

Cancers expressing EphA2 may include, but are not limited to, sarcomas such as rhabdomyosarcoma, osteosarcoma, and Ewing's sarcoma; breast, prostate, urinary bladder, skin cancers including melanoma, lung cancer, liver cancer, ovarian cancer, stomach cancer, colorectal cancer, thyroid cancer, head and neck cancer, cervical cancer, pancreatic cancer, endometrial cancer, and brain cancers.

The therapeutic methods described herein may include the steps of (i) isolating an immune effector cell from the subject or a donor; (ii) modifying an ASXL1 gene or gene product in the immune effector cell so that the expression and/or function of ASXL1 in the cell is reduced or eliminated; (iii) introducing the modified immune effector cell into the subject.

The therapeutic methods described herein may include the steps of (i) isolating an immune effector cell from the subject or a donor; (ii) modifying an ASXL1, a DNMT3A, and/or a TET2 gene or gene product in the immune effector cell so that the expression and/or function of ASXL1, DNMT3A, and/or TET2 in the cell is reduced or eliminated; (iii) introducing the modified immune effector cell into the subject.

Activating the STAT5 signaling pathway in the immune effector cell may be achieved by stimulating the immune effector cell with a signaling molecule either ex vivo or in vivo. For example, stimulating the immune effector cell with a signaling molecule may be carried out by mixing the immune effector cell directly with the signaling molecule, or with a carrier (e.g., nanoparticles) containing the signaling molecule ex vivo. Mixing of the immune effector cell with the signaling molecule, or with a carrier (e.g., nanoparticles) containing the signaling molecule may be carried out prior to administration, or during administration. In some embodiments, the immune effector cells may be administered with nanoparticle “backpacks” which are capable of carrying signaling molecules and attaching them to the immune effector cells. Such nanoparticle “backpacks” may selectively release the signaling molecules in response to certain stimuli, such as the activation of the immune effector cell (Tang L., Nat Biotechnol. 2018; 36(8):707-716, which is incorporated by reference in their entirety for all purposes).

Alternatively, signaling molecules may be provided to the modified immune effector cells in vivo by administration of the signaling molecule, for example systemically, to the subject such that the signaling molecule can ultimately contact the modified immune effector cells. Signaling molecules may also be provided to the modified immune effector cells in vivo using oncolytic viruses encoding the signaling molecule. Oncolytic viruses can selectively infect and/or lyse cancer or tumor cells as compared to normal cells. Exemplary oncolytic viruses include a herpes simplex virus-1, a herpes simplex virus-2, a vesicular stomatitis virus, and a vaccinia virus.

Activating the STAT5 signaling pathway in the immune effector cell may also be achieved by genetically modifying the immune effector cell to express a signaling molecule. The signaling molecule may be expressed from a transgene introduced into the immune effector cell. Alternatively, the STAT5 signaling pathway is activated by modifying the immune effector cell to express a constitutively active cytokine receptor or a switch receptor. Such constitutively active cytokine receptor may be a constitutively active IL7 receptor (C7R). Such switch receptor may be an IL-4/IL-7 receptor or an IL-4/IL-2 receptor.

In some embodiments, the therapeutic methods include genetically modifying the immune effector cell to express a chimeric antigen receptor (CAR) that is capable of binding specifically to an antigen. In some embodiments, the therapeutic methods include genetically modifying the immune effector cell to express a T cell receptor (TCR) that is capable of binding specifically to an antigen.

In some embodiments, the subject is human.

In cases where the immune effector cell is isolated from a donor, the method may further include a method to prevent graft-versus-host disease (GvHD) and the immune effector cell rejection.

In some embodiments of any of the therapeutic methods described above, the composition is administered in a therapeutically effective amount. The dosages of the composition administered in the methods of the invention will vary widely, depending upon the subject's physical parameters, the frequency of administration, the manner of administration, the clearance rate, and the like. The initial dose may be larger, and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of immune effector cells. It is also contemplated that a variety of doses will be effective to improve in vivo effector function of immune effector cells.

In some embodiments, compositions comprising the immune effector cells manufactured by the methods described herein may be administered at a dosage of 10² to 10¹⁰ cells/kg body weight, 10⁵ to 10⁹ cells/kg body weight, 10⁵ to 10⁸ cells/kg body weight, 105 to 10⁷ cells/kg body weight, 10⁷ to 10⁹ cells/kg body weight, or 10⁷ to 10⁸ cells/kg body weight, including all integer values within those ranges. The number of immune effector cells will depend on the therapeutic use for which the composition is intended for.

Modified immune effector cells may be administered multiple times at dosages listed above. The immune effector cells may be allogeneic, syngeneic, xenogeneic, or autologous to the patient undergoing therapy.

The compositions and methods described in the present disclosure may be utilized in conjunction with other types of therapy for cancer, such as chemotherapy, surgery, radiation, gene therapy, and so forth.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from immuno-suppressives, biologicals, probiotics, prebiotics, and cytokines (e.g., IFN or IL-2).

As a non-limiting example, the invention can be combined with other therapies that block inflammation (e.g., via blockage of IL1, INFα/β, IL6, TNF, IL23, etc.).

The methods and compositions of the invention can be combined with other immunomodulatory treatments such as, e.g., therapeutic vaccines (including, but not limited to, GVAX, DC-based vaccines, etc.), checkpoint inhibitors (including, but not limited to, agents that block CTLA4, PD1, LAG3, TIM3, etc.) or activators (including, but not limited to, agents that enhance 4-1BB, OX40, etc.). The methods of the invention can be also combined with other treatments that possess the ability to modulate NKT function or stability, including but not limited to, CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e). The methods of the invention can also be combined with other treatments such as midostaurin, enasidenib, or a combination thereof.

Therapeutic methods of the invention can be combined with additional immunotherapies and therapies. For example, when used for treating cancer, the compositions of the invention can be used in combination with conventional cancer therapies, such as, e.g., surgery, radiotherapy, chemotherapy or combinations thereof, depending on type of the tumor, patient condition, other health issues, and a variety of factors. In certain aspects, other therapeutic agents useful for combination with conventional cancer therapies include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the immune effector cells of the invention can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present invention include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the methods described herein, the subject is a human. The subject may be a juvenile or an adult, of any age or sex.

In accordance with the present invention there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. ASXL1 Undergoes Demethylation During Murine and Human CD8 T Cell Differentiation

Findings in murine models as well as human systems have shown that the progressive acquisition of DNA methylation is causal in establishing T cell exhaustion. DNA methylation occurs at CpG sites in mammals and is broadly used during cellular differentiation to repress transcription. The palindromic nature of the CpG substrate in the parental strand of DNA serves as a “methylation template” during DNA replication and allows for propagation of an acquired methylation program from parental cell to daughter cell during division. This heritable quality of DNA methylation programming provided the rationale for investigations of epigenetic mechanisms that reinforce the T cell exhaustion gene expression program of the present disclosure and provides the context for interpreting data described herein.

To determine if ASXL1 is involved in normal T cell differentiation, its locus for epigenetic hallmarks of reprogramming during mouse and human effector differentiation was first examined. Specifically, the DNA methylation status for ASXL1 was assessed. Indeed, the murine Asxl1 gene was mostly methylated in naïve CD8 T cells, undergoes demethylation in the effector and memory subsets, and remains primarily unmethylated in exhausted CD8 T cells at the differentially methylated regions highlighted in FIG. 1. Similarly, in human CD8 T cells, ASXL1 was mostly methylated in the naïve cells and became demethylated in the stem cell memory (Tscm), central memory (Tcm), and effector memory (Tem) subsets. These data document the demethylation of the ASXL1 promoter during CD8 T cell effector differentiation. This demethylated state was preserved in memory T cells. Further, these data showcase whole genome methylation profiling on both mouse and human samples.

### Example 2. Disruption of ASXL1 Preserves CD8 T Cell Proliferative Response to Immune Checkpoint Blockade (ICB)

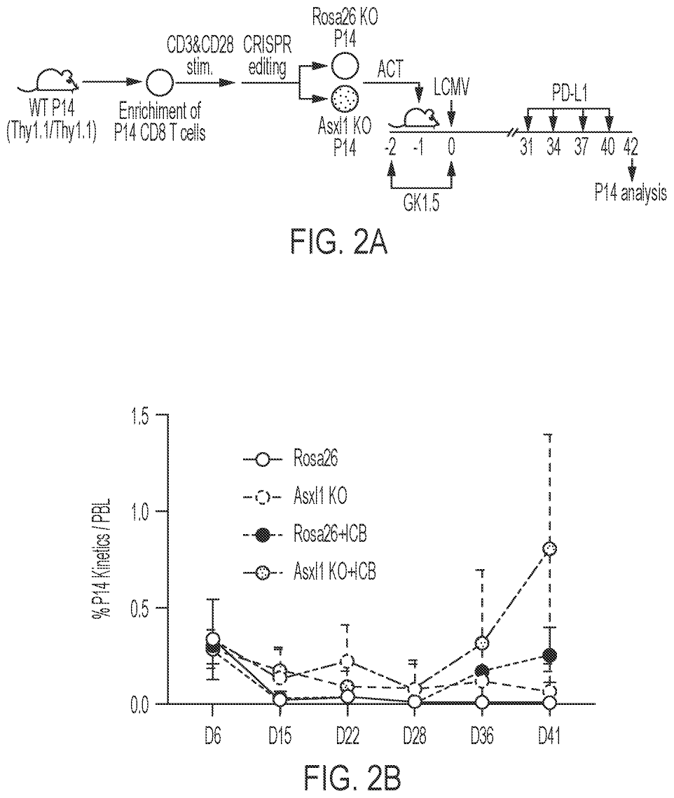

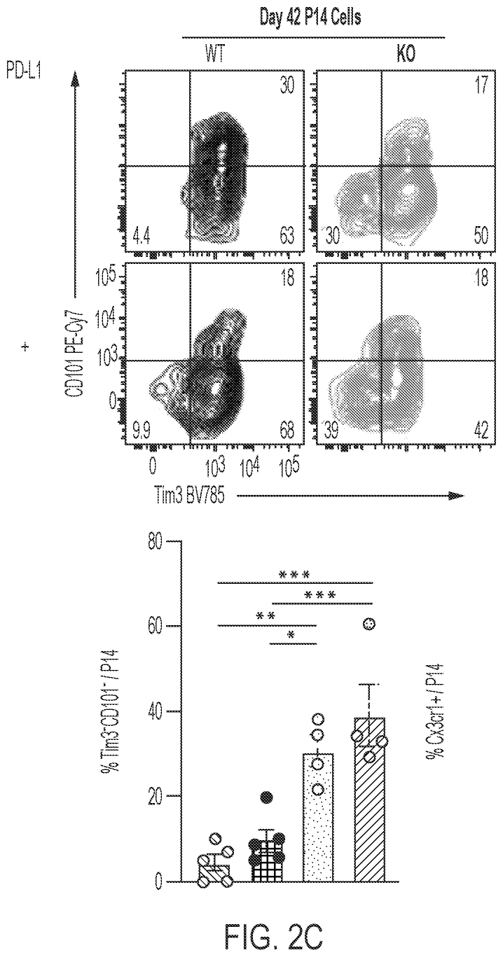

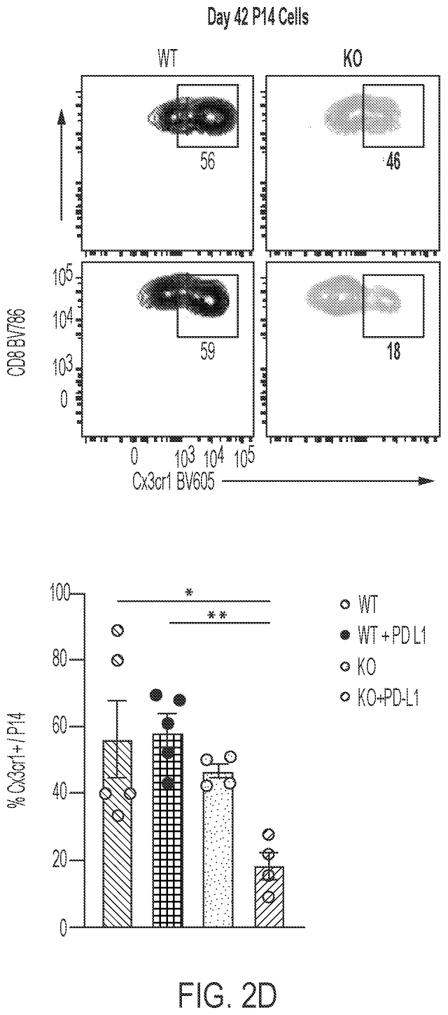

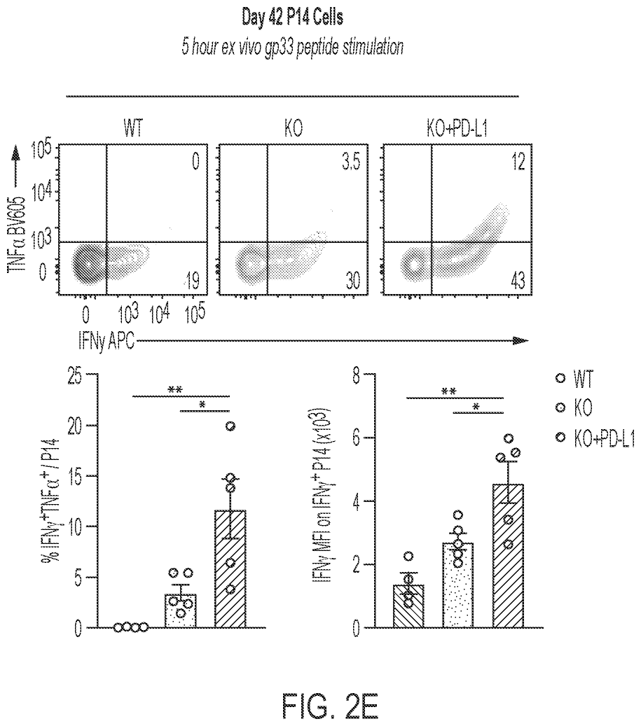

In the present Example, guide RNAs were optimized for disrupting exons 10 and 12 of the Asxl1 gene in P14 CD8 T cells. Synthego analysis of the PCR amplified target region confirmed efficient editing of Asxl1. These genetically modified P14 CD8 T cells were adoptively transferred into mice which were subsequently infected with chronic lymphocytic choriomeningitis virus (LCMV) (FIG. 2A). The gene edited P14 CD8 T cells were then adoptively transferred into mice that were subsequently infected with chronic LCMV clone 13. The percentage of P14 CD8 T cells was tracked longitudinally in the peripheral blood and after one month, the mice were treated with anti-PD-L1. The frequency of the Asxl1 KO P14 cells increased significantly after anti-PDL1 blockade (FIG. 2B) suggesting the disruption of Asxl1 preserved a stem-like state of T cell differentiation. To further assess for a stem-like phenotype, the expression of CD101, Tim3, and Cx3cr1 was examined among the wildtype (WT) and Asxl1 KO P14 CD8 T cells (37, 38). Phenotypic evaluation of CD8 T cells subsets revealed that the precursor of exhausted (Tpex) population (CD101-Tim3-) was most prevalent in the Asxl1 KO CD8 T cells (FIG. 2C). Furthermore, the stem-like Cx3cr1-subset of cells was enriched only among the Asxl1 KO P14s (FIG. 2D). The functional capacity of the cells was next assessed by measuring their ability to express effector cytokines during a 5-hour ex vivo peptide stimulation assay. Prior to anti-PDL1 blockade, the Asxl1 KOP14 cells expressed more interferon gamma (IFNg) and tumor necrosis factor alpha (TNFa) than the WT, which was further enhanced after anti-programmed death-ligand 1 (PDL1) blockade (FIG. 2E). Collectively these data demonstrate that disruption of Asxl1 preserves the T cells in a stem-like state that retains a proliferative and functional capacity. This observation is consistent with previous studies describing precursor exhausted cells as being responsible for the proliferative burst of T cells observed after blocking PD1 signaling (39, 40).

### Example 3. CRISPR-Mediated Knockout (KO) of Asxl1 in Murine Antigen-Specific T Cells

Clonal expansion of hematopoietic stem cells (HSCs) occurs in the majority of otherwise healthy adults with age (43). Such clonal hematopoiesis of indeterminate potential (CHIP) mainly results from mutations of epigenetic regulators, e.g., ASXL1. Importantly, these mutations in the stem cell compartment are enriched in the downstream cellular populations, including T cells. When leukemia patients receive donor stem cells that contain CH-associated mutations, their outcome appears to be linked to the type of donor cell mutations. As an example, recipients of stem cells with CH-associated DNMT3A mutations have a significant increase in overall survival. This survival appears to be coupled to T cell function since the enhance survival is lost in a cohort of patients that received post-transplant cyclophosphamide to prevent graft-versus-host disease (GvHD) by depleting T cells. The disparate clinical outcomes of these recipients suggest unique influences of cellular differentiation. Despite the increasing prevalence of CHIP with age, only a subset of patients develop cancer, raising the question as to what drives malignant transformation, and how epigenetic regulators specifically contribute to T cell homeostasis. The present Example, as well as, e.g., Example 4 (see below), will use the LCMV model system of chronic viral infection to assess the role of Asxl1 which allows for interrogation of antigen-specific T cells in a fully-intact animal model.

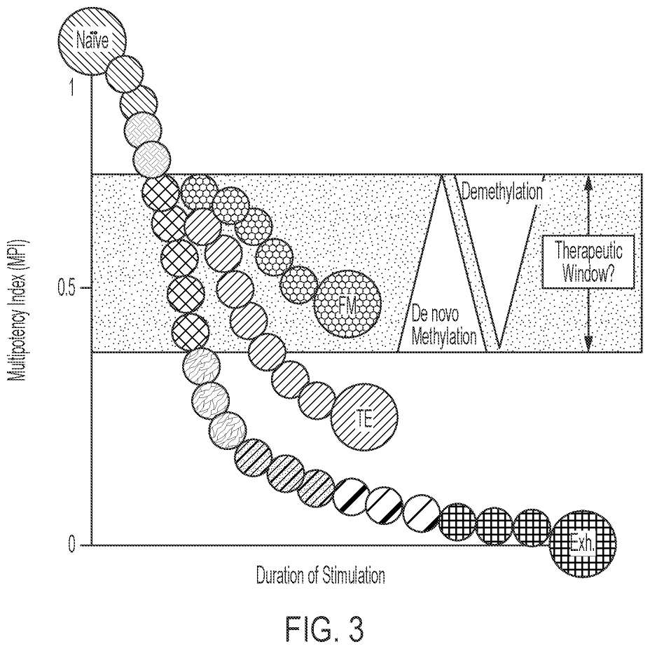

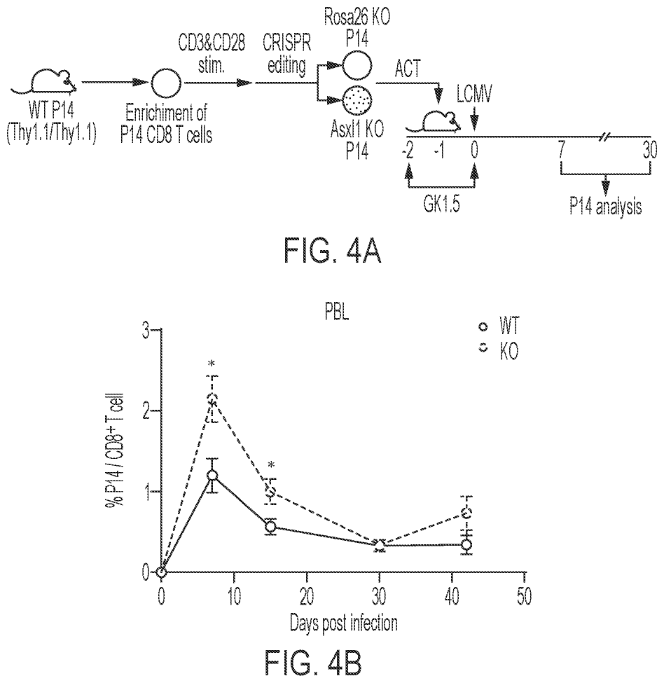

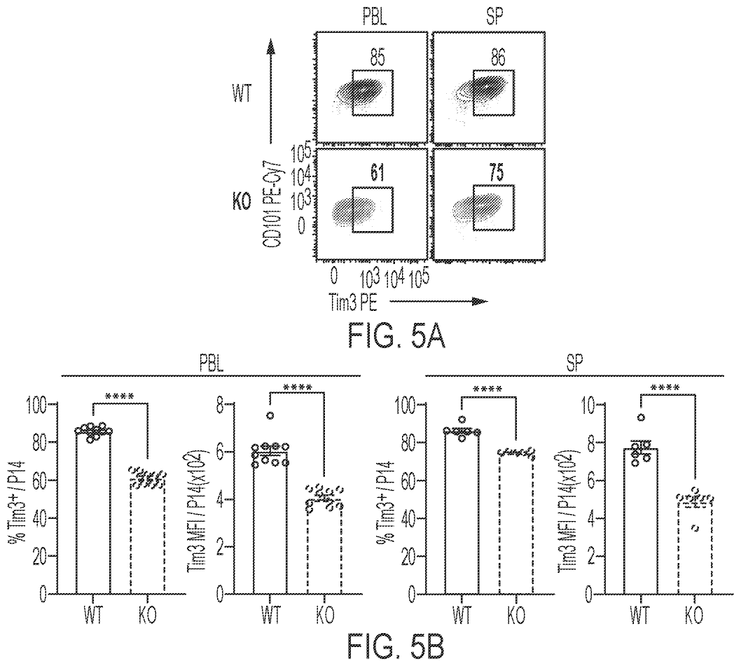

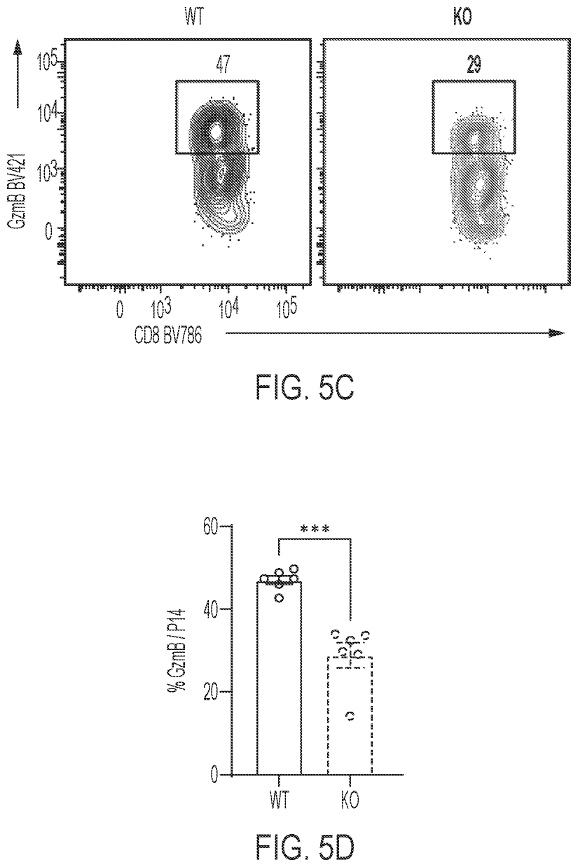

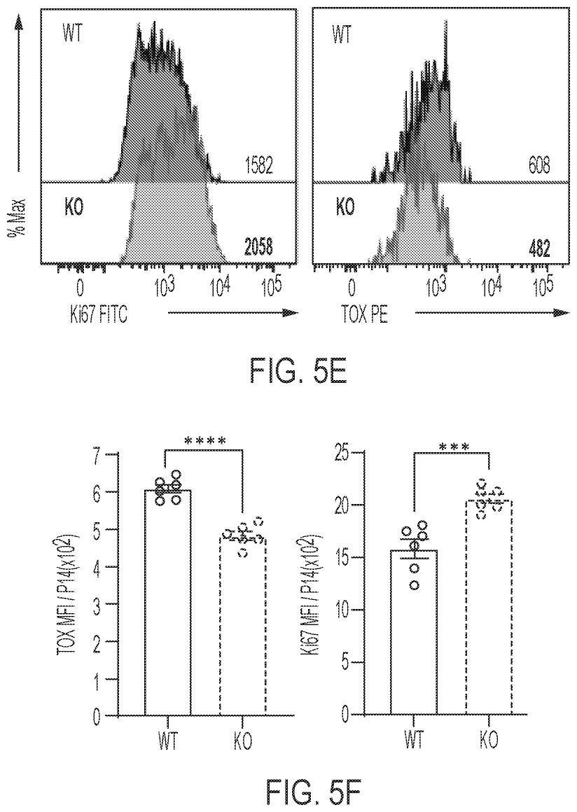

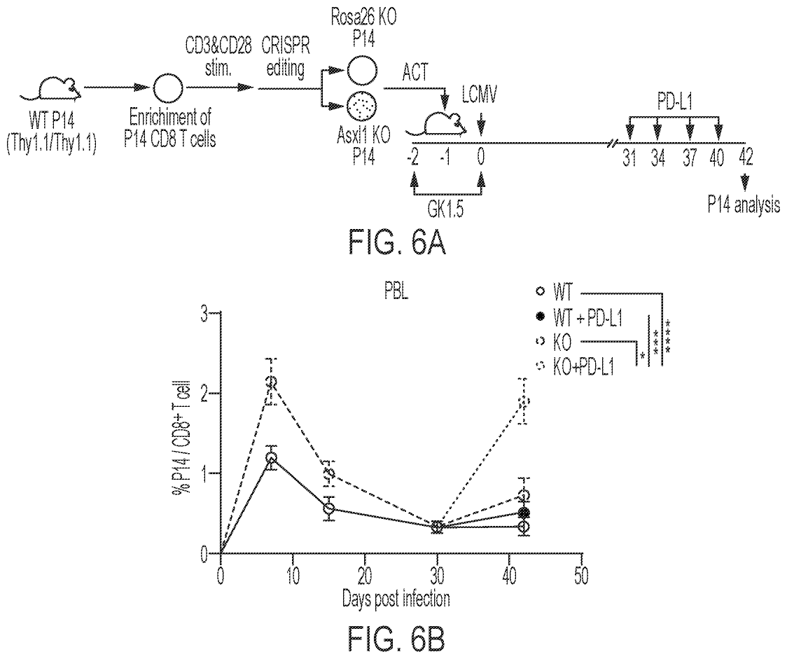

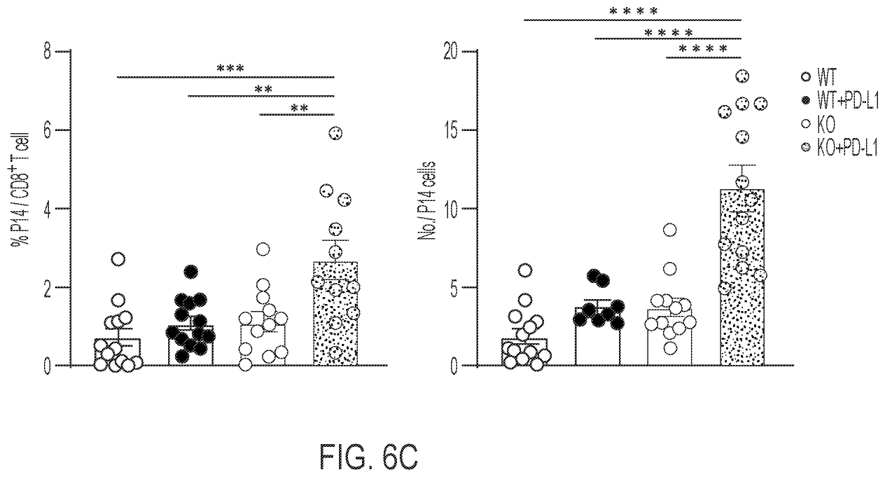

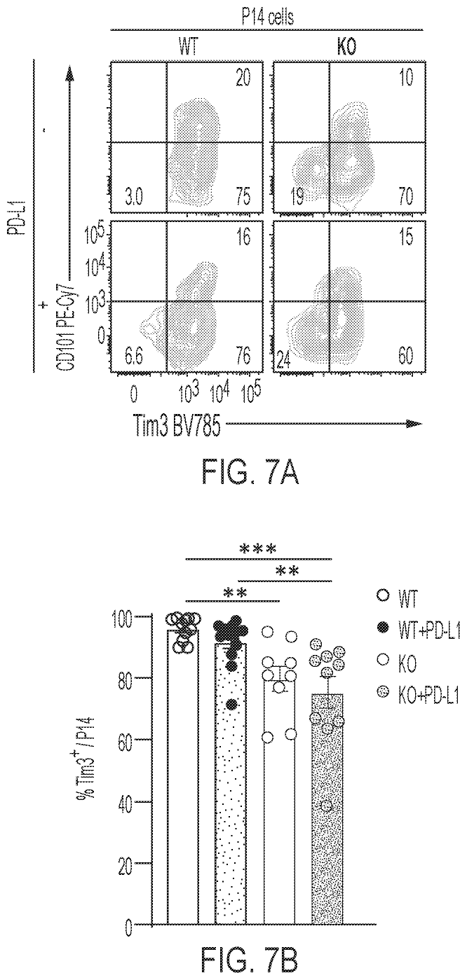

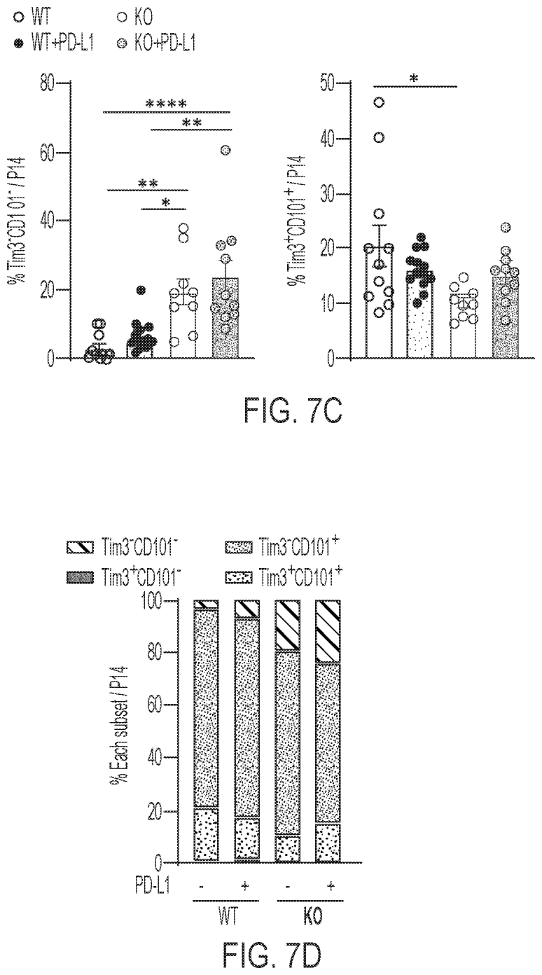

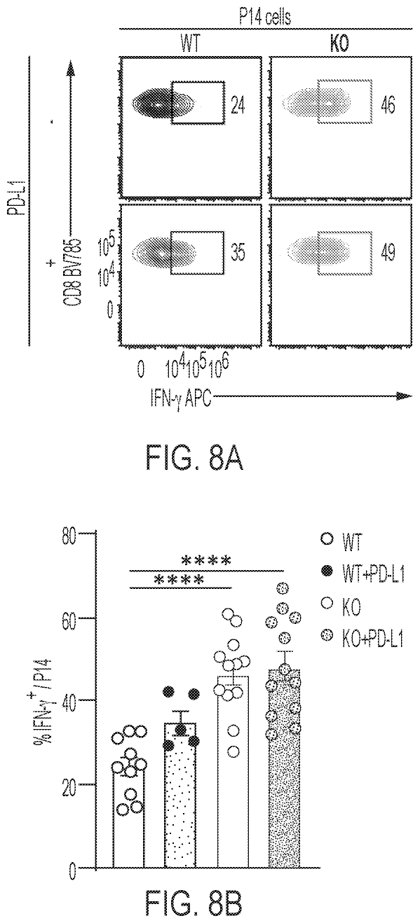

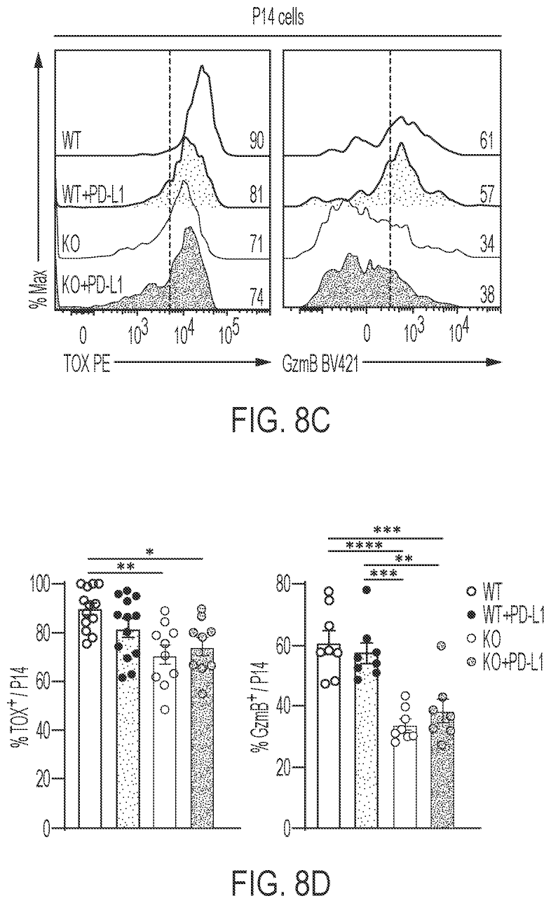

To identify the epigenetic programs controlled by CH-associated driver Asxl1 during T cell differentiation, CRISPR technology is used to knockout (KO) Asxl1 in murine antigen-specific T cells. Specifically, KO of Asxl1 is performed in P14 CD8 T cells which are T-cell receptor (TCR) transgenic cells that recognize lymphocytic choriomeningitis virus (LCMV). The genetically edited P14 CD8 T cells (Thy 1.1) will be injected into congenically distinct (Thy 1.2) WT B6 mice which will then be infected with LCMV. The P14 CD8 T cell immunological response will be tracked longitudinally during effector and memory T cell differentiation. Phenotyping will be performed to characterize T cell homing (CD103, CD69, & CD62L) and to examine the phenotypic transition between a stem-like state to fully exhausted (PD-1, Tim3, CD101, Cxcr3, and Slamf6). In addition, functional assays will be performed to assess proliferation (Cell Trace Violet [CTV] labeling and adoptive transfer of chronically stimulated P14s into infection matched animals) and effector function (ex vivo assessment of cytokine secretion after gp33 peptide stimulation). Fluorescence-activated Cell Sorting (FACS) purification of P14 T cells will allow for examination of their epigenetic heterogeneity through scRNAseq single-cell RNA sequencing (scRNAseq), single-cell assay for transposase-accessible chromatin using sequencing (scATACseq), and whole-genome methylation (WGM) profiling approaches as the T cells enter a homeostatic versus hyperproliferative state. MPI (23) (FIG. 3) will also be used to determine the differentiation status of the genetically modified P14 T cells and to correlate this multipotency score to persistence and expansion. A homeostatic state described herein may comprise an ability of a cell to continue to divide at a rate essentially to maintain a steady or stable population size. A hyperproliferative state described herein may comprise an increasing number of cellular divisions of a cell which can be associated with increasing cell populations. A multivariate linear regression model will be used to model the relationship between P14 T cell expansion and the multipotency score. Specifically, expansion is the dependent variable, multipotency score and other features such as viral load are explanatory variables.

By way of a non-limiting example, whole-genome methylation analysis will be performed by sequencing samples, e.g., to a depth of 30× coverage, giving the statistical power to discern changes in CpG methylation of ±20% (p=0.05 using Fisher's exact test with 2×2 contingency tables). Analysis of the WGBS data generated herein will include determining the quality of the sequence data including base score and library complexity, and the total percent of CpGs covered with sufficient power to discern at least a 50% change in methylation. Current protocol yields ˜80% of CpGs having 30× sequencing coverage. For example, without limitation, with a sample size of 6 and assuming 10,000 non-differentially methylated regions with 100 false positive differentially methylated regions (DMRs) and a mean difference of methylation being 0.2, the anticipated standard deviation of the methylation difference is 0.1 with a power of 0.8. To estimate sample size needed for the phentoypic and functional studies it can be calculated that 20 WT and 20 KO mice will allow for detection of differences among cell subsets with levels differing between WT and KO mice with a large effect size of 0.9, using a two-sample t test, with a power of 80% at a type 1 error level of 0.05. Both male and female mice will be used in equal numbers and results will be reported based on individual sex and combined.

### Example 4. Evaluation of Hyperproliferative State in the Setting of Asxl1 Gene Disruption

To determine if the hyperproliferative state that occurs in the setting of Asxl1 gene disruption in T cells requires persistent T cell receptor (TCR) engagement, Asxl1 KO P14 T cells from chronic LCMV infected mice will be adoptively transferred into one of two groups of WT B6 mice. One group of mice will have already been infected with acute LCMV and fully recovered. These immunized animals will clear any virus that is transferred along with the P14 cells, thus allowing for monitoring of cell numbers in the absence of antigen. The other group of mice will be infected with chronic LCMV in order for the P14 T cells to receive persistent TCR signaling. T cell expansion will be monitored longitudinally by measuring the percentage and absolute number of P14 CD8 T cells isolated from peripheral blood longitudinally. In addition, the Asxl1 KO P14 CD8 T cells will be CTV labeled prior to adoptive transfer into both the fully recovered and chronically infected mice to assess proliferative capacity. Epigenetic profiling (DNA methylation and ATACseq), e.g., using approaches similar to or the same as those described in Example 3, will be used to identify differences related to the presence or absence of persistent TCR engagement. The P14 CD8 T cell DNA methylation profiles will be assessed with MPI as described herein (see, e.g., Example 3). In addition, functional assays will be performed, including characterization of cytokine secretion.

Asxl1 gene disruption will be tested using a murine CAR T cell model(s) with an intact immune system. Additionally, targeted approaches will be developed using small molecules to specifically modify genes identified to be associated with CD8 T cell proliferative ability.

### Example 5. Evaluation of Imprint of Proliferation Checkpoints Prior to Memory Generation

Epigenetic programs can be coupled to the development of CAR T cell exhaustion in autologous CD19 CAR T cells isolated longitudinally from patients. The present Example is designed to test whether disruption of CH-associated gene(s), e.g., ASXL1, disrupts T cell homeostasis programs prior to the establishment of memory T cell gene expression programs. Further, whether mutations in ASXL1 predispose CD8 T cells to undergo antigen-independent proliferation will be determined.

Knockout (KO) of ASXL1 will be performed in CAR T cells generated from both naïve and memory T cell subsets. Naïve and memory T cells will be FACS purified based on the expression of CCR7 and CD45RO (4, 7, 17). The naïve and memory-derived Her2 and IL 13Rα2 CAR T KO CAR T cells will then be chronically stimulated using in vitro assays to assess proliferation, anti-tumor cytotoxicity, and cytokine expression. Furthermore, DNA will be extracted from and bisulfite-converted for WGBS methylation profiling. MPI (23) (FIG. 3) will be used to determine the multipotency score of the CAR T cells. A multivariate linear regression model will be used to model the relationship between CAR T cell expansion and the multipotency score. Specifically, expansion is the dependent variable, multipotency score and other features such as tumor burden are explanatory variables. The MPI score and expansion of CH-associated gene(s), e.g., ASXL1, disrupted CAR T cells will be isolated from naïve and memory CD8 T cell subsets.

Further approaches to examining the heterogeneity of CAR T cells generated from the naïve and memory T cell subsets will include examination of the TCR repertoire and single cell ATAC-seq. ASXL1 KO CAR T cells will be adoptively transferred into NOD-SCID IL2Rγnull (NSG) mice and antigen-independent proliferation anti-tumor response will be assessed in an in vivo setting. Practical application of these results includes clinical trials.

### Example 6. Identification of ASXL1 Mutation-Induced Antigen-Independent Cell Proliferation in Human T Cells

To identify whether ASXL1 mutations induce antigen-independent cell proliferation in human T cells, ASXL1 KO CAR T cells will be cultured in the presence and absence of antigen and monitored longitudinally for antigen-independent proliferation. To assess for antigen-independent homeostasis, the cells will be cultured in the presence of IL 7 and IL 15.

### Example 7. Evaluation of Mutant-ASXL1-Associated Enhanced Proliferative Potential in the Memory T Cell Compartment in Patients

Mutations in CH-associated genes, e.g., ASXL1, are found both in healthy individuals with CHIP, as well as patients with myelodysplastic syndrome (MDS). Samples from patients with confirmed mutant ASXL1 in both the myeloid and T cell compartments will be obtained. Naïve and memory T cells will be purified from the peripheral blood of myelodysplastic syndrome (MDS) patients and WGM profiling will be performed. The DNA methylation program(s) associated with ASXL1 mutations among the clinical samples will be used to determine if the above-described hyperproliferative-associated epigenetic signature (see, e.g., Examples 3-6) is enriched among naïve versus the memory CD8 T cell compartments. DNA methylation based MPI will be applied to determine if the T cells with, e.g., ASXL1 disruption, are predicted to have preserved developmental potential. From these studies it will be determined if naturally occurring ASXL1-associated mutation(s) modify CD8 T cell epigenetic programs in a manner that predicts a hyperproliferative state. Data from this analysis will provide a foundation for investigation of T cell-based therapeutics that exploit endogenous cells with, e.g., ASXL1 mutation(s) and/or prioritize which patient T cells may be considered for adoptive cellular therapy.

Proliferation-associated programs may be more prevalent in the memory CD8 T cell compartment as such cells have already undergone expansion in response to antigen. MPI may predict both murine and human CD8 T cell developmental potential (34, 35), and epigenetic programs may be conserved among species. Site-specific mutations that naturally occur in ASXL1 will be introduced into healthy donor T cells and the proliferative properties assessed.

### REFERENCES

1 Fraietta J A, Lacey S F, Orlando E J, Pruteanu-Malinici I, Gohil M, Lundh S, et al. Determinants of response and resistance to CD19 chimeric antigen receptor (CAR) T cell therapy of chronic lymphocytic leukemia. Nat Med. 2018; 24(5):563-71.
2. Zebley C C, Gottschalk S, Youngblood B. Rewriting History: Epigenetic Reprogramming of CD8(+) T Cell Differentiation to Enhance Immunotherapy. Trends Immunol. 2020; 41(8):665-75.
3. Ghoneim H E, Fan Y, Moustaki A, Abdelsamed H A, Dash P, Dogra P, et al. De Novo Epigenetic Programs Inhibit PD-1 Blockade-Mediated T Cell Rejuvenation. Cell. 2017; 170(1):142-57 e19.
4. Prinzing B, Zebley C C, Petersen C T, Fan Y, Anido A A, Yi Z, et al. Deleting DNMT3A in CAR T cells prevents exhaustion and enhances antitumor activity. Sci Transl Med. 2021; 13(620):eabh0272.
5. Jain N, Zhao Z, Iyer A S, Lopez M, Feucht J, Koche R P, et al. Loss of TET2 Uncouples Proliferative and Effector Functions in CAR T Cells. Blood. 2020; 136.
6. Carty S A, Gohil M, Banks L B, Cotton R M, Johnson M E, Stelekati E, et al. The Loss of TET2 Promotes CD8(+) T Cell Memory Differentiation. J Immunol. 2018; 200(1):82-91.
7. Abdelsamed H A, Moustaki A, Fan Y, Dogra P, Ghoneim H E, Zebley C C, et al. Human memory CD8 T cell effector potential is epigenetically preserved during in vivo homeostasis. The Journal of experimental medicine. 2017; 214(6):1593-606.
8. Flynn J K, Gorry P R. Stem memory T cells (TSCM)-their role in cancer and HIV immunotherapies. Clin Transl Immunology. 2014; 3(7):e20.
9. Gattinoni L, Lugli E, Ji Y, Pos Z, Paulos C M, Quigley M F, et al. A human memory T cell subset with stem cell-like properties. Nat Med. 2011; 17(10):1290-7.
10. Sabatino M, Hu J, Sommariva M, Gautam S, Fellowes V, Hocker J D, et al. Generation of clinical-grade CD19-specific CAR-modified CD8+ memory stem cells for the treatment of human B-cell malignancies. Blood. 2016; 128(4):519-28.
11. Klebanoff C A, Gattinoni L, Restifo N P. Sorting through subsets: which T-cell populations mediate highly effective adoptive immunotherapy? J Immunother. 2012; 35(9):651-60.
12. Kurtulus S, Madi A, Escobar G, Klapholz M, Nyman J, Christian E, et al. Checkpoint Blockade Immunotherapy Induces Dynamic Changes in PD-1(−)CD8(+) Tumor-Infiltrating T Cells. Immunity. 2019; 50(1):181-94 e6.
13. Krishna S, Lowery F J, Copeland A R, Bahadiroglu E, Mukherjee R, Jia L, et al. Stem-like CD8 T cells mediate response of adoptive cell immunotherapy against human cancer. Science. 2020; 370(6522):1328-34.
14. Zebley C C, Brown C, Mi T, Fan Y, Alli S, Boi S, et al. CD19-CAR T cells undergo exhaustion DNA methylation programming in patients with acute lymphoblastic leukemia. Cell Rep. 2021; 37(9):110079.
15. Reik W. Stability and flexibility of epigenetic gene regulation in mammalian development. Nature. 2007; 447(7143):425-32.
16. Abdel-Hakeem M S, Manne S, Beltra J C, Stelekati E, Chen Z, Nzingha K, et al. Epigenetic scarring of exhausted T cells hinders memory differentiation upon eliminating chronic antigenic stimulation. Nat Immunol. 2021; 22(8):1008-19.
17. Zebley C C, Abdelsamed H A, Ghoneim H E, Alli S, Brown C, Haydar D, et al. Proinflammatory cytokines promote TET2-mediated DNA demethylation during CD8 T cell effector differentiation. Cell Rep. 2021; 37(2):109796.
18. van den Akker E B, Makrodimitris S, Hulsman M, Brugman M H, Nikolic T, Bradley T, et al. Dynamic clonal hematopoiesis and functional T-cell immunity in a supercentenarian. Leukemia. 2021; 35(7):2125-9.
19. Hansen J W, Pedersen D A, Larsen L A, Husby S, Clemmensen S B, Hjelmborg J, et al. Clonal hematopoiesis in elderly twins: concordance, discordance, and mortality. Blood. 2020; 135(4):261-8.
20. Steensma D P, Bejar R, Jaiswal S, Lindsley R C, Sekeres M A, Hasserjian R P, et al. Clonal hematopoiesis of indeterminate potential and its distinction from myelodysplastic syndromes. Blood. 2015; 126(1):9-16.
21. Youngblood B, Hale J S, Kissick H T, Ahn E, Xu X, Wieland A, et al. Effector CD8 T cells dedifferentiate into long-lived memory cells. Nature. 2017; 552(7685):404-9.
22. Joseph A. Fraietta C L N, Morgan A. Sammons, Stefan Lundh, Shannon A. Carty, Tyler J. Reich, Alexandria P. Cogdill, Jennifer J. D. Morrissette, Jamie E. DeNizio, Shantan Reddy, Young Hwang, Mercy Gohil, Irina Kulikovskaya, Farzana Nazimuddin, Minnal Gupta, Fang Chen, John K. Everett, Katherine A. Alexander, Enrique Lin-Shiao, Marvin H. Gee, Xiaojun Liu, Regina M. Young, David Ambrose, Yan Wang, Jun Xu, Martha S. Jordan, Katherine T. Marcucci, Bruce L. Levine, K. Christopher Garcia, Yangbing Zhao, Michael Kalos, David L. Porter, Rahul M. Kohli, Simon F. Lacey, Shelley L. Berger, Frederic D. Bushman, Carl H. June & J. Joseph Melenhorst. Disruption of TET2 promotes the therapeutic efficacy of CD19-targeted T cells. Nature. 2018.
23. Abdelsamed H A, Zebley C C, Nguyen H, Rutishauser R L, Fan Y, Ghoneim H E, et al. Beta cell-specific CD8(+) T cells maintain stem cell memory-associated epigenetic programs during type 1 diabetes. Nat Immunol. 2020.
24. Fonseca R, Beura L K, Quarnstrom C F, Ghoneim H E, Fan Y, Zebley C C, et al. Developmental plasticity allows outside-in immune responses by resident memory T cells. Nat Immunol. 2020.
25. Petiti L, Pace L. The persistence of stemness. Nat Immunol. 2020.
26. Hale J S, Youngblood B, Latner D R, Mohammed A U, Ye L, Akondy R S, et al. Distinct memory CD4+ T cells with commitment to T follicular helper- and T helper 1-cell lineages are generated after acute viral infection. Immunity. 2013; 38(4):805-17.
27. Nakayama-Hosoya K, Ishida T, Youngblood B, Nakamura H, Hosoya N, Koga M, et al. Epigenetic Repression of Interleukin 2 Expression in Senescent CD4+ T Cells During Chronic HIV Type 1 Infection. J Infect Dis. 2014.
28. Scharer C D, Barwick B G, Youngblood B A, Ahmed R, Boss J M. Global DNA Methylation Remodeling Accompanies CD8 T Cell Effector Function. J Immunol. 2013.
29. Youngblood B, Hale J S, Akondy R. Using epigenetics to define vaccine-induced memory T cells. Curr Opin Virol. 2013; 3(3):371-6.
30. Youngblood B, Noto A, Porichis F, Akondy R S, Ndhlovu Z M, Austin J W, et al. Cutting edge: Prolonged exposure to HIV reinforces a poised epigenetic program for PD-1 expression in virus-specific CD8 T cells. J Immunol. 2013; 191(2):540-4.
31. Youngblood B, Oestreich K J, Ha S J, Duraiswamy J, Akondy R S, West E E, et al. Chronic virus infection enforces demethylation of the locus that encodes PD-1 in antigen-specific CD8+ T cells Immunity. 2011; 35(3):13.
32. Akondy R S, Fitch M, Edupuganti S, Yang S, Kissick H T, Li K W, et al. Origin and differentiation of human memory CD8 T cells after vaccination. Nature. 2017; 552(7685):362-7.
33. Abdelsamed H A, Moustaki A, Fan Y P, Dogra P, Ghoneim H E, Zebley C C, et al. Human memory CD8 T cell effector potential is epigenetically preserved during in vivo homeostasis. Journal of Experimental Medicine. 2017; 214(6):1593-606.
34 Abdelsamed H A, Zebley C C, Nguyen H, Rutishauser R L, Fan Y, Ghoneim H E, et al. Beta cell-specific CD8(+) T cells maintain stem cell memory-associated epigenetic programs during type 1 diabetes. Nat Immunol. 2020; 21(5):578-87.
35. Fonseca R, Beura L K, Quarnstrom C F, Ghoneim H E, Fan Y, Zebley C C, et al. Developmental plasticity allows outside-in immune responses by resident memory T cells. Nat Immunol. 2020; 21(4):412-21.
36. Genovese G, Kahler A K, Handsaker R E, Lindberg J, Rose S A, Bakhoum S F, et al. Clonal hematopoiesis and blood-cancer risk inferred from blood DNA sequence. N Engl J Med. 2014; 371(26):2477-87.
37. Hudson W H, Gensheimer J, Hashimoto M, Wieland A, Valanparambil R M, Li P, et al. Proliferating Transitory T Cells with an Effector-like Transcriptional Signature Emerge from PD-1(+) Stem-like CD8(+) T Cells during Chronic Infection. Immunity. 2019; 51(6):1043-58 e4.
38 Beltra J C, Manne S, Abdel-Hakeem M S, Kurachi M, Giles J R, Chen Z, et al. Developmental Relationships of Four Exhausted CD8(+) T Cell Subsets Reveals Underlying Transcriptional and Epigenetic Landscape Control Mechanisms. Immunity. 2020; 52(5):825-41 e8.
39 Im S J, Hashimoto M, Gerner M Y, Lee J, Kissick H T, Burger M C, et al. Defining CD8+ T cells that provide the proliferative burst after PD-1 therapy. Nature. 2016; 537(7620):417-21.
40. Kallies A, Zehn D, Utzschneider D T. Precursor exhausted T cells: key to successful immunotherapy? Nat Rev Immunol. 2020; 20(2):128-36.
41. Liao J, Karnik R, Gu H, Ziller M J, Clement K, Tsankov A M, et al. Targeted disruption of DNMT1, DNMT3A and DNMT3B in human embryonic stem cells. Nat Genet. 2015; 47(5):469-78.
42. Galletti G, De Simone G, Mazza E M C, Puccio S, Mezzanotte C, Bi T M, et al. Two subsets of stem-like CD8(+) memory T cell progenitors with distinct fate commitments in humans. Nat Immunol. 2020; 21(12):1552-62.
43 Park S J, Bejar R. Clonal Hematopoiesis in Aging. Curr Stem Cell Rep. 2018; 4(3):209-19.
44. Youngblood B, Reich N O. The early expressed HIV-1 genes regulate DNMT1 expression. Epigenetics. 2008; 3(3):149-56.
45. Landt S G, Marinov G K, Kundaje A, Kheradpour P, Pauli F, Batzoglou S, et al. ChIP-seq guidelines and practices of the ENCODE and modENCODE consortia. Genome Res. 2012; 22(9):1813-31.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

Listing of Sequences
scFvFRP5 + leader amino acid sequence
SEQ ID NO: 1
MDWIWRILFLVGAATGAHSEVQLQQSGPELKKPGETVKISCKASGYPFTNYG
MNWVKQAPGQGLKWMGWINTSTGESTFADDFKGRFDFSLETSANTAYLQINNLKS
EDMATYFCARWEVYHGYVPYWGQGTTVTVSSGGGGSGGGGSGGGGSDIQLTQSH
KFLSTSVGDRVSITCKASQDVYNAVAWYQQKPGQSPKLLIYSASSRYTGVPSRFTGS
GSGPDFTFTISSVQAEDLAVYFCQQHFRTPFTFGSGTKLEIKAL
scFvFRP5 + leader DNA sequence
SEQ ID NO: 2
ATGGACTGGATCTGGCGCATCCTGTTTCTCGTGGGAGCCGCCACAGGCGCCCATT
CTGAGGTACAACTGCAGCAGTCTGGACCTGAACTGAAGAAGCCTGGAGAGACAG
TCAAGATCTCCTGCAAGGCCTCTGGGTATCCTTTCACAAACTATGGAATGAACTG
GGTGAAGCAGGCTCCAGGACAGGGTTTAAAGTGGATGGGCTGGATTAACACCTC
CACTGGAGAGTCAACATTTGCTGATGACTTCAAGGGACGGTTTGACTTCTCTTTG
GAAACCTCTGCCAACACTGCCTATTTGCAGATCAACAACCTCAAAAGTGAAGAC
ATGGCTACATATTTCTGTGCAAGATGGGAGGTTTACCACGGCTACGTTCCTTACT
GGGGCCAAGGGACCACGGTCACCGTTTCCTCTGGCGGTGGCGGTTCTGGTGGCG
GTGGCTCCGGCGGTGGCGGTTCTGACATCCAGCTGACCCAGTCTCACAAATTCCT
GTCCACTTCAGTAGGAGACAGGGTCAGCATCACCTGCAAGGCCAGTCAGGATGT
GTATAATGCTGTTGCCTGGTATCAACAGAAACCAGGACAATCTCCTAAACTTCTG
ATTTACTCGGCATCCTCCCGGTACACTGGAGTCCCTTCTCGCTTCACTGGCAGTG
GCTCTGGGCCGGATTTCACTTTCACCATCAGCAGTGTGCAGGCTGAAGACCTGGC
AGTTTATTTCTGTCAGCAACATTTTCGTACTCCATTCACGTTCGGCTCGGGGACAA
AATTGGAGATCAAAGCTCTA
FRP5.zeta CAR amino acid sequence
SEQ ID NO: 3
MDWIWRILFLVGAATGAHSEVQLQQSGPELKKPGETVKISCKASGYPFTNYGMNWV
KQAPGQGLKWMGWINTSTGESTFADDFKGRFDFSLETSANTAYLQINNLKSEDMAT
YFCARWEVYHGYVPYWGQGTTVTVSSGGGGSGGGGSGGGGSDIQLTQSHKFLSTS
VGDRVSITCKASQDVYNAVAWYQQKPGQSPKLLIYSASSRYTGVPSRFTGSGSGPDF
TFTISSVQAEDLAVYFCQQHFRTPFTFGSGTKLEIKALDLEPKSCDKTHTCPPCPDPKL
CYLLDGILFIYGVILTALFLRVKFSRSADAPAYQQGQNQLYNELNLGRREEYDVLDK
RRGRDPEMGGKPRRKNPQEGLYNELQKDKMAEAYSEIGMKGERRRGKGHDGLYQ
GLSTATKDTYDALHMQALPPR
FRP5.zeta CAR DNA sequence
SEQ ID NO: 4
ATGGACTGGATCTGGCGCATCCTGTTTCTCGTGGGAGCCGCCACAGGCGCCCATT
CTGAGGTACAACTGCAGCAGTCTGGACCTGAACTGAAGAAGCCTGGAGAGACAG
TCAAGATCTCCTGCAAGGCCTCTGGGTATCCTTTCACAAACTATGGAATGAACTG
GGTGAAGCAGGCTCCAGGACAGGGTTTAAAGTGGATGGGCTGGATTAACACCTC
CACTGGAGAGTCAACATTTGCTGATGACTTCAAGGGACGGTTTGACTTCTCTTTG
GAAACCTCTGCCAACACTGCCTATTTGCAGATCAACAACCTCAAAAGTGAAGAC
ATGGCTACATATTTCTGTGCAAGATGGGAGGTTTACCACGGCTACGTTCCTTACT
GGGGCCAAGGGACCACGGTCACCGTTTCCTCTGGCGGTGGCGGTTCTGGTGGCG
GTGGCTCCGGCGGTGGCGGTTCTGACATCCAGCTGACCCAGTCTCACAAATTCCT
GTCCACTTCAGTAGGAGACAGGGTCAGCATCACCTGCAAGGCCAGTCAGGATGT
GTATAATGCTGTTGCCTGGTATCAACAGAAACCAGGACAATCTCCTAAACTTCTG
ATTTACTCGGCATCCTCCCGGTACACTGGAGTCCCTTCTCGCTTCACTGGCAGTG
GCTCTGGGCCGGATTTCACTTTCACCATCAGCAGTGTGCAGGCTGAAGACCTGGC
AGTTTATTTCTGTCAGCAACATTTTCGTACTCCATTCACGTTCGGCTCGGGGACAA
AATTGGAGATCAAAGCTCTAGATCTCGAGCCCAAATCTTGTGACAAAACTCACA
CATGCCCACCGTGCCCGGATCCCAAACTCTGCTACCTGCTGGATGGAATCCTCTT
CATCTATGGTGTCATTCTCACTGCCTTGTTCCTGAGAGTGAAGTTCAGCAGGAGC
GCAGACGCCCCCGCGTACCAGCAGGGCCAGAACCAGCTCTATAACGAGCTCAAT
CTAGGACGAAGAGAGGAGTACGATGTTTTGGACAAGAGACGTGGCCGGGACCCT
GAGATGGGGGGAAAGCCGAGAAGGAAGAACCCTCAGGAAGGCCTGTACAATGA
ACTGCAGAAAGATAAGATGGCGGAGGCCTACAGTGAGATTGGGATGAAAGGCG
AGCGCCGGAGGGGCAAGGGGCACGATGGCCTTTACCAGGGTCTCAGTACAGCCA
CCAAGGACACCTACGACGCCCTTCACATGCAGGCCCTGCCCCCTCGC
FRP5.CD28.zeta CAR amino acid sequence
SEQ ID NO: 5
MDWIWRILFLVGAATGAHSEVQLQQSGPELKKPGETVKISCKASGYPFTNYGMNWV
KQAPGQGLKWMGWINTSTGESTFADDFKGRFDFSLETSANTAYLQINNLKSEDMAT
YFCARWEVYHGYVPYWGQGTTVTVSSGGGGSGGGGSGGGGSDIQLTQSHKFLSTS
VGDRVSITCKASQDVYNAVAWYQQKPGQSPKLLIYSASSRYTGVPSRFTGSGSGPDF
TFTISSVQAEDLAVYFCQQHFRTPFTFGSGTKLEIKALDLEPKSCDKTHTCPPCPDPKF
WVLVVVGGVLACYSLLVTVAFIIFWVRSKRSRLLHSDYMNMTPRRPGPTRKHYQPY
APPRDFAAYRSRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATK
DTYDALHMQALPPR
FRP5.CD28.zeta CAR DNA sequence
SEQ ID NO: 6
ATGGACTGGATCTGGCGCATCCTGTTTCTCGTGGGAGCCGCCACAGGCGCCCATT
CTGAGGTACAACTGCAGCAGTCTGGACCTGAACTGAAGAAGCCTGGAGAGACAG
TCAAGATCTCCTGCAAGGCCTCTGGGTATCCTTTCACAAACTATGGAATGAACTG
GGTGAAGCAGGCTCCAGGACAGGGTTTAAAGTGGATGGGCTGGATTAACACCTC
CACTGGAGAGTCAACATTTGCTGATGACTTCAAGGGACGGTTTGACTTCTCTTTG
GAAACCTCTGCCAACACTGCCTATTTGCAGATCAACAACCTCAAAAGTGAAGAC
ATGGCTACATATTTCTGTGCAAGATGGGAGGTTTACCACGGCTACGTTCCTTACT
GGGGCCAAGGGACCACGGTCACCGTTTCCTCTGGCGGTGGCGGTTCTGGTGGCG
GTGGCTCCGGCGGTGGCGGTTCTGACATCCAGCTGACCCAGTCTCACAAATTCCT
GTCCACTTCAGTAGGAGACAGGGTCAGCATCACCTGCAAGGCCAGTCAGGATGT
GTATAATGCTGTTGCCTGGTATCAACAGAAACCAGGACAATCTCCTAAACTTCTG
ATTTACTCGGCATCCTCCCGGTACACTGGAGTCCCTTCTCGCTTCACTGGCAGTG
GCTCTGGGCCGGATTTCACTTTCACCATCAGCAGTGTGCAGGCTGAAGACCTGGC
AGTTTATTTCTGTCAGCAACATTTTCGTACTCCATTCACGTTCGGCTCGGGGACAA
AATTGGAGATCAAAGCTCTAGATCTCGAGCCCAAATCTTGTGACAAAACTCACA
CATGCCCACCGTGCCCGGATCCCAAATTTTGGGTGCTGGTGGTGGTTGGTGGAGT
CCTGGCTTGCTATAGCTTGCTAGTAACAGTGGCCTTTATTATTTTCTGGGTGAGGA
GTAAGAGGAGCAGGCTCCTGCACAGTGACTACATGAACATGACTCCCCGCCGCC
CCGGGCCCACCCGCAAGCATTACCAGCCCTATGCCCCACCACGCGACTTCGCAGC
CTATCGCTCCAGAGTGAAGTTCAGCAGGAGCGCAGACGCCCCCGCGTACCAGCA
GGGCCAGAACCAGCTCTATAACGAGCTCAATCTAGGACGAAGAGAGGAGTACGA
TGTTTTGGACAAGAGACGTGGCCGGGACCCTGAGATGGGGGGAAAGCCGAGAA
GGAAGAACCCTCAGGAAGGCCTGTACAATGAACTGCAGAAAGATAAGATGGCG
GAGGCCTACAGTGAGATTGGGATGAAAGGCGAGCGCCGGAGGGGCAAGGGGCA
CGATGGCCTTTACCAGGGTCTCAGTACAGCCACCAAGGACACCTACGACGCCCTT
CACATGCAGGCCCTGCCCCCTCGC
scFv47 + leader amino acid sequence
SEQ ID NO: 7
MDWIWRILFLVGAATGAHSQVQLQQPGAELVRPGASVKLSCKASGYTFSNYLMNW
VKQRPEQDLDWIGRIDPYDGDIDYNQNFKDKAILTVDKSSSTAYMQLSSLTSEDSAV
YYCARGYGTAYGVDYWGQGTSVTVSSAKTTPPKLEEGEFSEARVDIVLTQSPASLA
VSLGQRATISCRASESVDNYGISFMNWFQQKPGQPPKLLIYAASRQGSGVPARFSGS
GSGTDFSLNIHPMEEDDTAMYFCQQSKEVPWTFGGGTKLEIK
scFv47 + leader DNA sequence
SEQ ID NO: 8
ATGGACTGGATCTGGCGCATCCTGTTTCTCGTGGGAGCCGCCACAGGCGCCCATT
CTCAGGTGCAGCTGCAGCAGCCTGGCGCTGAACTCGTGCGGCCAGGCGCTTCTGT
GAAGCTGAGCTGTAAAGCCAGCGGCTACACCTTCAGCAACTACCTGATGAACTG
GGTCAAGCAGCGGCCCGAGCAGGACCTGGATTGGATCGGCAGAATCGACCCCTA
CGACGGCGACATCGACTACAACCAGAACTTCAAGGACAAGGCCATCCTGACCGT
GGACAAGAGCAGCAGCACCGCCTACATGCAGCTGTCCAGCCTGACCAGCGAGGA
CAGCGCCGTGTACTACTGCGCCAGAGGCTACGGCACAGCCTACGGCGTGGACTA
TTGGGGCCAGGGCACAAGCGTGACCGTGTCCAGCGCCAAGACCACCCCCCCTAA
GCTGGAAGAGGGCGAGTTCTCCGAGGCCCGGGTGGACATTGTGCTGACACAGTC
TCCAGCCAGCCTGGCCGTGTCCCTGGGACAGAGAGCCACCATCAGCTGTAGGGC
CAGCGAGAGCGTGGACAACTACGGCATCAGCTTCATGAATTGGTTCCAGCAGAA
GCCCGGCCAGCCCCCCAAGCTGCTGATCTATGCCGCCAGCAGACAGGGCAGCGG
AGTGCCTGCCAGATTTTCTGGCAGCGGCTCCGGCACCGACTTCAGCCTGAACATC
CACCCTATGGAAGAGGACGACACCGCCATGTACTTTTGCCAGCAGAGCAAAGAG
GTGCCCTGGACCTTTGGCGGAGGCACCAAGCTGGAAATCAAG
scFv47.SH.CD28.zeta CAR amino acid sequence
SEQ ID NO: 9
MDWIWRILFLVGAATGAHSQVQLQQPGAELVRPGASVKLSCKASGYTFSNYLMNW
VKQRPEQDLDWIGRIDPYDGDIDYNQNFKDKAILTVDKSSSTAYMQLSSLTSEDSAV
YYCARGYGTAYGVDYWGQGTSVTVSSAKTTPPKLEEGEFSEARVDIVLTQSPASLA
VSLGQRATISCRASESVDNYGISFMNWFQQKPGQPPKLLIYAASRQGSGVPARFSGS
GSGTDFSLNIHPMEEDDTAMYFCQQSKEVPWTFGGGTKLEIKDLEPKSCDKTHTCPP
CPDPKFWVLVVVGGVLACYSLLVTVAFIIFWVRSKRSRLLHSDYMNMTPRRPGPTR
KHYQPYAPPRDFAAYRSRVKFSRSADAPAYQQGQNQLYNELNLGRREEYDVLDKR
RGRDPEMGGKPRRKNPQEGLYNELQKDKMAEAYSEIGMKGERRRGKGHDGLYQG
LSTATKDTYDALHMQALPPR
scFv47.SH.CD28.zeta CAR DNA sequence
SEQ ID NO: 10
ATGGACTGGATCTGGCGCATCCTGTTTCTCGTGGGAGCCGCCACAGGCGCCCATT
CTCAGGTGCAGCTGCAGCAGCCTGGCGCTGAACTCGTGCGGCCAGGCGCTTCTGT
GAAGCTGAGCTGTAAAGCCAGCGGCTACACCTTCAGCAACTACCTGATGAACTG
GGTCAAGCAGCGGCCCGAGCAGGACCTGGATTGGATCGGCAGAATCGACCCCTA
CGACGGCGACATCGACTACAACCAGAACTTCAAGGACAAGGCCATCCTGACCGT
GGACAAGAGCAGCAGCACCGCCTACATGCAGCTGTCCAGCCTGACCAGCGAGGA
CAGCGCCGTGTACTACTGCGCCAGAGGCTACGGCACAGCCTACGGCGTGGACTA
TTGGGGCCAGGGCACAAGCGTGACCGTGTCCAGCGCCAAGACCACCCCCCCTAA
GCTGGAAGAGGGCGAGTTCTCCGAGGCCCGGGTGGACATTGTGCTGACACAGTC
TCCAGCCAGCCTGGCCGTGTCCCTGGGACAGAGAGCCACCATCAGCTGTAGGGC
CAGCGAGAGCGTGGACAACTACGGCATCAGCTTCATGAATTGGTTCCAGCAGAA
GCCCGGCCAGCCCCCCAAGCTGCTGATCTATGCCGCCAGCAGACAGGGCAGCGG
AGTGCCTGCCAGATTTTCTGGCAGCGGCTCCGGCACCGACTTCAGCCTGAACATC
CACCCTATGGAAGAGGACGACACCGCCATGTACTTTTGCCAGCAGAGCAAAGAG
GTGCCCTGGACCTTTGGCGGAGGCACCAAGCTGGAAATCAAGGATCTCGAGCCC
AAATCTTGTGACAAAACTCACACATGCCCACCGTGCCCGGATCCCAAATTTTGGG
TGCTGGTGGTGGTTGGTGGAGTCCTGGCTTGCTATAGCTTGCTAGTAACAGTGGC
CTTTATTATTTTCTGGGTGAGGAGTAAGAGGAGCAGGCTCCTGCACAGTGACTAC
ATGAACATGACTCCCCGCCGCCCCGGGCCCACCCGCAAGCATTACCAGCCCTATG
CCCCACCACGCGACTTCGCAGCCTATCGCTCCAGAGTGAAGTTCAGCAGGAGCG
CAGACGCCCCCGCGTACCAGCAGGGCCAGAACCAGCTCTATAACGAGCTCAATC
TAGGACGAAGAGAGGAGTACGATGTTTTGGACAAGAGACGTGGCCGGGACCCTG
AGATGGGGGGAAAGCCGAGAAGGAAGAACCCTCAGGAAGGCCTGTACAATGAA
CTGCAGAAAGATAAGATGGCGGAGGCCTACAGTGAGATTGGGATGAAAGGCGA
GCGCCGGAGGGGCAAGGGGCACGATGGCCTTTACCAGGGTCTCAGTACAGCCAC
CAAGGACACCTACGACGCCCTTCACATGCAGGCCCTGCCCCCTCGC
scFv47.SH.delta CAR amino acid sequence
SEQ ID NO: 11
MDWIWRILFLVGAATGAHSQVQLQQPGAELVRPGASVKLSCKASGYTFSNYLMNW
VKQRPEQDLDWIGRIDPYDGDIDYNQNFKDKAILTVDKSSSTAYMQLSSLTSEDSAV
YYCARGYGTAYGVDYWGQGTSVTVSSAKTTPPKLEEGEFSEARVDIVLTQSPASLA
VSLGQRATISCRASESVDNYGISFMNWFQQKPGQPPKLLIYAASRQGSGVPARFSGS
GSGTDFSLNIHPMEEDDTAMYFCQQSKEVPWTFGGGTKLEIKDLEPKSCDKTHTCPP
CPDPKFWVLVVVGGVLACYSLLVTVAFIIFWVRSKRSRLLH
scFv47.SH.delta CAR DNA sequence
SEQ ID NO: 12
ATGGACTGGATCTGGCGCATCCTGTTTCTCGTGGGAGCCGCCACAGGCGCCCATT
CTCAGGTGCAGCTGCAGCAGCCTGGCGCTGAACTCGTGCGGCCAGGCGCTTCTGT
GAAGCTGAGCTGTAAAGCCAGCGGCTACACCTTCAGCAACTACCTGATGAACTG
GGTCAAGCAGCGGCCCGAGCAGGACCTGGATTGGATCGGCAGAATCGACCCCTA
CGACGGCGACATCGACTACAACCAGAACTTCAAGGACAAGGCCATCCTGACCGT
GGACAAGAGCAGCAGCACCGCCTACATGCAGCTGTCCAGCCTGACCAGCGAGGA
CAGCGCCGTGTACTACTGCGCCAGAGGCTACGGCACAGCCTACGGCGTGGACTA
TTGGGGCCAGGGCACAAGCGTGACCGTGTCCAGCGCCAAGACCACCCCCCCTAA
GCTGGAAGAGGGCGAGTTCTCCGAGGCCCGGGTGGACATTGTGCTGACACAGTC
TCCAGCCAGCCTGGCCGTGTCCCTGGGACAGAGAGCCACCATCAGCTGTAGGGC
CAGCGAGAGCGTGGACAACTACGGCATCAGCTTCATGAATTGGTTCCAGCAGAA
GCCCGGCCAGCCCCCCAAGCTGCTGATCTATGCCGCCAGCAGACAGGGCAGCGG
AGTGCCTGCCAGATTTTCTGGCAGCGGCTCCGGCACCGACTTCAGCCTGAACATC
CACCCTATGGAAGAGGACGACACCGCCATGTACTTTTGCCAGCAGAGCAAAGAG
GTGCCCTGGACCTTTGGCGGAGGCACCAAGCTGGAAATCAAGGATCTCGAGCCC
AAATCTTGTGACAAAACTCACACATGCCCACCGTGCCCGGATCCCAAATTTTGGG
TGCTGGTGGTGGTTGGTGGAGTCCTGGCTTGCTATAGCTTGCTAGTAACAGTGGC
CTTTATTATTTTCTGGGTGAGGAGTAAGAGGAGCAGGCTCCTGCAC
4H5.CD28.z.2A.tCD19 amino acid sequence
SEQ ID NO: 13
MDWIWRILFLVGAATGAHSQVQLLESGGGLVQPGGSLRLSCAASGFTFSSYTMSWV
RQAPGQALEWMGTISSGGTYTYYPDSVKGRFTISRDNAKNSLYLQMNSLRAEDTAV
YYCAREAIFTYWGRGTLVTSSGGGGSGGGGSGGGGSDIQLTQSPSSLSASVGDRVTI
TCKASQDINNYLSWYQQKPGQAPRLLIYRANRLVDGVPDRFSGSGYGTDFTLTINNI
ESEDAAYYFCLKYDVFPYTFGQGTKVEIKDLEPKSCDKTHTCPPCPDPKFWVLVVVG
GVLACYSLLVTVAFIIFWVRSKRSRLLHSDYMNMTPRRPGPTRKHYQPYAPPRDFAA
YRSRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHM
QALPPRASRAEGRGSLLTCGDVEENPGPMPPPRLLFFLLFLTPMEVRPEEPLVVKVEE
GDNAVLQCLKGTSDGPTQQLTWSRESPLKPFLKLSLGLPGLGIHMRPLAIWLFIFNVS
QQMGGFYLCQPGPPSEKAWQPGWTVNVEGSGELFRWNVSDLGGLGCGLKNRSSEG
PSSPSGKLMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPRATAQDA
GKYYCHRGNLTMSFHLEITARPVLWHWLLRTGGWKVSAVTLAYLIFCLCSLVGILH
LQRALVLRRKRKRMTDPTRRF
4H5.CD28.z.2A.tCD19 DNA sequence
SEQ ID NO: 14
ATGGACTGGATCTGGCGGATTCTGTTCCTCGTGGGAGCCGCCACAGGCGCTCACT
CACAGGTGCAGCTGCTGGAATCTGGCGGCGGACTGGTGCAGCCTGGCGGCAGCC
TGAGACTGAGCTGCGCCGCCAGCGGCTTCACCTTCAGCAGCTACACCATGAGCTG
GGTCCGGCAGGCTCCTGGACAGGCCCTGGAATGGATGGGCACCATCAGCAGCGG
CGGCACCTACACCTACTACCCCGACAGCGTGAAGGGCCGGTTCACCATCAGCCG
GGACAACGCCAAGAACAGCCTGTACCTGCAGATGAACAGCCTGAGAGCCGAGG
ACACAGCCGTGTACTACTGCGCCAGAGAGGCCATCTTCACCTACTGGGGCAGAG
GCACCCTGGTCACAAGCAGCGGAGGCGGAGGAAGTGGAGGGGGAGGATCAGGC
GGCGGAGGCAGCGATATCCAGCTGACCCAGAGCCCTAGCAGCCTGAGCGCCAGC
GTGGGCGACAGAGTGACCATCACATGCAAGGCCAGCCAGGACATCAACAACTAC
CTGAGCTGGTATCAGCAGAAGCCCGGCCAGGCCCCCAGACTGCTGATCTACCGG
GCCAACAGACTGGTGGACGGCGTGCCCGATAGATTCAGCGGCAGCGGCTACGGC
ACCGACTTCACCCTGACCATCAACAACATCGAGTCCGAGGACGCCGCCTACTACT
TCTGCCTGAAGTACGACGTGTTCCCCTACACCTTCGGCCAGGGCACCAAGGTGGA
GATCAAGGATCTCGAGCCCAAATCTTGTGACAAAACTCACACATGCCCACCGTG
CCCGGATCCCAAGTTCTGGGTGCTGGTGGTCGTGGGCGGAGTGCTGGCCTGTTAC
AGCCTGCTCGTGACCGTGGCCTTCATCATCTTTTGGGTGCGCAGCAAGCGGAGCC
GGCTGCTGCACAGCGACTACATGAACATGACCCCCAGACGGCCTGGCCCCACCA
GAAAGCACTACCAGCCTTACGCCCCTCCCAGAGACTTCGCCGCCTACCGGTCCAG
AGTGAAGTTCAGCAGAAGCGCCGACGCCCCTGCCTATCAGCAGGGCCAGAACCA
GCTGTACAACGAGCTGAACCTGGGCAGACGGGAAGAGTACGACGTGCTGGACAA
GCGGAGAGGCAGGGACCCTGAGATGGGCGGCAAGCCCAGAAGAAAGAACCCCC
AGGAAGGCCTGTATAACGAACTGCAGAAAGACAAGATGGCCGAGGCCTACAGC
GAGATCGGCATGAAGGGCGAGCGGAGAAGAGGCAAGGGCCACGATGGCCTGTA
CCAGGGACTGAGCACCGCCACCAAGGACACCTACGACGCCCTGCACATGCAGGC
CCTGCCTCCAAGAGCCTCTAGAGCCGAGGGCAGAGGCAGCCTGCTGACATGTGG
CGACGTGGAAGAGAACCCAGGCCCCATGCCTCCCCCCAGACTGCTGTTCTTCCTG
CTGTTCCTGACCCCTATGGAAGTGCGGCCCGAGGAACCCCTGGTCGTGAAAGTG
GAAGAGGGCGACAACGCCGTGCTGCAGTGTCTGAAGGGCACCTCCGATGGCCCT
ACCCAGCAGCTGACCTGGTCCAGAGAGAGCCCCCTGAAGCCCTTCCTGAAGCTG
TCTCTGGGCCTGCCTGGCCTGGGCATCCATATGAGGCCACTGGCCATCTGGCTGT
TCATCTTCAACGTGTCCCAGCAGATGGGAGGCTTCTACCTGTGCCAGCCTGGCCC
ACCTTCTGAGAAGGCTTGGCAGCCTGGCTGGACCGTGAACGTGGAAGGATCTGG
CGAGCTGTTCCGGTGGAACGTGTCCGATCTGGGCGGCCTGGGATGCGGCCTGAA
GAACAGATCTAGCGAGGGCCCCAGCAGCCCCAGCGGCAAACTGATGAGCCCCAA
GCTGTACGTGTGGGCCAAGGACAGACCCGAGATTTGGGAGGGCGAGCCCCCTTG
CCTGCCCCCTAGAGATAGCCTGAACCAGAGCCTGAGCCAGGACCTGACAATGGC
CCCTGGCAGCACACTGTGGCTGAGCTGTGGCGTGCCACCCGACTCTGTGTCTAGA
GGCCCTCTGAGCTGGACCCACGTGCACCCTAAGGGCCCTAAGAGCCTGCTGTCCC
TGGAACTGAAGGACGACAGGCCCGCCAGAGATATGTGGGTCATGGAAACCGGCC
TGCTGCTGCCTAGAGCCACAGCCCAGGATGCCGGCAAGTACTACTGCCACAGAG
GCAACCTGACCATGAGCTTCCACCTGGAAATCACCGCCAGACCCGTGCTGTGGC
ACTGGCTGCTGAGAACCGGCGGATGGAAAGTGTCCGCCGTGACTCTGGCCTACC
TGATCTTCTGCCTGTGCTCCCTCGTGGGCATCCTGCATCTGCAGAGGGCTCTGGT
GCTGCGGCGGAAGCGGAAGAGAATGACCGACCCTACCCGGCGGTTCTAA
leader sequence amino acid sequence
SEQ ID NO: 15
MDWIWRILFLVGAATGAHS
leader sequence DNA sequence 1
SEQ ID NO: 16
ATGGACTGGATCTGGCGCATCCTGTTTCTCGTGGGAGCCGCCACAGGCGCCCATT
CT
scFvFRP5 amino acid sequence
SEQ ID NO: 17
EVQLQQSGPELKKPGETVKISCKASGYPFTNYGMNWVKQAPGQGLKWMGWINTST
GESTFADDFKGRFDFSLETSANTAYLQINNLKSEDMATYFCARWEVYHGYVPYWG
QGTTVTVSSGGGGSGGGGSGGGGSDIQLTQSHKFLSTSVGDRVSITCKASQDVYNAV
AWYQQKPGQSPKLLIYSASSRYTGVPSRFTGSGSGPDFTFTISSVQAEDLAVYFCQQH
FRTPFTFGSGTKLEIKAL
scFvFRP5 DNA sequence
SEQ ID NO: 18
GAGGTACAACTGCAGCAGTCTGGACCTGAACTGAAGAAGCCTGGAGAGACAGTC
AAGATCTCCTGCAAGGCCTCTGGGTATCCTTTCACAAACTATGGAATGAACTGGG
TGAAGCAGGCTCCAGGACAGGGTTTAAAGTGGATGGGCTGGATTAACACCTCCA
CTGGAGAGTCAACATTTGCTGATGACTTCAAGGGACGGTTTGACTTCTCTTTGGA
AACCTCTGCCAACACTGCCTATTTGCAGATCAACAACCTCAAAAGTGAAGACAT
GGCTACATATTTCTGTGCAAGATGGGAGGTTTACCACGGCTACGTTCCTTACTGG
GGCCAAGGGACCACGGTCACCGTTTCCTCTGGCGGTGGCGGTTCTGGTGGCGGTG
GCTCCGGCGGTGGCGGTTCTGACATCCAGCTGACCCAGTCTCACAAATTCCTGTC
CACTTCAGTAGGAGACAGGGTCAGCATCACCTGCAAGGCCAGTCAGGATGTGTA
TAATGCTGTTGCCTGGTATCAACAGAAACCAGGACAATCTCCTAAACTTCTGATT
TACTCGGCATCCTCCCGGTACACTGGAGTCCCTTCTCGCTTCACTGGCAGTGGCT
CTGGGCCGGATTTCACTTTCACCATCAGCAGTGTGCAGGCTGAAGACCTGGCAGT
TTATTTCTGTCAGCAACATTTTCGTACTCCATTCACGTTCGGCTCGGGGACAAAAT
TGGAGATCAAAGCTCTA
short hinge amino acid sequence
SEQ ID NO: 19
DLEPKSCDKTHTCPPCP
short hinge DNA sequence
SEQ ID NO: 20
GATCTCGAGCCCAAATCTTGTGACAAAACTCACACATGCCCACCGTGCCCG
linker domain amino acid sequence
SEQ ID NO: 21
DLEPKSCDKTHTCPPCPDPK
linker domain DNA sequence 1
SEQ ID NO: 22
GATCTCGAGCCCAAATCTTGTGACAAAACTCACACATGCCCACCGTGCCCGGATC
CCAAA
CD3 zeta transmembrane domain amino acid sequence
SEQ ID NO: 23
LCYLLDGILFIYGVILTALFL
CD3 zeta transmembrane domain DNA sequence
SEQ ID NO: 24
CTCTGCTACCTGCTGGATGGAATCCTCTTCATCTATGGTGTCATTCTCACTGCCTT
GTTCCTG
CD3 zeta cytoplasmic domain amino acid sequence
SEQ ID NO: 25
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR
CD3 zeta cytoplasmic domain DNA sequence 1
SEQ ID NO: 26
AGAGTGAAGTTCAGCAGGAGCGCAGACGCCCCCGCGTACCAGCAGGGCCAGAA
CCAGCTCTATAACGAGCTCAATCTAGGACGAAGAGAGGAGTACGATGTTTTGGA
CAAGAGACGTGGCCGGGACCCTGAGATGGGGGGAAAGCCGAGAAGGAAGAACC
CTCAGGAAGGCCTGTACAATGAACTGCAGAAAGATAAGATGGCGGAGGCCTACA
GTGAGATTGGGATGAAAGGCGAGCGCCGGAGGGGCAAGGGGCACGATGGCCTT
TACCAGGGTCTCAGTACAGCCACCAAGGACACCTACGACGCCCTTCACATGCAG
GCCCTGCCCCCTCGC
CD3 zeta transmembrane and cytoplasmic domain amino acid
sequence
SEQ ID NO: 27
LCYLLDGILFIYGVILTALFLRVKFSRSADAPAYQQGQNQLYNELNLGRREEYDVLD
KRRGRDPEMGGKPRRKNPQEGLYNELQKDKMAEAYSEIGMKGERRRGKGHDGLY
QGLSTATKDTYDALHMQALPPR
CD3 zeta transmembrane and cytoplasmic domain DNA
sequence
SEQ ID NO: 28
CTCTGCTACCTGCTGGATGGAATCCTCTTCATCTATGGTGTCATTCTCACTGCCTT
GTTCCTGAGAGTGAAGTTCAGCAGGAGCGCAGACGCCCCCGCGTACCAGCAGGG
CCAGAACCAGCTCTATAACGAGCTCAATCTAGGACGAAGAGAGGAGTACGATGT
TTTGGACAAGAGACGTGGCCGGGACCCTGAGATGGGGGGAAAGCCGAGAAGGA
AGAACCCTCAGGAAGGCCTGTACAATGAACTGCAGAAAGATAAGATGGCGGAG
GCCTACAGTGAGATTGGGATGAAAGGCGAGCGCCGGAGGGGCAAGGGGCACGA
TGGCCTTTACCAGGGTCTCAGTACAGCCACCAAGGACACCTACGACGCCCTTCAC
ATGCAGGCCCTGCCCCCTCGC
scFv47 amino acid sequence
SEQ ID NO: 29
QVQLQQPGAELVRPGASVKLSCKASGYTFSNYLMNWVKQRPEQDLDWIGRIDPYD
GDIDYNQNFKDKAILTVDKSSSTAYMQLSSLTSEDSAVYYCARGYGTAYGVDYWG
QGTSVTVSSAKTTPPKLEEGEFSEARVDIVLTQSPASLAVSLGQRATISCRASESVDN
YGISFMNWFQQKPGQPPKLLIYAASRQGSGVPARFSGSGSGTDFSLNIHPMEEDDTA
MYFCQQSKEVPWTFGGGTKLEIK
scFv47 DNA sequence
SEQ ID NO: 30
CAGGTGCAGCTGCAGCAGCCTGGCGCTGAACTCGTGCGGCCAGGCGCTTCTGTG
AAGCTGAGCTGTAAAGCCAGCGGCTACACCTTCAGCAACTACCTGATGAACTGG
GTCAAGCAGCGGCCCGAGCAGGACCTGGATTGGATCGGCAGAATCGACCCCTAC
GACGGCGACATCGACTACAACCAGAACTTCAAGGACAAGGCCATCCTGACCGTG
GACAAGAGCAGCAGCACCGCCTACATGCAGCTGTCCAGCCTGACCAGCGAGGAC
AGCGCCGTGTACTACTGCGCCAGAGGCTACGGCACAGCCTACGGCGTGGACTAT
TGGGGCCAGGGCACAAGCGTGACCGTGTCCAGCGCCAAGACCACCCCCCCTAAG
CTGGAAGAGGGCGAGTTCTCCGAGGCCCGGGTGGACATTGTGCTGACACAGTCT
CCAGCCAGCCTGGCCGTGTCCCTGGGACAGAGAGCCACCATCAGCTGTAGGGCC
AGCGAGAGCGTGGACAACTACGGCATCAGCTTCATGAATTGGTTCCAGCAGAAG
CCCGGCCAGCCCCCCAAGCTGCTGATCTATGCCGCCAGCAGACAGGGCAGCGGA
GTGCCTGCCAGATTTTCTGGCAGCGGCTCCGGCACCGACTTCAGCCTGAACATCC
ACCCTATGGAAGAGGACGACACCGCCATGTACTTTTGCCAGCAGAGCAAAGAGG
TGCCCTGGACCTTTGGCGGAGGCACCAAGCTGGAAATCAAG
CD28 transmembrane domain amino acid sequence
SEQ ID NO: 31
FWVLVVVGGVLACYSLLVTVAFIIFWV
CD28 transmembrane domain DNA sequence
SEQ ID NO: 32
TTTTGGGTGCTGGTGGTGGTTGGTGGAGTCCTGGCTTGCTATAGCTTGCTAGTAA
CAGTGGCCTTTATTATTTTCTGGGTG
CD28 co-stimulatory domain amino acid sequence
SEQ ID NO: 33
RSKRSRLLHSDYMNMTPRRPGPTRKHYQPYAPPRDFAAYRS
CD28 co-stimulatory domain DNA sequence
SEQ ID NO: 34
AGGAGTAAGAGGAGCAGGCTCCTGCACAGTGACTACATGAACATGACTCCCCGC
CGCCCCGGGCCCACCCGCAAGCATTACCAGCCCTATGCCCCACCACGCGACTTCG
CAGCCTATCGCTCC
CD28 transmembrane domain and co-stimulatory domain
amino acid sequence
SEQ ID NO: 35
FWVLVVVGGVLACYSLLVTVAFIIFWVRSKRSRLLHSDYMNMTPRRPGPTRKHYQP
YAPPRDFAAYRS
CD28 transmembrane domain and co-stimulatory domain DNA
sequence 1
SEQ ID NO: 36
TTTTGGGTGCTGGTGGTGGTTGGTGGAGTCCTGGCTTGCTATAGCTTGCTAGTAA
CAGTGGCCTTTATTATTTTCTGGGTGAGGAGTAAGAGGAGCAGGCTCCTGCACAG
TGACTACATGAACATGACTCCCCGCCGCCCCGGGCCCACCCGCAAGCATTACCA
GCCCTATGCCCCACCACGCGACTTCGCAGCCTATCGCTCC
Leader sequence DNA sequence 2
SEQ ID NO: 37
ATGGACTGGATCTGGCGGATTCTGTTCCTCGTGGGAGCCGCCACAGGCGCTCACT
CA
scFv 4H5 amino acid sequence
SEQ ID NO: 38
QVQLLESGGGLVQPGGSLRLSCAASGFTFSSYTMSWVRQAPGQALEWMGTISSGGT
YTYYPDSVKGRFTISRDNAKNSLYLQMNSLRAEDTAVYYCAREAIFTYWGRGTLVT
SSGGGGSGGGGSGGGGSDIQLTQSPSSLSASVGDRVTITCKASQDINNYLSWYQQKP
GQAPRLLIYRANRLVDGVPDRFSGSGYGTDFTLTINNIESEDAAYYFCLKYDVFPYTF
GQGTKVEIK
scFv 4H5 DNA sequence
SEQ ID NO: 39
CAGGTGCAGCTGCTGGAATCTGGCGGCGGACTGGTGCAGCCTGGCGGCAGCCTG
AGACTGAGCTGCGCCGCCAGCGGCTTCACCTTCAGCAGCTACACCATGAGCTGG
GTCCGGCAGGCTCCTGGACAGGCCCTGGAATGGATGGGCACCATCAGCAGCGGC
GGCACCTACACCTACTACCCCGACAGCGTGAAGGGCCGGTTCACCATCAGCCGG
GACAACGCCAAGAACAGCCTGTACCTGCAGATGAACAGCCTGAGAGCCGAGGAC
ACAGCCGTGTACTACTGCGCCAGAGAGGCCATCTTCACCTACTGGGGCAGAGGC
ACCCTGGTCACAAGCAGCGGAGGCGGAGGAAGTGGAGGGGGAGGATCAGGCGG
CGGAGGCAGCGATATCCAGCTGACCCAGAGCCCTAGCAGCCTGAGCGCCAGCGT
GGGCGACAGAGTGACCATCACATGCAAGGCCAGCCAGGACATCAACAACTACCT
GAGCTGGTATCAGCAGAAGCCCGGCCAGGCCCCCAGACTGCTGATCTACCGGGC
CAACAGACTGGTGGACGGCGTGCCCGATAGATTCAGCGGCAGCGGCTACGGCAC
CGACTTCACCCTGACCATCAACAACATCGAGTCCGAGGACGCCGCCTACTACTTC
TGCCTGAAGTACGACGTGTTCCCCTACACCTTCGGCCAGGGCACCAAGGTGGAG
ATCAAG
IgG1 Short Hinge amino acid sequence
SEQ ID NO: 40
EPKSCDKTHTCPPCP
IgG1 Short Hinge DNA sequence
SEQ ID NO: 41
GAGCCCAAATCTTGTGACAAAACTCACACATGCCCACCGTGCCCG
linker domain DNA sequence 2
SEQ ID NO: 42
GATCTCGAGCCCAAATCTTGTGACAAAACTCACACATGCCCACCGTGCCCGGATC
CCAAG
CD28 transmembrane domain and co-stimulatory domain DNA
sequence 2
SEQ ID NO: 43
TTCTGGGTGCTGGTGGTCGTGGGCGGAGTGCTGGCCTGTTACAGCCTGCTCGTGA
CCGTGGCCTTCATCATCTTTTGGGTGCGCAGCAAGCGGAGCCGGCTGCTGCACAG
CGACTACATGAACATGACCCCCAGACGGCCTGGCCCCACCAGAAAGCACTACCA
GCCTTACGCCCCTCCCAGAGACTTCGCCGCCTACCGGTCC
CD3 zeta cytoplasmic domain DNA sequence 2
SEQ ID NO: 44
AGAGTGAAGTTCAGCAGAAGCGCCGACGCCCCTGCCTATCAGCAGGGCCAGAAC
CAGCTGTACAACGAGCTGAACCTGGGCAGACGGGAAGAGTACGACGTGCTGGAC
AAGCGGAGAGGCAGGGACCCTGAGATGGGCGGCAAGCCCAGAAGAAAGAACCC
CCAGGAAGGCCTGTATAACGAACTGCAGAAAGACAAGATGGCCGAGGCCTACA
GCGAGATCGGCATGAAGGGCGAGCGGAGAAGAGGCAAGGGCCACGATGGCCTG
TACCAGGGACTGAGCACCGCCACCAAGGACACCTACGACGCCCTGCACATGCAG
GCCCTGCCTCCAAGA
T2A amino acid sequence
SEQ ID NO: 45
EGRGSLLTCGDVEENPGP
T2A DNA sequence
SEQ ID NO: 46
GAGGGCAGAGGCAGCCTGCTGACATGTGGCGACGTGGAAGAGAACCCAGGCCC
C
Truncated CD19 amino acid sequence
SEQ ID NO: 47
MPPPRLLFFLLFLTPMEVRPEEPLVVKVEEGDNAVLQCLKGTSDGPTQQLTWSRESP
LKPFLKLSLGLPGLGIHMRPLAIWLFIFNVSQQMGGFYLCQPGPPSEKAWQPGWTVN
VEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSL
ELKDDRPARDMWVMETGLLLPRATAQDAGKYYCHRGNLTMSFHLEITARPVLWH
WLLRTGGWKVSAVTLAYLIFCLCSLVGILHLQRALVLRRKRKRMTDPTRRF
Truncated CD19 DNA sequence
SEQ ID NO: 48
ATGCCTCCCCCCAGACTGCTGTTCTTCCTGCTGTTCCTGACCCCTATGGAAGTGCG
GCCCGAGGAACCCCTGGTCGTGAAAGTGGAAGAGGGCGACAACGCCGTGCTGCA
GTGTCTGAAGGGCACCTCCGATGGCCCTACCCAGCAGCTGACCTGGTCCAGAGA
GAGCCCCCTGAAGCCCTTCCTGAAGCTGTCTCTGGGCCTGCCTGGCCTGGGCATC
CATATGAGGCCACTGGCCATCTGGCTGTTCATCTTCAACGTGTCCCAGCAGATGG
GAGGCTTCTACCTGTGCCAGCCTGGCCCACCTTCTGAGAAGGCTTGGCAGCCTGG
CTGGACCGTGAACGTGGAAGGATCTGGCGAGCTGTTCCGGTGGAACGTGTCCGA
TCTGGGCGGCCTGGGATGCGGCCTGAAGAACAGATCTAGCGAGGGCCCCAGCAG
CCCCAGCGGCAAACTGATGAGCCCCAAGCTGTACGTGTGGGCCAAGGACAGACC
CGAGATTTGGGAGGGCGAGCCCCCTTGCCTGCCCCCTAGAGATAGCCTGAACCA
GAGCCTGAGCCAGGACCTGACAATGGCCCCTGGCAGCACACTGTGGCTGAGCTG
TGGCGTGCCACCCGACTCTGTGTCTAGAGGCCCTCTGAGCTGGACCCACGTGCAC
CCTAAGGGCCCTAAGAGCCTGCTGTCCCTGGAACTGAAGGACGACAGGCCCGCC
AGAGATATGTGGGTCATGGAAACCGGCCTGCTGCTGCCTAGAGCCACAGCCCAG
GATGCCGGCAAGTACTACTGCCACAGAGGCAACCTGACCATGAGCTTCCACCTG
GAAATCACCGCCAGACCCGTGCTGTGGCACTGGCTGCTGAGAACCGGCGGATGG
AAAGTGTCCGCCGTGACTCTGGCCTACCTGATCTTCTGCCTGTGCTCCCTCGTGG
GCATCCTGCATCTGCAGAGGGCTCTGGTGCTGCGGCGGAAGCGGAAGAGAATGA
CCGACCCTACCCGGCGGTTC
CD8a amino acid sequence
SEQ ID NO: 49
CDIYIWAPLAGTCGVLLLSLVITLYCNHRN
CD8a DNA sequence
SEQ ID NO: 50
TGCGACATCTACATCTGGGCCCCTCTGGCCGGCACATGTGGCGTGCTGCTGCTGA
GCCTCGTGATCACCCTGTACTGCAACCACCGGAAC
CD4 amino acid sequence
SEQ ID NO: 51
QPMALIVLGGVAGLLLFIGLGIFFCVRCRHR
CD4 DNA sequence
SEQ ID NO: 52
CAGCCAATGGCCCTGATTGTGCTGGGGGGCGTCGCCGGCCTCCTGCTTTTCATTG
GGCTAGGCATCTTCTTCTGTGTCAGGTGCCGGCACCGA
Thosea asigna virus 2A amino acid sequence
SEQ ID NO: 53
GSGEGRGSLLTCGDVEENPGP
FMDV2A amino acid sequence
SEQ ID NO: 54
GSGSRVTELLYRMKRAETYCPRPLLAIHPTEARHKQKIVAPVKQLLNFDLLKLAGDV
ESNPGP
Sponge 2A amino acid sequence
SEQ ID NO: 55
LLCFLLLLLSGDVELNPGP
Sponge 2A amino acid sequence
SEQ ID NO: 56
HHFMFLLLLLAGDIELNPGP
Acorn Worm 2A amino acid sequence
SEQ ID NO: 57
WFLVLLSFILSGDIEVNPGP
Amphioxus 2A amino acid sequence
SEQ ID NO: 58
KNCAMYMLLLSGDVETNPGP
Amphioxus 2A amino acid sequence
SEQ ID NO: 59
MVISQLMLKLAGDVEENPGP
Porcine Teschovirus-1 2A amino acid sequence
SEQ ID NO: 60
GSGATNFSLLKQAGDVEENPGP
Equine Rhinitis A Virus 2A amino acid sequence
SEQ ID NO: 61
GSGQCTNYALLKLAGDVESNPGP
2A consensus sequence, in which X is any amino acid residue
SEQ ID NO: 62
D-X-E-X-NPGP
DNMT3a sgRNA sequence g2, in which N is A, T, C, or G
SEQ ID NO: 63
CCTGCATGATGCGCGGCCCANGG
mCherry sgRNA guide 17, in which N is A, T, C, or G
SEQ ID NO: 64
CAAGTAGTCGGGGATGTCGGNGG
mCherry sgRNA guide 19, in which N is A, T, C, or G
SEQ ID NO: 65
AGTAGTCGGGGATGTCGGCGNGG
CD28 co-stimulatory domain (partial) amino acid sequence
SEQ ID NO: 66
RSKRSRLLH
CD28 co-stimulatory domain (partial) DNA sequence
SEQ ID NO: 67
AGGAGTAAGAGGAGCAGGCTCCTGCAC
DNMT3a sgRNA sequence g3, in which N is A, T, C, or G
SEQ ID NO: 68
GCATGATGCGCGGCCCAAGGNGG
Forward Primer
SEQ ID NO: 69
CACTCTTTCCCTACACGACGCTCTTCCGATCTTCCCGATGACCCTGTCTTCCCGTG
C
Reverse Primer
SEQ ID NO: 70
GTGACTGGAGTTCAGACGTGTGCTCTTCCGATCTAGGTAGAAGCCATTAGTGAGC
TGGC
Leader
SEQ ID NO: 71
ATGGACTGGATCTGGCGGATCCTGTTTCTTGTGGGAGCCGCCACAGGCGCCCATT
CT
scFv MGA271 vH
SEQ ID NO: 72
EVQLVESGGGLVQPGGSLRLSCAASGFTFSSFGMHWVRQAPGKGLEWVAYISSDSS
AIYYADTVKGRFTISRDNAKNSLYLQMNSLRDEDTAVYYCGRGRENIYYGSRLDYW
GQGTTVTVS
scFv MGA271 vH
SEQ ID NO: 73
GAAGTTCAGCTGGTTGAGTCTGGCGGCGGACTGGTTCAACCAGGCGGATCTCTG
AGACTGAGCTGTGCCGCCAGCGGCTTCACCTTTAGCAGCTTTGGCATGCACTGGG
TCCGACAGGCCCCTGGCAAAGGACTTGAGTGGGTCGCCTACATCAGCAGCGATA
GCAGCGCCATCTACTACGCCGACACCGTGAAGGGCAGATTCACCATCAGCCGGG
ACAACGCCAAGAACAGCCTGTACCTGCAGATGAACTCCCTGCGCGACGAGGATA
CCGCCGTGTACTATTGTGGCAGAGGCAGAGAGAACATCTATTACGGCAGCAGAC
TGGACTACTGGGGCCAGGGAACAACCGTGACAGTCTCT
scFv linker
SEQ ID NO: 74
SGGGGSGGGGSGGGGS
scFv linker
SEQ ID NO: 75
TCTGGTGGCGGAGGAAGCGGAGGCGGAGGTTCAGGCGGCGGAGGATCT
scFv MGA271 vL
SEQ ID NO: 76
DIQLTQSPSFLSASVGDRVTITCKASQNVDTNVAWYQQKPGKAPKALIYSASYRYSG
VPSRFSGSGSGTDFTLTISSLQPEDFATYYCQQYNNYPFTFGQGTKLEIK
scFv MGA271 vL
SEQ ID NO: 77
GATATTCAGCTGACTCAGAGCCCCAGCTTCCTGAGCGCCTCTGTGGGAGACAGA
GTGACCATCACATGCAAGGCCAGCCAGAACGTGGACACCAACGTGGCCTGGTAT
CAGCAGAAGCCTGGCAAGGCTCCCAAGGCTCTGATCTACAGCGCCAGCTACAGA
TACAGCGGCGTGCCCAGCAGATTTTCTGGCAGCGGCTCTGGCACCGACTTCACCC
TGACCATATCTAGCCTGCAGCCTGAGGACTTCGCCACCTACTACTGCCAGCAGTA
CAACAACTACCCCTTCACCTTCGGCCAGGGCACCAAGCTGGAAATCAAG
CD8a Hinge
SEQ ID NO: 78
TTTPAPRPPTPAPTIASQPLSLRPEACRPAAGGAVHTRGLDFACD
CD8a Hinge
SEQ ID NO: 79
ACCACCACACCAGCTCCTCGGCCTCCAACTCCTGCTCCTACAATTGCCAGCCAGC
CTCTGTCTCTGAGGCCCGAAGCTTGTAGACCTGCTGCTGGCGGAGCCGTGCATAC
AAGAGGACTGGATTTCGCCTGCGAC
CD28 Hinge
SEQ ID NO: 80
IEVMYPPPYLDNEKSNGTIIHVKGKHLCPSPLFPGPSKP
CD28 Hinge
SEQ ID NO: 81
ATCGAAGTGATGTACCCGCCTCCTTACCTGGACAACGAGAAGTCCAACGGCACC
ATCATCCACGTGAAGGGAAAGCACCTGTGTCCTTCTCCACTGTTCCCCGGACCTA
GCAAGCCT
CD8a Transmembrane Domain
SEQ ID NO: 82
IYIWAPLAGTCGVLLLSLVITLYC
CD8a Transmembrane Domain
SEQ ID NO: 83
ATCTACATCTGGGCCCCTCTGGCTGGAACATGTGGCGTTCTGCTGCTGAGCCTGG
TCATCACCCTGTACTGC
CD28 Transmembrane Domain
SEQ ID NO: 84
TTCTGGGTGCTCGTTGTTGTTGGCGGCGTGCTGGCCTGTTACAGCCTGCTGGTTAC
CGTGGCCTTCATCATCTTTTGGGTC
CD28 Costimulatory Domain
SEQ ID NO: 85
CGGTCCAAGAGAAGCAGACTGCTGCACAGCGACTACATGAACATGACCCCTAGA
CGGCCCGGACCTACCAGAAAGCACTACCAGCCTTACGCTCCTCCTAGAGATTTCG
CCGCCTACCGGTCC
41BB Costimulatory Domain
SEQ ID NO: 86
KRGRKKLLYIFKQPFMRPVQTTQEEDGCSCRFPEEEEGGCEL
41BB Costimulatory Domain
SEQ ID NO: 87
AAGCGGGGCAGAAAGAAGCTGCTGTACATCTTCAAGCAGCCCTTCATGCGGCCC
GTGCAGACCACACAAGAGGAAGATGGCTGCTCCTGCAGATTCCCCGAGGAAGAA
GAAGGCGGCTGCGAGCTG
CD3z
SEQ ID NO: 88
AGAGTGAAGTTCTCCAGATCCGCCGATGCTCCCGCCTATCAGCAGGGACAGAAC
CAGCTGTACAACGAGCTGAACCTGGGGAGAAGAGAAGAGTACGACGTGCTGGA
CAAGCGGAGAGGCAGGGATCCTGAGATGGGCGGCAAGCCCAGACGGAAGAATC
CTCAAGAGGGCCTGTATAATGAGCTGCAGAAAGACAAGATGGCCGAGGCCTACA
GCGAGATCGGAATGAAGGGCGAGCGCAGAAGAGGCAAGGGACACGATGGACTG
TACCAGGGCCTGAGCACCGCCACCAAGGATACCTATGATGCCCTGCACATGCAG
GCCCTGCCTCCAAGA
Delta
SEQ ID NO: 89
KRGR
Delta
SEQ ID NO: 90
AAGCGGGGCAGA
scFv MGA271
SEQ ID NO: 91
EVQLVESGGGLVQPGGSLRLSCAASGFTFSSFGMHWVRQAPGKGLEWVAYISSDSS
AIYYADTVKGRFTISRDNAKNSLYLQMNSLRDEDTAVYYCGRGRENIYYGSRLDYW
GQGTTVTVSSGGGGSGGGGSGGGGSDIQLTQSPSFLSASVGDRVTITCKASQNVDTN
VAWYQQKPGKAPKALIYSASYRYSGVPSRFSGSGSGTDFTLTISSLQPEDFATYYCQ
QYNNYPFTFGQGTKLEIK
scFv MGA271
SEQ ID NO: 92
GAAGTTCAGCTGGTTGAGTCTGGCGGCGGACTGGTTCAACCAGGCGGATCTCTG
AGACTGAGCTGTGCCGCCAGCGGCTTCACCTTTAGCAGCTTTGGCATGCACTGGG
TCCGACAGGCCCCTGGCAAAGGACTTGAGTGGGTCGCCTACATCAGCAGCGATA
GCAGCGCCATCTACTACGCCGACACCGTGAAGGGCAGATTCACCATCAGCCGGG
ACAACGCCAAGAACAGCCTGTACCTGCAGATGAACTCCCTGCGCGACGAGGATA
CCGCCGTGTACTATTGTGGCAGAGGCAGAGAGAACATCTATTACGGCAGCAGAC
TGGACTACTGGGGCCAGGGAACAACCGTGACAGTCTCTTCTGGTGGCGGAGGAA
GCGGAGGCGGAGGTTCAGGCGGCGGAGGATCTGATATTCAGCTGACTCAGAGCC
CCAGCTTCCTGAGCGCCTCTGTGGGAGACAGAGTGACCATCACATGCAAGGCCA
GCCAGAACGTGGACACCAACGTGGCCTGGTATCAGCAGAAGCCTGGCAAGGCTC
CCAAGGCTCTGATCTACAGCGCCAGCTACAGATACAGCGGCGTGCCCAGCAGAT
TTTCTGGCAGCGGCTCTGGCACCGACTTCACCCTGACCATATCTAGCCTGCAGCC
TGAGGACTTCGCCACCTACTACTGCCAGCAGTACAACAACTACCCCTTCACCTTC
GGCCAGGGCACCAAGCTGGAAATCAAG
extracellular target-binding domain
SEQ ID NO: 93
MDWIWRILFLVGAATGAHSEVQLVESGGGLVQPGGSLRLSCAASGFTFSSFGMHWV
RQAPGKGLEWVAYISSDSSAIYYADTVKGRFTISRDNAKNSLYLQMNSLRDEDTAV
YYCGRGRENIYYGSRLDYWGQGTTVTVSSGGGGSGGGGSGGGGSDIQLTQSPSFLS
ASVGDRVTITCKASQNVDTNVAWYQQKPGKAPKALIYSASYRYSGVPSRFSGSGSG
TDFTLTISSLQPEDFATYYCQQYNNYPFTFGQGTKLEIK
extracellular target-binding domain
SEQ ID NO: 94
ATGGACTGGATCTGGCGGATCCTGTTTCTTGTGGGAGCCGCCACAGGCGCCCATT
CTGAAGTTCAGCTGGTTGAGTCTGGCGGCGGACTGGTTCAACCAGGCGGATCTCT
GAGACTGAGCTGTGCCGCCAGCGGCTTCACCTTTAGCAGCTTTGGCATGCACTGG
GTCCGACAGGCCCCTGGCAAAGGACTTGAGTGGGTCGCCTACATCAGCAGCGAT
AGCAGCGCCATCTACTACGCCGACACCGTGAAGGGCAGATTCACCATCAGCCGG
GACAACGCCAAGAACAGCCTGTACCTGCAGATGAACTCCCTGCGCGACGAGGAT
ACCGCCGTGTACTATTGTGGCAGAGGCAGAGAGAACATCTATTACGGCAGCAGA
CTGGACTACTGGGGCCAGGGAACAACCGTGACAGTCTCTTCTGGTGGCGGAGGA
AGCGGAGGCGGAGGTTCAGGCGGCGGAGGATCTGATATTCAGCTGACTCAGAGC
CCCAGCTTCCTGAGCGCCTCTGTGGGAGACAGAGTGACCATCACATGCAAGGCC
AGCCAGAACGTGGACACCAACGTGGCCTGGTATCAGCAGAAGCCTGGCAAGGCT
CCCAAGGCTCTGATCTACAGCGCCAGCTACAGATACAGCGGCGTGCCCAGCAGA
TTTTCTGGCAGCGGCTCTGGCACCGACTTCACCCTGACCATATCTAGCCTGCAGC
CTGAGGACTTCGCCACCTACTACTGCCAGCAGTACAACAACTACCCCTTCACCTT
CGGCCAGGGCACCAAGCTGGAAATCAAG
CD8a Hinge + Transmembrane Domain
SEQ ID NO: 95
TTTPAPRPPTPAPTIASQPLSLRPEACRPAAGGAVHTRGLDFACDIYIWAPLAGTCGV
LLLSLVITLYC
CD8a Hinge + Transmembrane Domain
SEQ ID NO: 96
ACCACCACACCAGCTCCTCGGCCTCCAACTCCTGCTCCTACAATTGCCAGCCAGC
CTCTGTCTCTGAGGCCCGAAGCTTGTAGACCTGCTGCTGGCGGAGCCGTGCATAC
AAGAGGACTGGATTTCGCCTGCGACATCTACATCTGGGCCCCTCTGGCTGGAACA
TGTGGCGTTCTGCTGCTGAGCCTGGTCATCACCCTGTACTGC
CD28 Hinge + Transmembrane Domain
SEQ ID NO: 97
IEVMYPPPYLDNEKSNGTIIHVKGKHLCPSPLFPGPSKPFWVLVVVGGVLACYSLLVT
VAFIIFWV
CD28 Hinge + Transmembrane Domain
SEQ ID NO: 98
ATCGAAGTGATGTACCCGCCTCCTTACCTGGACAACGAGAAGTCCAACGGCACC
ATCATCCACGTGAAGGGAAAGCACCTGTGTCCTTCTCCACTGTTCCCCGGACCTA
GCAAGCCTTTCTGGGTGCTCGTTGTTGTTGGCGGCGTGCTGGCCTGTTACAGCCT
GCTGGTTACCGTGGCCTTCATCATCTTTTGGGTC
cytoplasmic domain 1
SEQ ID NO: 99
RSKRSRLLHSDYMNMTPRRPGPTRKHYQPYAPPRDFAAYRSRVKFSRSADAPAYQQ
GQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
cytoplasmic domain 1
SEQ ID NO: 100
CGGTCCAAGAGAAGCAGACTGCTGCACAGCGACTACATGAACATGACCCCTAGA
CGGCCCGGACCTACCAGAAAGCACTACCAGCCTTACGCTCCTCCTAGAGATTTCG
CCGCCTACCGGTCCAGAGTGAAGTTCTCCAGATCCGCCGATGCTCCCGCCTATCA
GCAGGGACAGAACCAGCTGTACAACGAGCTGAACCTGGGGAGAAGAGAAGAGT
ACGACGTGCTGGACAAGCGGAGAGGCAGGGATCCTGAGATGGGCGGCAAGCCC
AGACGGAAGAATCCTCAAGAGGGCCTGTATAATGAGCTGCAGAAAGACAAGAT
GGCCGAGGCCTACAGCGAGATCGGAATGAAGGGCGAGCGCAGAAGAGGCAAGG
GACACGATGGACTGTACCAGGGCCTGAGCACCGCCACCAAGGATACCTATGATG
CCCTGCACATGCAGGCCCTGCCTCCAAGA
cytoplasmic domain 2
SEQ ID NO: 101
KRGRKKLLYIFKQPFMRPVQTTQEEDGCSCRFPEEEEGGCELRVKFSRSADAPAYQQ
GQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
cytoplasmic domain 2
SEQ ID NO: 102
AAGCGGGGCAGAAAGAAGCTGCTGTACATCTTCAAGCAGCCCTTCATGCGGCCC
GTGCAGACCACACAAGAGGAAGATGGCTGCTCCTGCAGATTCCCCGAGGAAGAA
GAAGGCGGCTGCGAGCTGAGAGTGAAGTTCTCCAGATCCGCCGACGCTCCTGCC
TATCAGCAGGGACAGAACCAGCTGTACAACGAGCTGAACCTGGGGAGAAGAGA
AGAGTACGACGTGCTGGACAAGCGGAGAGGCAGGGATCCTGAGATGGGCGGCA
AGCCCAGACGGAAGAATCCTCAAGAGGGCCTGTATAATGAGCTGCAGAAAGACA
AGATGGCCGAGGCCTACAGCGAGATCGGAATGAAGGGCGAGCGCAGAAGAGGC
AAGGGACACGATGGACTGTACCAGGGCCTGAGCACCGCCACCAAGGATACCTAT
GATGCCCTGCACATGCAGGCCCTGCCTCCAAGA
cytoplasmic domain 3
SEQ ID NO: 103
RSKRSRLLHSDYMNMTPRRPGPTRKHYQPYAPPRDFAAYRSKRGRKKLLYIFKQPF
MRPVQTTQEEDGCSCRFPEEEEGGCELRVKFSRSADAPAYQQGQNQLYNELNLGRR
EEYDVLDKRRGRDPEMGGKPRRKNPQEGLYNELQKDKMAEAYSEIGMKGERRRGK
GHDGLYQGLSTATKDTYDALHMQALPPR
cytoplasmic domain 3
SEQ ID NO: 104
CGGTCCAAGAGAAGCAGACTGCTGCACAGCGACTACATGAACATGACCCCTAGA
CGGCCCGGACCTACCAGAAAGCACTACCAGCCTTACGCTCCTCCTAGAGATTTCG
CCGCCTACCGGTCCAAGCGGGGCAGAAAGAAGCTGCTGTACATCTTCAAGCAGC
CCTTCATGCGGCCCGTGCAGACCACACAAGAGGAAGATGGCTGCTCCTGCAGAT
TCCCCGAGGAAGAAGAAGGCGGCTGCGAGCTGAGAGTGAAGTTCTCCAGATCCG
CCGATGCTCCCGCCTATCAGCAGGGACAGAACCAGCTGTACAACGAGCTGAACC
TGGGGAGAAGAGAAGAGTACGACGTGCTGGACAAGCGGAGAGGCAGGGATCCT
GAGATGGGCGGCAAGCCCAGACGGAAGAATCCTCAAGAGGGCCTGTATAATGA
GCTGCAGAAAGACAAGATGGCCGAGGCCTACAGCGAGATCGGAATGAAGGGCG
AGCGCAGAAGAGGCAAGGGACACGATGGACTGTACCAGGGCCTGAGCACCGCC
ACCAAGGATACCTATGATGCCCTGCACATGCAGGCCCTGCCTCCAAGA
MGA271.CD8a.CD28.z
SEQ ID NO: 105
MDWIWRILFLVGAATGAHSEVQLVESGGGLVQPGGSLRLSCAASGFTFSSFGMHWV
RQAPGKGLEWVAYISSDSSAIYYADTVKGRFTISRDNAKNSLYLQMNSLRDEDTAV
YYCGRGRENIYYGSRLDYWGQGTTVTVSSGGGGSGGGGSGGGGSDIQLTQSPSFLS
ASVGDRVTITCKASQNVDTNVAWYQQKPGKAPKALIYSASYRYSGVPSRFSGSGSG
TDFTLTISSLQPEDFATYYCQQYNNYPFTFGQGTKLEIKTTTPAPRPPTPAPTIASQPLS
LRPEACRPAAGGAVHTRGLDFACDIYIWAPLAGTCGVLLLSLVITLYCRSKRSRLLHS
DYMNMTPRRPGPTRKHYQPYAPPRDFAAYRSRVKFSRSADAPAYQQGQNQLYNEL
NLGRREEYDVLDKRRGRDPEMGGKPRRKNPQEGLYNELQKDKMAEAYSEIGMKGE
RRRGKGHDGLYQGLSTATKDTYDALHMQALPPR
MGA271.CD8a.CD28.z
SEQ ID NO: 106
ATGGACTGGATCTGGCGGATCCTGTTTCTTGTGGGAGCCGCCACAGGCGCCCATT
CTGAAGTTCAGCTGGTTGAGTCTGGCGGCGGACTGGTTCAACCAGGCGGATCTCT
GAGACTGAGCTGTGCCGCCAGCGGCTTCACCTTTAGCAGCTTTGGCATGCACTGG
GTCCGACAGGCCCCTGGCAAAGGACTTGAGTGGGTCGCCTACATCAGCAGCGAT
AGCAGCGCCATCTACTACGCCGACACCGTGAAGGGCAGATTCACCATCAGCCGG
GACAACGCCAAGAACAGCCTGTACCTGCAGATGAACTCCCTGCGCGACGAGGAT
ACCGCCGTGTACTATTGTGGCAGAGGCAGAGAGAACATCTATTACGGCAGCAGA
CTGGACTACTGGGGCCAGGGAACAACCGTGACAGTCTCTTCTGGTGGCGGAGGA
AGCGGAGGCGGAGGTTCAGGCGGCGGAGGATCTGATATTCAGCTGACTCAGAGC
CCCAGCTTCCTGAGCGCCTCTGTGGGAGACAGAGTGACCATCACATGCAAGGCC
AGCCAGAACGTGGACACCAACGTGGCCTGGTATCAGCAGAAGCCTGGCAAGGCT
CCCAAGGCTCTGATCTACAGCGCCAGCTACAGATACAGCGGCGTGCCCAGCAGA
TTTTCTGGCAGCGGCTCTGGCACCGACTTCACCCTGACCATATCTAGCCTGCAGC
CTGAGGACTTCGCCACCTACTACTGCCAGCAGTACAACAACTACCCCTTCACCTT
CGGCCAGGGCACCAAGCTGGAAATCAAGACCACCACACCAGCTCCTCGGCCTCC
AACTCCTGCTCCTACAATTGCCAGCCAGCCTCTGTCTCTGAGGCCCGAAGCTTGT
AGACCTGCTGCTGGCGGAGCCGTGCATACAAGAGGACTGGATTTCGCCTGCGAC
ATCTACATCTGGGCCCCTCTGGCTGGAACATGTGGCGTTCTGCTGCTGAGCCTGG
TCATCACCCTGTACTGCCGGTCCAAGAGAAGCAGACTGCTGCACAGCGACTACA
TGAACATGACCCCTAGACGGCCCGGACCTACCAGAAAGCACTACCAGCCTTACG
CTCCTCCTAGAGATTTCGCCGCCTACCGGTCCAGAGTGAAGTTCTCCAGATCCGC
CGATGCTCCCGCCTATCAGCAGGGACAGAACCAGCTGTACAACGAGCTGAACCT
GGGGAGAAGAGAAGAGTACGACGTGCTGGACAAGCGGAGAGGCAGGGATCCTG
AGATGGGCGGCAAGCCCAGACGGAAGAATCCTCAAGAGGGCCTGTATAATGAGC
TGCAGAAAGACAAGATGGCCGAGGCCTACAGCGAGATCGGAATGAAGGGCGAG
CGCAGAAGAGGCAAGGGACACGATGGACTGTACCAGGGCCTGAGCACCGCCAC
CAAGGATACCTATGATGCCCTGCACATGCAGGCCCTGCCTCCAAGA
MGA271.CD8a.41BB.z
SEQ ID NO: 107
MDWIWRILFLVGAATGAHSEVQLVESGGGLVQPGGSLRLSCAASGFTFSSFGMHWV
RQAPGKGLEWVAYISSDSSAIYYADTVKGRFTISRDNAKNSLYLQMNSLRDEDTAV
YYCGRGRENIYYGSRLDYWGQGTTVTVSSGGGGSGGGGSGGGGSDIQLTQSPSFLS
ASVGDRVTITCKASQNVDTNVAWYQQKPGKAPKALIYSASYRYSGVPSRFSGSGSG
TDFTLTISSLQPEDFATYYCQQYNNYPFTFGQGTKLEIKTTTPAPRPPTPAPTIASQPLS
LRPEACRPAAGGAVHTRGLDFACDIYIWAPLAGTCGVLLLSLVITLYCKRGRKKLLY
IFKQPFMRPVQTTQEEDGCSCRFPEEEEGGCELRVKFSRSADAPAYQQGQNQLYNEL
NLGRREEYDVLDKRRGRDPEMGGKPRRKNPQEGLYNELQKDKMAEAYSEIGMKGE
RRRGKGHDGLYQGLSTATKDTYDALHMQALPPR
MGA271.CD8a.41BB.z
SEQ ID NO: 108
ATGGACTGGATCTGGCGGATCCTGTTTCTTGTGGGAGCCGCCACAGGCGCCCATT
CTGAAGTTCAGCTGGTTGAGTCTGGCGGCGGACTGGTTCAACCAGGCGGATCTCT
GAGACTGAGCTGTGCCGCCAGCGGCTTCACCTTTAGCAGCTTTGGCATGCACTGG
GTCCGACAGGCCCCTGGCAAAGGACTTGAGTGGGTCGCCTACATCAGCAGCGAT
AGCAGCGCCATCTACTACGCCGACACCGTGAAGGGCAGATTCACCATCAGCCGG
GACAACGCCAAGAACAGCCTGTACCTGCAGATGAACTCCCTGCGCGACGAGGAT
ACCGCCGTGTACTATTGTGGCAGAGGCAGAGAGAACATCTATTACGGCAGCAGA
CTGGACTACTGGGGCCAGGGAACAACCGTGACAGTCTCTTCTGGTGGCGGAGGA
AGCGGAGGCGGAGGTTCAGGCGGCGGAGGATCTGATATTCAGCTGACTCAGAGC
CCCAGCTTCCTGAGCGCCTCTGTGGGAGACAGAGTGACCATCACATGCAAGGCC
AGCCAGAACGTGGACACCAACGTGGCCTGGTATCAGCAGAAGCCTGGCAAGGCT
CCCAAGGCTCTGATCTACAGCGCCAGCTACAGATACAGCGGCGTGCCCAGCAGA
TTTTCTGGCAGCGGCTCTGGCACCGACTTCACCCTGACCATATCTAGCCTGCAGC
CTGAGGACTTCGCCACCTACTACTGCCAGCAGTACAACAACTACCCCTTCACCTT
CGGCCAGGGCACCAAGCTGGAAATCAAGACCACCACACCAGCTCCTCGGCCTCC
AACTCCTGCTCCTACAATTGCCAGCCAGCCTCTGTCTCTGAGGCCCGAAGCTTGT
AGACCTGCTGCTGGCGGAGCCGTGCATACAAGAGGACTGGATTTCGCCTGCGAC
ATCTACATCTGGGCCCCTCTGGCTGGAACATGTGGCGTTCTGCTGCTGAGCCTGG
TCATCACCCTGTACTGCAAGCGGGGCAGAAAGAAGCTGCTGTACATCTTCAAGC
AGCCCTTCATGCGGCCCGTGCAGACCACACAAGAGGAAGATGGCTGCTCCTGCA
GATTCCCCGAGGAAGAAGAAGGCGGCTGCGAGCTGAGAGTGAAGTTCTCCAGAT
CCGCCGACGCTCCTGCCTATCAGCAGGGACAGAACCAGCTGTACAACGAGCTGA
ACCTGGGGAGAAGAGAAGAGTACGACGTGCTGGACAAGCGGAGAGGCAGGGAT
CCTGAGATGGGCGGCAAGCCCAGACGGAAGAATCCTCAAGAGGGCCTGTATAAT
GAGCTGCAGAAAGACAAGATGGCCGAGGCCTACAGCGAGATCGGAATGAAGGG
CGAGCGCAGAAGAGGCAAGGGACACGATGGACTGTACCAGGGCCTGAGCACCG
CCACCAAGGATACCTATGATGCCCTGCACATGCAGGCCCTGCCTCCAAGA
MGA271.CD28.CD28.z
SEQ ID NO: 109
MDWIWRILFLVGAATGAHSEVQLVESGGGLVQPGGSLRLSCAASGFTFSSFGMHWV
RQAPGKGLEWVAYISSDSSAIYYADTVKGRFTISRDNAKNSLYLQMNSLRDEDTAV
YYCGRGRENIYYGSRLDYWGQGTTVTVSSGGGGSGGGGSGGGGSDIQLTQSPSFLS
ASVGDRVTITCKASQNVDTNVAWYQQKPGKAPKALIYSASYRYSGVPSRFSGSGSG
TDFTLTISSLQPEDFATYYCQQYNNYPFTFGQGTKLEIKIEVMYPPPYLDNEKSNGTII
HVKGKHLCPSPLFPGPSKPFWVLVVVGGVLACYSLLVTVAFIIFWVRSKRSRLLHSD
YMNMTPRRPGPTRKHYQPYAPPRDFAAYRSRVKFSRSADAPAYQQGQNQLYNELN
LGRREEYDVLDKRRGRDPEMGGKPRRKNPQEGLYNELQKDKMAEAYSEIGMKGER
RRGKGHDGLYQGLSTATKDTYDALHMQALPPR
MGA271.CD28.CD28.z
SEQ ID NO: 110
ATGGACTGGATCTGGCGGATCCTGTTTCTTGTGGGAGCCGCCACAGGCGCCCATT
CTGAAGTTCAGCTGGTTGAGTCTGGCGGCGGACTGGTTCAACCAGGCGGATCTCT
GAGACTGAGCTGTGCCGCCAGCGGCTTCACCTTTAGCAGCTTTGGCATGCACTGG
GTCCGACAGGCCCCTGGCAAAGGACTTGAGTGGGTCGCCTACATCAGCAGCGAT
AGCAGCGCCATCTACTACGCCGACACCGTGAAGGGCAGATTCACCATCAGCCGG
GACAACGCCAAGAACAGCCTGTACCTGCAGATGAACTCCCTGCGCGACGAGGAT
ACCGCCGTGTACTATTGTGGCAGAGGCAGAGAGAACATCTATTACGGCAGCAGA
CTGGACTACTGGGGCCAGGGAACAACCGTGACAGTCTCTTCTGGTGGCGGAGGA
AGCGGAGGCGGAGGTTCAGGCGGCGGAGGATCTGATATTCAGCTGACTCAGAGC
CCCAGCTTCCTGAGCGCCTCTGTGGGAGACAGAGTGACCATCACATGCAAGGCC
AGCCAGAACGTGGACACCAACGTGGCCTGGTATCAGCAGAAGCCTGGCAAGGCT
CCCAAGGCTCTGATCTACAGCGCCAGCTACAGATACAGCGGCGTGCCCAGCAGA
TTTTCTGGCAGCGGCTCTGGCACCGACTTCACCCTGACCATATCTAGCCTGCAGC
CTGAGGACTTCGCCACCTACTACTGCCAGCAGTACAACAACTACCCCTTCACCTT
CGGCCAGGGCACCAAGCTGGAAATCAAGATCGAAGTGATGTACCCGCCTCCTTA
CCTGGACAACGAGAAGTCCAACGGCACCATCATCCACGTGAAGGGAAAGCACCT
GTGTCCTTCTCCACTGTTCCCCGGACCTAGCAAGCCTTTCTGGGTGCTCGTTGTTG
TTGGCGGCGTGCTGGCCTGTTACAGCCTGCTGGTTACCGTGGCCTTCATCATCTTT
TGGGTCCGAAGCAAGCGGAGCCGGCTGCTGCACAGCGACTACATGAACATGACC
CCTAGACGGCCCGGACCAACCAGAAAGCACTACCAGCCTTACGCTCCTCCTAGA
GATTTCGCCGCCTACCGGTCCAGAGTGAAGTTCTCCAGATCCGCCGATGCTCCCG
CCTATCAGCAGGGACAGAACCAGCTGTACAACGAGCTGAACCTGGGGAGAAGA
GAAGAGTACGACGTGCTGGACAAGCGGAGAGGCAGGGATCCTGAGATGGGCGG
CAAGCCCAGACGGAAGAATCCTCAAGAGGGCCTGTATAATGAGCTGCAGAAAGA
CAAGATGGCCGAGGCCTACAGCGAGATCGGAATGAAGGGCGAGCGCAGAAGAG
GCAAGGGACACGATGGACTGTACCAGGGACTGAGCACCGCCACCAAGGATACCT
ATGACGCCCTGCACATGCAGGCCCTGCCTCCAAGA
MGA271.CD28.41BB.z
SEQ ID NO: 111
MDWIWRILFLVGAATGAHSEVQLVESGGGLVQPGGSLRLSCAASGFTFSSFGMHWV
RQAPGKGLEWVAYISSDSSAIYYADTVKGRFTISRDNAKNSLYLQMNSLRDEDTAV
YYCGRGRENIYYGSRLDYWGQGTTVTVSSGGGGSGGGGSGGGGSDIQLTQSPSFLS
ASVGDRVTITCKASQNVDTNVAWYQQKPGKAPKALIYSASYRYSGVPSRFSGSGSG
TDFTLTISSLQPEDFATYYCQQYNNYPFTFGQGTKLEIKIEVMYPPPYLDNEKSNGTII
HVKGKHLCPSPLFPGPSKPFWVLVVVGGVLACYSLLVTVAFIIFWVKRGRKKLLYIF
KQPFMRPVQTTQEEDGCSCRFPEEEEGGCELRVKFSRSADAPAYQQGQNQLYNELN
LGRREEYDVLDKRRGRDPEMGGKPRRKNPQEGLYNELQKDKMAEAYSEIGMKGER
RRGKGHDGLYQGLSTATKDTYDALHMQALPPR
MGA271.CD28.41BB.z
SEQ ID NO: 112
ATGGACTGGATCTGGCGGATCCTGTTTCTTGTGGGAGCCGCCACAGGCGCCCATT
CTGAAGTTCAGCTGGTTGAGTCTGGCGGCGGACTGGTTCAACCAGGCGGATCTCT
GAGACTGAGCTGTGCCGCCAGCGGCTTCACCTTTAGCAGCTTTGGCATGCACTGG
GTCCGACAGGCCCCTGGCAAAGGACTTGAGTGGGTCGCCTACATCAGCAGCGAT
AGCAGCGCCATCTACTACGCCGACACCGTGAAGGGCAGATTCACCATCAGCCGG
GACAACGCCAAGAACAGCCTGTACCTGCAGATGAACTCCCTGCGCGACGAGGAT
ACCGCCGTGTACTATTGTGGCAGAGGCAGAGAGAACATCTATTACGGCAGCAGA
CTGGACTACTGGGGCCAGGGAACAACCGTGACAGTCTCTTCTGGTGGCGGAGGA
AGCGGAGGCGGAGGTTCAGGCGGCGGAGGATCTGATATTCAGCTGACTCAGAGC
CCCAGCTTCCTGAGCGCCTCTGTGGGAGACAGAGTGACCATCACATGCAAGGCC
AGCCAGAACGTGGACACCAACGTGGCCTGGTATCAGCAGAAGCCTGGCAAGGCT
CCCAAGGCTCTGATCTACAGCGCCAGCTACAGATACAGCGGCGTGCCCAGCAGA
TTTTCTGGCAGCGGCTCTGGCACCGACTTCACCCTGACCATATCTAGCCTGCAGC
CTGAGGACTTCGCCACCTACTACTGCCAGCAGTACAACAACTACCCCTTCACCTT
CGGCCAGGGCACCAAGCTGGAAATCAAGATCGAAGTGATGTACCCGCCTCCTTA
CCTGGACAACGAGAAGTCCAACGGCACCATCATCCACGTGAAGGGAAAGCACCT
GTGTCCTTCTCCACTGTTCCCCGGACCTAGCAAGCCTTTCTGGGTGCTCGTTGTTG
TTGGCGGCGTGCTGGCCTGTTACAGCCTGCTGGTTACCGTGGCCTTCATCATCTTT
TGGGTCAAGCGGGGCAGAAAGAAGCTGCTGTACATCTTCAAGCAGCCCTTCATG
CGGCCCGTGCAGACCACACAAGAGGAAGATGGCTGCTCCTGCAGATTCCCCGAG
GAAGAAGAAGGCGGCTGCGAGCTGAGAGTGAAGTTCTCCAGATCCGCCGACGCT
CCTGCCTATCAGCAGGGACAGAACCAGCTGTACAACGAGCTGAACCTGGGGAGA
AGAGAAGAGTACGACGTGCTGGACAAGCGGAGAGGCAGGGATCCTGAGATGGG
CGGCAAGCCCAGACGGAAGAATCCTCAAGAGGGCCTGTATAATGAGCTGCAGAA
AGACAAGATGGCCGAGGCCTACAGCGAGATCGGAATGAAGGGCGAGCGCAGAA
GAGGCAAGGGACACGATGGACTGTACCAGGGACTGAGCACCGCCACCAAGGAT
ACCTATGACGCCCTGCACATGCAGGCCCTGCCTCCAAGA
MGA271.CD8a.Delta
SEQ ID NO: 113
MDWIWRILFLVGAATGAHSEVQLVESGGGLVQPGGSLRLSCAASGFTFSSFGMHWV
RQAPGKGLEWVAYISSDSSAIYYADTVKGRFTISRDNAKNSLYLQMNSLRDEDTAV
YYCGRGRENIYYGSRLDYWGQGTTVTVSSGGGGSGGGGSGGGGSDIQLTQSPSFLS
ASVGDRVTITCKASQNVDTNVAWYQQKPGKAPKALIYSASYRYSGVPSRFSGSGSG
TDFTLTISSLQPEDFATYYCQQYNNYPFTFGQGTKLEIKTTTPAPRPPTPAPTIASQPLS
LRPEACRPAAGGAVHTRGLDFACDIYIWAPLAGTCGVLLLSLVITLYCKRGR
MGA271.CD8a.Delta
SEQ ID NO: 114
ATGGACTGGATCTGGCGGATCCTGTTTCTTGTGGGAGCCGCCACAGGCGCCCATT
CTGAAGTTCAGCTGGTTGAGTCTGGCGGCGGACTGGTTCAACCAGGCGGATCTCT
GAGACTGAGCTGTGCCGCCAGCGGCTTCACCTTTAGCAGCTTTGGCATGCACTGG
GTCCGACAGGCCCCTGGCAAAGGACTTGAGTGGGTCGCCTACATCAGCAGCGAT
AGCAGCGCCATCTACTACGCCGACACCGTGAAGGGCAGATTCACCATCAGCCGG
GACAACGCCAAGAACAGCCTGTACCTGCAGATGAACTCCCTGCGCGACGAGGAT
ACCGCCGTGTACTATTGTGGCAGAGGCAGAGAGAACATCTATTACGGCAGCAGA
CTGGACTACTGGGGCCAGGGAACAACCGTGACAGTCTCTTCTGGTGGCGGAGGA
AGCGGAGGCGGAGGTTCAGGCGGCGGAGGATCTGATATTCAGCTGACTCAGAGC
CCCAGCTTCCTGAGCGCCTCTGTGGGAGACAGAGTGACCATCACATGCAAGGCC
AGCCAGAACGTGGACACCAACGTGGCCTGGTATCAGCAGAAGCCTGGCAAGGCT
CCCAAGGCTCTGATCTACAGCGCCAGCTACAGATACAGCGGCGTGCCCAGCAGA
TTTTCTGGCAGCGGCTCTGGCACCGACTTCACCCTGACCATATCTAGCCTGCAGC
CTGAGGACTTCGCCACCTACTACTGCCAGCAGTACAACAACTACCCCTTCACCTT
CGGCCAGGGCACCAAGCTGGAAATCAAGACCACCACACCAGCTCCTCGGCCTCC
AACTCCTGCTCCTACAATTGCCAGCCAGCCTCTGTCTCTGAGGCCCGAAGCTTGT
AGACCTGCTGCTGGCGGAGCCGTGCATACAAGAGGACTGGATTTCGCCTGCGAC
ATCTACATCTGGGCCCCTCTGGCTGGAACATGTGGCGTTCTGCTGCTGAGCCTGG
TCATCACCCTGTACTGCAAGCGGGGCAGA
MGA271.CD8a.CD28.41BB.z
SEQ ID NO: 115
MDWIWRILFLVGAATGAHSEVQLVESGGGLVQPGGSLRLSCAASGFTFSSFGMHWV
RQAPGKGLEWVAYISSDSSAIYYADTVKGRFTISRDNAKNSLYLQMNSLRDEDTAV
YYCGRGRENIYYGSRLDYWGQGTTVTVSSGGGGSGGGGSGGGGSDIQLTQSPSFLS
ASVGDRVTITCKASQNVDTNVAWYQQKPGKAPKALIYSASYRYSGVPSRFSGSGSG
TDFTLTISSLQPEDFATYYCQQYNNYPFTFGQGTKLEIKTTTPAPRPPTPAPTIASQPLS
LRPEACRPAAGGAVHTRGLDFACDIYIWAPLAGTCGVLLLSLVITLYCRSKRSRLLHS
DYMNMTPRRPGPTRKHYQPYAPPRDFAAYRSKRGRKKLLYIFKQPFMRPVQTTQEE
DGCCRFPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGLYQGLS
TATKDTYDALHMQALPPR
MGA271.CD8a.CD28.41BB.z
SEQ ID NO: 116
ATGGACTGGATCTGGCGGATCCTGTTTCTTGTGGGAGCCGCCACAGGCGCCCATT
CTGAAGTTCAGCTGGTTGAGTCTGGCGGCGGACTGGTTCAACCAGGCGGATCTCT
GAGACTGAGCTGTGCCGCCAGCGGCTTCACCTTTAGCAGCTTTGGCATGCACTGG
GTCCGACAGGCCCCTGGCAAAGGACTTGAGTGGGTCGCCTACATCAGCAGCGAT
AGCAGCGCCATCTACTACGCCGACACCGTGAAGGGCAGATTCACCATCAGCCGG
GACAACGCCAAGAACAGCCTGTACCTGCAGATGAACTCCCTGCGCGACGAGGAT
ACCGCCGTGTACTATTGTGGCAGAGGCAGAGAGAACATCTATTACGGCAGCAGA
CTGGACTACTGGGGCCAGGGAACAACCGTGACAGTCTCTTCTGGTGGCGGAGGA
AGCGGAGGCGGAGGTTCAGGCGGCGGAGGATCTGATATTCAGCTGACTCAGAGC
CCCAGCTTCCTGAGCGCCTCTGTGGGAGACAGAGTGACCATCACATGCAAGGCC
AGCCAGAACGTGGACACCAACGTGGCCTGGTATCAGCAGAAGCCTGGCAAGGCT
CCCAAGGCTCTGATCTACAGCGCCAGCTACAGATACAGCGGCGTGCCCAGCAGA
TTTTCTGGCAGCGGCTCTGGCACCGACTTCACCCTGACCATATCTAGCCTGCAGC
CTGAGGACTTCGCCACCTACTACTGCCAGCAGTACAACAACTACCCCTTCACCTT
CGGCCAGGGCACCAAGCTGGAAATCAAGACCACCACACCAGCTCCTCGGCCTCC
AACTCCTGCTCCTACAATTGCCAGCCAGCCTCTGTCTCTGAGGCCCGAAGCTTGT
AGACCTGCTGCTGGCGGAGCCGTGCATACAAGAGGACTGGATTTCGCCTGCGAC
ATCTACATCTGGGCCCCTCTGGCTGGAACATGTGGCGTTCTGCTGCTGAGCCTGG
TCATCACCCTGTACTGCCGGTCCAAGAGAAGCAGACTGCTGCACAGCGACTACA
TGAACATGACCCCTAGACGGCCCGGACCTACCAGAAAGCACTACCAGCCTTACG
CTCCTCCTAGAGATTTCGCCGCCTACCGGTCCAAGCGGGGCAGAAAGAAGCTGC
TGTACATCTTCAAGCAGCCCTTCATGCGGCCCGTGCAGACCACACAAGAGGAAG
ATGGCTGCTCCTGCAGATTCCCCGAGGAAGAAGAAGGCGGCTGCGAGCTGAGAG
TGAAGTTCTCCAGATCCGCCGATGCTCCCGCCTATCAGCAGGGACAGAACCAGCT
GTACAACGAGCTGAACCTGGGGAGAAGAGAAGAGTACGACGTGCTGGACAAGC
GGAGAGGCAGGGATCCTGAGATGGGCGGCAAGCCCAGACGGAAGAATCCTCAA
GAGGGCCTGTATAATGAGCTGCAGAAAGACAAGATGGCCGAGGCCTACAGCGA
GATCGGAATGAAGGGCGAGCGCAGAAGAGGCAAGGGACACGATGGACTGTACC
AGGGCCTGAGCACCGCCACCAAGGATACCTATGATGCCCTGCACATGCAGGCCC
TGCCTCCAAGA
P2A
SEQ ID NO: 117
ATNFSLLKQAGDVEENPGP
P2A
SEQ ID NO: 118
GCCACCAATTTCAGCCTGCTGAAACAGGCCGGCGACGTGGAAGAGAATCCTGGA
CCT
linker
GSG
linker
GGCAGCGGC
linker
SEQ ID NO: 121
GGGSGGGS
linker
SEQ ID NO: 122
GGGSGGGSGGGS
linker
SEQ ID NO: 123
GGGSGGGSGGGSGGGS
linker
SEQ ID NO: 124
GGGSGGGSGGGSGGGSGGGS
linker
SEQ ID NO: 125
GGGGSGGGGS
linker
SEQ ID NO: 126
GGGGSGGGGSGGGGSGGGGS
linker
SEQ ID NO: 127
GGGGSGGGGSGGGGSGGGGSGGGGS
scFv 8H9 vH
SEQ ID NO: 128
QVKLQQSGAELVKPGASVKLSCKASGYTFTNYDINWVRQRPEQGLEWIGWIFPGDG
STQYNEKFKGKATLTTDTSSSTAYMQLSRLTSEDSAVYFCARQTTATWFAYWGQGT
TVTVSSD
scFv 8H9 vH
SEQ ID NO: 129
CAAGTGAAACTGCAGCAGAGCGGAGCCGAACTCGTGAAGCCAGGCGCCAGCGT
GAAGCTGTCTTGCAAGGCCTCCGGCTATACCTTTACCAACTACGACATCAACTGG
GTGCGCCAGAGGCCCGAGCAGGGACTGGAATGGATTGGATGGATCTTCCCCGGC
GACGGCAGCACCCAGTACAATGAGAAGTTTAAGGGGAAGGCTACACTGACAACC
GATACCAGCAGCTCCACAGCTTATATGCAGCTGTCCCGGCTGACCTCCGAGGACT
CCGCTGTGTACTTCTGTGCCAGACAGACCACCGCCACTTGGTTTGCCTATTGGGG
ACAGGGAACCACTGTGACCGTGTCCTCTGAT
scFv linker 2
SEQ ID NO: 130
GGGGSGGGGSGGGGS
scFv linker 2
SEQ ID NO: 131
GGCGGAGGCAGCGGGGGAGGGGGCTCAGGGGGCGGAGGCTCT
scFv 8H9 vL
SEQ ID NO: 132
DIELTQSPTTLSVTPGDRVSLSCRASQSISDYLHWYQQKSHESPRLLIKYASQSISGIPS
RFSGSGSGSDFTLSINSVEPEDVGVYYCQNGHSFPLTFGAGTKLELKQAA
scFv 8H9 vL
SEQ ID NO: 133
GATATTGAGCTGACACAGTCCCCCACCACCCTGAGCGTGACCCCTGGCGATAGA
GTGTCCCTGAGCTGTAGGGCCAGCCAGAGCATCAGCGACTACCTGCATTGGTATC
AGCAGAAAAGCCACGAGAGCCCTCGGCTGCTGATCAAATACGCCAGCCAGTCCA
TCTCCGGCATCCCCAGCAGATTCAGCGGCTCTGGAAGCGGCAGCGACTTCACCCT
GTCCATCAACAGCGTGGAACCTGAGGATGTGGGCGTGTACTATTGCCAGAACGG
CCACAGCTTCCCACTGACCTTCGGCGCTGGAACAAAACTGGAACTGAAACAGGC
CGCC
scFv 8H9
SEQ ID NO: 134
QVKLQQSGAELVKPGASVKLSCKASGYTFTNYDINWVRQRPEQGLEWIGWIFPGDG
STQYNEKFKGKATLTTDTSSSTAYMQLSRLTSEDSAVYFCARQTTATWFAYWGQGT
TVTVSSDGGGGSGGGGSGGGGSDIELTQSPTTLSVTPGDRVSLSCRASQSISDYLHW
YQQKSHESPRLLIKYASQSISGIPSRFSGSGSGSDFTLSINSVEPEDVGVYYCQNGHSFP
LTFGAGTKLELKQAA
scFv 8H9
SEQ ID NO: 135
CAAGTGAAACTGCAGCAGAGCGGAGCCGAACTCGTGAAGCCAGGCGCCAGCGT
GAAGCTGTCTTGCAAGGCCTCCGGCTATACCTTTACCAACTACGACATCAACTGG
GTGCGCCAGAGGCCCGAGCAGGGACTGGAATGGATTGGATGGATCTTCCCCGGC
GACGGCAGCACCCAGTACAATGAGAAGTTTAAGGGGAAGGCTACACTGACAACC
GATACCAGCAGCTCCACAGCTTATATGCAGCTGTCCCGGCTGACCTCCGAGGACT
CCGCTGTGTACTTCTGTGCCAGACAGACCACCGCCACTTGGTTTGCCTATTGGGG
ACAGGGAACCACTGTGACCGTGTCCTCTGATGGCGGAGGCAGCGGGGGAGGGGG
CTCAGGGGGCGGAGGCTCTGATATTGAGCTGACACAGTCCCCCACCACCCTGAG
CGTGACCCCTGGCGATAGAGTGTCCCTGAGCTGTAGGGCCAGCCAGAGCATCAG
CGACTACCTGCATTGGTATCAGCAGAAAAGCCACGAGAGCCCTCGGCTGCTGAT
CAAATACGCCAGCCAGTCCATCTCCGGCATCCCCAGCAGATTCAGCGGCTCTGGA
AGCGGCAGCGACTTCACCCTGTCCATCAACAGCGTGGAACCTGAGGATGTGGGC
GTGTACTATTGCCAGAACGGCCACAGCTTCCCACTGACCTTCGGCGCTGGAACAA
AACTGGAACTGAAACAGGCCGCC
scFv mAb 376.96 vH
SEQ ID NO: 136
EVQLVESGGGLVKPGGSLKLSCEASRFTFSSYAMSWVRQTPEKRLEWVAAISGGGR
YTYYPDSMKGRFTISRDNAKNFLYLQMSSLRSEDTAMYYCARHYDGYLDYWGQGT
TLTVSS
scFv mAb 376.96 vH
SEQ ID NO: 137
GAAGTGCAGCTGGTGGAGTCTGGGGGAGGCTTAGTGAAGCCTGGAGGGTCCCTG
AAACTCTCCTGTGAAGCCTCTAGATTCACTTTCAGTAGCTATGCCATGTCTTGGGT
TCGCCAGACTCCGGAGAAGAGGCTGGAGTGGGTCGCAGCCATTAGTGGAGGTGG
TAGGTACACCTACTATCCAGACAGTATGAAGGGTCGATTCACCATCTCCAGAGAC
AATGCCAAGAATTTCCTGTACCTGCAAATGAGCAGTCTGAGGTCTGAGGACACG
GCCATGTATTACTGTGCAAGACACTATGATGGTTATCTTGACTACTGGGGCCAAG
GCACCACTCTCACAGTCTCCTCA
scFv mAb 376.96 vL
SEQ ID NO: 138
DIVMTQSHKFMSTSIGARVSITCKASQDVRTAVAWYQQKPGQSPKLLIYSASYRYTG
VPDRFTGSGSGTDFTFTISSVQAEDLAVYYCQQHYGTPPWTFGGGTKLEIK
scFv mAb 376.96 vL
SEQ ID NO: 139
GACATTGTGATGACCCAGTCTCACAAATTCATGTCCACATCAATTGGAGCCAGGG
TCAGCATCACCTGCAAGGCCAGTCAGGATGTGAGAACTGCTGTAGCCTGGTATC
AACAGAAACCAGGCCAGTCTCCTAAACTACTAATTTACTCGGCATCCTACCGGTA
CACTGGAGTCCCTGATCGCTTCACTGGCAGTGGATCTGGGACGGATTTCACTTTC
ACCATCAGCAGTGTGCAGGCTGAAGACCTGGCAGTTTATTACTGTCAGCAACATT
ATGGTACTCCTCCGTGGACGTTCGGTGGAGGCACCAAGCTGGAAATCAAA
scFv mAb 376.96
SEQ ID NO: 140
DIVMTQSHKFMSTSIGARVSITCKASQDVRTAVAWYQQKPGQSPKLLIYSASYRYTG
VPDRFTGSGSGTDFTFTISSVQAEDLAVYYCQQHYGTPPWTFGGGTKLEIKGGGGSG
GGGSGGGGSEVQLVESGGGLVKPGGSLKLSCEASRFTFSSYAMSWVRQTPEKRLEW
VAAISGGGRYTYYPDSMKGRFTISRDNAKNFLYLQMSSLRSEDTAMYYCARHYDGY
LDYWGQGTTLTVSS
scFv mAb 376.96
SEQ ID NO: 141
GACATTGTGATGACCCAGTCTCACAAATTCATGTCCACATCAATTGGAGCCAGGG
TCAGCATCACCTGCAAGGCCAGTCAGGATGTGAGAACTGCTGTAGCCTGGTATC
AACAGAAACCAGGCCAGTCTCCTAAACTACTAATTTACTCGGCATCCTACCGGTA
CACTGGAGTCCCTGATCGCTTCACTGGCAGTGGATCTGGGACGGATTTCACTTTC
ACCATCAGCAGTGTGCAGGCTGAAGACCTGGCAGTTTATTACTGTCAGCAACATT
ATGGTACTCCTCCGTGGACGTTCGGTGGAGGCACCAAGCTGGAAATCAAAGGCG
GCGGAGGATCTGGCGGAGGCGGAAGTGGCGGAGGGGGCTCTGAAGTGCAGCTG
GTGGAGTCTGGGGGAGGCTTAGTGAAGCCTGGAGGGTCCCTGAAACTCTCCTGT
GAAGCCTCTAGATTCACTTTCAGTAGCTATGCCATGTCTTGGGTTCGCCAGACTC
CGGAGAAGAGGCTGGAGTGGGTCGCAGCCATTAGTGGAGGTGGTAGGTACACCT
ACTATCCAGACAGTATGAAGGGTCGATTCACCATCTCCAGAGACAATGCCAAGA
ATTTCCTGTACCTGCAAATGAGCAGTCTGAGGTCTGAGGACACGGCCATGTATTA
CTGTGCAAGACACTATGATGGTTATCTTGACTACTGGGGCCAAGGCACCACTCTC
ACAGTCTCCTCA
Mouse ASXL1 guide RNA, Exon 12 of 12
SEQ ID NO: 142
CCACTTACCAGATATGCCCC
Mouse ASXL1 guide RNA, Exon 12 of 12
SEQ ID NO: 143
CCACUUACCAGAUAUGCCCC
IgG2 short hinge sequence
SEQ ID NO: 144
ERKCCVECPPCP
IgG3 short hinge sequence
SEQ ID NO: 145
ELKTPLGDTTHTCPRCP(EPKSCDTPPPCPRCP)3
IgG4 short hinge sequence
SEQ ID NO: 146
ESKYGPPCPSCP
scFv (292) amino acid sequence
SEQ ID NO: 147
QVQLQQPGAELVRPGASVKLSCKASGYTFTSYWMNWVKQRPDQGLEWIGRIDPYD
SETHYNQKFKDKAILTVDKSSSTAYMQLSSLTSEDSAVYYCARGNWDDYWGQGTT
LTVSSGGGGSGGGGSGGGGSDVQITQSPSYLAASPGETITINCRASKSISKDLAWYQE
KPGKTNKLLIYSGSTLQSGIPSRFSGSGSGTDFTLTISSLEPEDFAMYYCQQHNKYPYT
FGGGTKLEIKS
scFv (292) DNA sequence
SEQ ID NO: 148
CAGGTGCAGCTGCAGCAGCCTGGCGCTGAACTCGTGCGGCCAGGCGCTTCTGTG
AAGCTGAGCTGTAAAGCCAGCGGCTACACCTTCACCAGCTACTGGATGAACTGG
GTCAAGCAGCGGCCCGACCAGGGCCTGGAATGGATCGGAAGAATCGACCCCTAC
GACAGCGAGACACACTACAACCAGAAGTTCAAGGACAAGGCCATCCTGACCGTG
GACAAGAGCAGCAGCACCGCCTACATGCAGCTGTCCAGCCTGACCAGCGAGGAC
AGCGCCGTGTACTACTGCGCCAGAGGCAACTGGGACGACTACTGGGGCCAGGGC
ACAACCCTGACAGTGTCTAGCGGAGGCGGAGGATCAGGCGGCGGAGGAAGTGG
GGGAGGCGGATCTGATGTGCAGATTACCCAGTCCCCCAGCTACCTGGCCGCCTCT
CCTGGCGAGACAATCACCATCAACTGCCGGGCCAGCAAGAGCATCTCCAAGGAC
CTGGCCTGGTATCAGGAAAAGCCCGGCAAGACCAACAAGCTGCTGATCTACAGC
GGCTCCACCCTGCAGTCCGGCATCCCCAGCAGATTTTCCGGCAGCGGCTCTGGCA
CCGACTTCACCCTGACCATCAGCTCCCTGGAACCCGAGGACTTTGCCATGTACTA
TTGCCAGCAGCACAACAAGTACCCTTACACCTTCGGCGGAGGCACCAAGCTGGA
AATCAAGAGC
scFv (716) amino acid sequence
SEQ ID NO: 149
QIQLVQSGPELKKPGETVKISCKASGYIFTNYGMNWVKQAPGKSFKWMGWINTYTG
ESTYSADFKGRFAFSLETSASTAYLHINDLKNEDTATYFCARSGGYDPMDYWGQGT
SVTVSSGGGGSGGGGSGGGGSDIVLTQSPASLAVSLGQRATISCRASESVDNYGNTF
MHWYQQKPGQPPKLLIYRASNLESGIPARFSGSGSRTDFTLTINPVEADDVATYYCQ
QSNEDPPTFGAGTKLELK
scFv (716) DNA sequence
SEQ ID NO: 150
CAGATTCAGCTGGTGCAGTCTGGCCCCGAGCTGAAGAAACCCGGCGAGACAGTG
AAGATCAGCTGCAAGGCCAGCGGCTACATCTTCACCAACTACGGCATGAACTGG
GTCAAGCAGGCCCCTGGCAAGAGCTTCAAGTGGATGGGCTGGATCAACACCTAC
ACCGGCGAGAGCACCTACAGCGCCGACTTCAAGGGCAGATTCGCCTTCAGCCTG
GAAACCAGCGCCAGCACCGCCTACCTGCACATCAACGACCTGAAGAACGAGGAC
ACCGCCACCTACTTTTGCGCCAGAAGCGGCGGCTACGACCCTATGGATTATTGGG
GCCAGGGCACCAGCGTGACCGTGTCTAGCGGAGGCGGAGGAAGTGGCGGCGGA
GGATCTGGGGGAGGCGGATCTGATATCGTGCTGACCCAGAGCCCTGCCAGCCTG
GCTGTGTCTCTGGGACAGAGAGCCACCATCAGCTGTCGGGCCAGCGAGAGCGTG
GACAATTACGGCAACACCTTCATGCACTGGTATCAGCAGAAGCCCGGCCAGCCC
CCCAAGCTGCTGATCTACAGAGCCAGCAACCTGGAAAGCGGCATCCCCGCCAGA
TTTTCCGGCAGCGGCAGCAGAACCGACTTCACCCTGACCATCAACCCCGTGGAA
GCCGACGACGTGGCCACCTATTACTGCCAGCAGAGCAACGAGGACCCCCCTACC
TTTGGAGCCGGCACCAAGCTGGAACTGAAG
292.CD8HTM.41BBz CAR amino acid sequence
SEQ ID NO: 151
MALPVTALLLPLALLLHAARPQVQLQQPGAELVRPGASVKLSCKASGYTFTSYWMN
WVKQRPDQGLEWIGRIDPYDSETHYNQKFKDKAILTVDKSSSTAYMQLSSLTSEDSA
VYYCARGNWDDYWGQGTTLTVSSGGGGSGGGGSGGGGSDVQITQSPSYLAASPGE
TITINCRASKSISKDLAWYQEKPGKTNKLLIYSGSTLQSGIPSRFSGSGSGTDFTLTISSL
EPEDFAMYYCQQHNKYPYTFGGGTKLEIKSTTTPAPRPPTPAPTIASQPLSLRPEACRP
AAGGAVHTRGLDFACDIYIWAPLAGTCGVLLLSLVITLYCKRGRKKLLYIFKQPFMR
PVQTTQEEDGCSCRFPEEEEGGCELRVKFSRSADAPAYQQGQNQLYNELNLGRREE
YDVLDKRRGRDPEMGGKPRRKNPQEGLYNELQKDKMAEAYSEIGMKGERRRGKG
HDGLYQGLSTATKDTYDALHMQALPPR
292.CD8HTM.41BBz CAR DNA sequence
SEQ ID NO: 152
ATGGCCTTACCAGTGACCGCCTTGCTCCTGCCGCTGGCCTTGCTGCTCCACGCCG
CCAGGCCGCAGGTGCAGCTGCAGCAGCCTGGCGCTGAACTCGTGCGGCCAGGCG
CTTCTGTGAAGCTGAGCTGTAAAGCCAGCGGCTACACCTTCACCAGCTACTGGAT
GAACTGGGTCAAGCAGCGGCCCGACCAGGGCCTGGAATGGATCGGAAGAATCG
ACCCCTACGACAGCGAGACACACTACAACCAGAAGTTCAAGGACAAGGCCATCC
TGACCGTGGACAAGAGCAGCAGCACCGCCTACATGCAGCTGTCCAGCCTGACCA
GCGAGGACAGCGCCGTGTACTACTGCGCCAGAGGCAACTGGGACGACTACTGGG
GCCAGGGCACAACCCTGACAGTGTCTAGCGGAGGCGGAGGATCAGGCGGCGGA
GGAAGTGGGGGAGGCGGATCTGATGTGCAGATTACCCAGTCCCCCAGCTACCTG
GCCGCCTCTCCTGGCGAGACAATCACCATCAACTGCCGGGCCAGCAAGAGCATC
TCCAAGGACCTGGCCTGGTATCAGGAAAAGCCCGGCAAGACCAACAAGCTGCTG
ATCTACAGCGGCTCCACCCTGCAGTCCGGCATCCCCAGCAGATTTTCCGGCAGCG
GCTCTGGCACCGACTTCACCCTGACCATCAGCTCCCTGGAACCCGAGGACTTTGC
CATGTACTATTGCCAGCAGCACAACAAGTACCCTTACACCTTCGGCGGAGGCACC
AAGCTGGAAATCAAGAGCACCACGACGCCAGCGCCGCGACCACCAACgCCGGCG
CCCACCATCGCGTCGCAGCCCCTGTCCCTGCGCCCAGAGGCGTGCCGGCCAGCG
GCGGGGGGCGCAGTGCACACGAGGGGGCTGGACTTCGCCTGTGATATCTACATC
TGGGCGCCCTTGGCCGGGACTTGTGGGGTCCTTCTCCTGTCACTGGTTATCACCCT
TTACTGCAAACGGGGCAGAAAGAAACTCCTGTATATATTCAAACAACCATTTATG
AGACCAGTACAAACTACTCAAGAGGAAGATGGCTGTAGCTGCCGATTTCCAGAA
GAAGAAGAAGGAGGATGTGAACTGAGAGTGAAGTTCAGCAGGAGCGCAGACGC
CCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGG
GGGAAAGCCGAGAAGGAAGAACCCTCAGGAAGGCCTGTACAATGAACTGCAGA
AAGATAAGATGGCGGAGGCCTACAGTGAGATTGGGATGAAAGGCGAGCGCCGG
AGGGGCAAGGGGCACGATGGCCTTTACCAGGGTCTCAGTACAGCCACCAAGGAC
ACCTACGACGCCCTTCACATGCAGGCCCTGCCCCCTCGCTAA
292.CD8HTM.CD28z CAR amino acid sequence
SEQ ID NO: 153
MALPVTALLLPLALLLHAARPQVQLQQPGAELVRPGASVKLSCKASGYTFTSYWMN
WVKQRPDQGLEWIGRIDPYDSETHYNQKFKDKAILTVDKSSSTAYMQLSSLTSEDSA
VYYCARGNWDDYWGQGTTLTVSSGGGGSGGGGSGGGGSDVQITQSPSYLAASPGE
TITINCRASKSISKDLAWYQEKPGKTNKLLIYSGSTLQSGIPSRFSGSGSGTDFTLTISSL
EPEDFAMYYCQQHNKYPYTFGGGTKLEIKSTTTPAPRPPTPAPTIASQPLSLRPEACRP
AAGGAVHTRGLDFACDIYIWAPLAGTCGVLLLSLVITLYCRSKRSRLLHSDYMNMTP
RRPGPTRKHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERRRGKGH
DGLYQGLSTATKDTYDALHMQALPPR
292.CD8HTM.CD28z CAR DNA sequence
SEQ ID NO: 154
ATGGCCTTACCAGTGACCGCCTTGCTCCTGCCGCTGGCCTTGCTGCTCCACGCCG
CCAGGCCGCAGGTGCAGCTGCAGCAGCCTGGCGCTGAACTCGTGCGGCCAGGCG
CTTCTGTGAAGCTGAGCTGTAAAGCCAGCGGCTACACCTTCACCAGCTACTGGAT
GAACTGGGTCAAGCAGCGGCCCGACCAGGGCCTGGAATGGATCGGAAGAATCG
ACCCCTACGACAGCGAGACACACTACAACCAGAAGTTCAAGGACAAGGCCATCC
TGACCGTGGACAAGAGCAGCAGCACCGCCTACATGCAGCTGTCCAGCCTGACCA
GCGAGGACAGCGCCGTGTACTACTGCGCCAGAGGCAACTGGGACGACTACTGGG
GCCAGGGCACAACCCTGACAGTGTCTAGCGGAGGCGGAGGATCAGGCGGCGGA
GGAAGTGGGGGAGGCGGATCTGATGTGCAGATTACCCAGTCCCCCAGCTACCTG
GCCGCCTCTCCTGGCGAGACAATCACCATCAACTGCCGGGCCAGCAAGAGCATC
TCCAAGGACCTGGCCTGGTATCAGGAAAAGCCCGGCAAGACCAACAAGCTGCTG
ATCTACAGCGGCTCCACCCTGCAGTCCGGCATCCCCAGCAGATTTTCCGGCAGCG
GCTCTGGCACCGACTTCACCCTGACCATCAGCTCCCTGGAACCCGAGGACTTTGC
CATGTACTATTGCCAGCAGCACAACAAGTACCCTTACACCTTCGGCGGAGGCACC
AAGCTGGAAATCAAGAGCACCACGACGCCAGCGCCGCGACCACCAACgCCGGCG
CCCACCATCGCGTCGCAGCCCCTGTCCCTGCGCCCAGAGGCGTGCCGGCCAGCG
GCGGGGGGCGCAGTGCACACGAGGGGGCTGGACTTCGCCTGTGATATCTACATC
TGGGCGCCCTTGGCCGGGACTTGTGGGGTCCTTCTCCTGTCACTGGTTATCACCCT
TTACTGCCGAAGCAAGCGGAGCCGGCTGCTGCACAGCGACTACATGAACATGAC
CCCTAGACGGCCCGGACCAACCAGAAAGCACTACCAGCCTTACGCTCCTCCTAG
AGATTTCGCCGCCTACCGGTCCAGAGTGAAGTTCAGCAGGAGCGCAGACGCCCC
CGCGTACCAGCAGGGCCAGAACCAGCTCTATAACGAGCTCAATCTAGGACGAAG
AGAGGAGTACGATGTTTTGGACAAGAGACGTGGCCGGGACCCTGAGATGGGGGG
AAAGCCGAGAAGGAAGAACCCTCAGGAAGGCCTGTACAATGAACTGCAGAAAG
ATAAGATGGCGGAGGCCTACAGTGAGATTGGGATGAAAGGCGAGCGCCGGAGG
GGCAAGGGGCACGATGGCCTTTACCAGGGTCTCAGTACAGCCACCAAGGACACC
TACGACGCCCTTCACATGCAGGCCCTGCCCCCTCGCTAA
292.CD28HTM.CD28.41BBz CAR amino acid sequence
SEQ ID NO: 155
MALPVTALLLPLALLLHAARPQVQLQQPGAELVRPGASVKLSCKASGYTFTSYWMN
WVKQRPDQGLEWIGRIDPYDSETHYNQKFKDKAILTVDKSSSTAYMQLSSLTSEDSA
VYYCARGNWDDYWGQGTTLTVSSGGGGSGGGGSGGGGSDVQITQSPSYLAASPGE
TITINCRASKSISKDLAWYQEKPGKTNKLLIYSGSTLQSGIPSRFSGSGSGTDFTLTISSL
EPEDFAMYYCQQHNKYPYTFGGGTKLEIKSIEVMYPPPYLDNEKSNGTIIHVKGKHL
CPSPLFPGPSKPFWVLVVVGGVLACYSLLVTVAFIIFWVRSKRSRLLHSDYMNMTPR
RPGPTRKHYQPYAPPRDFAAYRSKRGRKKLLYIFKQPFMRPVQTTQEEDGCSCRFPE
EEEGGCELRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTATKDTYD
ALHMQALPPR
292.CD28HTM.CD28.41BBz CAR DNA sequence
SEQ ID NO: 156
ATGGCCTTACCAGTGACCGCCTTGCTCCTGCCGCTGGCCTTGCTGCTCCACGCCG
CCAGGCCGCAGGTGCAGCTGCAGCAGCCTGGCGCTGAACTCGTGCGGCCAGGCG
CTTCTGTGAAGCTGAGCTGTAAAGCCAGCGGCTACACCTTCACCAGCTACTGGAT
GAACTGGGTCAAGCAGCGGCCCGACCAGGGCCTGGAATGGATCGGAAGAATCG
ACCCCTACGACAGCGAGACACACTACAACCAGAAGTTCAAGGACAAGGCCATCC
TGACCGTGGACAAGAGCAGCAGCACCGCCTACATGCAGCTGTCCAGCCTGACCA
GCGAGGACAGCGCCGTGTACTACTGCGCCAGAGGCAACTGGGACGACTACTGGG
GCCAGGGCACAACCCTGACAGTGTCTAGCGGAGGCGGAGGATCAGGCGGCGGA
GGAAGTGGGGGAGGCGGATCTGATGTGCAGATTACCCAGTCCCCCAGCTACCTG
GCCGCCTCTCCTGGCGAGACAATCACCATCAACTGCCGGGCCAGCAAGAGCATC
TCCAAGGACCTGGCCTGGTATCAGGAAAAGCCCGGCAAGACCAACAAGCTGCTG
ATCTACAGCGGCTCCACCCTGCAGTCCGGCATCCCCAGCAGATTTTCCGGCAGCG
GCTCTGGCACCGACTTCACCCTGACCATCAGCTCCCTGGAACCCGAGGACTTTGC
CATGTACTATTGCCAGCAGCACAACAAGTACCCTTACACCTTCGGCGGAGGCACC
AAGCTGGAAATCAAGAGCATCGAAGTGATGTACCCGCCTCCTTACCTGGACAAC
GAGAAGTCCAACGGCACCATCATCCACGTGAAGGGAAAGCACCTGTGTCCTTCT
CCACTGTTCCCCGGACCTAGCAAGCCTTTCTGGGTGCTCGTTGTTGTTGGCGGCG
TGCTGGCCTGTTACAGCCTGCTGGTTACCGTGGCCTTCATCATCTTTTGGGTCCGA
AGCAAGCGGAGCCGGCTGCTGCACAGCGACTACATGAACATGACCCCTAGACGG
CCCGGACCAACCAGAAAGCACTACCAGCCTTACGCTCCTCCTAGAGATTTCGCCG
CCTACCGGTCCAAACGGGGCAGAAAGAAACTCCTGTATATATTCAAACAACCAT
TTATGAGACCAGTACAAACTACTCAAGAGGAAGATGGCTGTAGCTGCCGATTTC
CAGAAGAAGAAGAAGGAGGATGTGAACTGAGAGTGAAGTTCAGCAGGAGCGCA
GACGCCCCCGCGTACCAGCAGGGCCAGAACCAGCTCTATAACGAGCTCAATCTA
GGACGAAGAGAGGAGTACGATGTTTTGGACAAGAGACGTGGCCGGGACCCTGA
GATGGGGGGAAAGCCGAGAAGGAAGAACCCTCAGGAAGGCCTGTACAATGAAC
TGCAGAAAGATAAGATGGCGGAGGCCTACAGTGAGATTGGGATGAAAGGCGAG
CGCCGGAGGGGCAAGGGGCACGATGGCCTTTACCAGGGTCTCAGTACAGCCACC
AAGGACACCTACGACGCCCTTCACATGCAGGCCCTGCCCCCTCGCTAA
292.CD28HTM.CD28.z CAR amino acid sequence
SEQ ID NO: 157
MALPVTALLLPLALLLHAARPQVQLQQPGAELVRPGASVKLSCKASGYTFTSYWMN
WVKQRPDQGLEWIGRIDPYDSETHYNQKFKDKAILTVDKSSSTAYMQLSSLTSEDSA
VYYCARGNWDDYWGQGTTLTVSSGGGGSGGGGSGGGGSDVQITQSPSYLAASPGE
TITINCRASKSISKDLAWYQEKPGKTNKLLIYSGSTLQSGIPSRFSGSGSGTDFTLTISSL
EPEDFAMYYCQQHNKYPYTFGGGTKLEIKSIEVMYPPPYLDNEKSNGTIIHVKGKHL
CPSPLFPGPSKPFWVLVVVGGVLACYSLLVTVAFIIFWVRSKRSRLLHSDYMNMTPR
RPGPTRKHYQPYAPPRDFAAYRSRVKFSRSADAPAYQQGQNQLYNELNLGRREEYD
VLDKRRGRDPEMGGKPRRKNPQEGLYNELQKDKMAEAYSEIGMKGERRRGKGHD
GLYQGLSTATKDTYDALHMQALPPR
292.CD28HTM.CD28.z CAR DNA sequence
SEQ ID NO: 158
ATGGCCTTACCAGTGACCGCCTTGCTCCTGCCGCTGGCCTTGCTGCTCCACGCCG
CCAGGCCGCAGGTGCAGCTGCAGCAGCCTGGCGCTGAACTCGTGCGGCCAGGCG
CTTCTGTGAAGCTGAGCTGTAAAGCCAGCGGCTACACCTTCACCAGCTACTGGAT
GAACTGGGTCAAGCAGCGGCCCGACCAGGGCCTGGAATGGATCGGAAGAATCG
ACCCCTACGACAGCGAGACACACTACAACCAGAAGTTCAAGGACAAGGCCATCC
TGACCGTGGACAAGAGCAGCAGCACCGCCTACATGCAGCTGTCCAGCCTGACCA
GCGAGGACAGCGCCGTGTACTACTGCGCCAGAGGCAACTGGGACGACTACTGGG
GCCAGGGCACAACCCTGACAGTGTCTAGCGGAGGCGGAGGATCAGGCGGCGGA
GGAAGTGGGGGAGGCGGATCTGATGTGCAGATTACCCAGTCCCCCAGCTACCTG
GCCGCCTCTCCTGGCGAGACAATCACCATCAACTGCCGGGCCAGCAAGAGCATC
TCCAAGGACCTGGCCTGGTATCAGGAAAAGCCCGGCAAGACCAACAAGCTGCTG
ATCTACAGCGGCTCCACCCTGCAGTCCGGCATCCCCAGCAGATTTTCCGGCAGCG
GCTCTGGCACCGACTTCACCCTGACCATCAGCTCCCTGGAACCCGAGGACTTTGC
CATGTACTATTGCCAGCAGCACAACAAGTACCCTTACACCTTCGGCGGAGGCACC
AAGCTGGAAATCAAGAGCATCGAAGTGATGTACCCGCCTCCTTACCTGGACAAC
GAGAAGTCCAACGGCACCATCATCCACGTGAAGGGAAAGCACCTGTGTCCTTCT
CCACTGTTCCCCGGACCTAGCAAGCCTTTCTGGGTGCTCGTTGTTGTTGGCGGCG
TGCTGGCCTGTTACAGCCTGCTGGTTACCGTGGCCTTCATCATCTTTTGGGTCCGA
AGCAAGCGGAGCCGGCTGCTGCACAGCGACTACATGAACATGACCCCTAGACGG
CCCGGACCAACCAGAAAGCACTACCAGCCTTACGCTCCTCCTAGAGATTTCGCCG
CCTACCGGTCCAGAGTGAAGTTCAGCAGGAGCGCAGACGCCCCCGCGTACCAGC
AGGGCCAGAACCAGCTCTATAACGAGCTCAATCTAGGACGAAGAGAGGAGTACG
ATGTTTTGGACAAGAGACGTGGCCGGGACCCTGAGATGGGGGGAAAGCCGAGA
AGGAAGAACCCTCAGGAAGGCCTGTACAATGAACTGCAGAAAGATAAGATGGC
GGAGGCCTACAGTGAGATTGGGATGAAAGGCGAGCGCCGGAGGGGCAAGGGGC
ACGATGGCCTTTACCAGGGTCTCAGTACAGCCACCAAGGACACCTACGACGCCC
TTCACATGCAGGCCCTGCCCCCTCGCTAA
716.CD8aHTM.CD28.z CAR amino acid sequence
SEQ ID NO: 159
MALPVTALLLPLALLLHAARPQIQLVQSGPELKKPGETVKISCKASGYIFTNYGMNW
VKQAPGKSFKWMGWINTYTGESTYSADFKGRFAFSLETSASTAYLHINDLKNEDTA
TYFCARSGGYDPMDYWGQGTSVTVSSGGGGGGGGSGGGGSDIVLTQSPASLAVSL
GQRATISCRASESVDNYGNTFMHWYQQKPGQPPKLLIYRASNLESGIPARFSGSGSRT
DFTLTINPVEADDVATYYCQQSNEDPPTFGAGTKLELKTTTPAPRPPTPAPTIASQPLS
LRPEACRPAAGGAVHTRGLDFACDIYIWAPLAGTCGVLLLSLVITLYCRSKRSRLLHS
DYMNMTPRRPGPTRKHYQPYAPPRDFAAYRSRVKFSRSADAPAYQQGQNQLYNEL
NLGRREEYDVLDKRRGRDPEMGGKPRRKNPQEGLYNELQKDKMAEAYSEIGMKGE
RRRGKGHDGLYQGLSTATKDTYDALHMQALPPR
716.CD8aHTM.CD28.z CAR DNA sequence
SEQ ID NO: 160
ATGGCCTTACCAGTGACCGCCTTGCTCCTGCCGCTGGCCTTGCTGCTCCACGCCG
CCAGGCCGCAGATTCAGCTGGTGCAGTCTGGCCCCGAGCTGAAGAAACCCGGCG
AGACAGTGAAGATCAGCTGCAAGGCCAGCGGCTACATCTTCACCAACTACGGCA
TGAACTGGGTCAAGCAGGCCCCTGGCAAGAGCTTCAAGTGGATGGGCTGGATCA
ACACCTACACCGGCGAGAGCACCTACAGCGCCGACTTCAAGGGCAGATTCGCCT
TCAGCCTGGAAACCAGCGCCAGCACCGCCTACCTGCACATCAACGACCTGAAGA
ACGAGGACACCGCCACCTACTTTTGCGCCAGAAGCGGCGGCTACGACCCTATGG
ATTATTGGGGCCAGGGCACCAGCGTGACCGTGTCTAGCGGAGGCGGAGGAAGTG
GCGGCGGAGGATCTGGGGGAGGCGGATCTGATATCGTGCTGACCCAGAGCCCTG
CCAGCCTGGCTGTGTCTCTGGGACAGAGAGCCACCATCAGCTGTCGGGCCAGCG
AGAGCGTGGACAATTACGGCAACACCTTCATGCACTGGTATCAGCAGAAGCCCG
GCCAGCCCCCCAAGCTGCTGATCTACAGAGCCAGCAACCTGGAAAGCGGCATCC
CCGCCAGATTTTCCGGCAGCGGCAGCAGAACCGACTTCACCCTGACCATCAACCC
CGTGGAAGCCGACGACGTGGCCACCTATTACTGCCAGCAGAGCAACGAGGACCC
CCCTACCTTTGGAGCCGGCACCAAGCTGGAACTGAAGACCACGACGCCAGCGCC
GCGACCACCAACgCCGGCGCCCACCATCGCGTCGCAGCCCCTGTCCCTGCGCCCA
GAGGCGTGCCGGCCAGCGGCGGGGGGCGCAGTGCACACGAGGGGGCTGGACTT
CGCCTGTGATATCTACATCTGGGCGCCCTTGGCCGGGACTTGTGGGGTCCTTCTC
CTGTCACTGGTTATCACCCTTTACTGCCGGTCCAAGAGAAGCAGACTGCTGCACA
GCGACTACATGAACATGACCCCTAGACGGCCCGGACCTACCAGAAAGCACTACC
AGCCTTACGCTCCTCCTAGAGATTTCGCCGCCTACCGGTCCAGAGTGAAGTTCAG
CAGGAGCGCAGACGCCCCCGCGTACCAGCAGGGCCAGAACCAGCTCTATAACGA
GCTCAATCTAGGACGAAGAGAGGAGTACGATGTTTTGGACAAGAGACGTGGCCG
GGACCCTGAGATGGGGGGAAAGCCGAGAAGGAAGAACCCTCAGGAAGGCCTGT
ACAATGAACTGCAGAAAGATAAGATGGCGGAGGCCTACAGTGAGATTGGGATG
AAAGGCGAGCGCCGGAGGGGCAAGGGGCACGATGGCCTTTACCAGGGTCTCAGT
ACAGCCACCAAGGACACCTACGACGCCCTTCACATGCAGGCCCTGCCCCCTCGCT
AA
Mouse ASXL1 guide RNA, Exon 12 of 12
SEQ ID NO: 161
CCATTGGGAGATCTATTAGG
Mouse ASXL1 guide RNA, Exon 12 of 12
SEQ ID NO: 162
CCAUUGGGAGAUCUAUUAGG
Mouse ASXL1 guide RNA, Exon 10 of 12
SEQ ID NO: 163
GATGCAAGTCAGGCTAAGAC
Mouse ASXL1 guide RNA, Exon 10 of 12
SEQ ID NO: 164
GAUGCAAGUCAGGCUAAGAC

1. A modified immune effector cell, wherein an Additional Sex Combs Like Transcriptional Regulator 1 (ASXL1) gene or gene product is modified in the cell so that the expression and/or function of ASXL1 in the cell is reduced or eliminated.

2. The modified immune effector cell of claim 1, wherein the level of functional ASXL1 protein in the cell is reduced by 50% or more.

3. The modified immune effector cell of claim 1, wherein the ASXL1 gene is deleted so that no detectable functional ASXL1 protein is produced.

4. The modified immune effector cell of any one of claims 1-3, wherein the immune effector cell is a T cell.

5. The modified immune effector cell of claim 4, wherein the T cell is selected from a CD8+ T cell, a CD4+ T cell, a cytotoxic T cell, an αβ T cell, a natural killer T (NKT) cell, a γδ T cell, a memory T cell, a T-helper cell, and a regulatory T cell (Treg).

6. The modified immune effector cell of any one of claims 1-3, wherein the immune effector cell is a stem cell that is capable of differentiating into an immune cell.

7. The modified immune effector cell of claim 6, wherein the stem cell is an induced pluripotent stem cell (iPSC).

8. The modified immune effector cell of any one of claims 1-3, wherein the immune effector cell is a natural killer (NK) cell.

9. The modified immune effector cell of any one of claims 1-8, wherein the cell further comprises at least one surface molecule capable of binding specifically to an antigen.

10. The modified immune effector cell of claim 9, wherein the antigen is selected from a tumor antigen, a viral antigen, a bacterial antigen, a fungal antigen, a parasite antigen, a prion antigen, and an antigen associated with an inflammation or an autoimmune disease.

11. The modified immune effector cell of claim 10, wherein the tumor antigen is human epidermal growth factor receptor 2 (HER2), IL13Rα2, erythropoietin-producing human hepatocellular receptor A2 (EphA2), B7 homolog 3 protein (B7-H3), Cluster of Differentiation (CD) 19 (CD19), CD22, or CD123.

12. The modified immune effector cell of any one of claims 1-11, wherein the cell further comprises a chimeric antigen receptor (CAR), an antigen specific T-cell receptor, or a bispecific antibody.

13. The modified immune effector cell of claim 12, wherein the cell further comprises a CAR.

14. The modified immune effector cell of claim 13, wherein the CAR comprises (i) an extracellular antigen-binding domain, (ii) a transmembrane domain, and (iii) a cytoplasmic domain.

15. The modified immune effector cell of claim 14, wherein the extracellular antigen-binding domain comprises an antibody or an antibody fragment.

16. The modified immune effector cell of claim 15, wherein the extracellular antigen binding domain comprises an scFv capable of binding to HER2, IL13Rα2, EphA2, B7-H3, CD19, CD22, or CD123.

17. The modified immune effector cell of any one of claims 14-16, wherein the extracellular antigen-binding domain further comprises a leader sequence.

18. The modified immune effector cell of any one of claims 14-17, wherein the transmembrane domain is derived from CD3ζ, CD28, CD4, or CD8α.

19. The modified immune effector cell of any one of claims 14-18, wherein the CAR further comprises a linker domain between the extracellular antigen-binding domain and the transmembrane domain.

20. The modified immune effector cell of claim 19, wherein the linker domain comprises a hinge region.

21. The modified immune effector cell of any one of claims 14-20, wherein the CAR cytoplasmic domain comprises one or more lymphocyte activation domains.

22. The modified immune effector cell of claim 21, wherein the lymphocyte activation domain is derived from DAP10, DAP12, Fc epsilon receptor I γ chain (FCER1G), CD3δ, CD3ε, CD3γ, CD3ζ, CD27, CD28, CD40, CD134, CD137, CD226, CD79A, ICOS, or MyD88.

23. The modified immune effector cell of any one of claims 14-22, wherein the CAR cytoplasmic domain comprises one or more co-stimulatory domains.

24. The modified immune effector cell of any one of claims 1-23, wherein a DNA (cytosine-5)-methyltransferase 3A (DNMT3A) gene or gene product is modified in the cell so that the expression and/or function of DNMT3A in the cell is reduced or eliminated.

25. The modified immune effector cell of any one of claims 1-24, wherein a TET2 (Tet Methylcytosine Dioxygenase 2) gene or gene product is modified in the cell so that the expression and/or function of TET2 in the cell is reduced or eliminated.

26. The modified immune effector cell of any one of claims 1-25, wherein the immune effector cell has been activated and/or expanded ex vivo.

27. The modified immune effector cell of any one of claims 1-26, wherein the immune effector cell is an allogeneic cell.

28. The modified immune effector cell of any one of claims 1-26, wherein the immune effector cell is an autologous cell.

29. The modified immune effector cell of any one of claims 1-26, wherein the immune effector cell is isolated from a subject having a disease.

30. The modified immune effector cell of claim 29, wherein the disease is a cancer, an infectious disease, an inflammatory disorder, or an autoimmune disease.

31. The modified immune effector cell of claim 30, wherein the cancer is a cancer expressing HER2, IL13Rα2, EphA2, B7-H3, CD19, CD22, or CD123.

32. The modified immune effector cell of claim 31, wherein the cancer is a HER2-positive breast cancer.

33. The modified immune effector cell of claim 31, wherein the cancer is an IL13Rα2-positive glioblastoma.

34. The modified immune effector cell of any one of claims 1-31, wherein the immune effector cell is derived from a blood, marrow, tissue, or a tumor sample.

35. A pharmaceutical composition comprising the modified immune effector cell of any one of claims 1-34 and a pharmaceutically acceptable carrier and/or excipient.

36. A method for generating the modified immune effector cell of any one of claims 1-34, said method comprising modifying an ASXL1 gene or gene product in the cell so that the expression and/or function of ASXL1 in the cell is reduced or eliminated.

37. A method of preserving developmental potential of an immune effector cell, said method comprising modifying an ASXL1 gene or gene product in the cell so that the expression and/or function of ASXL1 in the cell is reduced or eliminated.

38. The method of claim 36 or claim 37, wherein the immune effector cell is a T cell.

39. The method of claim 38, wherein the T cell is selected from a CD8+ T cell, a CD4+ T cell, a cytotoxic T cell, an αβ T cell, a natural killer T (NKT) cell, a γδ T cell, a memory T cell, a T-helper cell, and a regulatory T cell (Treg).

40. The method of any one of claims 36-39, wherein the method further comprises modifying the immune effector cell to express a chimeric antigen receptor (CAR) that is capable of binding to an antigen.

41. The method of any one of claims 36-40, wherein the ASXL1 gene in the immune effector cell is modified as a result of an activity of a site-specific nuclease.

42. The method of claim 41, wherein the site-specific nuclease is an RNA-guided endonuclease.

43. The method of claim 42, wherein the RNA-guided endonuclease is a Cas9 protein, Cpf1 (Cas12a) protein, C2c1 protein, C2c3 protein, or C2c2 protein.

44. The method of claim 43, wherein the RNA-guided endonuclease is a Cas9 protein.

45. The method of claim 44, wherein the Cas9 protein is programmed with a guide RNA (gRNA) that comprises a nucleotide sequence of CCACUUACCAGAUAUGCCCC (SEQ ID NO: 143), CCAUUGGGAGAUCUAUUAGG (SEQ ID NO: 162), or GAUGCAAGUCAGGCUAAGAC (SEQ ID NO: 164).

46. The method of claim 41, wherein the site-specific nuclease is a zinc finger nuclease, a TALEN nuclease, or mega-TALEN nuclease.

47. The method of claim 36, wherein the ASXL1 gene product in the immune effector cell is modified as a result of an activity of an RNA interference (RNAi) molecule or an antisense oligonucleotide.

48. The method of claim 47, wherein the RNAi molecule is a small interfering RNA (siRNA) or a small hairpin RNA (shRNA).

49. The method of any one of claims 41-48, wherein the site-specific nuclease or the RNAi molecule or the antisense oligonucleotide is introduced into the immune effector cell via a viral vector, a non-viral vector or a physical means.

50. The method of any one of claims 40-49, wherein the CAR is expressed from a transgene introduced into the immune effector cell.

51. The method of claim 50, wherein the CAR-expressing transgene is introduced into the immune effector cell using a viral vector, a non-viral vector or a physical means.

52. The method of claim 49 or claim 51, wherein the viral vector is a retroviral vector, an adenoviral vector, an adeno-associated viral (AAV) vector, a herpes viral vector, or a baculoviral vector.

53. The method of claim 52, wherein the retroviral vector is a lentiviral vector.

54. The method of any claim 49 or claim 51, wherein the non-viral vector is a transposon.

55. The method of claim 54, wherein the transposon is a sleeping beauty transposon or PiggyBac transposon.

56. The method of claim 49 or claim 51, wherein the physical means is electroporation, microinjection, magnetofection, ultrasound, a ballistic or hydrodynamic method, or a combination thereof.

57. The method of any one of claims 36-56, wherein the modified immune effector cell is activated and/or expanded ex vivo.

58. A method of treating a disease in a subject in need thereof comprising administering to the subject an effective amount of the modified immune effector cell of any one of claims 1-34 or the pharmaceutical composition of claim 35.

59. The method of claim 58, wherein the modified immune effector cell is an autologous cell.

60. The method of claim 58, wherein the modified immune effector cell is an allogeneic cell.

61. The method of any one of claims 58-60, wherein the disease is a cancer, an infectious disease, an inflammatory disorder, or an autoimmune disease.

62. The method of claim 61, wherein the cancer is a solid tumor.

63. The method of claim 61, wherein the cancer is a hematologic cancer.

64. The method of claim 62, wherein the cancer is a cancer expressing HER2, IL13Rα2, EphA2, B7-H3, CD19, CD22, or CD123.

65. The method of any one of claims 58-64, wherein the method comprises:
i. isolating an immune effector cell from the subject or a donor;
ii. modifying an ASXL1 gene or gene product in the immune effector cell so that the expression and/or function of ASXL1 in the cell is reduced or eliminated; and
iii. introducing the modified immune effector cell into the subject.

66. The method of claim 65, wherein the method further comprises modifying the immune effector cell to express a chimeric antigen receptor (CAR) that is capable of binding specifically to an antigen.

67. The method of any one of claims 58-66, wherein the subject is a human or a mouse.

68. A guide RNA (gRNA) targeting ASXL1 comprising a nucleotide sequence of CCACUUACCAGAUAUGCCCC (SEQ ID NO: 143), CCAUUGGGAGAUCUAUUAGG (SEQ ID NO: 162), or GAUGCAAGUCAGGCUAAGAC (SEQ ID NO: 164).

69. A ribonucleoprotein complex comprising the gRNA of claim 68 and a Cas9 protein.
